(19) United States
(12) Patent Application Publication
(10) Pub. No.: US 2025/0034166 A1
(43) Pub. Date: Jan. 30, 2025

(54) POLYCYCLIC FUSED RING DERIVATIVES AND USE THEREOF

(71) Applicant: Jacobio Pharmaceuticals Co., Ltd., Beijing (CN)

(72) Inventors: Hongwei YANG, Beijing (CN); Cunbo MA, Beijing (CN); Panliang GAO, Beijing (CN); Huifeng HAN, Beijing (CN); Peng WANG, Beijing (CN); Runze LI, Beijing (CN); Wei ZHANG, Beijing (CN); Xiaoyu LIU, Beijing (CN); Yanping WANG, Beijing (CN); Wei LONG, Beijing (CN)

(21) Appl. No.: 18/695,384

(22) PCT Filed: Sep. 26, 2022
(86) PCT No.: PCT/CN2022/121202

§ 371 Date: Mar. 26, 2024

(30) Foreign Application Priority Data
Sep. 27, 2021 (WO) PCT/CN2021/120863
Mar. 18, 2022 (WO) PCT/CN2022/081601
Jun. 29, 2022 (WO) PCT/CN2022/102280
Sep. 21, 2022 (WO) PCT/CN2022/120205

(51) Int. Cl. C07D 519/00

(52) U.S. Cl. CPC C07D 519/00

(57) ABSTRACT

Provided are K-Ras mutant protein inhibitors of formula (I), a composition containing the same and the use thereof.

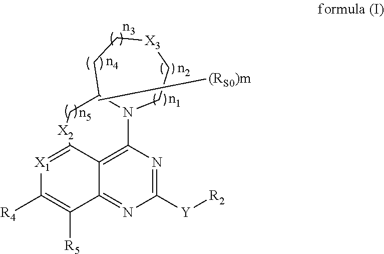

### CROSS-REFERENCES TO RELATED APPLICATIONS

This application claims the benefit of priority to PCT/CN2021/120863, filed on Sep. 27, 2021; PCT/CN2022/081601, filed on Mar. 18, 2022; PCT/CN2022/102280, filed on Jun. 29, 2022; PCT/CN2022/120205, filed on Sep. 21, 2022; all of which are hereby incorporated herein by reference in their entireties.

### TECHNICAL FIELD

The invention relates to compounds that inhibit the activity of multiple forms of K-Ras protein including K-Ras wild type and K-Ras mutant types, compositions comprising the same, and the methods of using the same.

### BACKGROUND ART

Kirsten Rat Sarcoma 2 Viral Oncogene Homolog (“K-Ras”) is a small GTPase and a member of the RAS family of oncogenes. K-Ras serves as a molecular switch cycling between inactive (GDP-bound) and active (GTP-bound) states to transduce upstream cellular signals received from multiple tyrosine kinases to downstream effectors to regulate a wide variety of processes, including cellular proliferation. Aberrant expression of K-Ras accounts for up to ˜20% of all cancers and oncogenic K-Ras mutations that stabilize GTP binding and lead to constitutive activation of K-Ras. K-Ras mutations at codons 12, 13, 61 and other positions of the K-Ras primary amino acid sequence are present in 88% of all pancreatic adenocarcinoma patients, 50% of all colorectal adenocarcinoma patients, and 32% lung adenocarcinoma patients. A recent publication also suggested wild type K-Ras inhibition could be a viable therapeutic strategy to treat K-Ras wild type dependent cancers.

Allele-specific K-Ras G12C inhibitors, such as sotorasib (AMG510) or adagrasib (MRTX849), are currently changing the treatment paradigm for patients with K-Ras G12C mutated non-small cell lung cancer and colorectal cancer. The success of addressing a previously elusive K-Ras allele has fueled drug discovery efforts for all K-Ras mutants. Multiple K-Ras inhibitors have the potential to address broad patient populations, including K-Ras G12C, K-Ras G12D, K-Ras G12V, K-Ras G13D, K-Ras G12R, K-Ras G12S, K-Ras G12A, K-Ras Q61H mutant and K-Ras wild type amplified cancers.

Therefore, there are unmet needs to develop new multiple K-Ras inhibitors for treating K-Ras mediated cancers.

### SUMMARY OF THE INVENTION

Provided herein is a compound of formula (I), a stereoisomer thereof, a pharmaceutically acceptable salt thereof, a pharmaceutically acceptable salt of the stereoisomer thereof, a prodrug thereof, a deuterated molecule thereof or a conjugated form thereof:

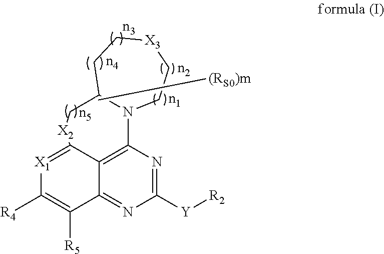

Wherein, the definition of each of variables is as below.

Also provided herein is a pharmaceutical composition comprising a therapeutically effective amount of a compound of formula (I), a stereoisomer thereof, a pharmaceutically acceptable salt thereof, a pharmaceutically acceptable salt of the stereoisomer thereof, a prodrug thereof, a deuterated molecule thereof or a conjugated form thereof as defined herein; and a pharmaceutically acceptable excipient.

Also provided herein is a method for treating cancer in a subject comprising administering a therapeutically effective amount of a compound of formula (I), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein to a subject in need thereof.

Also provided herein is a method for treating cancer in a subject in need thereof, the method comprising (a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and (b) if so, administering a therapeutically effective amount of a compound of formula (I), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein to the subject in need thereof.

Also provided herein is a compound of formula (I), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for use in therapy.

Also provided herein is a compound of formula (I), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for use as a medicament.

Also provided herein is a compound of formula (I), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for use in a method for the treatment of cancer.

Also provided herein is a use of a compound of formula (I), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for the treatment of cancer.

Also provided herein is a use of a compound of formula (I), a stereoisomer thereof, a pharmaceutically acceptable salt thereof, a pharmaceutically acceptable salt of the stereoisomer thereof, a prodrug thereof, a deuterated molecule thereof, a conjugated form thereof, or a pharmaceutical composition as defined herein for the manufacture of a medicament for the treatment of cancer.

Also provided herein is a process for preparing a compound of formula (I) as defined herein.

Also provided herein is an intermediate for preparing a compound of formula (I) as defined herein.

### DETAILED DESCRIPTION

Provided herein are the following disclosures:

[1]. A compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof:

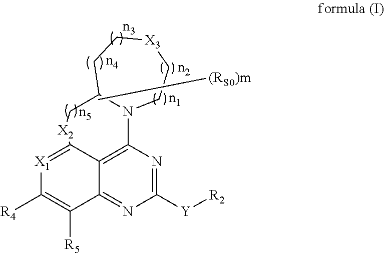

Wherein,
X₁ is selected from CR₃ or N;
R₃ is selected from hydrogen, deuterium, halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆ alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered heterocyclyl, 6-10 membered aryl or 5-10 membered heteroaryl, is independently optionally substituted with 1, 2 or 3 R₃ₐ;
each of R₃ₐ at each occurrence is independently selected from deuterium, halogen, —C₁₋₃alkyl, haloC₁₋₃alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), 3-6 membered cycloalkyl, 3-6 membered heterocyclyl, phenyl or 5-6 membered heteroaryl;
X₂ is selected from NR₁, O or S;
R₁ is selected from hydrogen, deuterium, —C₁₋₆alkyl or 3-6 membered cycloalkyl; said —C₁₋₆alkyl and 3-6 membered cycloalkyl are each independently optionally substituted with one or more (such as 1, 2, 3, 4, 5 or 6) substituents selected from —OH, deuterium, halogen, —CN, oxo, —C₁₋₆alkoxy, —NH₂, —NHC₁₋₆alkyl, or —N(C₁₋₆ alkyl)₂;
X₃ is selected from CR₇₁R₇₂, C═O, NR₇₁, O, S, S═O, or O=S═O;
Each of (R₇₁, R₇₂) is independently selected from hydrogen, deuterium, —C₁₋₆alkyl or 3-6 membered cycloalkyl; said —C₁₋₆alkyl and 3-6 membered cycloalkyl are each independently optionally substituted with 1, 2, 3, 4, 5 or 6 substituents selected from —OH, deuterium, halogen, —CN, oxo, —C₁₋₆alkoxy, —NH₂, —NHC₁₋₆alkyl, or —N(C₁₋₆ alkyl)₂;
Each of (n₁, n₂, n₃, n₄ and n₅) at each occurrence is independently selected from 0, 1, 2 or 3;
Each of R_{S0} at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)H, —C(═O)(C₁₋₆alkyl), —C(═O)OH, —C(═O)(OC₁₋₆alkyl), —OC(═O)(C₁₋₆alkyl), —C(═O)NH₂, —NO₂, —C(═O)NH(C₁₋₆alkyl), —C(═O)N(C₁₋₆ alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆ alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 R₁ₐ;
Optionally, two R_{S0} together with the carbon atom to which they are both attached form

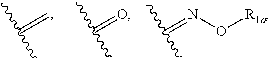

a 3-10 membered carbocyclic ring or a 3-10 heterocyclic ring; wherein, said

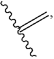

3-10 membered carbocyclic ring or 3-10 heterocyclic ring is optionally substituted with one or more R₁ₐ; Optionally, two adjacent R_{S0} together with the carbon atoms to which they are respectively attached form a 3-10 membered carbocyclic ring or 3-10 membered heterocyclic ring, wherein, each of rings is independently optionally substituted with one or more R₁ₐ;
Each of R₁ₐ is independently selected from deuterium, halogen, —C₁₋₆alkyl, haloC₁₋₆alkyl, —CN, oxo, —OH, —NH₂, —NH(C₁₋₆alkyl), —N(C₁₋₆alkyl)₂, —OC₁₋₆alkyl, or —C₁₋₆alkyl substituted with 1, 2 or 3 substituents selected form deuterium, halogen, haloC₁₋₆alkyl, —CN, —OH, —NH₂, —NH(C₁₋₆alkyl), —N(C₁₋₆alkyl)₂, —OC₁₋₆alkyl or cyclopropyl;
m is selected from 0, 1, 2, 3, 4, 5 or 6;
Y is a bond, O, S, S(═O), S(═O)₂ or NR₈₁;
R₂ is selected from -L-(3-12 membered heterocyclyl), -L-(3-12 membered cycloalkyl), -L-(6-12 member aryl), -L-(5-12 membered heteroaryl), -L-NR₉₁R₉₂,

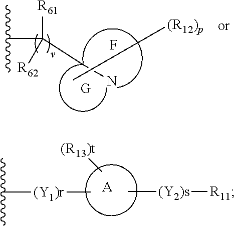

Each L is independently selected from a bond or C₁₋₁₀ alkylene optionally substituted with one or more R₁₆;
Each (R₉₁, R₉₂) is independently selected from hydrogen, deuterium, or —C₁₋₁₀alkyl optionally substituted with one or more R₁₆;
Said 3-12 membered heterocyclyl in -L-(3-12 membered heterocyclyl) is optionally substituted with one or more R₁₆;
Said 3-12 membered cycloalkyl in -L-(3-12 membered cycloalkyl) is optionally substituted with one or more R₁₆;
Said 6-12 member aryl in -L-(6-12 member aryl) is optionally substituted with one or more R₁₆;
Said 5-12 membered heteroaryl in -L-(5-12 membered heteroaryl) is optionally substituted with one or more R₁₆;
Y₁ or Y₂ at each occurrence is independently selected from —C(R₆₁R₆₂)—;
Ring F or ring G is a 3-10 membered heterocyclic ring which is optionally further contains 1, 2, or 3 heteroatoms selected from N, O or S;
Ring A is a 3-10 membered carbocyclic ring or a 3-10 membered heterocyclic ring; wherein the moiety of —(Y₁)ᵣ— and —(Y₂)ₛ—R₁₁ are attached to the same atom or different atoms of the ring A;
R₁₁ is selected from —NR₈₁R₈₂, —OR₈₁, —SR₈₁, 3-10 membered heterocyclyl, or 5-10 membered heteroaryl, wherein said 3-10 membered heterocyclyl or 5-10 membered heteroaryl is optionally independently substituted with one or more R₁₆;
Each of R₁₂ and R₁₃ at each occurrence is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R₁₂ together with the carbon atom to which they are both attached form

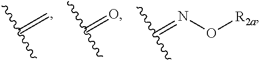

a 3-10 membered carbocyclic ring or a 3-10 heterocyclic ring; wherein, said

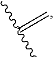

3-10 membered carbocyclic ring or 3-10 heterocyclic ring is optionally substituted with one or more R₂ₐ;
Optionally, two adjacent R₁₂ together with the carbon atoms to which they are respectively attached form 3-10 membered carbocyclic ring, 3-10 membered heterocyclic ring, 6-10 membered aryl ring or 5-10 membered heteroaryl ring, wherein, each of rings is independently optionally substituted with one or more R₂ₐ;
Each of R₂ₐ is independently selected from deuterium; halogen; —C₁₋₆alkyl; haloC₁₋₆alkyl; —CN; oxo; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form deuterium, halogen, haloC₁₋₆alkyl, —CN, —OH, —NH₂, —NH(C₁₋₆alkyl), —N(C₁₋₆alkyl)₂, —OC₁₋₆alkyl or cyclopropyl;
Each of (r, s, t, p and v) at each occurrence is independently selected from 0, 1, 2, 3, 4, 5 or 6;
R₄ is selected from 6-10 membered aryl, 5-10 membered heteroaryl,

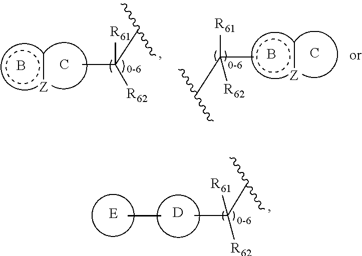

wherein said 6-10 membered aryl, 5-10 membered heteroaryl,

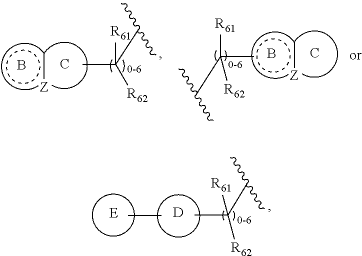

is optionally independently substituted with one or more R₄₁;
Z at each occurrence is independently selected from C or N;
Ring B at each occurrence is independently selected from a 6 membered aryl ring or a 5-6 membered heteroaryl ring and ring C at each occurrence is a 3-10 membered carbocyclic ring or a 3-10 membered heterocyclic ring when Z is selected from C;
Ring B at each occurrence is selected from a 5-6 membered heteroaryl ring and ring C at each occurrence is a 3-10 membered heterocyclic ring when Z is selected from N;
Ring D at each occurrence is selected from a 3-10 membered carbocyclic ring, a 3-10 membered heterocyclic ring, a 6 membered aryl ring or a 5-6 membered heteroaryl ring;
Ring E at each occurrence is selected from a 3-10 membered carbocyclic ring, a 3-10 membered heterocyclic ring, a 6-membered aryl ring or a 5-6 membered heteroaryl ring;
R₄₁ at each occurrence is independently selected from deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-10 membered cycloalkyl, 3-10 membered cycloalkenyl, 3-10 membered cycloalkynyl, 3-10 membered heterocyclyl, 6-10 membered aryl or 5-10 membered heteroaryl or R₄ₐ, wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
R₄ₐ is a structure capable of forming a prodrug;
R₅ is selected from hydrogen, deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
R₆₁ or R₆₂ at each occurrence is independently selected from hydrogen, deuterium, halogen, —C₁₋₁₀alkyl, haloC₁₋₁₀alkyl, haloC₁₋₁₀alkoxy, —C₂₋₁₀alkenyl, —C₂₋₁₀alkynyl, —CN, —NO₂, —N₃, oxo, —NRₐR_{b}, —ORₐ, —SRₐ, —S(═O)Rₐ, —S(═O)₂Rₐ, —C(═O)Rₐ, —C(═O)ORₐ, —OC(═O)Rₐ, —C(═O)NRₐR_{b}, —NRₐC(═O)R_{b}, —OC(═O)ORₐ, —NRₐC(═O)OR_{b}, —OC(═O)NRₐR_{b}, —NRₐC(═O)NRₐR_{b}, —S(═O)ORₐ, —OS(═O)Rₐ, —S(═O)NRₐR_{b}, —NRₐS(═O)R_{b}, —S(═O)₂ORₐ, —OS(═O)₂Rₐ, —S(═O)₂NRₐR_{b}, —NRₐS(═O)₂R_{b}, —OS(═O)₂ORₐ, —NRₐS(═O)₂OR_{b}, —OS(═O)₂NRₐ, —NRₐS(═O)₂NRₐR_{b}, —PRₐR_{b}, —P(═O)RₐR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NR_{c}R_{d}, —OR_{c}, —SR_{c}, —S(═O)R_{c}, —S(═O)₂R_{c}, —C(═O)R_{c}, —C(═O)OR_{c}, —OC(═O)R_{c}, —C(═O)NR_{c}R_{d}, —NR_{c}C(═O)R_{d}, —OC(═O)OR_{c}, —NR_{c}C(═O)OR_{d}, —OC(═O)NR_{c}R_{d}, —NR_{c}C(═O)NR_{c}R_{d}, —S(═O)OR_{c}, —OS(═O)R_{c}, —S(═O)NR_{c}R_{d}, —NR_{c}S(═O)R_{d}, —S(═O)₂OR_{c}, —OS(═O)₂R_{c}, —S(═O)₂NR_{c}R_{d}, —NR_{c}S(═O)₂R_{d}, —OS(═O)₂OR_{c}, —NR_{c}S(═O)₂OR_{d}, —OS(═O)₂NR_{c}, —NR_{c}S(═O)₂NR_{c}R_{d}, —PR_{c}R_{d}, —P(═O)R_{c}R_{d}, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, R₆₁ and R₆₂ together with the carbon atom to which they are both attached form a 3-10 membered carbocyclic ring or a 3-10 membered heterocyclic ring, wherein, said 3-10 membered carbocyclic ring or 3-10 membered heterocyclic ring is optionally independently substituted with one or more R₁₆;
R₈₁ or R₈₂ at each occurrence is independently selected from hydrogen, deuterium, —C₁₋₁₀alkyl, haloC₁₋₁₀alkyl, haloC₁₋₁₀alkoxy, —C₂₋₁₀alkenyl, —C₂₋₁₀alkynyl, —S(═O)Rₐ, —S(═O)₂Rₐ, —C(═O)Rₐ, —C(═O)ORₐ, —C(═O)NRₐR_{b}, —S(═O)ORₐ, —S(═O)NRₐR_{b}, —S(═O)₂ORₐ, —S(═O)₂NRₐR_{b}, —P(═O)RₐR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆ alkoxy, —C₂₋₆alkenyl, —C₂₋₆alkynyl, —CN, —NO₂, —N₃, oxo, —NR_{c}R_{d}, —OR_{c}, —SR_{c}, —S(═O)R_{c}, —S(═O)₂R_{c}, —C(═O)R_{c}, —C(═O)OR_{c}, —OC(═O)R_{c}, —C(═O)NR_{c}R_{d}, —NR_{c}C(═O)R_{d}, —OC(═O)OR_{c}, —NR_{c}C(═O)OR_{d}, —OC(═O)NR_{c}R_{d}, —NR_{c}C(═O)NR_{c}R_{d}, —S(═O)OR_{c}, —OS(═O)R_{c}, —S(═O)NR_{c}R_{d}, —NR_{c}S(═O)R_{d}, —S(═O)₂OR_{c}, —OS(═O)₂R_{c}, —S(═O)₂NR_{c}R_{d}, —NR_{c}S(═O)₂R_{d}, —OS(═O)₂OR_{c}, —NR_{c}S(═O)₂OR_{d}, —OS(═O)₂NR_{c}, —NR_{c}S(═O)₂NR_{c}R_{d}, —PR_{c}R_{d}, —P(═O)R_{c}R_{d}, 3-6 membered cycloalkyl, 3-6 membered cycloalkenyl, 3-6 membered cycloalkynyl, 3-10 membered heterocyclyl, 6-10 membered aryl or 5-10 membered heteroaryl;
Optionally, R₈₁ and R₈₂ together with the nitrogen atom to which they are both attached form a 3-10 membered heterocyclic ring or a 5-10 membered heteroaryl ring, wherein, said 3-10 membered heterocyclic ring or 5-10 membered heteroaryl ring is optionally independently substituted with one or more R₁₆;
Rₐ, R_{b}, R_{c} or R_{d} at each occurrence is independently selected from hydrogen, deuterium, —C₁₋₆alkyl, haloC₁₋₆ alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₁₆;
Optionally, (Rₐ and R_{b}) or (R_{c} and R_{d}) together the atom to which they are both attached form a 3-6 membered heterocyclic ring, wherein said 3-6 membered heterocyclic ring is independently optionally substituted with one or more R₁₆;
R₁₆ at each occurrence is independently selected from deuterium, halogen, —C₁₋₆alkyl, haloC₁₋₆alkyl, haloC₁₋₆ alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆ alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆ alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆ alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆ alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆ alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;
Each of (heterocyclyl and heteroaryl) at each occurrence is independently contain 1, 2, 3 or 4 heteroatoms selected from N, O, S, S(═O) or S(═O)₂.

[2]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1], wherein, X₁ is N.

[3]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1], wherein, X₁ is CR₃.

[4]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1] or [3], wherein, R₃ is selected from —H, deuterium, —F, —Cl, —CH₃, —CH₂CH₃, —CH₂CH₂CH₃, —CH(CH₃)₂, —CN, —COOH, —CH₂OH, —OH, —OCH₃, —OCH₂CH₃, —CF₃, —CHF₂, —NH₂, —NHCH₃, —N(CH₃)₂, —CH₂NH₂, —CH₂CH₂NH₂, —CH₂OH, —CH₂CH₂OH, —SH, —S—CH₃, —S—CHF₂, —S—CF₃, —CH₂SH, —CH₂CH₂SH, —CH═CH₂, —C≡CH, —CHCH═CH₂, —OCF₃, —OCHF₂, —C(═O)NH₂, —C(═O)OCH₃,

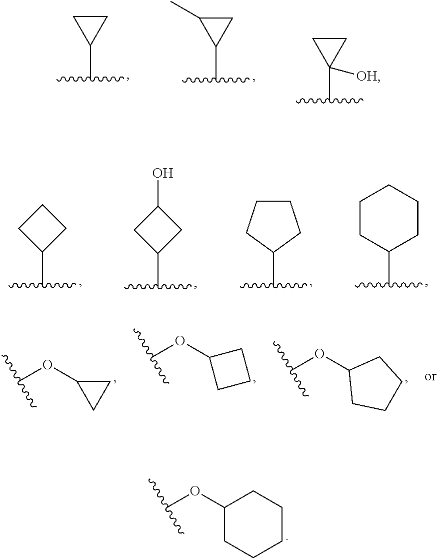

[5]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1], [3] or [4], wherein, R₃ is selected from —H, deuterium, —F, —Cl, —CH₃, —CH(CH₃)₂, —CF₃, —S—CF₃ or

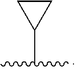 [SMILES: *C1CC1]

[6]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1], wherein, X₂ is O, S, NH or NCH₃.

[7]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of [1], wherein, X₃ is selected from CR₇₁R₇₂ or O.

[8]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [7], wherein, each of (R₇₁ and R₇₂) is independently selected from hydrogen, deuterium, —CH₃, —CH₂CH₃, —CH₂CH₂CH₃, —CH(CH₃)₂, —F, —Cl, —CN, —CH₂OH, —OH, —OCH₃, —OCH₂CH₃, —CF₃, —CHF₂, —S—CH₃, —S—CHF₂, —S—CF₃,

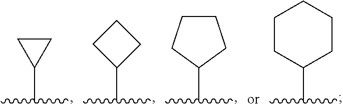

Preferably, R₇₁, R₇₂ are hydrogen.

[9]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [8], wherein, n₁, n₄, and n₅ are 1 respectively, or n₁ and n₄ are 1 respectively and n₅ is 0.

[10]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [9], wherein, n₂ and n₃ are 1 respectively, or n₃ is 0 and n₂ is 2, or n₃ is 0 and n₂ is 1, or n₃ is 1 and n₂ is 0, or n₃ is 0 and n₂ is 0.

[11]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [10], wherein, each of R_{S0} at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)H, —C(═O)(C₁₋₆ alkyl), —C(═O)OH, —C(═O)(OC₁₋₆alkyl), —OC(═O)(C₁₋₆alkyl), —C(═O)NH₂, —NO₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆ alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 R₁ₐ;

Optionally, two R_{S0} together with the carbon atom to which they are both attached form

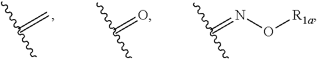

a 3-10 membered carbocyclic ring or a 3-10 heterocyclic ring; wherein, said

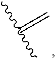

3-10 membered carbocyclic ring or 3-10 heterocyclic ring is optionally substituted with one or more R₁ₐ;
Optionally, two adjacent R_{S0} together with the carbon atoms to which they are respectively attached form a 3-10 membered carbocyclic ring or 3-10 membered heterocyclic ring, wherein, each of rings is independently optionally substituted with one or more R₁ₐ;
Each of R₁ₐ is independently selected from deuterium, halogen, —C₁₋₆alkyl, haloC₁₋₆alkyl, —CN, oxo, —OH, —NH₂, —NH(C₁₋₆alkyl), —N(C₁₋₆alkyl)₂, —OC₁₋₆alkyl, or —C₁₋₆alkyl substituted with 1, 2 or 3 substituents selected form deuterium, halogen, haloC₁₋₆alkyl, —CN, —OH, —NH₂, —NH(C₁₋₆alkyl), —N(C₁₋₆alkyl)₂, —OC₁₋₆alkyl or cyclopropyl;
qₘ is independently selected from 0, 1, 2 or 3.

[12]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [11], wherein, the compound is selected from the formulas in Table 1:

TABLE 1

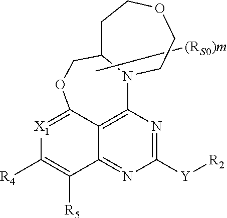

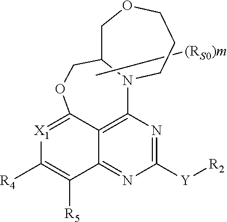

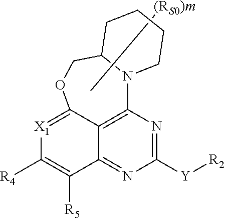

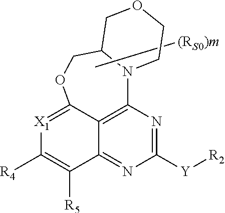

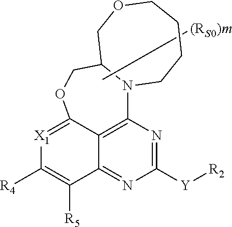

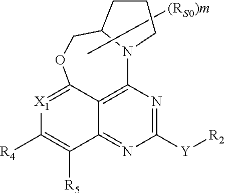

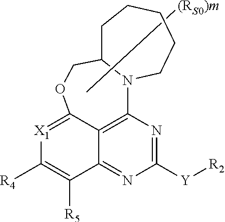

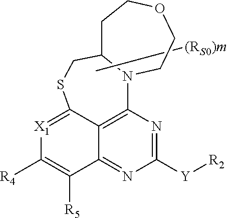

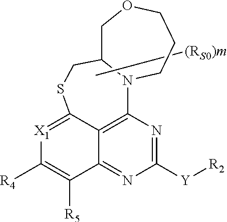

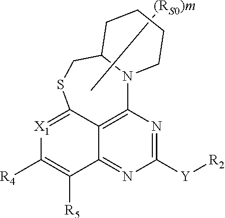

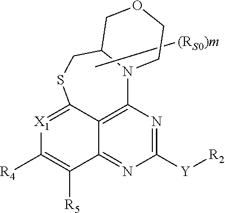

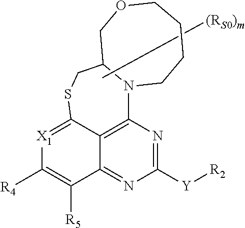

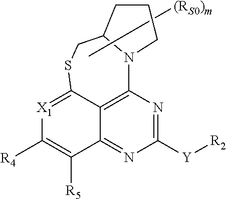

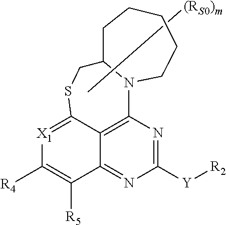

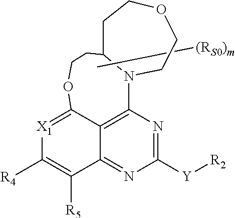

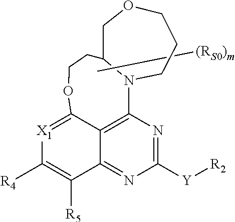

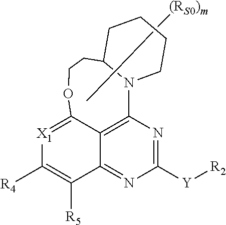

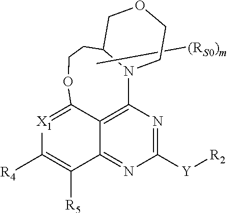

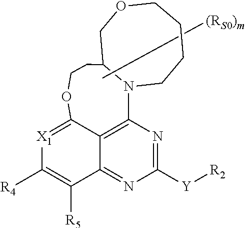

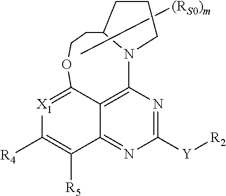

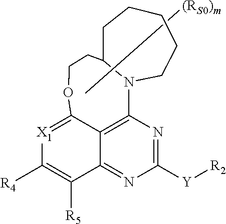

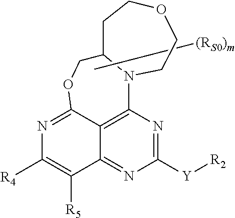

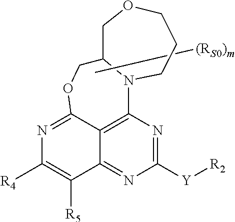

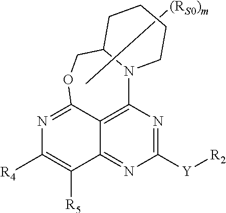

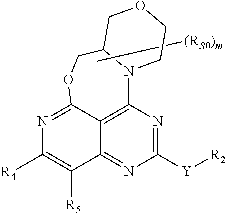

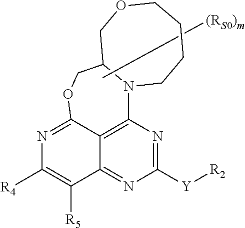

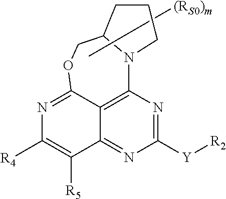

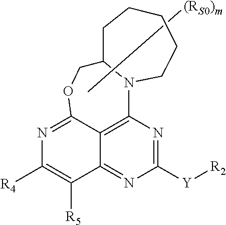

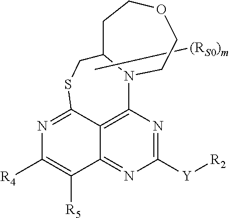

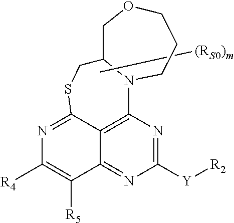

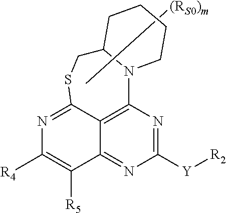

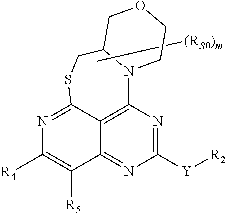

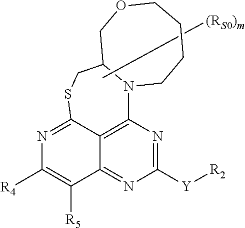

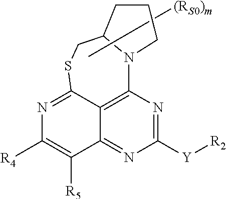

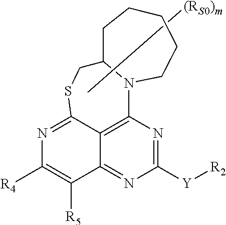

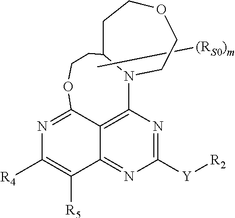

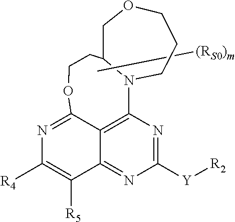

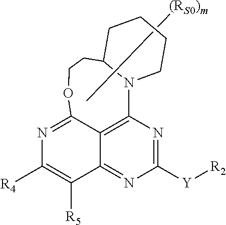

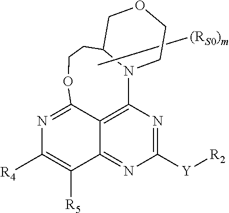

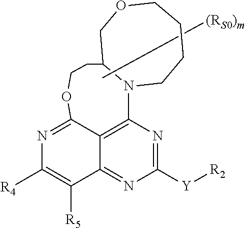

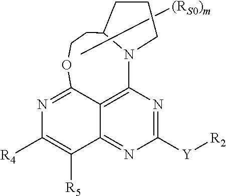

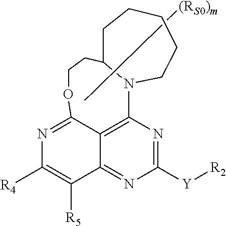

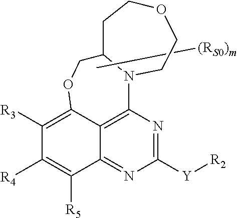

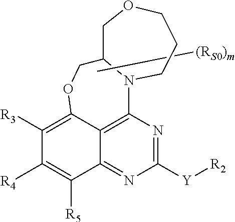

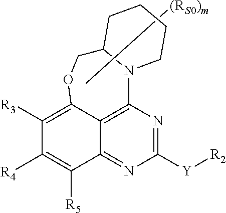

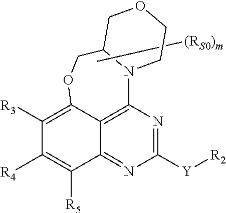

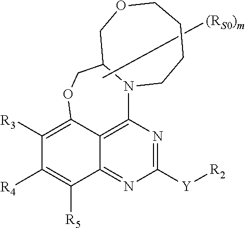

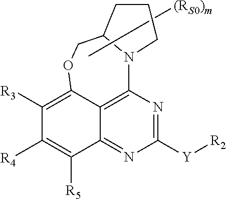

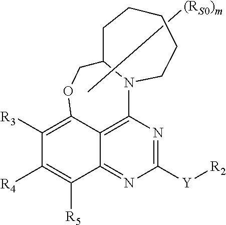

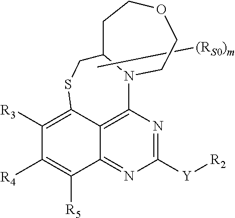

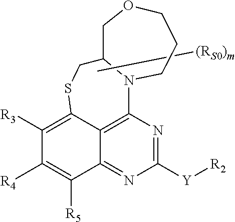

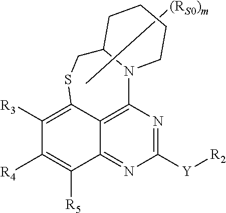

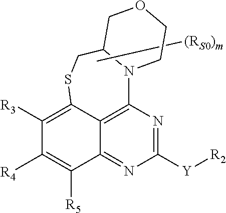

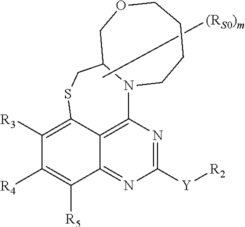

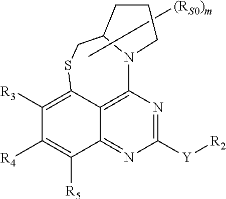

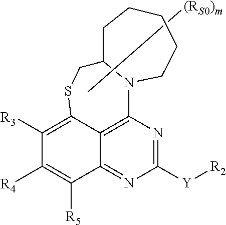

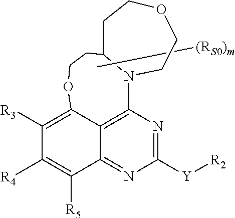

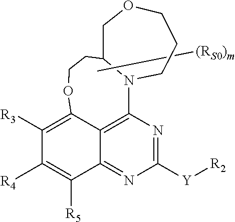

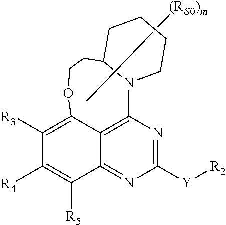

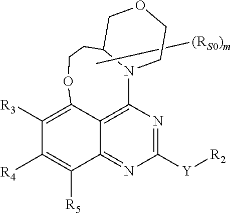

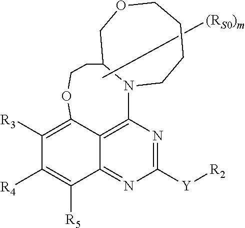

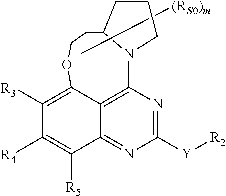

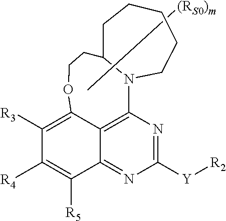

[13]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [12], wherein, the moiety of —Y—R₂ is selected from

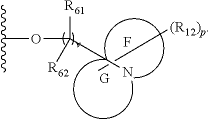

[14]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [13], wherein, the compound is of formula (II):

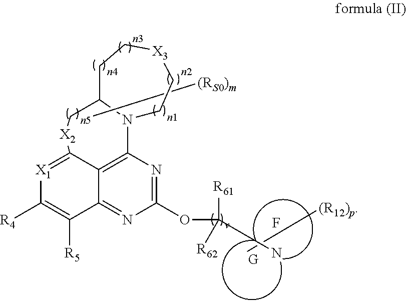

[15]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [14], wherein, the moiety of —Y—R₂ or

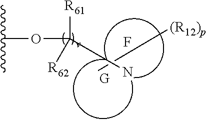

is selected from

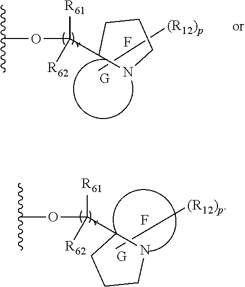

[16]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [15], wherein, the moiety of —Y—R₂ or

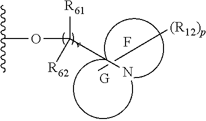

is selected from

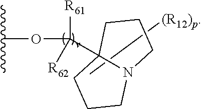

[17]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [16], wherein, the moiety of

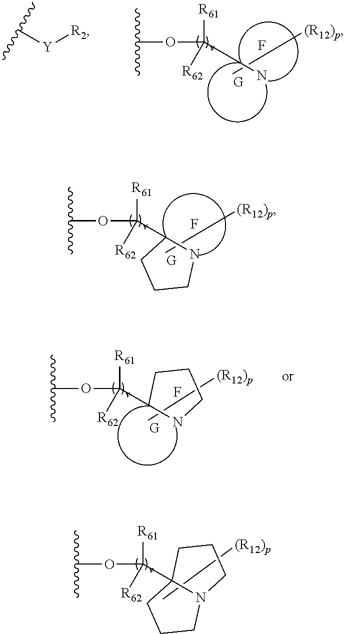

or is selected from any one moiety in the Table 2:

TABLE 2

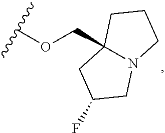 [SMILES: *OC[C@@]12CCCN1C[C@H](F)C2]

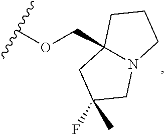 [SMILES: *OC[C@@]12CCCN1C[C@](C)(F)C2]

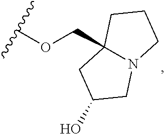 [SMILES: *OC[C@@]12CCCN1C[C@H](O)C2]

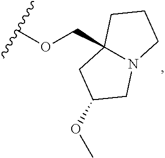 [SMILES: *OC[C@@]12CCCN1C[C@H](OC)C2]

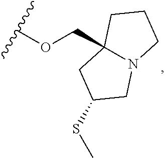 [SMILES: *OC[C@@]12CCCN1C[C@H](SC)C2]

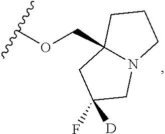 [SMILES: *OC[C@@]12CCCN1C[C@]([2H])(F)C2]

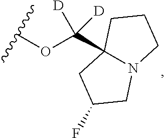 [SMILES: *OC([2H])([2H])[C@@]12CCCN1C[C@H](F)C2]

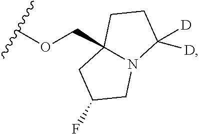

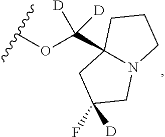 [SMILES: *OC([2H])([2H])[C@@]12CCCN1C[C@]([2H])(F)C2]

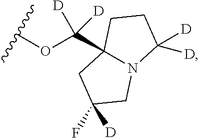

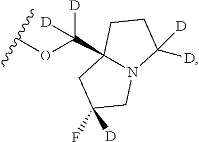

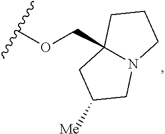 [SMILES: *OC[C@@]12CCCN1C[C@H](C)C2]

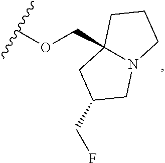 [SMILES: *OC[C@@]12CCCN1C[C@H](CF)C2]

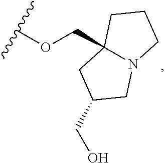 [SMILES: *OC[C@@]12CCCN1C[C@H](CO)C2]

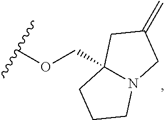 [SMILES: *OC[C@@]12CCCN1CC(=C)C2]

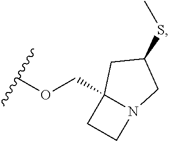 [SMILES: *OC[C@@]12CCN1C[C@H](SC)C2]

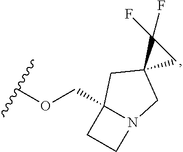 [SMILES: *OC[C@@]12CCN1C[C@@]1(CC1(F)F)C2]

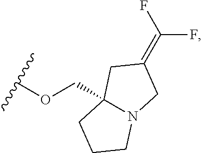 [SMILES: *OC[C@@]12CCCN1CC(=C(F)F)C2]

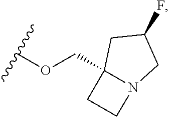 [SMILES: *OC[C@@]12CCN1C[C@H](F)C2]

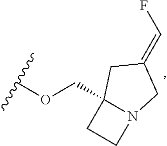 [SMILES: *OC[C@@]12CCN1C/C(=C/F)C2]

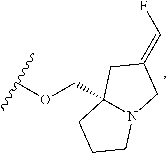 [SMILES: *OC[C@@]12CCCN1C/C(=C/F)C2]

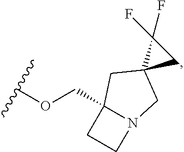

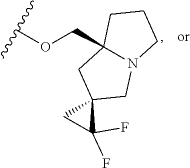 [SMILES: *OC[C@@]12CCCN1C[C@]1(CC1(F)F)C2]

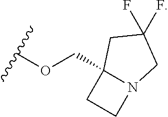 [SMILES: *OC[C@@]12CCN1CC(F)(F)C2]

[18]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [17], wherein, the moiety of

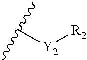

is selected from

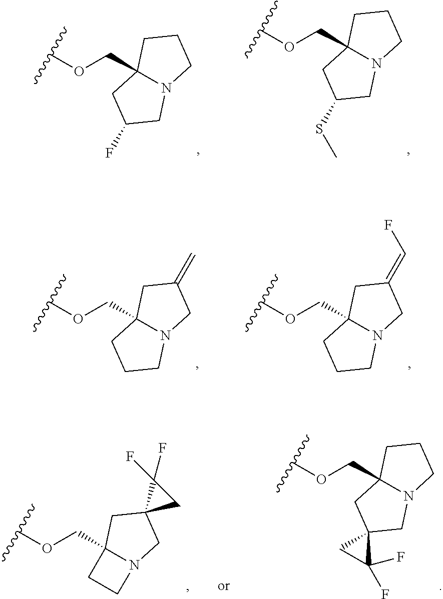 [SMILES: *OC[C@@]12CCCN1C[C@H](F)C2 ; *OC[C@@]12CCCN1C[C@H](SC)C2 ; *OC[C@@]12CCCN1CC(=C)C2 ; *OC[C@@]12CCCN1C/C(=C/F)C2 ; *OC[C@@]12CCN1C[C@]1(CC1(F)F)C2 ; *OC[C@@]12CCCN1C[C@]1(CC1(F)F)C2]

[19]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [18], wherein, the compound is selected from the formulas in Table 3:

TABLE 3

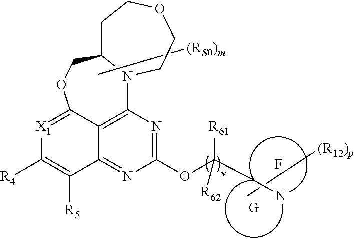

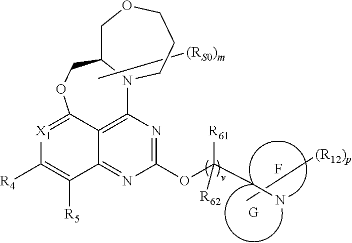

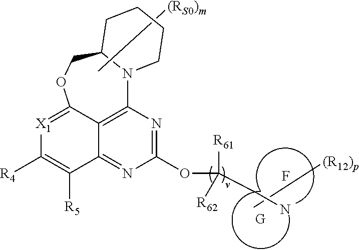

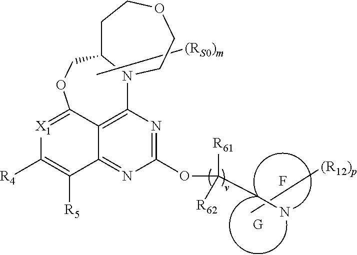

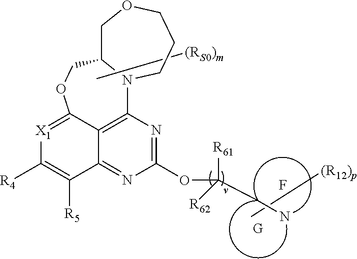

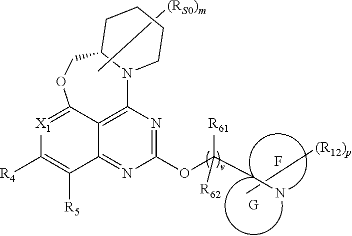

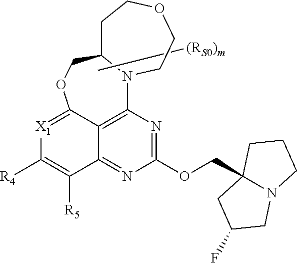

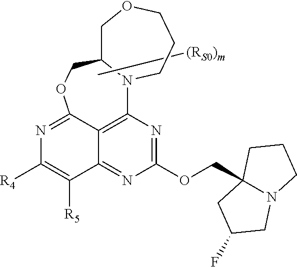

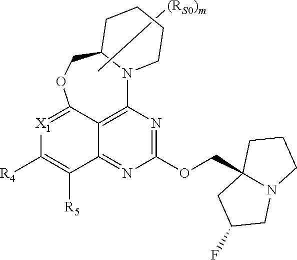

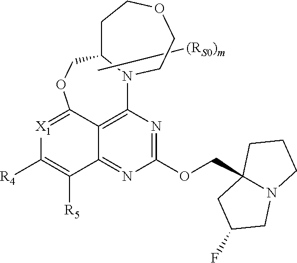

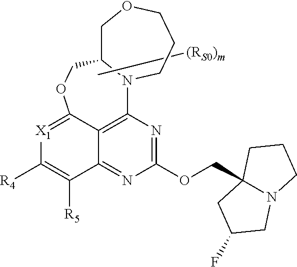

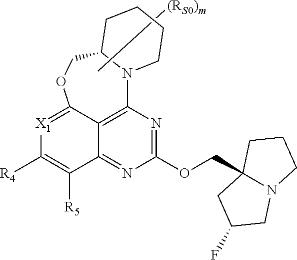

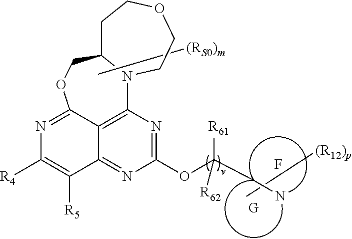

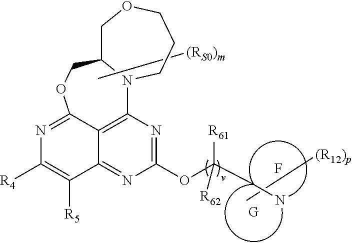

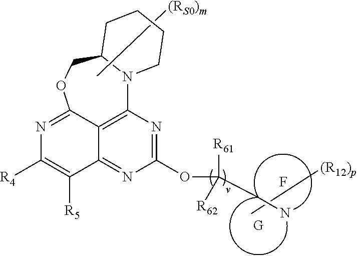

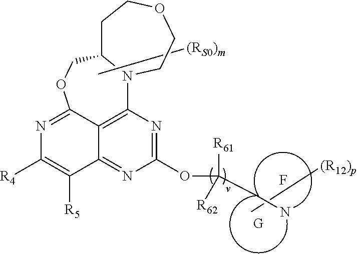

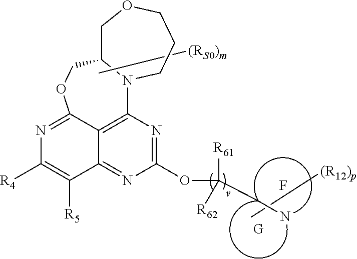

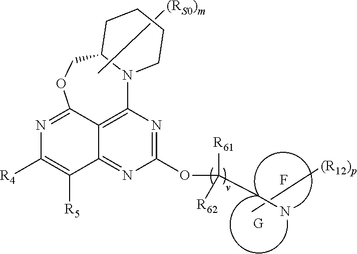

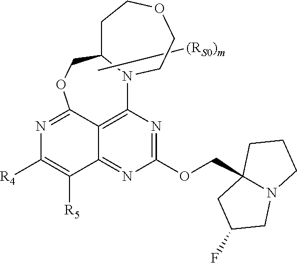

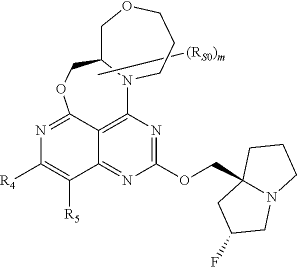

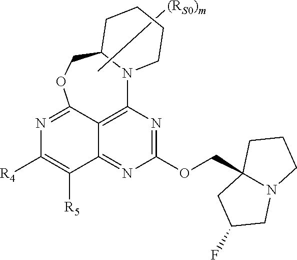

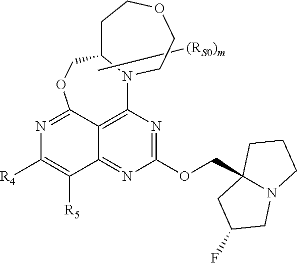

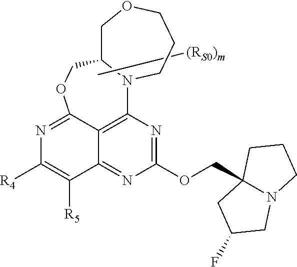

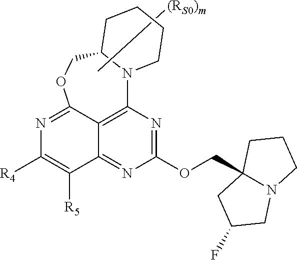

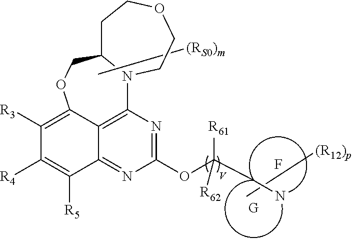

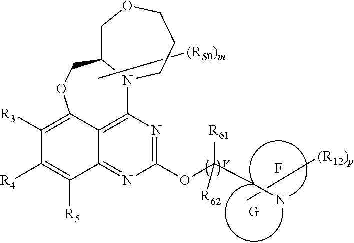

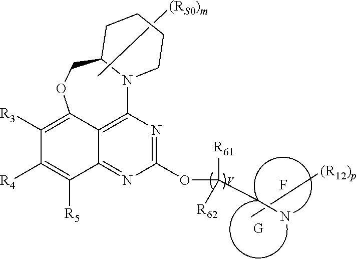

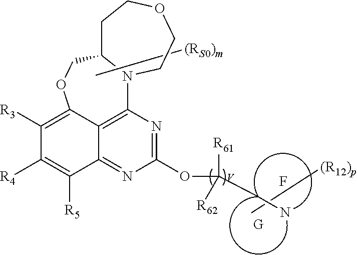

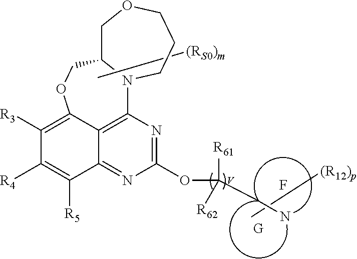

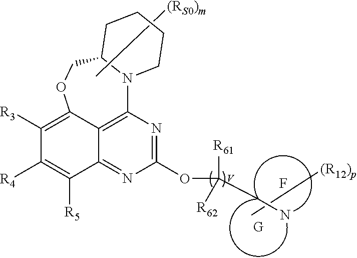

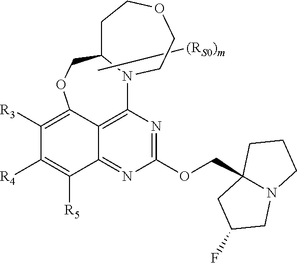

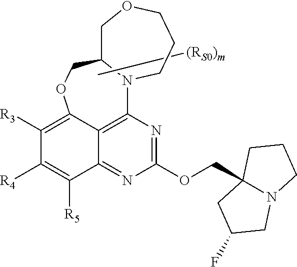

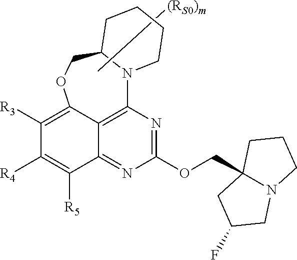

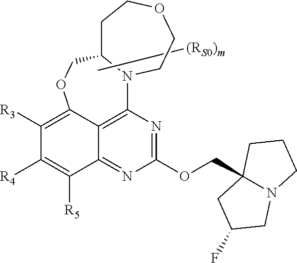

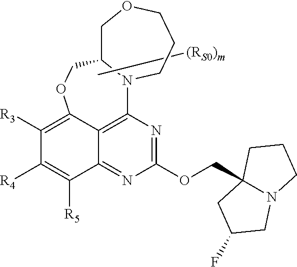

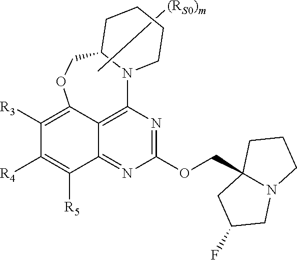

[20]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [19], wherein, R₄ is selected from any one moiety in the Table 4:

TABLE 4

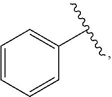 [SMILES: *c1ccccc1]

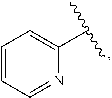 [SMILES: *c1ccccn1]

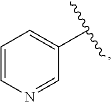 [SMILES: *c1cccnc1]

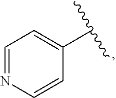 [SMILES: *c1ccncc1]

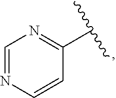 [SMILES: *c1ccncn1]

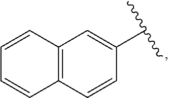 [SMILES: *c1ccc2ccccc2c1]

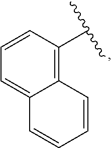 [SMILES: *c1cccc2ccccc12]

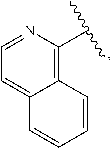 [SMILES: *c1nccc2ccccc12]

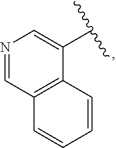 [SMILES: *c1cncc2ccccc12]

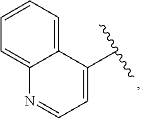 [SMILES: *c1ccnc2ccccc12]

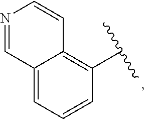 [SMILES: *c1cccc2cnccc12]

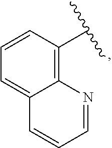 [SMILES: *c1cccc2cccnc12]

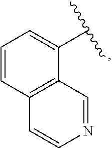 [SMILES: *c1cccc2ccncc12]

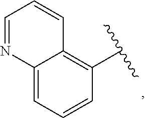 [SMILES: *c1cccc2ncccc12]

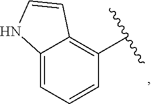 [SMILES: *c1cccc2[nH]ccc12]

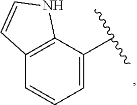 [SMILES: *c1cccc2cc[nH]c12]

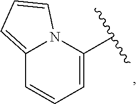 [SMILES: *c1cccc2cccn12]

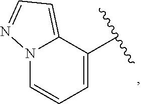 [SMILES: *c1cccn2nccc12]

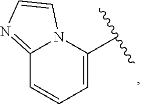 [SMILES: *c1cccc2nccn12]

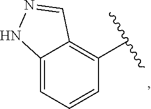 [SMILES: *c1cccc2[nH]ncc12]

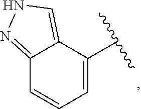 [SMILES: *c1cccc2n[nH]cc12]

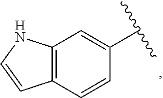 [SMILES: *c1ccc2cc[nH]c2c1]

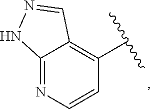 [SMILES: *c1ccnc2[nH]ncc12]

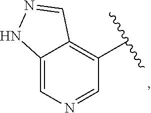 [SMILES: *c1cncc2[nH]ncc12]

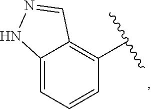 [SMILES: *c1cccc2[nH]ncc12]

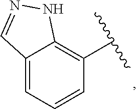 [SMILES: *c1cccc2cn[nH]c12]

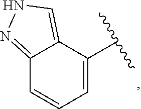 [SMILES: *c1cccc2n[nH]cc12]

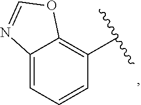 [SMILES: *c1cccc2ncoc12]

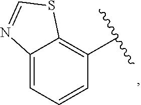 [SMILES: *c1cccc2ncsc12]

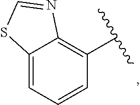 [SMILES: *c1cccc2scnc12]

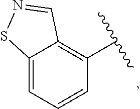 [SMILES: *c1cccc2sncc12]

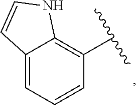 [SMILES: *c1cccc2cc[nH]c12]

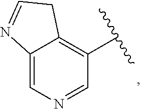 [SMILES: *c1cncc2c1CC=N2]

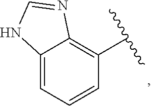 [SMILES: *c1cccc2[nH]cnc12]

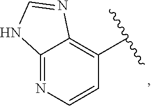 [SMILES: *c1ccnc2[nH]cnc12]

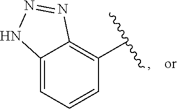 [SMILES: *c1cccc2[nH]nnc12]

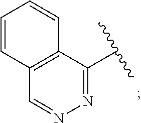 [SMILES: *c1nncc2ccccc12]

Wherein said R₄ is independently optionally substituted with 1, 2, 3, 4, 5 or 6 R₄₁;

Each of R₄₁ is independently selected from deuterium, —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), 3-6 membered cycloalkyl or 3-6 membered heterocyclyl or R₄ₐ, wherein said —C₁₋₃alkyl, haloC₁₋₃alkyl, haloC₁₋₃alkoxy, —C₂₋₃alkenyl, —C₂₋₆alkynyl, 3-6 membered cycloalkyl or 3-6 membered heterocyclyl is independently optionally substituted with 1, 2 or 3 substituents selected from —F; —C₁₋₃alkyl; haloC₁₋₃alkyl; —CN; —OH; —NH₂; —NH(C₁₋₃alkyl); —NH(C₁₋₃alkyl)₂; —OC₁₋₃alkyl; or —C₁₋₃alkyl substituted with 1, 2 or 3 substituents selected from —F, haloC₁₋₃alkyl, —CN, —OH, —NH₂, —NH(C₁₋₃alkyl), —NH(C₁₋₃alkyl)_{z} or —OC₁₋₃alkyl.

[21]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [20], wherein, R₄ is selected from any one moiety in the Table 5:

TABLE 5

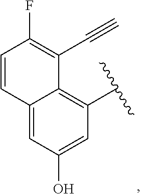 [SMILES: *c1cc(O)cc2ccc(F)c(C#C)c12]

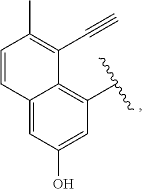 [SMILES: *c1cc(O)cc2ccc(C)c(C#C)c12]

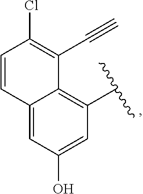 [SMILES: *c1cc(O)cc2ccc(Cl)c(C#C)c12]

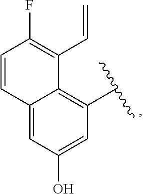 [SMILES: *c1cc(O)cc2ccc(F)c(C=C)c12]

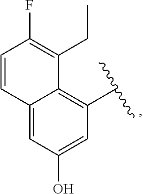 [SMILES: *c1cc(O)cc2ccc(F)c(CC)c12]

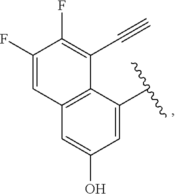 [SMILES: *c1cc(O)cc2cc(F)c(F)c(C#C)c12]

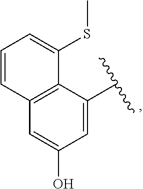 [SMILES: *c1cc(O)cc2cccc(SC)c12]

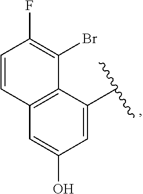 [SMILES: *c1cc(O)cc2ccc(F)c(Br)c12]

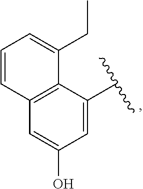 [SMILES: *c1cc(O)cc2cccc(CC)c12]

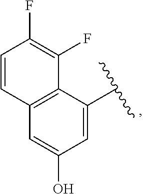 [SMILES: *c1cc(O)cc2ccc(F)c(F)c12]

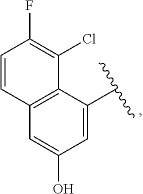 [SMILES: *c1cc(O)cc2ccc(F)c(Cl)c12]

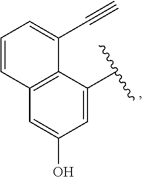 [SMILES: *c1cc(O)cc2cccc(C#C)c12]

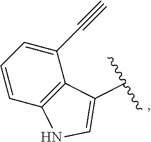 [SMILES: *c1c[nH]c2cccc(C#C)c12]

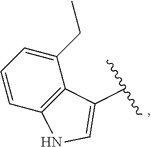 [SMILES: *c1c[nH]c2cccc(CC)c12]

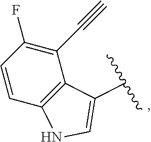 [SMILES: *c1c[nH]c2ccc(F)c(C#C)c12]

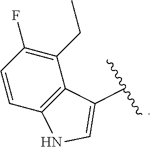 [SMILES: *c1c[nH]c2ccc(F)c(CC)c12]

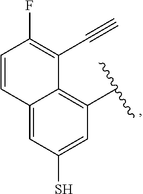 [SMILES: *c1cc(S)cc2ccc(F)c(C#C)c12]

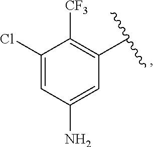 [SMILES: *c1cc(N)cc(Cl)c1C(F)(F)F]

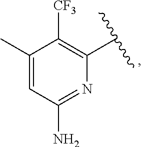 [SMILES: *c1nc(N)cc(C)c1C(F)(F)F]

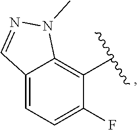 [SMILES: *c1c(F)ccc2cnn(C)c12]

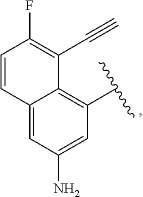 [SMILES: *c1cc(N)cc2ccc(F)c(C#C)c12]

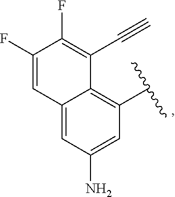 [SMILES: *c1cc(N)cc2cc(F)c(F)c(C#C)c12]

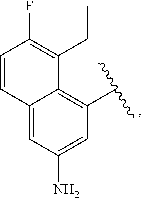 [SMILES: *c1cc(N)cc2ccc(F)c(CC)c12]

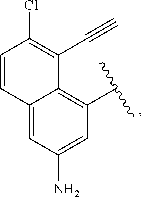 [SMILES: *c1cc(N)cc2ccc(Cl)c(C#C)c12]

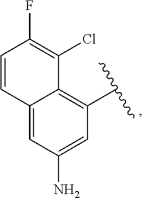 [SMILES: *c1cc(N)cc2ccc(F)c(Cl)c12]

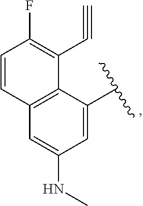 [SMILES: *c1cc(NC)cc2ccc(F)c(C#C)c12]

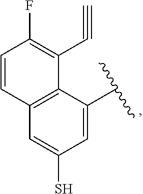 [SMILES: *c1cc(S)cc2ccc(F)c(C#C)c12]

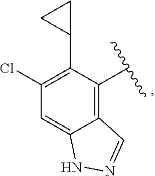 [SMILES: *c1c(C2CC2)c(Cl)cc2[nH]ncc12]

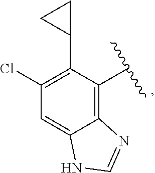 [SMILES: *c1c(C2CC2)c(Cl)cc2[nH]cnc12]

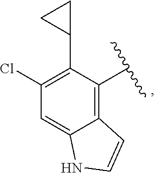 [SMILES: *c1c(C2CC2)c(Cl)cc2[nH]ccc12]

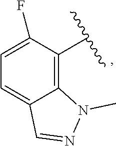 [SMILES: *c1c(F)ccc2cnn(C)c12]

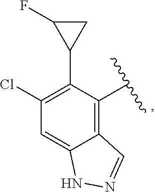 [SMILES: *c1c(C2CC2F)c(Cl)cc2[nH]ncc12]

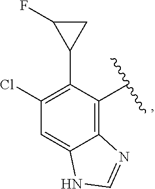 [SMILES: *c1c(C2CC2F)c(Cl)cc2[nH]cnc12]

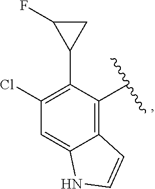 [SMILES: *c1c(C2CC2F)c(Cl)cc2[nH]ccc12]

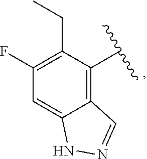 [SMILES: *c1c(CC)c(F)cc2[nH]ncc12]

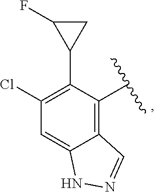 [SMILES: *c1c(C2CC2F)c(Cl)cc2[nH]ncc12]

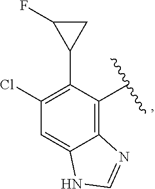 [SMILES: *c1c(C2CC2F)c(Cl)cc2[nH]cnc12]

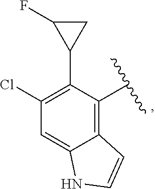 [SMILES: *c1c(C2CC2F)c(Cl)cc2[nH]ccc12]

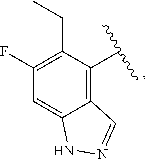 [SMILES: *c1c(CC)c(F)cc2[nH]ncc12]

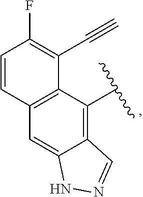 [SMILES: *c1c2cn[nH]c2cc2ccc(F)c(C#C)c12]

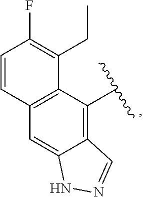 [SMILES: *c1c2cn[nH]c2cc2ccc(F)c(CC)c12]

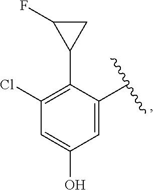 [SMILES: *c1cc(O)cc(Cl)c1C1CC1F]

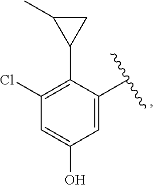 [SMILES: *c1cc(O)cc(Cl)c1C1CC1C]

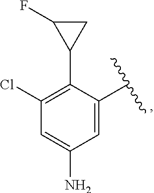 [SMILES: *c1cc(N)cc(Cl)c1C1CC1F]

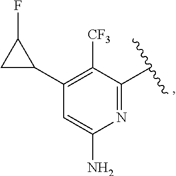 [SMILES: *c1nc(N)cc(C2CC2F)c1C(F)(F)F]

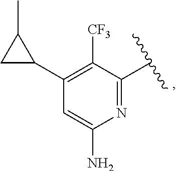 [SMILES: *c1nc(N)cc(C2CC2C)c1C(F)(F)F]

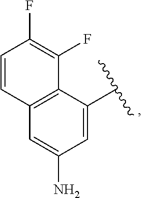 [SMILES: *c1cc(N)cc2ccc(F)c(F)c12]

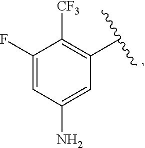 [SMILES: *c1cc(N)cc(F)c1C(F)(F)F]

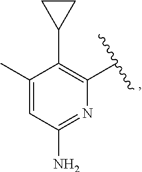 [SMILES: *c1nc(N)cc(C)c1C1CC1]

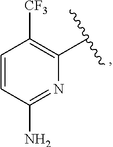 [SMILES: *c1nc(N)ccc1C(F)(F)F]

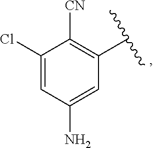 [SMILES: *c1cc(N)cc(Cl)c1C#N]

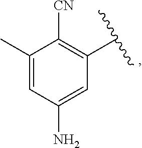 [SMILES: *c1cc(N)cc(C)c1C#N]

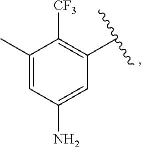 [SMILES: *c1cc(N)cc(C)c1C(F)(F)F]

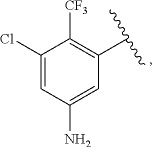 [SMILES: *c1cc(N)cc(Cl)c1C(F)(F)F]

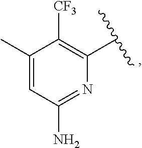 [SMILES: *c1nc(N)cc(C)c1C(F)(F)F]

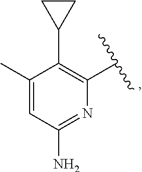 [SMILES: *c1nc(N)cc(C)c1C1CC1]

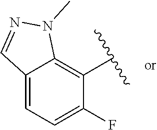 [SMILES: *c1c(F)ccc2cnn(C)c12]

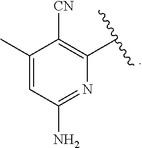 [SMILES: *c1nc(N)cc(C)c1C#N]

[22]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [21], wherein, R₄ is

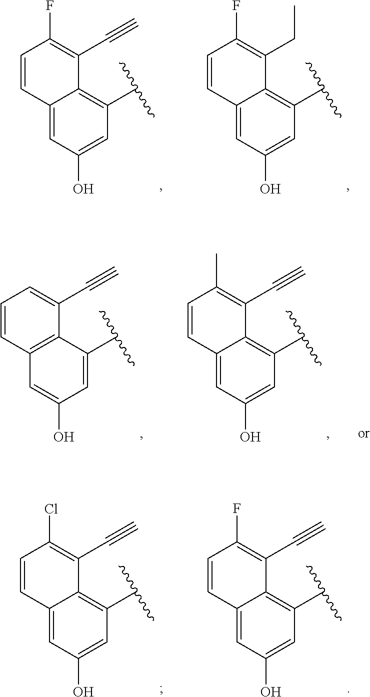 [SMILES: *c1cc(O)cc2ccc(F)c(C#C)c12 ; *c1cc(O)cc2ccc(F)c(CC)c12 ; *c1cc(O)cc2cccc(C#C)c12 ; *c1cc(O)cc2ccc(C)c(C#C)c12 ; *c1cc(O)cc2ccc(Cl)c(C#C)c12 ; *c1cc(O)cc2ccc(F)c(C#C)c12]

Preferably, R₄ is

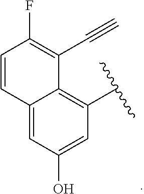 [SMILES: *c1cc(O)cc2ccc(F)c(C#C)c12]

[23]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [22], wherein, the compound is selected from the formulas in Table 6:

TABLE 6

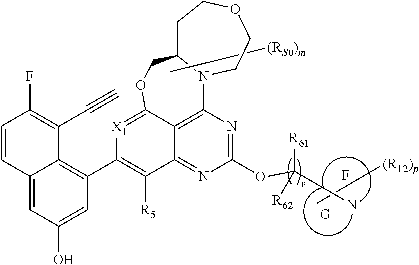

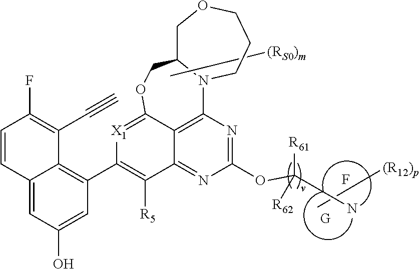

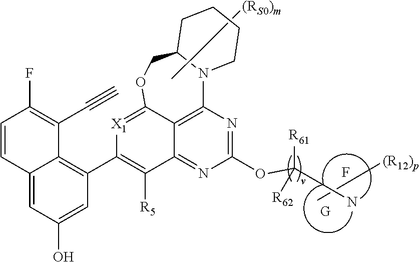

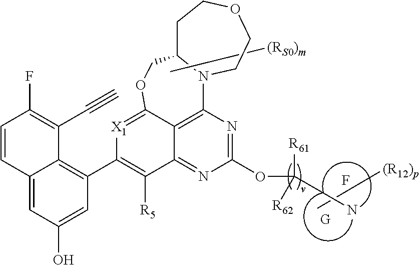

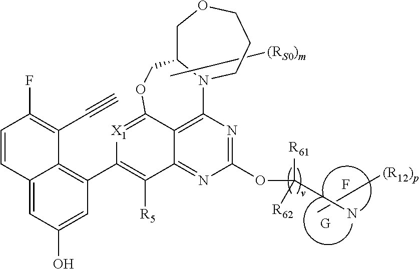

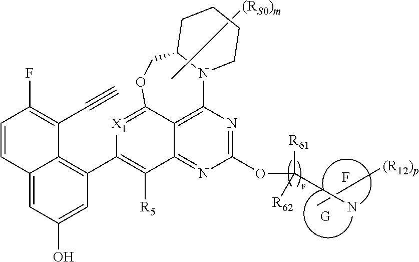

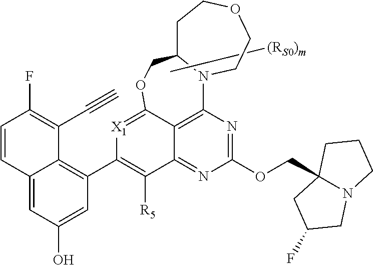

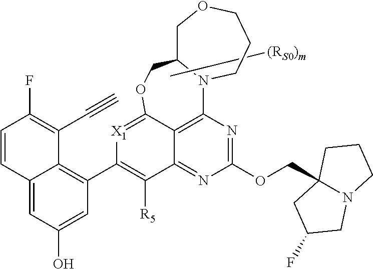

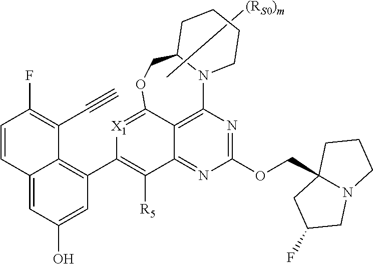

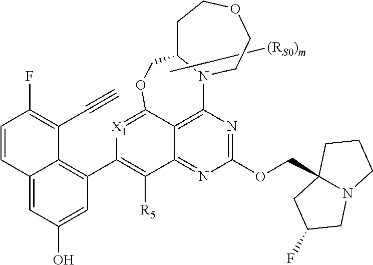

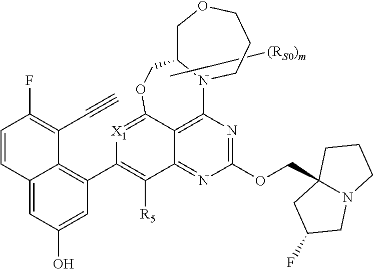

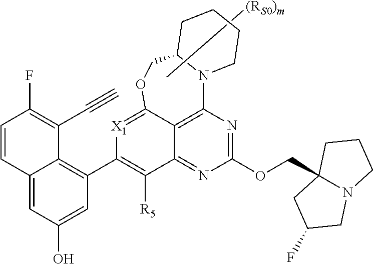

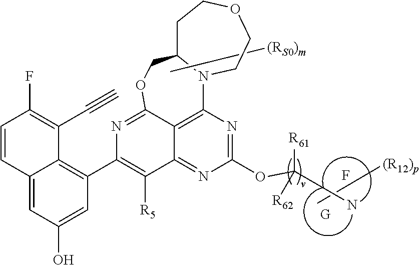

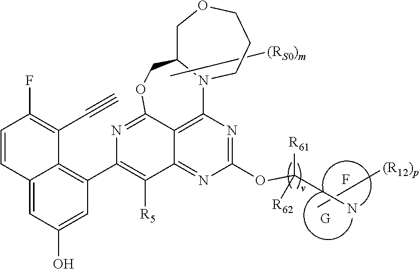

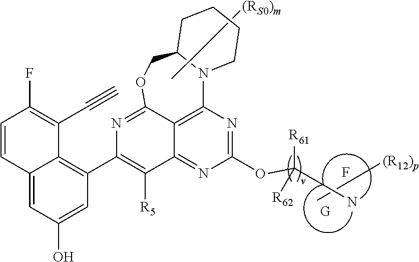

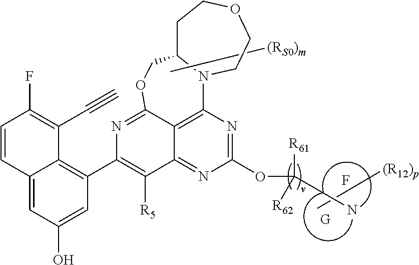

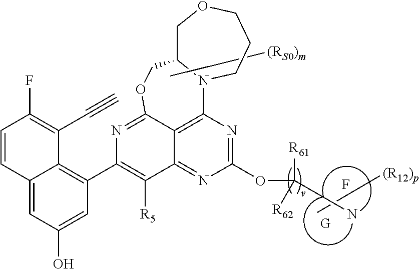

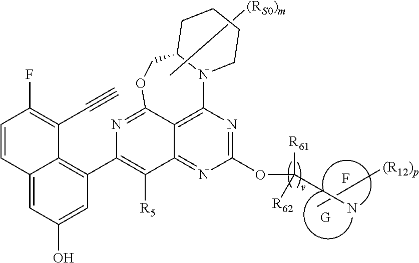

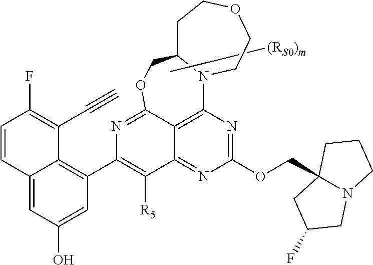

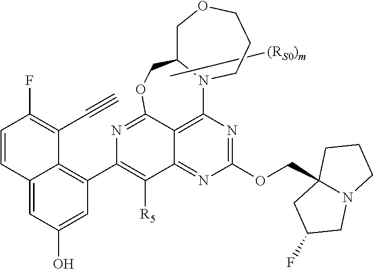

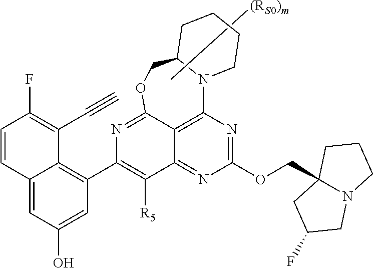

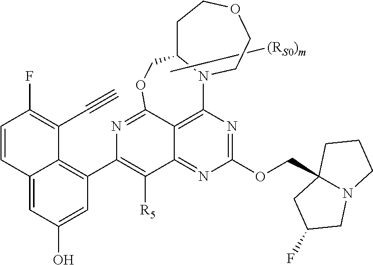

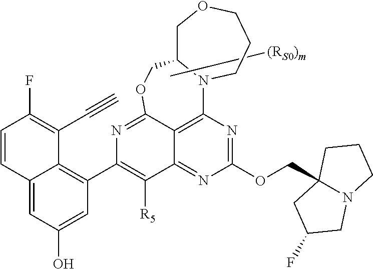

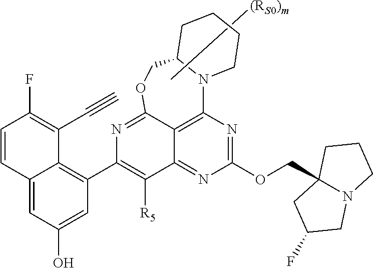

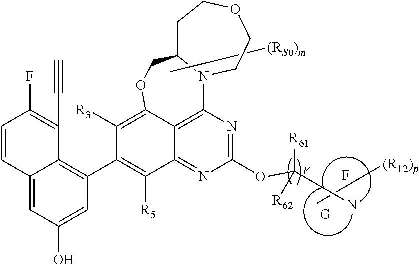

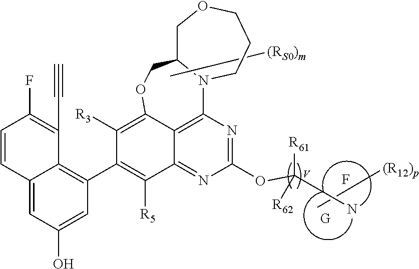

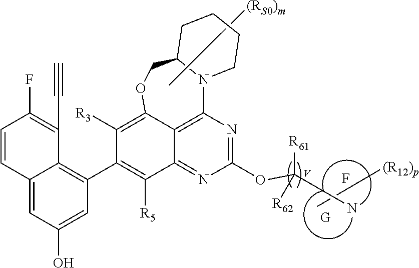

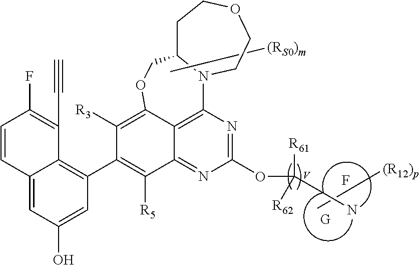

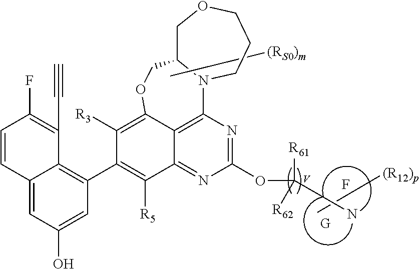

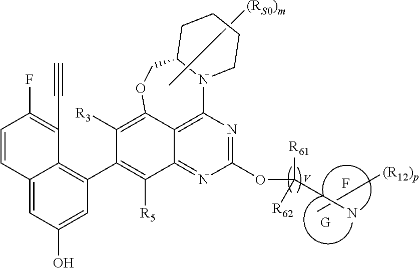

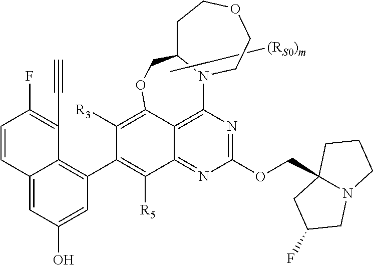

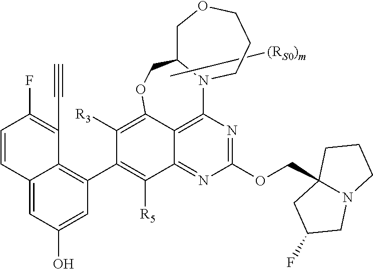

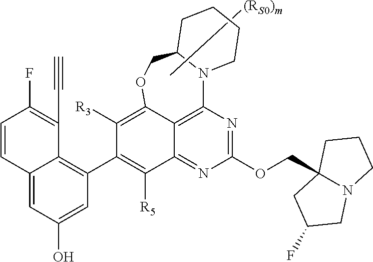

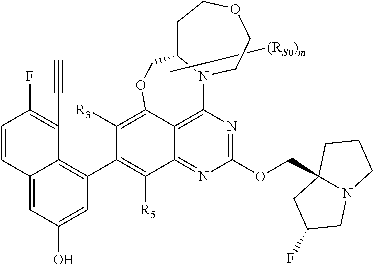

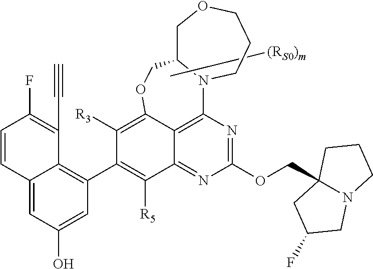

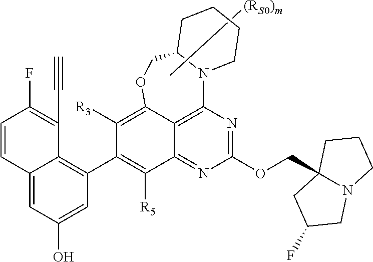

[24]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [23], wherein, R₅ is selected from deuterium, halogen, preferably, R₅ is selected from —F.

[25]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [24], wherein, the compound is selected from the formulas in Table 7:

TABLE 7

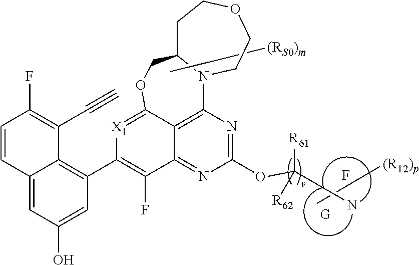

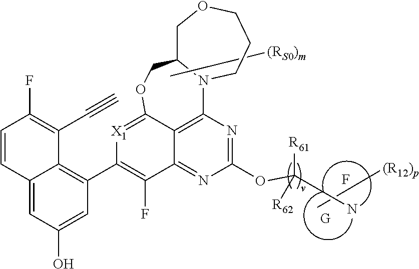

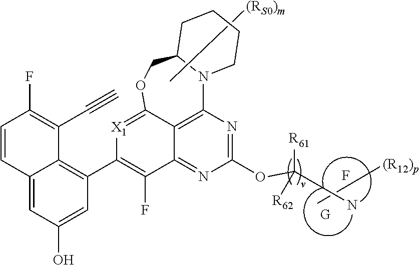

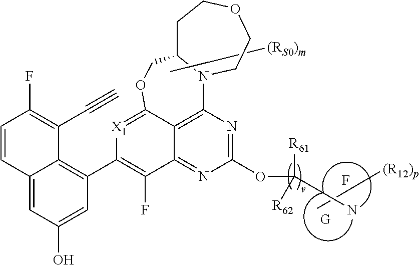

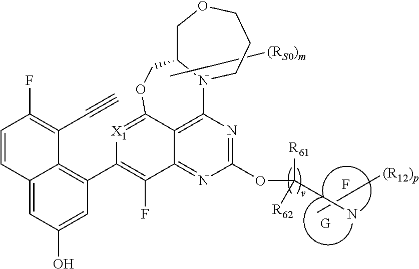

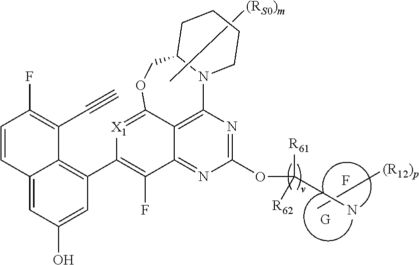

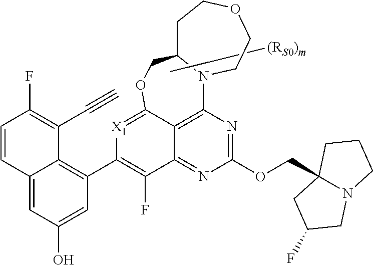

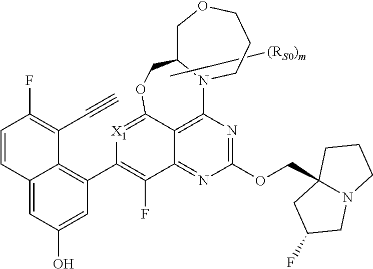

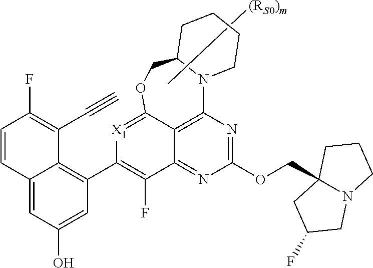

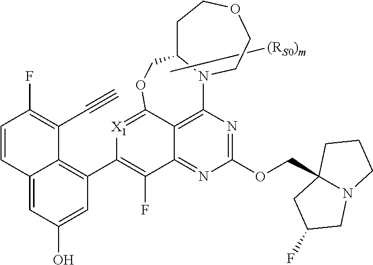

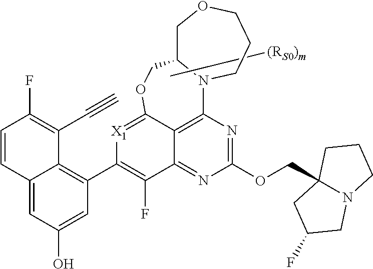

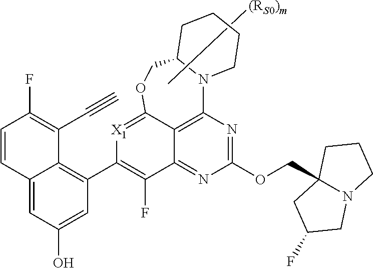

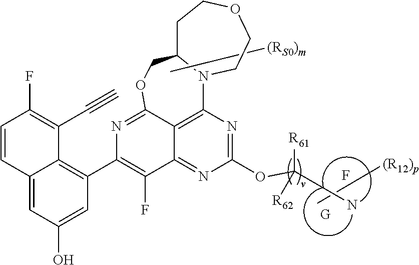

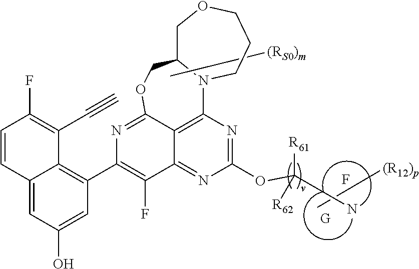

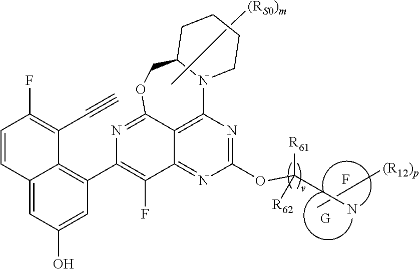

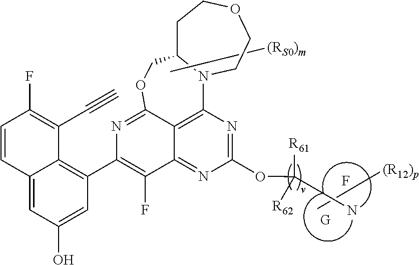

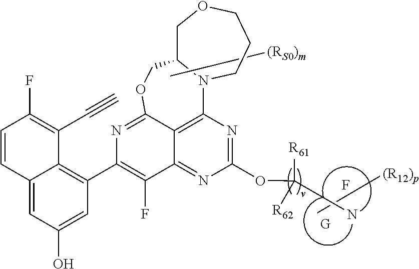

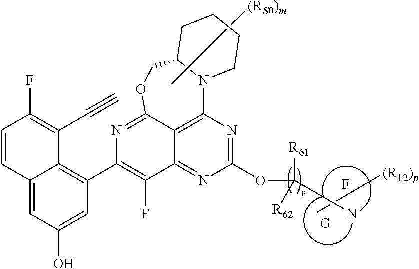

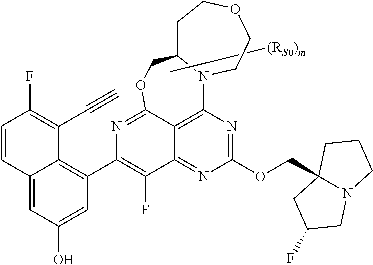

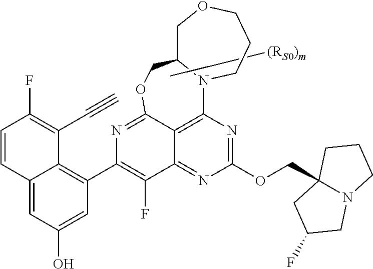

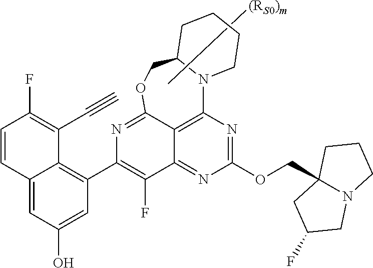

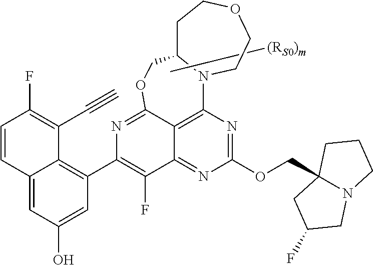

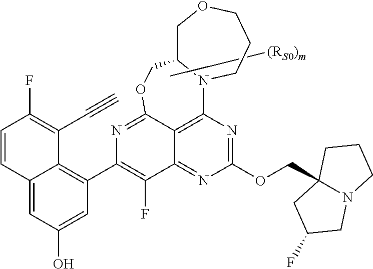

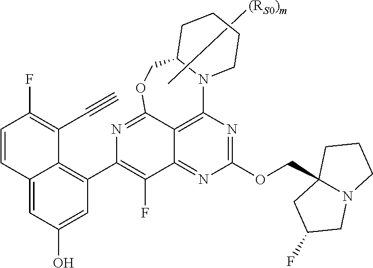

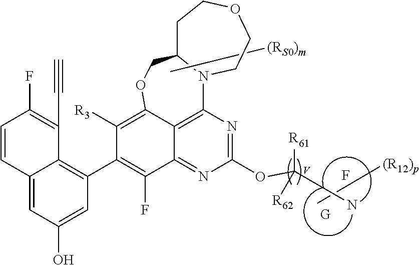

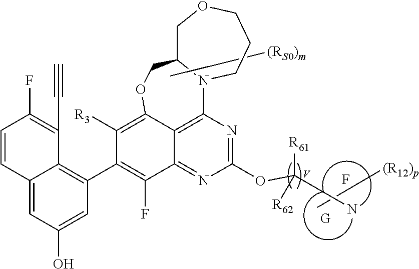

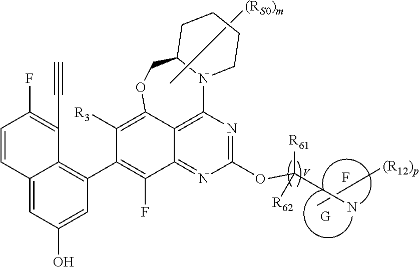

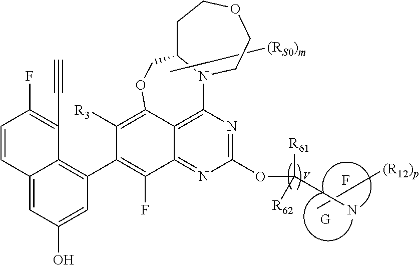

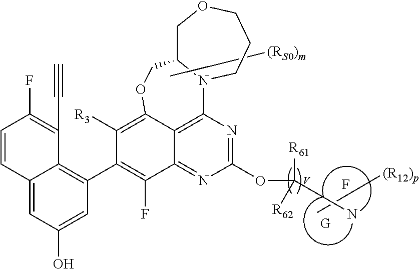

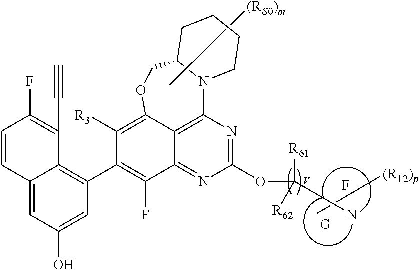

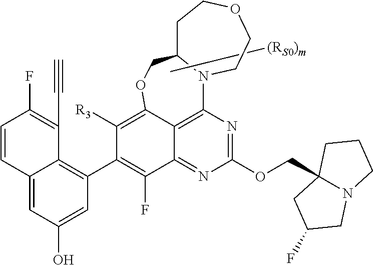

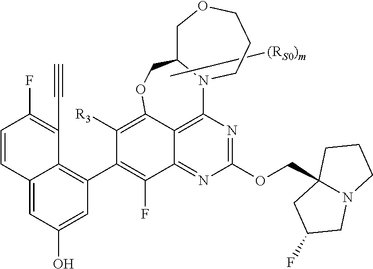

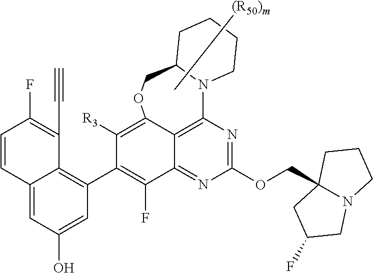

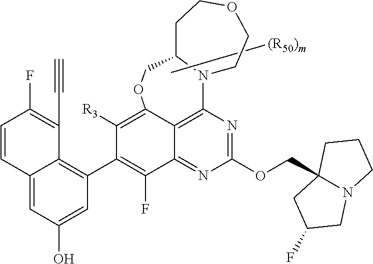

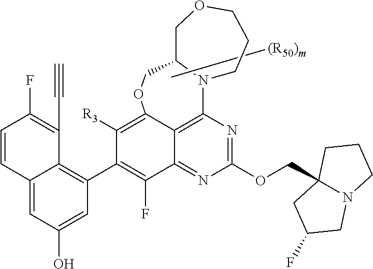

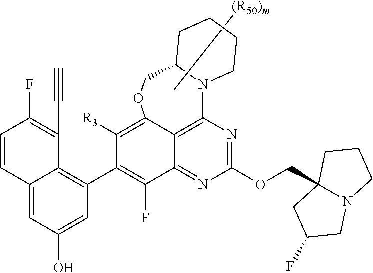

[26]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [25], wherein, the compound is selected from any one in Table 8:

TABLE 8

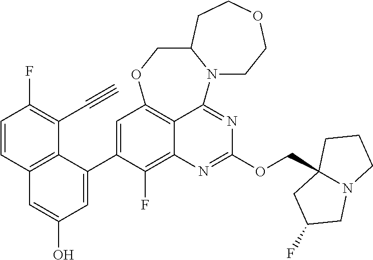 [SMILES: C#Cc1c(F)ccc2cc(O)cc(-c3cc4c5c(nc(OC[C@@]67CCCN6C[C@H](F)C7)nc5c3F)N3CCOCCC3CO4)c12]

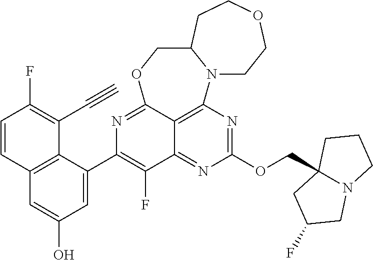 [SMILES: C#Cc1c(F)ccc2cc(O)cc(-c3nc4c5c(nc(OC[C@@]67CCCN6C[C@H](F)C7)nc5c3F)N3CCOCCC3CO4)c12]

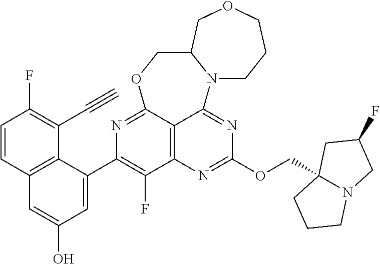 [SMILES: C#Cc1c(F)ccc2cc(O)cc(-c3nc4c5c(nc(OC[C@@]67CCCN6C[C@H](F)C7)nc5c3F)N3CCCOCC3CO4)c12]

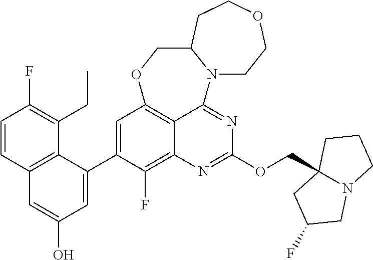 [SMILES: CCc1c(F)ccc2cc(O)cc(-c3cc4c5c(nc(OC[C@@]67CCCN6C[C@H](F)C7)nc5c3F)N3CCOCCC3CO4)c12]

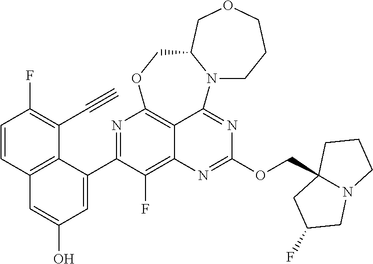 [SMILES: C#Cc1c(F)ccc2cc(O)cc(-c3nc4c5c(nc(OC[C@@]67CCCN6C[C@H](F)C7)nc5c3F)N3CCCOC[C@H]3CO4)c12]

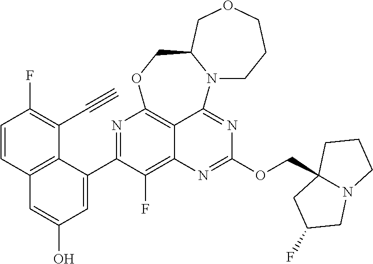 [SMILES: C#Cc1c(F)ccc2cc(O)cc(-c3nc4c5c(nc(OC[C@@]67CCCN6C[C@H](F)C7)nc5c3F)N3CCCOC[C@@H]3CO4)c12]

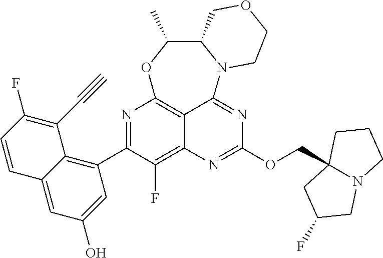 [SMILES: C#Cc1c(F)ccc2cc(O)cc(-c3nc4c5c(nc(OC[C@@]67CCCN6C[C@H](F)C7)nc5c3F)N3CCOC[C@H]3[C@@H](C)O4)c12]

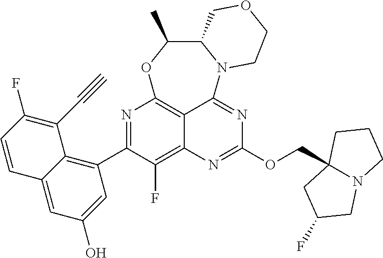 [SMILES: C#Cc1c(F)ccc2cc(O)cc(-c3nc4c5c(nc(OC[C@@]67CCCN6C[C@H](F)C7)nc5c3F)N3CCOC[C@H]3[C@H](C)O4)c12]

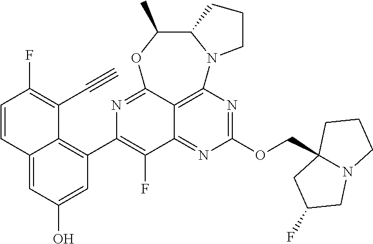 [SMILES: C#Cc1c(F)ccc2cc(O)cc(-c3nc4c5c(nc(OC[C@@]67CCCN6C[C@H](F)C7)nc5c3F)N3CCC[C@H]3[C@H](C)O4)c12]

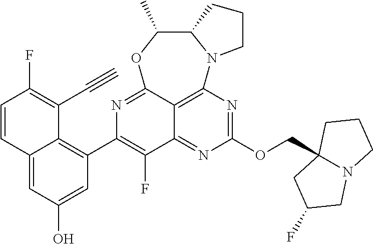 [SMILES: C#Cc1c(F)ccc2cc(O)cc(-c3nc4c5c(nc(OC[C@@]67CCCN6C[C@H](F)C7)nc5c3F)N3CCC[C@H]3[C@@H](C)O4)c12]

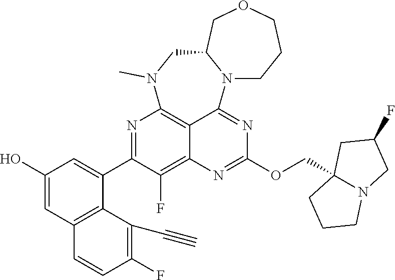 [SMILES: C#Cc1c(F)ccc2cc(O)cc(-c3nc4c5c(nc(OC[C@@]67CCCN6C[C@H](F)C7)nc5c3F)N3CCCOC[C@H]3CN4C)c12]

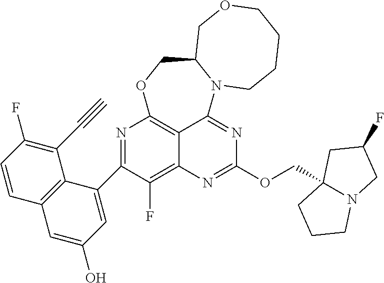 [SMILES: C#Cc1c(F)ccc2cc(O)cc(-c3nc4c5c(nc(OC[C@@]67CCCN6C[C@H](F)C7)nc5c3F)N3CCCCOC[C@@H]3CO4)c12]

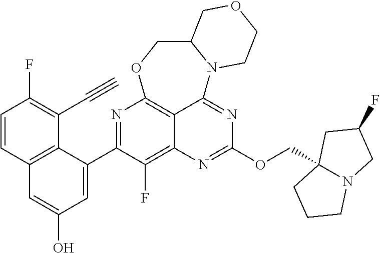 [SMILES: C#Cc1c(F)ccc2cc(O)cc(-c3nc4c5c(nc(OC[C@@]67CCCN6C[C@H](F)C7)nc5c3F)N3CCOCC3CO4)c12]

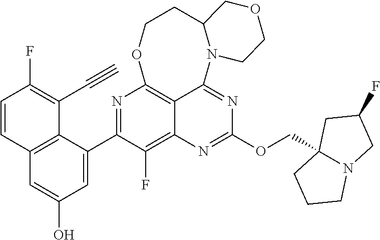 [SMILES: C#Cc1c(F)ccc2cc(O)cc(-c3nc4c5c(nc(OC[C@@]67CCCN6C[C@H](F)C7)nc5c3F)N3CCOCC3CCO4)c12]

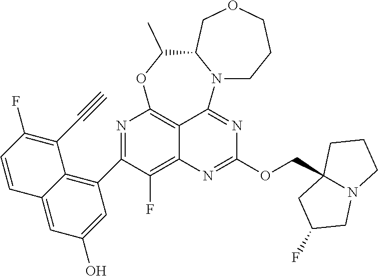

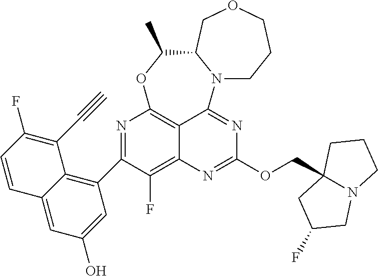 [SMILES: C#Cc1c(F)ccc2cc(O)cc(-c3nc4c5c(nc(OC[C@@]67CCCN6C[C@H](F)C7)nc5c3F)N3CCCOC[C@H]3[C@H](C)O4)c12]

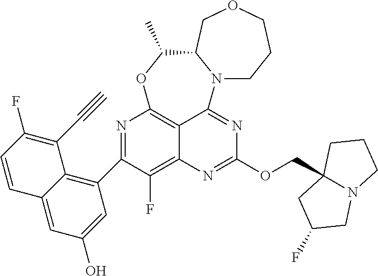 [SMILES: C#Cc1c(F)ccc2cc(O)cc(-c3nc4c5c(nc(OC[C@@]67CCCN6C[C@H](F)C7)nc5c3F)N3CCCOC[C@H]3[C@@H](C)O4)c12]

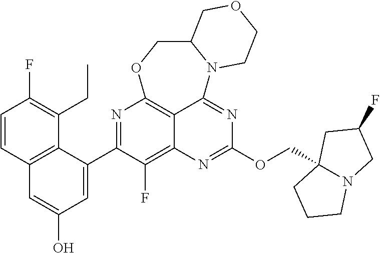 [SMILES: CCc1c(F)ccc2cc(O)cc(-c3nc4c5c(nc(OC[C@@]67CCCN6C[C@H](F)C7)nc5c3F)N3CCOCC3CO4)c12]

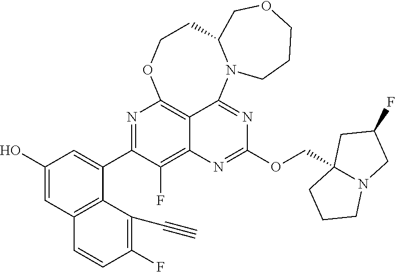 [SMILES: C#Cc1c(F)ccc2cc(O)cc(-c3nc4c5c(nc(OC[C@@]67CCCN6C[C@H](F)C7)nc5c3F)N3CCCOC[C@H]3CCO4)c12]

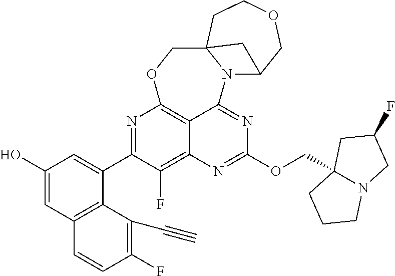 [SMILES: C#Cc1c(F)ccc2cc(O)cc(-c3nc4c5c(nc(OC[C@@]67CCCN6C[C@H](F)C7)nc5c3F)N3C5COCCC3(CO4)C5)c12]

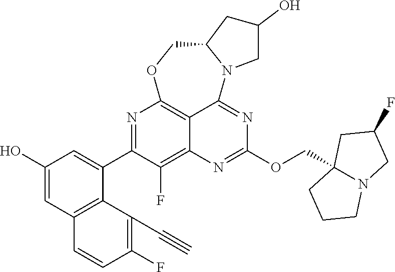 [SMILES: C#Cc1c(F)ccc2cc(O)cc(-c3nc4c5c(nc(OC[C@@]67CCCN6C[C@H](F)C7)nc5c3F)N3CC(O)C[C@H]3CO4)c12]

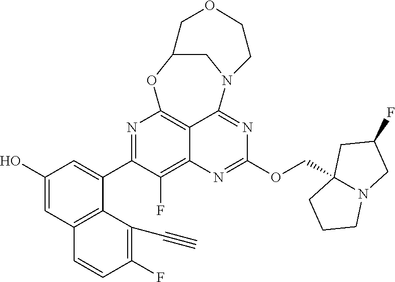 [SMILES: C#Cc1c(F)ccc2cc(O)cc(-c3nc4c5c(nc(OC[C@@]67CCCN6C[C@H](F)C7)nc5c3F)N3CCOCC(C3)O4)c12]

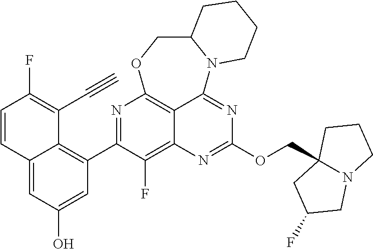 [SMILES: C#Cc1c(F)ccc2cc(O)cc(-c3nc4c5c(nc(OC[C@@]67CCCN6C[C@H](F)C7)nc5c3F)N3CCCCC3CO4)c12]

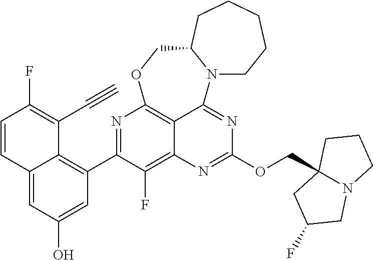 [SMILES: C#Cc1c(F)ccc2cc(O)cc(-c3nc4c5c(nc(OC[C@@]67CCCN6C[C@H](F)C7)nc5c3F)N3CCCCC[C@H]3CO4)c12]

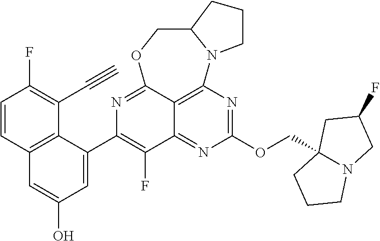 [SMILES: C#Cc1c(F)ccc2cc(O)cc(-c3nc4c5c(nc(OC[C@@]67CCCN6C[C@H](F)C7)nc5c3F)N3CCCC3CO4)c12]

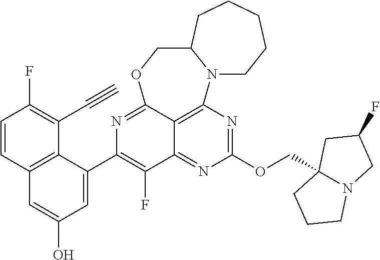 [SMILES: C#Cc1c(F)ccc2cc(O)cc(-c3nc4c5c(nc(OC[C@@]67CCCN6C[C@H](F)C7)nc5c3F)N3CCCCCC3CO4)c12]

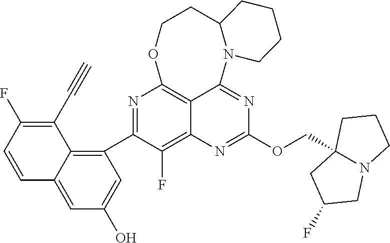 [SMILES: C#Cc1c(F)ccc2cc(O)cc(-c3nc4c5c(nc(OC[C@]67CCCN6C[C@H](F)C7)nc5c3F)N3CCCCC3CCO4)c12]

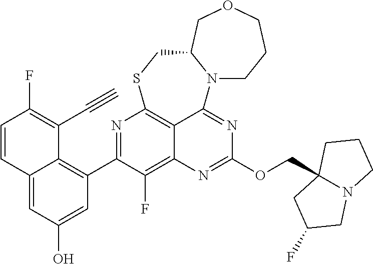 [SMILES: C#Cc1c(F)ccc2cc(O)cc(-c3nc4c5c(nc(OC[C@@]67CCCN6C[C@H](F)C7)nc5c3F)N3CCCOC[C@H]3CS4)c12]

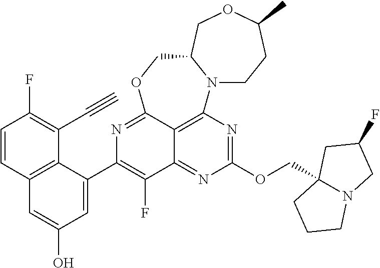 [SMILES: C#Cc1c(F)ccc2cc(O)cc(-c3nc4c5c(nc(OC[C@@]67CCCN6C[C@H](F)C7)nc5c3F)N3CC[C@H](C)OC[C@H]3CO4)c12]

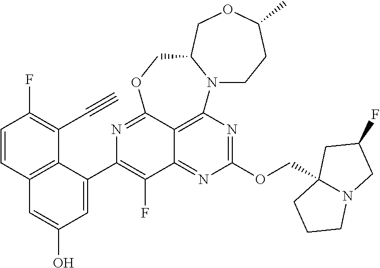 [SMILES: C#Cc1c(F)ccc2cc(O)cc(-c3nc4c5c(nc(OC[C@@]67CCCN6C[C@H](F)C7)nc5c3F)N3CC[C@@H](C)OC[C@H]3CO4)c12]

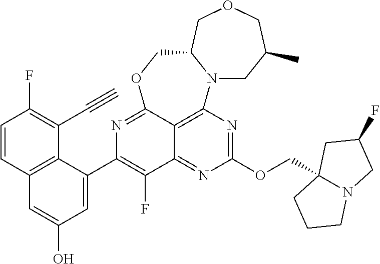 [SMILES: C#Cc1c(F)ccc2cc(O)cc(-c3nc4c5c(nc(OC[C@@]67CCCN6C[C@H](F)C7)nc5c3F)N3C[C@H](C)COC[C@H]3CO4)c12]

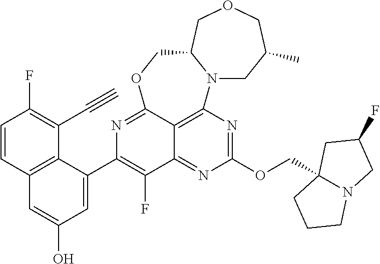 [SMILES: C#Cc1c(F)ccc2cc(O)cc(-c3nc4c5c(nc(OC[C@@]67CCCN6C[C@H](F)C7)nc5c3F)N3C[C@@H](C)COC[C@H]3CO4)c12]

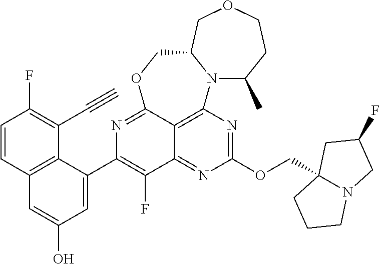 [SMILES: C#Cc1c(F)ccc2cc(O)cc(-c3nc4c5c(nc(OC[C@@]67CCCN6C[C@H](F)C7)nc5c3F)N3[C@@H](COCC[C@H]3C)CO4)c12]

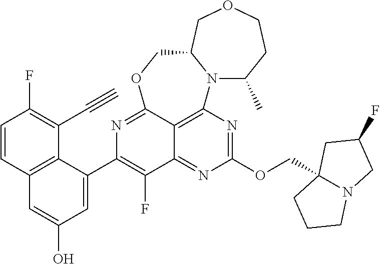 [SMILES: C#Cc1c(F)ccc2cc(O)cc(-c3nc4c5c(nc(OC[C@@]67CCCN6C[C@H](F)C7)nc5c3F)N3[C@@H](COCC[C@@H]3C)CO4)c12]

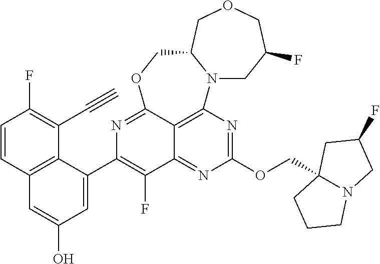 [SMILES: C#Cc1c(F)ccc2cc(O)cc(-c3nc4c5c(nc(OC[C@@]67CCCN6C[C@H](F)C7)nc5c3F)N3C[C@H](F)COC[C@H]3CO4)c12]

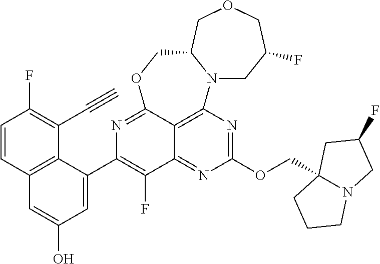 [SMILES: C#Cc1c(F)ccc2cc(O)cc(-c3nc4c5c(nc(OC[C@@]67CCCN6C[C@H](F)C7)nc5c3F)N3C[C@@H](F)COC[C@H]3CO4)c12]

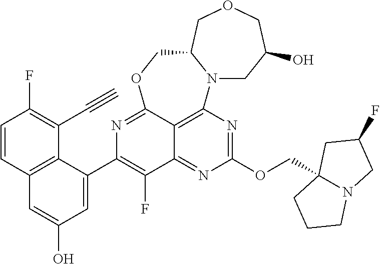 [SMILES: C#Cc1c(F)ccc2cc(O)cc(-c3nc4c5c(nc(OC[C@@]67CCCN6C[C@H](F)C7)nc5c3F)N3C[C@H](O)COC[C@H]3CO4)c12]

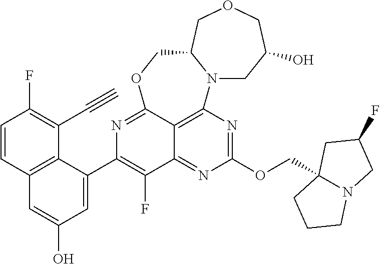 [SMILES: C#Cc1c(F)ccc2cc(O)cc(-c3nc4c5c(nc(OC[C@@]67CCCN6C[C@H](F)C7)nc5c3F)N3C[C@@H](O)COC[C@H]3CO4)c12]

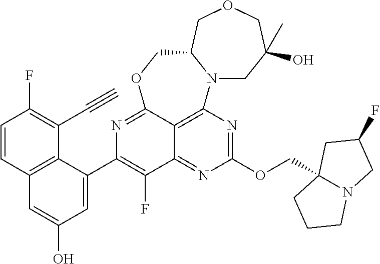 [SMILES: C#Cc1c(F)ccc2cc(O)cc(-c3nc4c5c(nc(OC[C@@]67CCCN6C[C@H](F)C7)nc5c3F)N3C[C@](C)(O)COC[C@H]3CO4)c12]

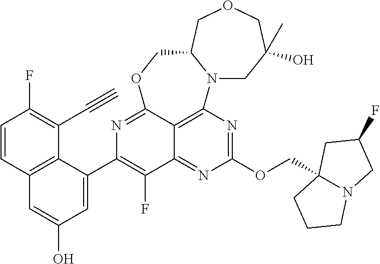 [SMILES: C#Cc1c(F)ccc2cc(O)cc(-c3nc4c5c(nc(OC[C@@]67CCCN6C[C@H](F)C7)nc5c3F)N3C[C@@](C)(O)COC[C@H]3CO4)c12]

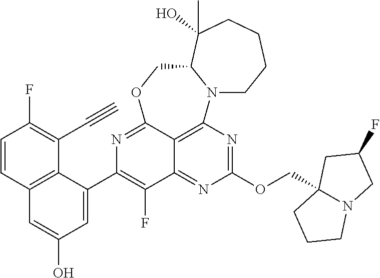 [SMILES: C#Cc1c(F)ccc2cc(O)cc(-c3nc4c5c(nc(OC[C@@]67CCCN6C[C@H](F)C7)nc5c3F)N3CCCC[C@@](C)(O)[C@H]3CO4)c12]

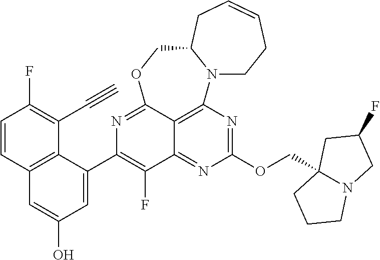 [SMILES: C#Cc1c(F)ccc2cc(O)cc(-c3nc4c5c(nc(OC[C@@]67CCCN6C[C@H](F)C7)nc5c3F)N3CCC=CC[C@H]3CO4)c12]

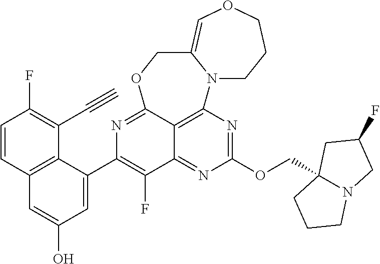 [SMILES: C#Cc1c(F)ccc2cc(O)cc(-c3nc4c5c(nc(OC[C@@]67CCCN6C[C@H](F)C7)nc5c3F)N3CCCOC=C3CO4)c12]

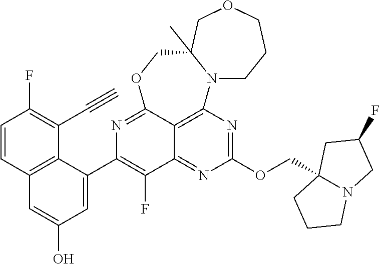 [SMILES: C#Cc1c(F)ccc2cc(O)cc(-c3nc4c5c(nc(OC[C@@]67CCCN6C[C@H](F)C7)nc5c3F)N3CCCOC[C@@]3(C)CO4)c12]

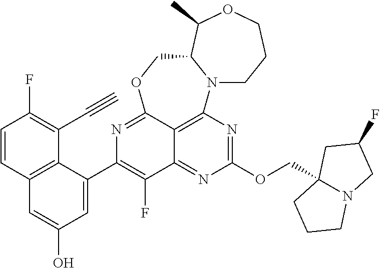 [SMILES: C#Cc1c(F)ccc2cc(O)cc(-c3nc4c5c(nc(OC[C@@]67CCCN6C[C@H](F)C7)nc5c3F)N3CCCO[C@H](C)[C@H]3CO4)c12]

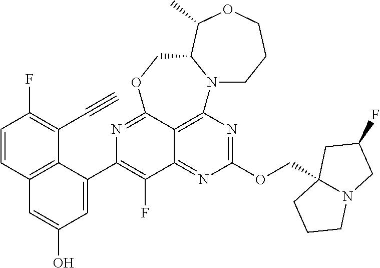 [SMILES: C#Cc1c(F)ccc2cc(O)cc(-c3nc4c5c(nc(OC[C@@]67CCCN6C[C@H](F)C7)nc5c3F)N3CCCO[C@@H](C)[C@H]3CO4)c12]

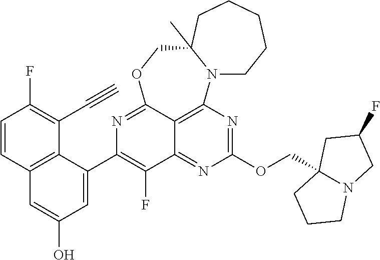 [SMILES: C#Cc1c(F)ccc2cc(O)cc(-c3nc4c5c(nc(OC[C@@]67CCCN6C[C@H](F)C7)nc5c3F)N3CCCCC[C@@]3(C)CO4)c12]

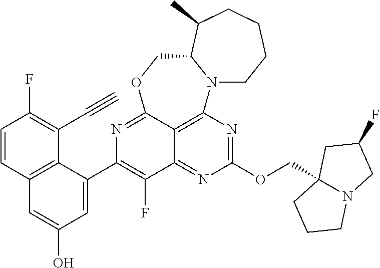 [SMILES: C#Cc1c(F)ccc2cc(O)cc(-c3nc4c5c(nc(OC[C@@]67CCCN6C[C@H](F)C7)nc5c3F)N3CCCC[C@H](C)[C@H]3CO4)c12]

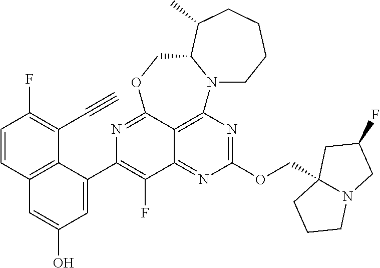 [SMILES: C#Cc1c(F)ccc2cc(O)cc(-c3nc4c5c(nc(OC[C@@]67CCCN6C[C@H](F)C7)nc5c3F)N3CCCC[C@@H](C)[C@H]3CO4)c12]

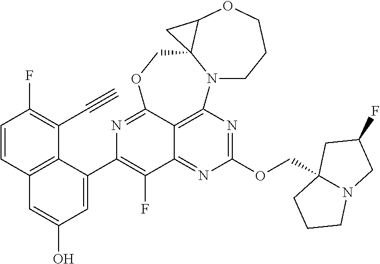 [SMILES: C#Cc1c(F)ccc2cc(O)cc(-c3nc4c5c(nc(OC[C@@]67CCCN6C[C@H](F)C7)nc5c3F)N3CCCOC5C[C@@]53CO4)c12]

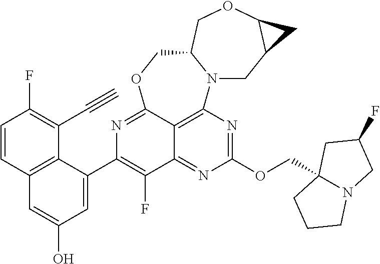 [SMILES: C#Cc1c(F)ccc2cc(O)cc(-c3nc4c5c(nc(OC[C@@]67CCCN6C[C@H](F)C7)nc5c3F)N3C[C@@H]5C[C@@H]5OC[C@H]3CO4)c12]

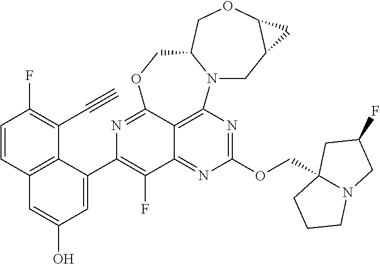 [SMILES: C#Cc1c(F)ccc2cc(O)cc(-c3nc4c5c(nc(OC[C@@]67CCCN6C[C@H](F)C7)nc5c3F)N3C[C@H]5C[C@H]5OC[C@H]3CO4)c12]

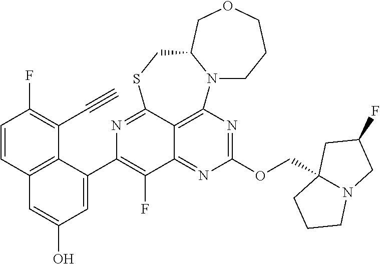 [SMILES: C#Cc1c(F)ccc2cc(O)cc(-c3nc4c5c(nc(OC[C@@]67CCCN6C[C@H](F)C7)nc5c3F)N3CCCOC[C@H]3CS4)c12]

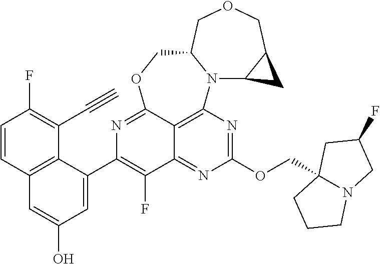 [SMILES: C#Cc1c(F)ccc2cc(O)cc(-c3nc4c5c(nc(OC[C@@]67CCCN6C[C@H](F)C7)nc5c3F)N3[C@@H](COC[C@H]5C[C@H]53)CO4)c12]

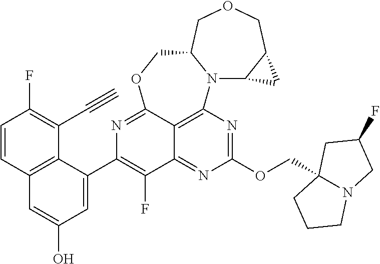 [SMILES: C#Cc1c(F)ccc2cc(O)cc(-c3nc4c5c(nc(OC[C@@]67CCCN6C[C@H](F)C7)nc5c3F)N3[C@@H](COC[C@@H]5C[C@@H]53)CO4)c12]

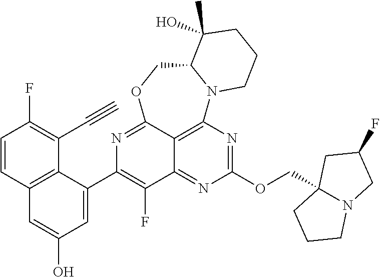 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@H]3CO4)c12]

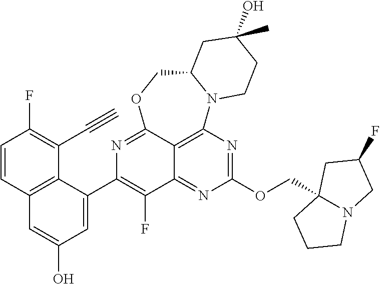 [SMILES: C#Cc1c(F)ccc2cc(O)cc(-c3nc4c5c(nc(OC[C@@]67CCCN6C[C@H](F)C7)nc5c3F)N3CC[C@@](C)(O)C[C@H]3CO4)c12]

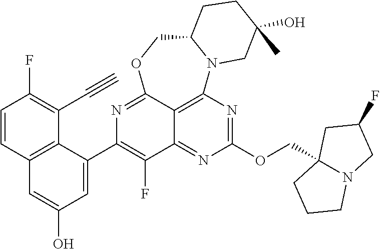 [SMILES: C#Cc1c(F)ccc2cc(O)cc(-c3nc4c5c(nc(OC[C@@]67CCCN6C[C@H](F)C7)nc5c3F)N3C[C@@](C)(O)CC[C@H]3CO4)c12]

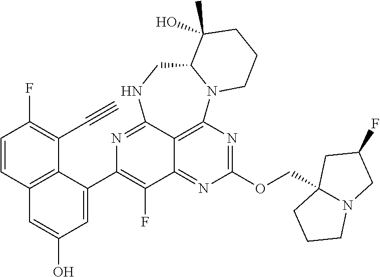 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@H]3CN4)c12]

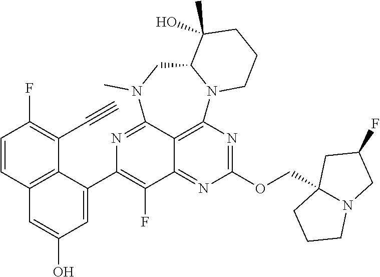 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@H]3CN4C)c12]

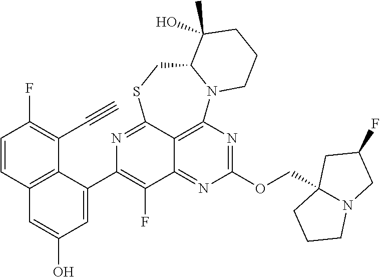 [SMILES: C#Cc1c(F)ccc2cc(O)cc(-c3nc4c5c(nc(OC[C@@]67CCCN6C[C@H](F)C7)nc5c3F)N3CCC[C@@](C)(O)[C@H]3CS4)c12]

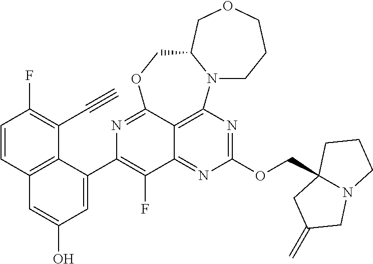 [SMILES: C#Cc1c(F)ccc2cc(O)cc(-c3nc4c5c(nc(OC[C@@]67CCCN6CC(=C)C7)nc5c3F)N3CCCOC[C@H]3CO4)c12]

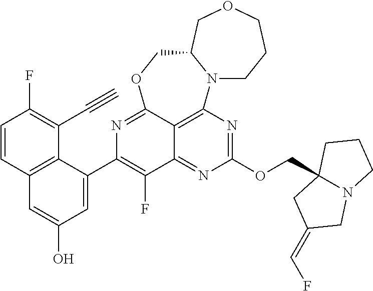 [SMILES: C#Cc1c(F)ccc2cc(O)cc(-c3nc4c5c(nc(OC[C@@]67CCCN6C/C(=C\F)C7)nc5c3F)N3CCCOC[C@H]3CO4)c12]

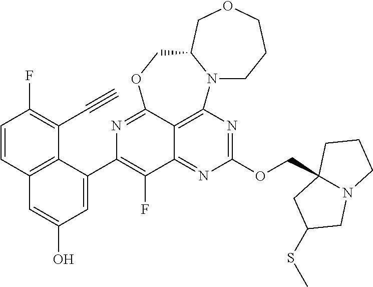 [SMILES: C#Cc1c(F)ccc2cc(O)cc(-c3nc4c5c(nc(OC[C@@]67CCCN6CC(SC)C7)nc5c3F)N3CCCOC[C@H]3CO4)c12]

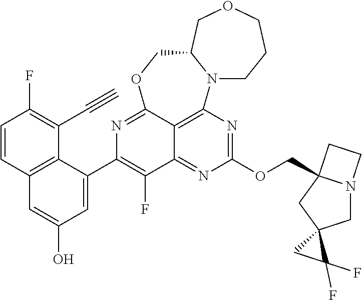 [SMILES: C#Cc1c(F)ccc2cc(O)cc(-c3nc4c5c(nc(OC[C@@]67CCN6C[C@]6(CC6(F)F)C7)nc5c3F)N3CCCOC[C@H]3CO4)c12]

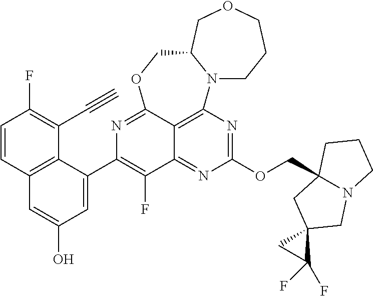 [SMILES: C#Cc1c(F)ccc2cc(O)cc(-c3nc4c5c(nc(OC[C@@]67CCCN6C[C@]6(CC6(F)F)C7)nc5c3F)N3CCCOC[C@H]3CO4)c12]

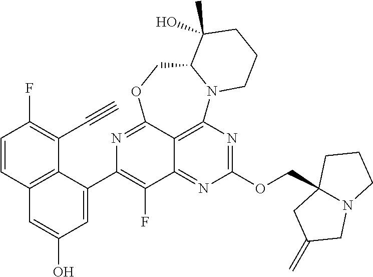 [SMILES: C#Cc1c(F)ccc2cc(O)cc(-c3nc4c5c(nc(OC[C@@]67CCCN6CC(=C)C7)nc5c3F)N3CCC[C@@](C)(O)[C@H]3CO4)c12]

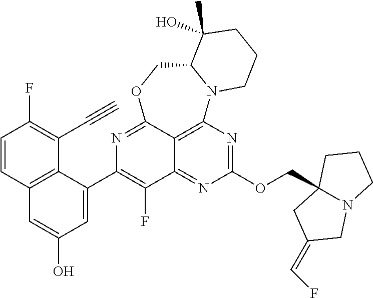 [SMILES: C#Cc1c(F)ccc2cc(O)cc(-c3nc4c5c(nc(OC[C@@]67CCCN6C/C(=C\F)C7)nc5c3F)N3CCC[C@@](C)(O)[C@H]3CO4)c12]

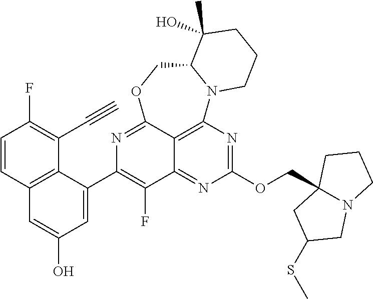 [SMILES: C#Cc1c(F)ccc2cc(O)cc(-c3nc4c5c(nc(OC[C@@]67CCCN6CC(SC)C7)nc5c3F)N3CCC[C@@](C)(O)[C@H]3CO4)c12]

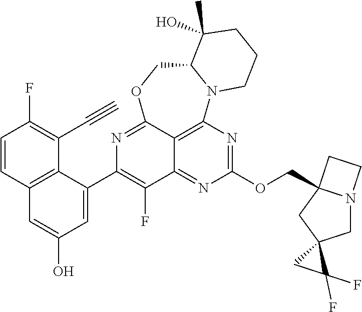 [SMILES: C#Cc1c(F)ccc2cc(O)cc(-c3nc4c5c(nc(OC[C@@]67CCN6C[C@]6(CC6(F)F)C7)nc5c3F)N3CCC[C@@](C)(O)[C@H]3CO4)c12]

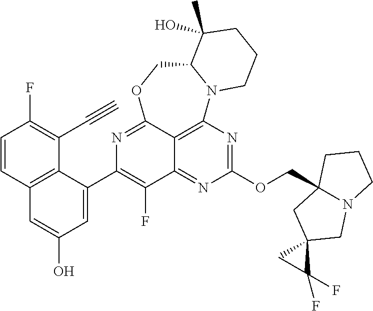 [SMILES: C#Cc1c(F)ccc2cc(O)cc(-c3nc4c5c(nc(OC[C@@]67CCCN6C[C@]6(CC6(F)F)C7)nc5c3F)N3CCC[C@@](C)(O)[C@H]3CO4)c12]

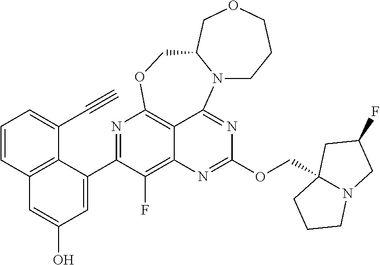 [SMILES: C#Cc1cccc2cc(O)cc(-c3nc4c5c(nc(OC[C@@]67CCCN6C[C@H](F)C7)nc5c3F)N3CCCOC[C@H]3CO4)c12]

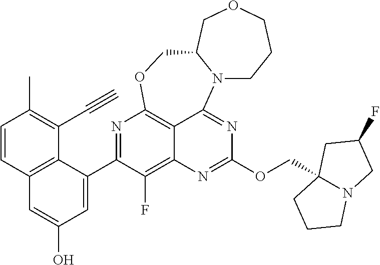 [SMILES: C#Cc1c(C)ccc2cc(O)cc(-c3nc4c5c(nc(OC[C@@]67CCCN6C[C@H](F)C7)nc5c3F)N3CCCOC[C@H]3CO4)c12]

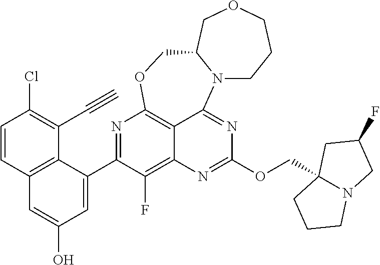 [SMILES: C#Cc1c(Cl)ccc2cc(O)cc(-c3nc4c5c(nc(OC[C@@]67CCCN6C[C@H](F)C7)nc5c3F)N3CCCOC[C@H]3CO4)c12]

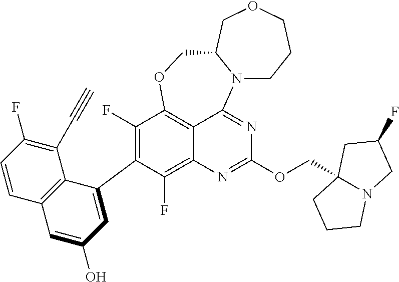

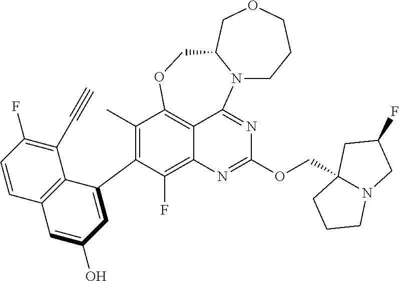

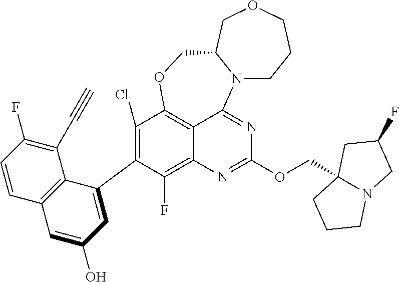

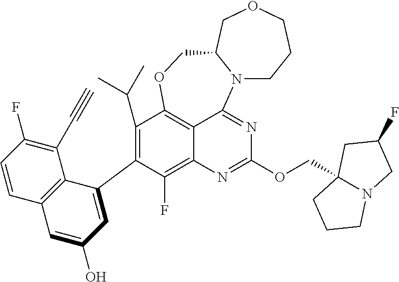

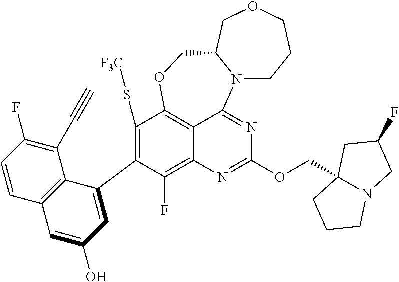

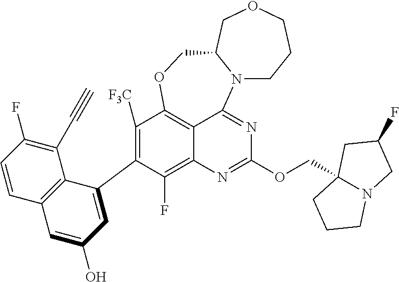

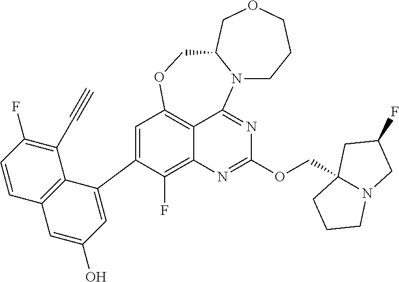 [SMILES: C#Cc1c(F)ccc2cc(O)cc(-c3cc4c5c(nc(OC[C@@]67CCCN6C[C@H](F)C7)nc5c3F)N3CCCOC[C@H]3CO4)c12]

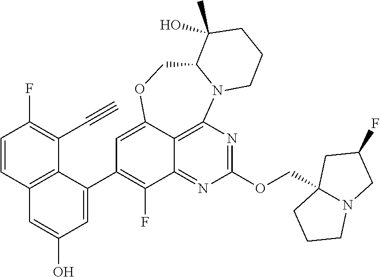 [SMILES: C#Cc1c(F)ccc2cc(O)cc(-c3cc4c5c(nc(OC[C@@]67CCCN6C[C@H](F)C7)nc5c3F)N3CCC[C@@](C)(O)[C@H]3CO4)c12]

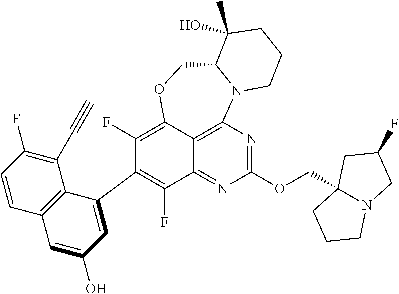

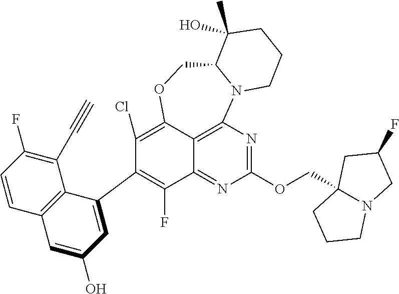

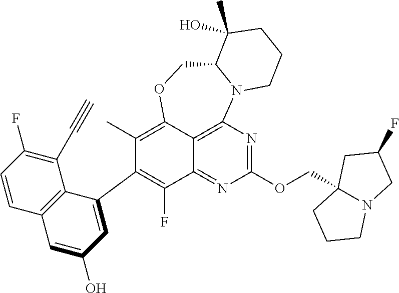

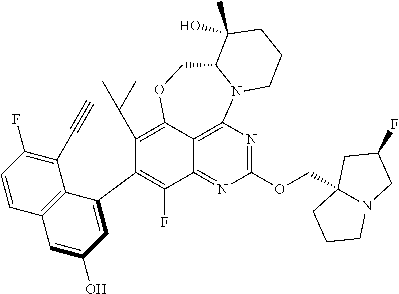

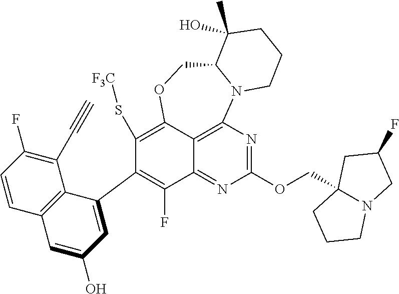

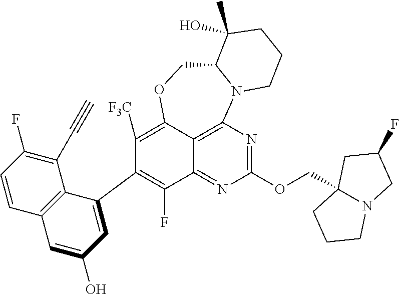

[27]. The compound of formula (I), a stereoisomer thereof, an atropisomer thereof, a pharmaceutically acceptable salt thereof, a pharmaceutically acceptable salt of the stereoisomer, or a pharmaceutically acceptable salt of the atropisomer thereof, a prodrug thereof, a deuterated molecule thereof or a conjugated form thereof of any one of [1] to [26], wherein, the conjugated form is a PROTAC molecule.

[28]. An intermediate for preparing the compound of Formula (I), wherein the intermediate is selected from any compound in Table 9:

TABLE 9

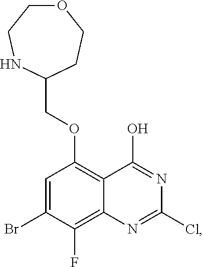 [SMILES: Oc1nc(Cl)nc2c(F)c(Br)cc(OCC3CCOCCN3)c12]

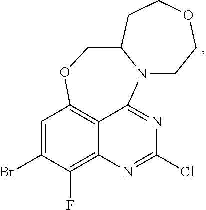 [SMILES: Fc1c(Br)cc2c3c(nc(Cl)nc13)N1CCOCCC1CO2]

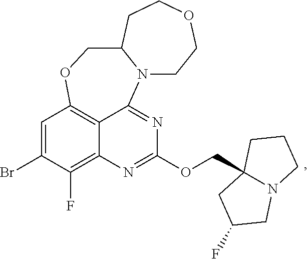 [SMILES: Fc1c(Br)cc2c3c(nc(OC[C@@]45CCCN4C[C@H](F)C5)nc13)N1CCOCCC1CO2]

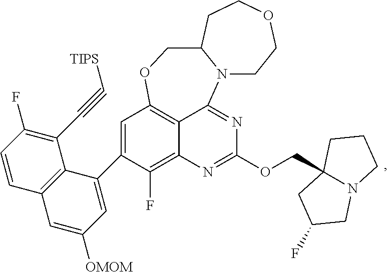

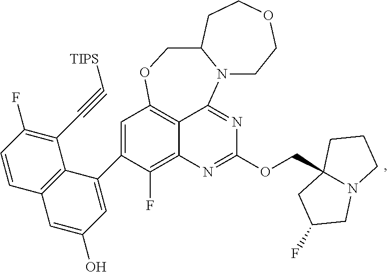

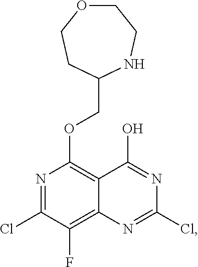 [SMILES: Oc1nc(Cl)nc2c(F)c(Cl)nc(OCC3CCOCCN3)c12]

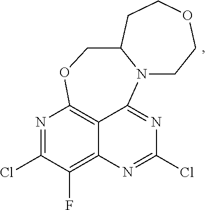 [SMILES: Fc1c(Cl)nc2c3c(nc(Cl)nc13)N1CCOCCC1CO2]

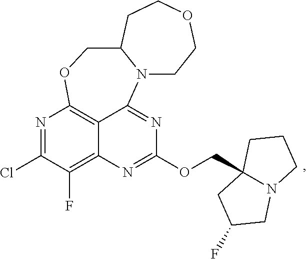 [SMILES: Fc1c(Cl)nc2c3c(nc(OC[C@@]45CCCN4C[C@H](F)C5)nc13)N1CCOCCC1CO2]

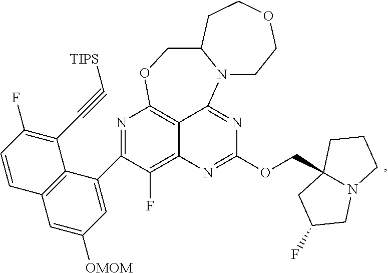

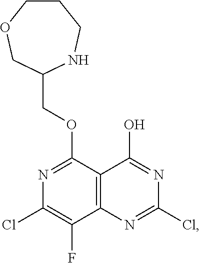 [SMILES: Oc1nc(Cl)nc2c(F)c(Cl)nc(OCC3COCCCN3)c12]

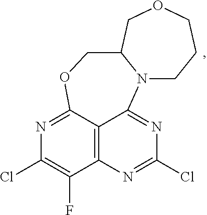 [SMILES: Fc1c(Cl)nc2c3c(nc(Cl)nc13)N1CCCOCC1CO2]

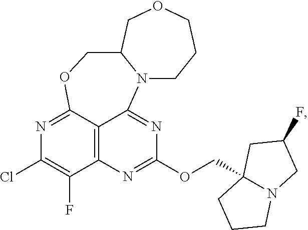 [SMILES: Fc1c(Cl)nc2c3c(nc(OC[C@@]45CCCN4C[C@H](F)C5)nc13)N1CCCOCC1CO2]

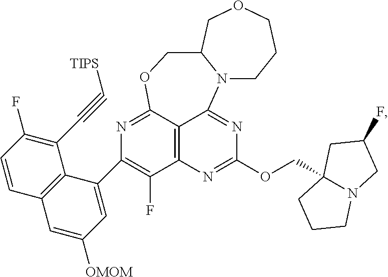

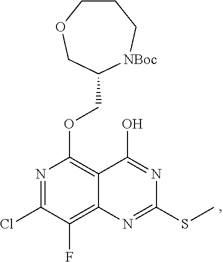 [SMILES: CSc1nc(O)c2c(OC[C@@H]3COCCCN3C(=O)OC(C)(C)C)nc(Cl)c(F)c2n1]

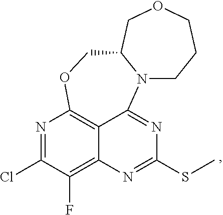 [SMILES: CSc1nc2c3c(nc(Cl)c(F)c3n1)OC[C@@H]1COCCCN21]

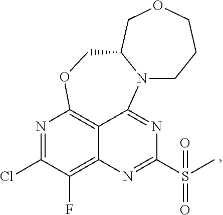 [SMILES: CS(=O)(=O)c1nc2c3c(nc(Cl)c(F)c3n1)OC[C@@H]1COCCCN21]

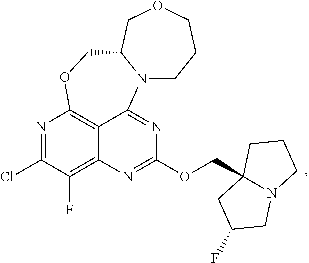 [SMILES: Fc1c(Cl)nc2c3c(nc(OC[C@@]45CCCN4C[C@H](F)C5)nc13)N1CCCOC[C@H]1CO2]

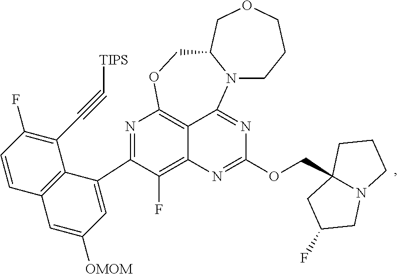

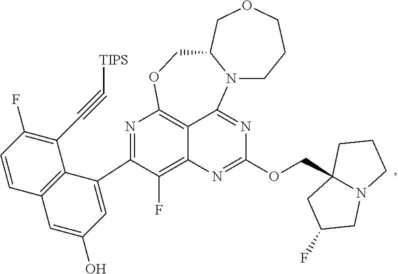

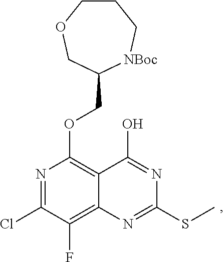 [SMILES: CSc1nc(O)c2c(OC[C@H]3COCCCN3C(=O)OC(C)(C)C)nc(Cl)c(F)c2n1]

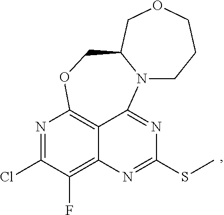 [SMILES: CSc1nc2c3c(nc(Cl)c(F)c3n1)OC[C@H]1COCCCN21]

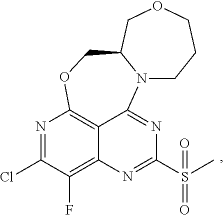 [SMILES: CS(=O)(=O)c1nc2c3c(nc(Cl)c(F)c3n1)OC[C@H]1COCCCN21]

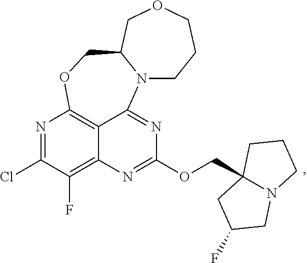 [SMILES: Fc1c(Cl)nc2c3c(nc(OC[C@@]45CCCN4C[C@H](F)C5)nc13)N1CCCOC[C@@H]1CO2]

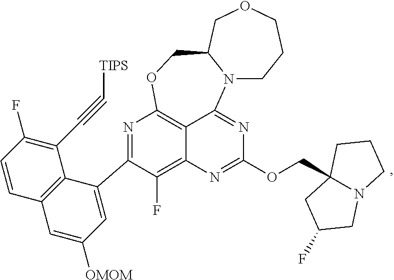

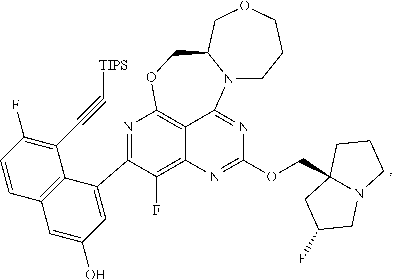

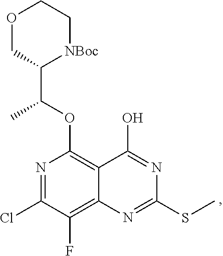 [SMILES: CSc1nc(O)c2c(O[C@H](C)[C@@H]3COCCN3C(=O)OC(C)(C)C)nc(Cl)c(F)c2n1]

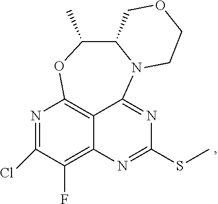 [SMILES: CSc1nc2c3c(nc(Cl)c(F)c3n1)O[C@H](C)[C@@H]1COCCN21]

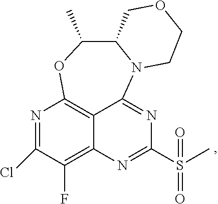 [SMILES: C[C@H]1Oc2nc(Cl)c(F)c3nc(S(C)(=O)=O)nc(c23)N2CCOC[C@@H]12]

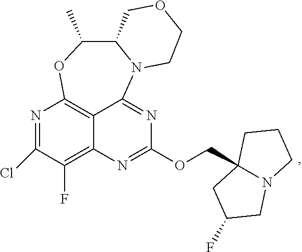 [SMILES: C[C@H]1Oc2nc(Cl)c(F)c3nc(OC[C@@]45CCCN4C[C@H](F)C5)nc(c23)N2CCOC[C@@H]12]

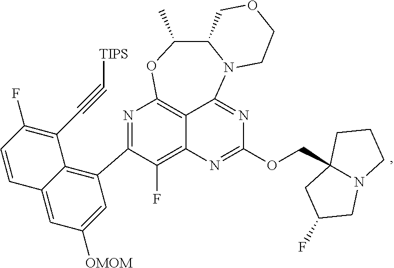

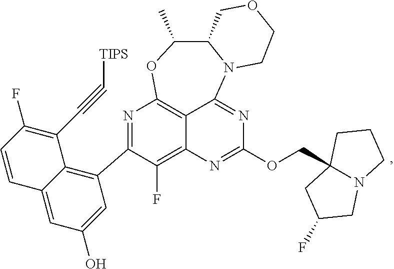

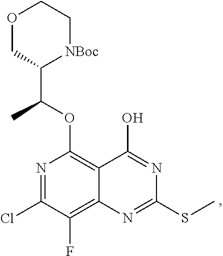 [SMILES: CSc1nc(O)c2c(O[C@@H](C)[C@@H]3COCCN3C(=O)OC(C)(C)C)nc(Cl)c(F)c2n1]

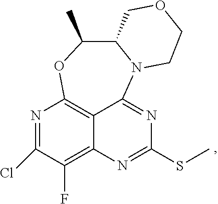 [SMILES: CSc1nc2c3c(nc(Cl)c(F)c3n1)O[C@@H](C)[C@@H]1COCCN21]

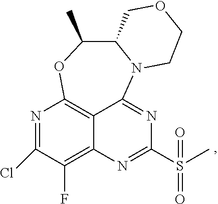 [SMILES: C[C@@H]1Oc2nc(Cl)c(F)c3nc(S(C)(=O)=O)nc(c23)N2CCOC[C@@H]12]

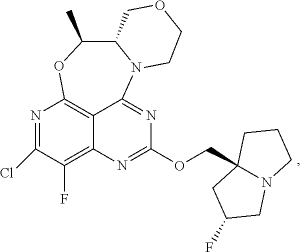 [SMILES: C[C@@H]1Oc2nc(Cl)c(F)c3nc(OC[C@@]45CCCN4C[C@H](F)C5)nc(c23)N2CCOC[C@@H]12]

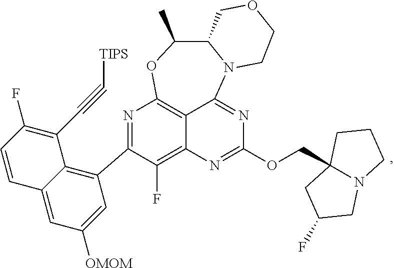

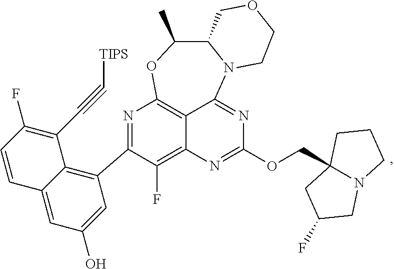

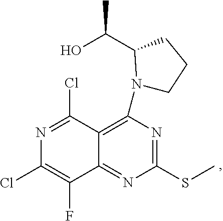 [SMILES: CSc1nc(N2CCC[C@H]2[C@H](C)O)c2c(Cl)nc(Cl)c(F)c2n1]

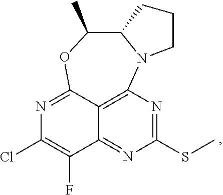 [SMILES: CSc1nc2c3c(nc(Cl)c(F)c3n1)O[C@@H](C)[C@@H]1CCCN21]

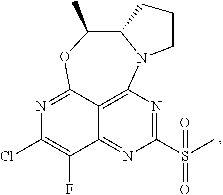 [SMILES: C[C@@H]1Oc2nc(Cl)c(F)c3nc(S(C)(=O)=O)nc(c23)N2CCC[C@@H]12]

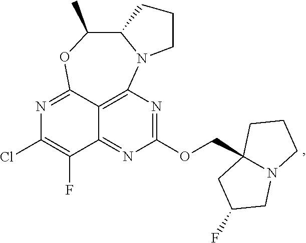 [SMILES: C[C@@H]1Oc2nc(Cl)c(F)c3nc(OC[C@@]45CCCN4C[C@H](F)C5)nc(c23)N2CCC[C@@H]12]

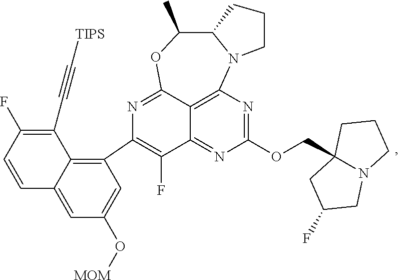

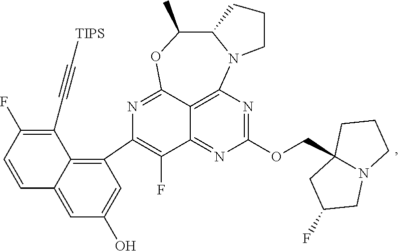

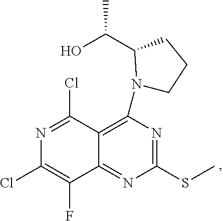 [SMILES: CSc1nc(N2CCC[C@H]2[C@@H](C)O)c2c(Cl)nc(Cl)c(F)c2n1]

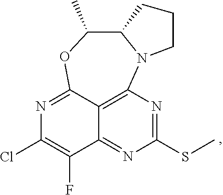 [SMILES: CSc1nc2c3c(nc(Cl)c(F)c3n1)O[C@H](C)[C@@H]1CCCN21]

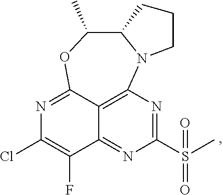 [SMILES: C[C@H]1Oc2nc(Cl)c(F)c3nc(S(C)(=O)=O)nc(c23)N2CCC[C@@H]12]

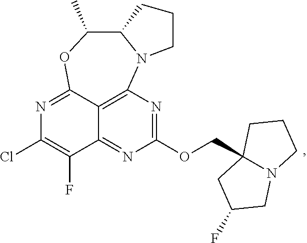 [SMILES: C[C@H]1Oc2nc(Cl)c(F)c3nc(OC[C@@]45CCCN4C[C@H](F)C5)nc(c23)N2CCC[C@@H]12]

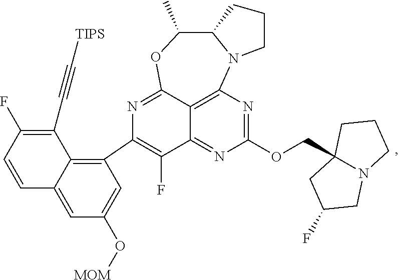

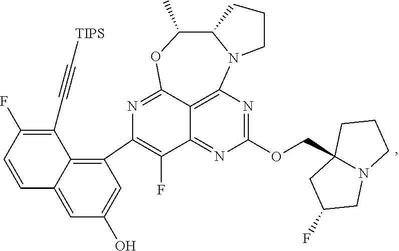

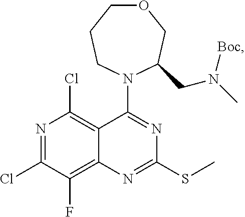 [SMILES: CSc1nc(N2CCCOC[C@H]2CN(C)C(=O)OC(C)(C)C)c2c(Cl)nc(Cl)c(F)c2n1]

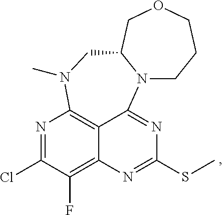 [SMILES: CSc1nc2c3c(nc(Cl)c(F)c3n1)N(C)C[C@@H]1COCCCN21]

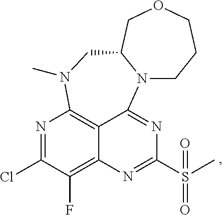 [SMILES: CN1C[C@@H]2COCCCN2c2nc(S(C)(=O)=O)nc3c(F)c(Cl)nc1c23]

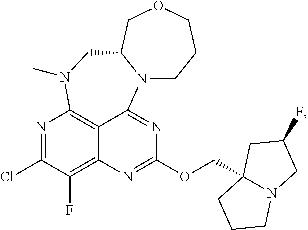 [SMILES: CN1C[C@@H]2COCCCN2c2nc(OC[C@@]34CCCN3C[C@H](F)C4)nc3c(F)c(Cl)nc1c23]

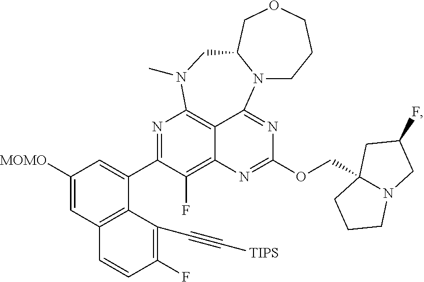

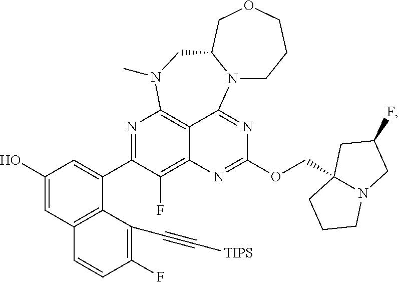

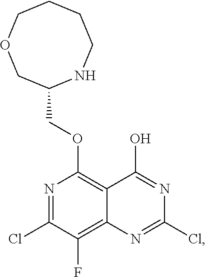 [SMILES: Oc1nc(Cl)nc2c(F)c(Cl)nc(OC[C@@H]3COCCCCN3)c12]

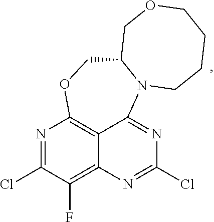 [SMILES: Fc1c(Cl)nc2c3c(nc(Cl)nc13)N1CCCCOC[C@H]1CO2]

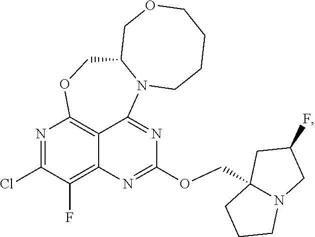 [SMILES: Fc1c(Cl)nc2c3c(nc(OC[C@@]45CCCN4C[C@H](F)C5)nc13)N1CCCCOC[C@H]1CO2]

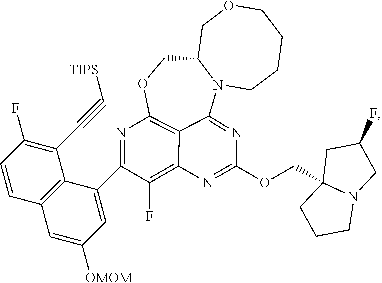

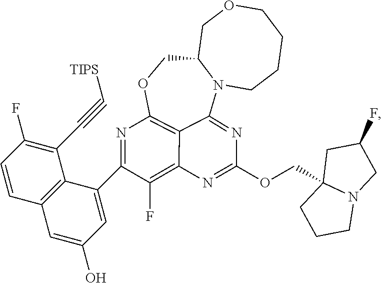

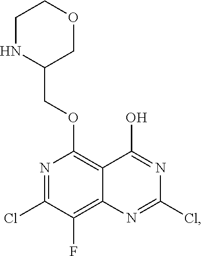 [SMILES: Oc1nc(Cl)nc2c(F)c(Cl)nc(OCC3COCCN3)c12]

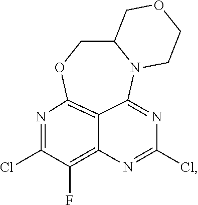 [SMILES: Fc1c(Cl)nc2c3c(nc(Cl)nc13)N1CCOCC1CO2]

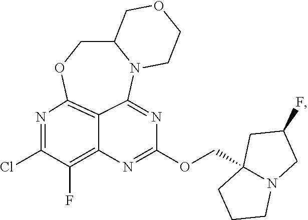 [SMILES: Fc1c(Cl)nc2c3c(nc(OC[C@@]45CCCN4C[C@H](F)C5)nc13)N1CCOCC1CO2]

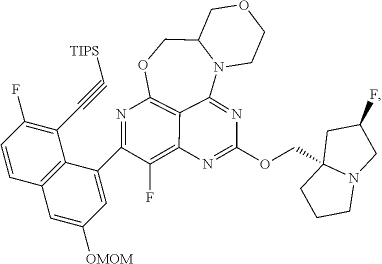

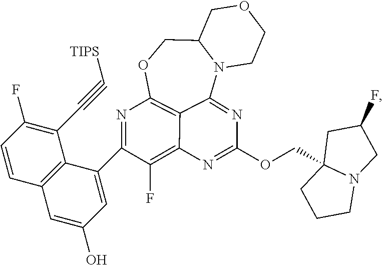

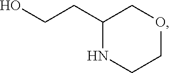

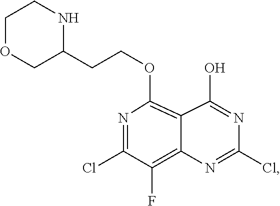 [SMILES: Oc1nc(Cl)nc2c(F)c(Cl)nc(OCCC3COCCN3)c12]

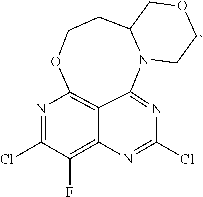 [SMILES: Fc1c(Cl)nc2c3c(nc(Cl)nc13)N1CCOCC1CCO2]

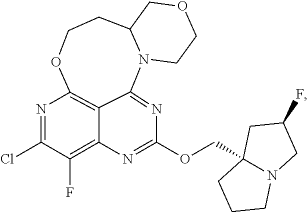 [SMILES: Fc1c(Cl)nc2c3c(nc(OC[C@@]45CCCN4C[C@H](F)C5)nc13)N1CCOCC1CCO2]

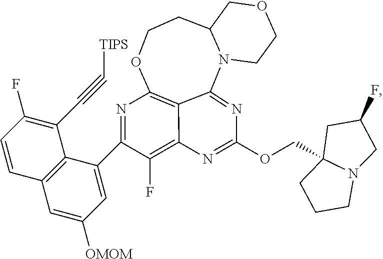

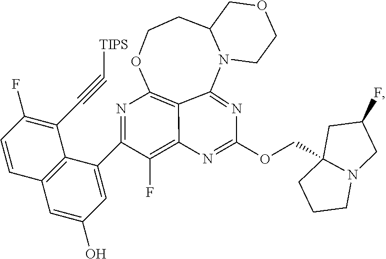

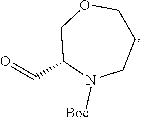 [SMILES: CC(C)(C)OC(=O)N1CCCOC[C@H]1C=O]

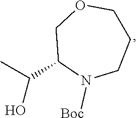 [SMILES: CC(O)[C@@H]1COCCCN1C(=O)OC(C)(C)C]

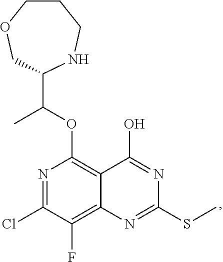 [SMILES: CSc1nc(O)c2c(OC(C)[C@@H]3COCCCN3)nc(Cl)c(F)c2n1]

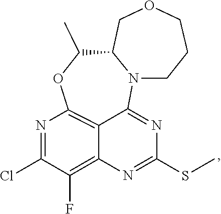

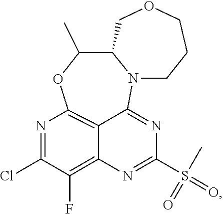 [SMILES: CC1Oc2nc(Cl)c(F)c3nc(S(C)(=O)=O)nc(c23)N2CCCOC[C@@H]12]

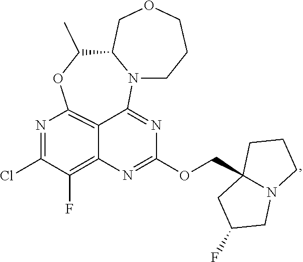

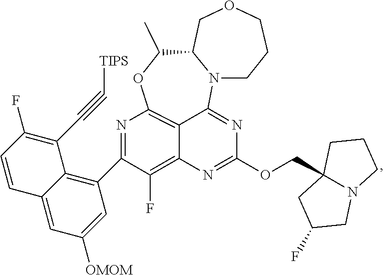

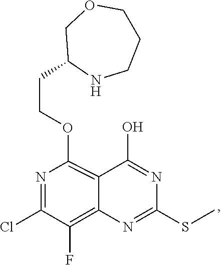 [SMILES: CSc1nc(O)c2c(OCC[C@@H]3COCCCN3)nc(Cl)c(F)c2n1]

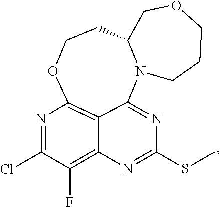 [SMILES: CSc1nc2c3c(nc(Cl)c(F)c3n1)OCC[C@@H]1COCCCN21]

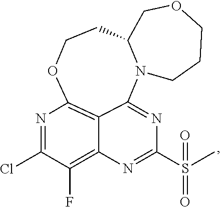 [SMILES: CS(=O)(=O)c1nc2c3c(nc(Cl)c(F)c3n1)OCC[C@@H]1COCCCN21]

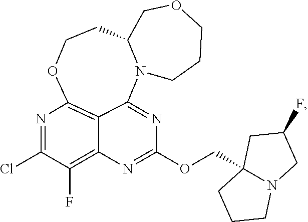 [SMILES: Fc1c(Cl)nc2c3c(nc(OC[C@@]45CCCN4C[C@H](F)C5)nc13)N1CCCOC[C@H]1CCO2]

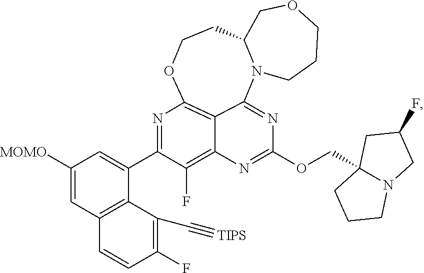

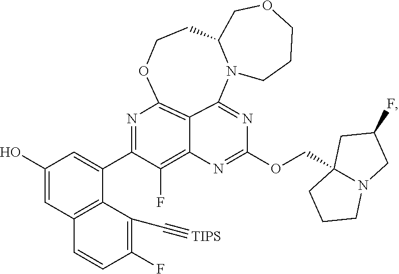

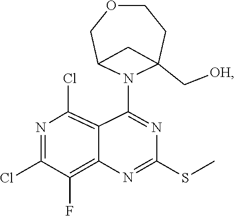 [SMILES: CSc1nc(N2C3COCCC2(CO)C3)c2c(Cl)nc(Cl)c(F)c2n1]

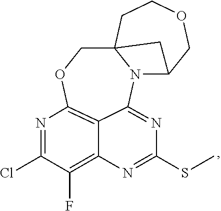 [SMILES: CSc1nc2c3c(nc(Cl)c(F)c3n1)OCC13CCOCC(C1)N23]

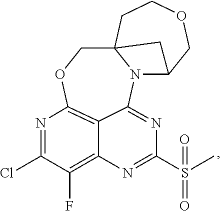 [SMILES: CS(=O)(=O)c1nc2c3c(nc(Cl)c(F)c3n1)OCC13CCOCC(C1)N23]

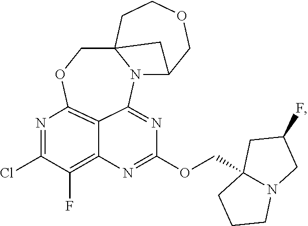 [SMILES: Fc1c(Cl)nc2c3c(nc(OC[C@@]45CCCN4C[C@H](F)C5)nc13)N1C3COCCC1(CO2)C3]

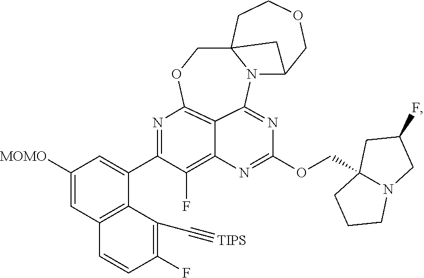

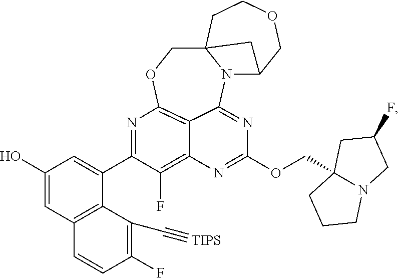

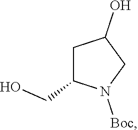 [SMILES: CC(C)(C)OC(=O)N1CC(O)C[C@H]1CO]

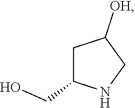

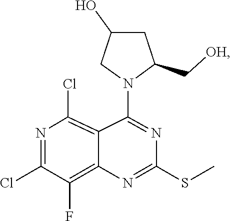 [SMILES: CSc1nc(N2CC(O)C[C@H]2CO)c2c(Cl)nc(Cl)c(F)c2n1]

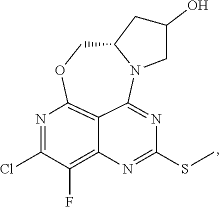 [SMILES: CSc1nc2c3c(nc(Cl)c(F)c3n1)OC[C@@H]1CC(O)CN21]

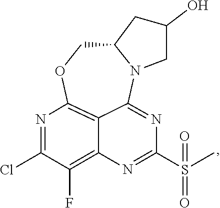 [SMILES: CS(=O)(=O)c1nc2c3c(nc(Cl)c(F)c3n1)OC[C@@H]1CC(O)CN21]

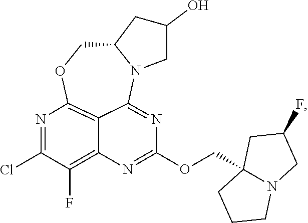 [SMILES: OC1C[C@H]2COc3nc(Cl)c(F)c4nc(OC[C@@]56CCCN5C[C@H](F)C6)nc(c34)N2C1]

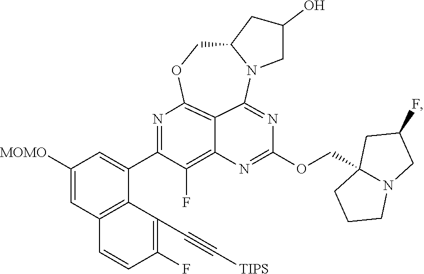

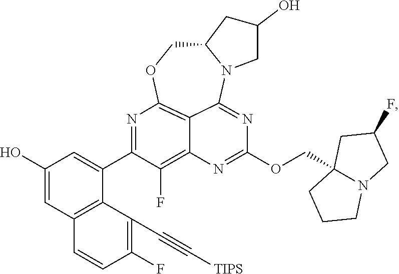

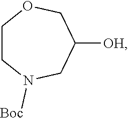 [SMILES: CC(C)(C)OC(=O)N1CCOCC(O)C1]

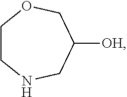

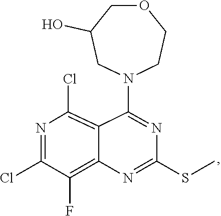 [SMILES: CSc1nc(N2CCOCC(O)C2)c2c(Cl)nc(Cl)c(F)c2n1]

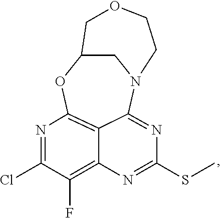 [SMILES: CSc1nc2c3c(nc(Cl)c(F)c3n1)OC1COCCN2C1]

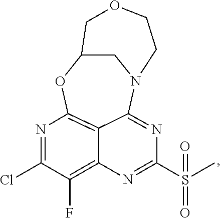 [SMILES: CS(=O)(=O)c1nc2c3c(nc(Cl)c(F)c3n1)OC1COCCN2C1]

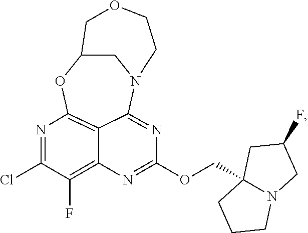 [SMILES: Fc1c(Cl)nc2c3c(nc(OC[C@@]45CCCN4C[C@H](F)C5)nc13)N1CCOCC(C1)O2]

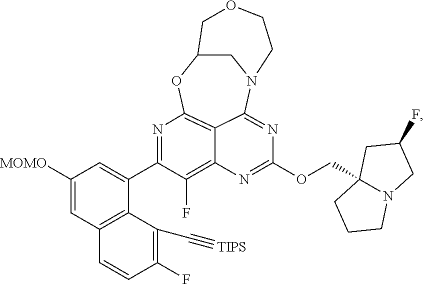

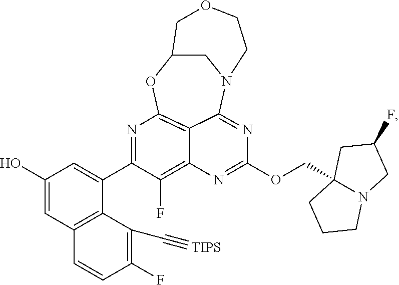

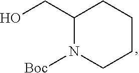 [SMILES: CC(C)(C)OC(=O)N1CCCCC1CO]

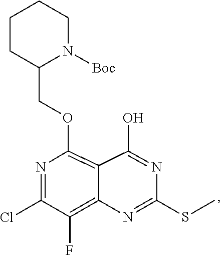 [SMILES: CSc1nc(O)c2c(OCC3CCCCN3C(=O)OC(C)(C)C)nc(Cl)c(F)c2n1]

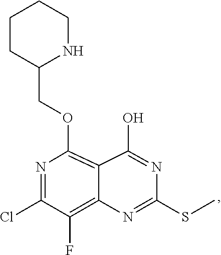 [SMILES: CSc1nc(O)c2c(OCC3CCCCN3)nc(Cl)c(F)c2n1]

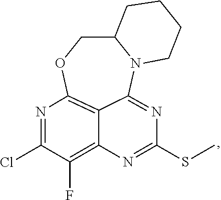 [SMILES: CSc1nc2c3c(nc(Cl)c(F)c3n1)OCC1CCCCN21]

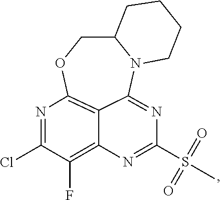 [SMILES: CS(=O)(=O)c1nc2c3c(nc(Cl)c(F)c3n1)OCC1CCCCN21]

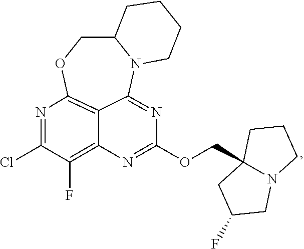 [SMILES: Fc1c(Cl)nc2c3c(nc(OC[C@@]45CCCN4C[C@H](F)C5)nc13)N1CCCCC1CO2]

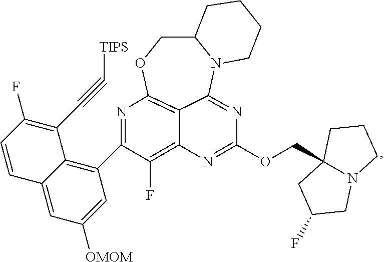

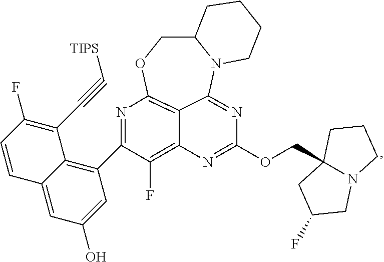

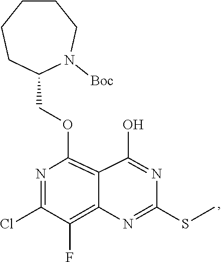 [SMILES: CSc1nc(O)c2c(OC[C@@H]3CCCCCN3C(=O)OC(C)(C)C)nc(Cl)c(F)c2n1]

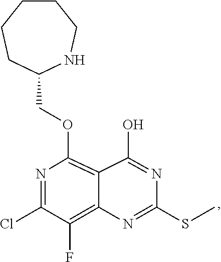 [SMILES: CSc1nc(O)c2c(OC[C@@H]3CCCCCN3)nc(Cl)c(F)c2n1]

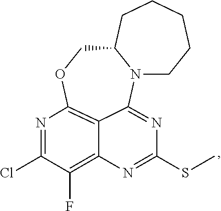 [SMILES: CSc1nc2c3c(nc(Cl)c(F)c3n1)OC[C@@H]1CCCCCN21]

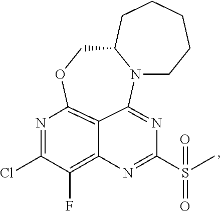 [SMILES: CS(=O)(=O)c1nc2c3c(nc(Cl)c(F)c3n1)OC[C@@H]1CCCCCN21]

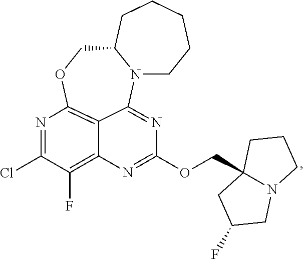 [SMILES: Fc1c(Cl)nc2c3c(nc(OC[C@@]45CCCN4C[C@H](F)C5)nc13)N1CCCCC[C@H]1CO2]

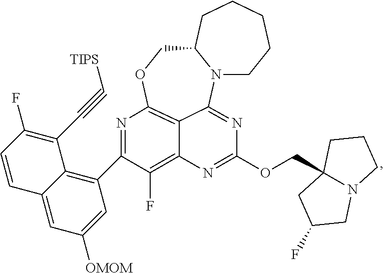

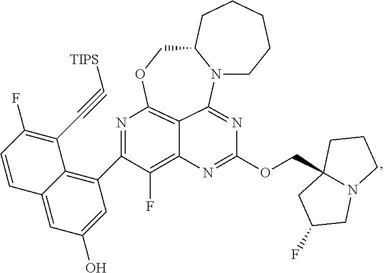

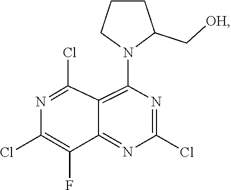 [SMILES: OCC1CCCN1c1nc(Cl)nc2c(F)c(Cl)nc(Cl)c12]

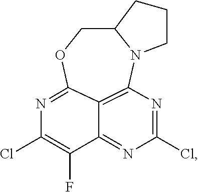 [SMILES: Fc1c(Cl)nc2c3c(nc(Cl)nc13)N1CCCC1CO2]

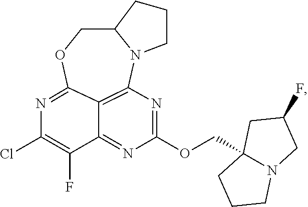 [SMILES: Fc1c(Cl)nc2c3c(nc(OC[C@@]45CCCN4C[C@H](F)C5)nc13)N1CCCC1CO2]

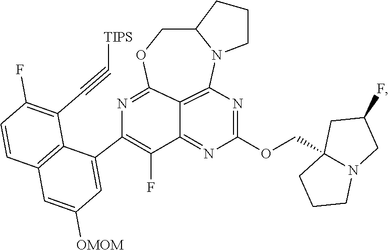

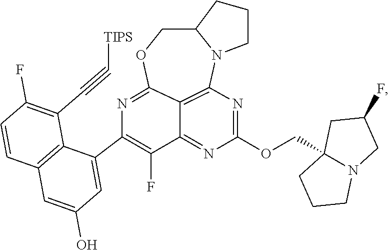

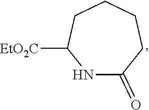 [SMILES: CCOC(=O)C1CCCCC(=O)N1]

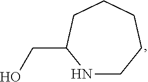

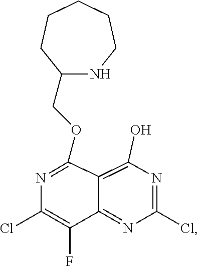 [SMILES: Oc1nc(Cl)nc2c(F)c(Cl)nc(OCC3CCCCCN3)c12]

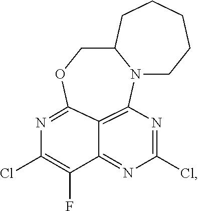 [SMILES: Fc1c(Cl)nc2c3c(nc(Cl)nc13)N1CCCCCC1CO2]

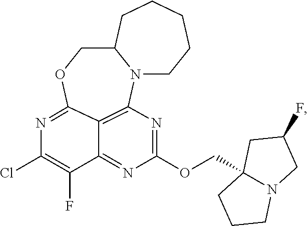 [SMILES: Fc1c(Cl)nc2c3c(nc(OC[C@@]45CCCN4C[C@H](F)C5)nc13)N1CCCCCC1CO2]

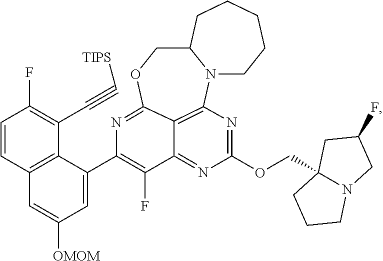

or

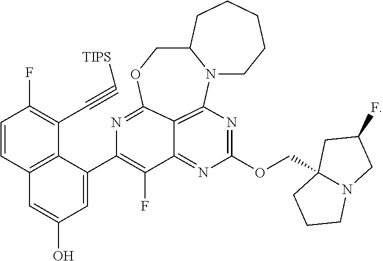

[29]. A pharmaceutical composition, comprising a therapeutically effective amount of the compound of formula (I), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [28], and a pharmaceutically acceptable excipient.

[30]. A method for treating cancer in a subject comprising administering a therapeutically effective amount of the compound of formula (I), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [28], or the pharmaceutical composition of [29] to a subject in need thereof.

[31]. A method for treating cancer in a subject in need thereof, the method comprising:
(a) determining whether the cancer is associated with K-Ras G12C, K-Ras G12D), K-Ras G12V, K-Ras G13D, K-Ras G12R, K-Ras G12S, K-Ras G12A, K-Ras Q61H mutation and/or K-Ras wild type amplification; and
(b) if so, administering a therapeutically effective amount of the compound of formula (I), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [28], or the pharmaceutical composition of [29] to the subject in need thereof.

[32]. The compound of formula (I), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [28], or the pharmaceutical composition of [29] for use in therapy.

[33]. The compound of formula (I), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [28], or the pharmaceutical composition of [29] for use as a medicament.

[34]. The compound of formula (I), the stereoisomer thereof, the pharmaceutically acceptable salt thereof, the pharmaceutically acceptable salt of the stereoisomer thereof, the prodrug thereof, the deuterated molecule thereof or the conjugated form thereof of any one of [1] to [28], or the pharmaceutical composition of [29] for use in a method for the treatment of cancer.

[35]. A use of the compound of formula (I), a stereoisomer thereof, a pharmaceutically acceptable salt thereof, a pharmaceutically acceptable salt of the stereoisomer thereof, a prodrug thereof, a deuterated molecule thereof or a conjugated form thereof of any one of [1] to [28], or the pharmaceutical composition of [29] for the treatment of cancer.

[36]. A use of the compound of formula (I), a stereoisomer thereof, a pharmaceutically acceptable salt thereof, a pharmaceutically acceptable salt of the stereoisomer thereof, a prodrug thereof, a deuterated molecule thereof or a conjugated form thereof of any one of [1] to [28], or the pharmaceutical composition of [29] for the manufacture of a medicament for the treatment of cancer.

[37]. The method for treating cancer of [30], the use in a method for the treatment of cancer of [34], the use for the treatment of cancer of [35], or the use for the manufacture of a medicament for the treatment of cancer of [36], wherein, said cancer is selected from pancreatic carcinoma, colorectal carcinoma, lung carcinoma (such as non-small cell lung cancer), breast carcinoma, large intestine carcinoma, stomach carcinoma, endometrial carcinoma, esophageal carcinoma or gastroesophageal junction carcinoma.

[38]. The method for treating cancer of [30] or [37], the use in a method for the treatment of cancer of [34] or [37], the use for the treatment of cancer of [35] or [37], or the use for the manufacture of a medicament for the treatment of cancer of [36] or [37], wherein, the cancer is associated with at least one of K-Ras G12C, K-Ras G12D, K-Ras G12V, K-Ras G13D, K-Ras G12R, K-Ras G12S, K-Ras G12A, K-Ras Q61H mutation and/or K-Ras wild type amplification.

[39]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras G12C associated cancer.

[40]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras G12D associated cancer.

[41]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras G12V associated cancer.

[42]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras G13D associated cancer.

[43]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras G12R associated cancer.

[44]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras G12S associated cancer.

[45]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras G12A associated cancer.

[46]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras Q61H associated cancer.

[47]. The method for treating cancer of [30], [37] or [38], the use in a method for the treatment of cancer of [34], [37] or [38], the use for the treatment of cancer of [35], [37] or [38], or the use for the manufacture of a medicament for the treatment of cancer of [36], [37] or [38], wherein, the cancer a K-Ras wild type amplification associated cancer.

### Definitions

Unless defined otherwise, all technical and scientific terms used herein have the same meaning as is commonly understood by one of skill in the art to which this invention belongs. All patents, patent applications, and publications referred to herein are incorporated by reference.

The term “a”, “an”, “the” and similar terms, as used herein, unless otherwise indicated, are to be construed to cover both the singular and plural.

The term “halogen” or “halo”, as used interchangeably herein, unless otherwise indicated, refers to fluoro, chloro, bromo or iodo. The preferred halogen groups include —F, —Cl and —Br.

The term “alkyl”, as used herein, unless otherwise indicated, refers to saturated monovalent hydrocarbon radicals having straight or branched arrangement. C₁₋₁₀ in —C₁₋₁₀alkyl is defined to identify the group having 1, 2, 3, 4, 5, 6, 7, 8, 9 or 10 carbon atoms in a linear or branched arrangement. Non-limiting alkyl includes methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl.

The term “haloalkyl”, as used herein, unless otherwise indicated, refers to the above-mentioned alkyl substituted with one or more (for example 1, 2, 3, 4, 5, or 6) halogen (such as —F, —Cl or —Br). In some embodiments, the haloalkyl is interchangeable —C₁₋₁₀haloalkyl or haloC₁₋₁₀alkyl, wherein, C₁₋₁₀ in the —C₁₋₁₀haloaklyl or haloC₁₋₁₀alkyl indicates that the total carbon atoms of the alkyl are 1 to 10. In some embodiments, the —C₁₋₁₀haloalkyl is the —C₁₋₆haloalkyl. In some embodiments, the —C₁₋₆haloalkyl is the —C₁₋₃haloalkyl. In some embodiments, the —C₁₋₃haloalkyl is (methyl, ethyl, propyl or isopropyl) substituted with 1, 2, 3, 4, 5, or 6 —F; preferably, the —C₁₋₃haloalkyl is —CF₃.

The term “alkylene”, as used herein, unless otherwise indicated, refers to a divalent group obtained by removal of an additional hydrogen atom from an alkyl group defined above. In some embodiments, the alkylene is C₀₋₆alkylene. In some embodiments, the C₀₋₆alkylene is C₀₋₃alkylene. The C₀₋₆ in the front of the alkylene indicates the total carbon atoms in the alkylene are 0 to 6 and C₀ indicates the two ends of the alkylene are connected directly. Non-limiting alkylene includes methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl”, as used herein, unless otherwise indicated, refers to a straight or branch-chained hydrocarbon radical containing one or more double bonds and typically from 2 to 20 carbon atoms in length. In some embodiments, the alkenyl is —C₂₋₁₀alkenyl. In some embodiments, the —C₂₋₁₀alkenyl is —C₂₋₆alkenyl which contains from 2 to 6 carbon atoms. Non-limiting alkenyl includes ethenyl, propenyl, butenyl, 2-methyl-2-buten-1-yl, hepetenyl, octenyl and the like.

The term “haloalkenyl”, as used herein, unless otherwise indicated, refers to the above-mentioned alkenyl substituted with one or more (for example 1, 2, 3, 4, 5, or 6) halogen (such as —F, —Cl or —Br). In some embodiments, the haloalkenyl is interchangeable —C₂₋₁₀haloalkenyl or haloC₂₋₁₀alkenyl, wherein, C₂₋₁₀ in the —C₂₋₁₀haloaklenyl or haloC₂₋₁₀alkenyl indicates that the total carbon atoms of the alkenyl are 2 to 10. In some embodiments, the —C₂₋₁₀haloalkenyl is the —C₂₋₆haloalkenyl. In some embodiments, the —C₂₋₆haloalkenyl is the —C₂₋₃haloalkenyl. In some embodiments, the —C₂₋₃haloalkenyl is (ethenyl or propenyl) substituted with 1, 2, 3, 4, 5, or 6 —F.

The term “alkynyl”, as used herein, unless otherwise indicated, refers to a straight or branch-chained hydrocarbon radical containing one or more triple bonds and typically from 2 to 20 carbon atoms in length. In some embodiments, the alkynyl is —C₂₋₁₀alkynyl. In some embodiments, the —C₂₋₁₀alkynyl is —C₂₋₆alkynyl which contains from 2 to 6 carbon atoms. Non-limiting alkynyl includes ethynyl, 1-propynyl, 1-butynyl, heptynyl, octynyl and the like.

The term “haloalkynyl”, as used herein, unless otherwise indicated, refers to the above-mentioned alkynyl substituted with one or more (for example 1, 2, 3, 4, 5, or 6) halogen (such as —F, —Cl or —Br). In some embodiments, the haloalkynyl is interchangeable —C₂₋₁₀haloalkynyl or haloC₂₋₁₀alkynyl, wherein, C₂₋₁₀ in the —C₂₋₁₀haloaklynyl or haloC₂₋₁₀alkynyl indicates that the total carbon atoms of the alkynyl are 2 to 10. In some embodiments, the —C₂₋₁₀haloalkynyl is the —C₂₋₆haloalkynyl. In some embodiments, the —C₂₋₆haloalkynyl is the —C₂₋₃haloalkynyl. In some embodiments, the —C₂₋₃haloalkynyl is (ethynyl or propynyl) substituted with 1, 2, 3, 4, 5, or 6 —F.

The term “alkoxy”, as used herein, unless otherwise indicated, refers to oxygen ethers formed from the previously described alkyl groups.

The term “haloalkoxy”, as used herein, unless otherwise indicated, refers to the above-mentioned alkoxy substituted with one or more (for 1, 2, 3, 4, 5, or 6) halogen (—F, —Cl or —Br). In some embodiment, the haloalkoxy is interchangeable —C₁₋₁₀haloalkoxy or haloC₁₋₁₀alkoxy. In some embodiments, the haloalkoxy is interchangeable —C₁₋₆haloalkoxy or haloC₁₋₆alkoxy, wherein, C₁₋₆ in the —C₁₋₆haloakloxy or haloC₁₋₆alkoxy indicates that the total carbon atoms of the alkoxy are 1 to 6. In some embodiments, the —C₁₋₆haloalkoxy is the —C₁₋₃haloalkoxy. In some embodiments, the —C₁₋₃haloalkoxy is (methoxy, ethoxy, propoxy or isopropoxy) substituted with 1, 2, 3, 4, 5, or 6 —F; preferably, the —C₁₋₃haloalkoxy is —OCF₃.

The term “carbocyclic ring”, as used herein, unless otherwise indicated, refers to a totally saturated or partially saturated monocyclic, bicyclic, bridged, fused, or spiro non-aromatic ring only containing carbon atoms as ring members. The term “carbocyclyl” as used herein, unless otherwise indicated, means a monovalent group obtained by removal of a hydrogen atom on the ring carbon atom from the carbocyclic ring defined in the present invention. The carbocyclic ring is interchangeable with the carbocyclyl ring in the present invention. In some embodiments, the carbocyclic ring is a three to twenty membered (such as 3-, 4-, 5-, 6-, 7-, 8-, 9-, 10-, 11-, 12-, 13-, 14-, 15-, 16-, 17-, 18-, 19- or 20-membered) carbocyclic ring and is either fully saturated or has one or more degrees of unsaturation. Multiple degrees of substitution, for example, one, two, three, four, five or six, are included within the present definition. The carbocyclic ring includes a cycloalkyl ring in which all ring carbon atoms are saturated, a cycloalkenyl ring which contains at least one double bond (preferred contain one double bond), and a cycloalkynyl ring which contains at least one triple bond (preferred contain one triple bond). Cycloalkyl includes but not limited to cyclopropyl, cyclobutyl, cyclopentyl, cyclohexyl, cycloheptyl, cyclooctyl, cyclononyl, cyclodecyl and the like. Cycloalkenyl includes but not limited to cyclopentenyl, cyclohexenyl, cycloheptenyl, cyclooctenyl, cyclononenyl, cyclodecenyl and the like. The carbocyclyl ring includes a monocyclic carbocyclyl ring, and a bicyclic or polycyclic carbocyclyl ring in which one, two or three or more atoms are shared between the rings. The term “spirocyclic carbocyclic ring” refers to a carbocyclic ring in which each of the rings only shares one ring atom with the other ring. In some embodiments, the spirocyclic ring is bicyclic spirocyclic ring. The spirocyclic carbocyclic ring includes a spirocyclic cycloalkyl ring and a spirocyclic cycloalkenyl ring and a spirocyclic cycloalkynyl ring. The term “fused carbocyclic ring” refers to a carbocyclic ring in which each of the rings shares two adjacent ring atoms with the other ring. In some embodiments, the fused ring is a bicyclic fused ring. The fused carbocyclic ring includes a fused cycloalkyl ring and a fused cycloalkenyl ring and a fused cycloalkynyl ring. A monocyclic carbocyclic ring fused with an aromatic ring (such as phenyl) is included in the definition of the fused carbocyclic ring. The term “bridged carbocyclic ring” refers to a carbocyclic ring that includes at least two bridgehead carbon ring atoms and at least one bridging carbon atom. In some embodiments, the bridged ring is bicyclic bridged ring. The bridged carbocyclic ring includes a bicyclic bridged carbocyclic ring which includes two bridgehead carbon atoms and a polycyclic bridged carbocyclic ring which includes more than two bridgehead carbon atoms. The bridged carbocyclic ring includes a bridged cycloalkyl ring, a bridged cycloalkenyl ring and a bridged cycloalkynyl ring. Examples of monocyclic carbocyclyl and bicyclic carbocyclyl include but not limit to cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, and 1-cyclohex-3-enyl.

The term “heterocyclic ring”, as used herein, unless otherwise indicated, refers to a totally saturated or partially saturated monocyclic, bicyclic, bridged, fused, or spiro non-aromatic ring containing not only carbon atoms as ring members and but also containing one or more (such as 1, 2, 3, 4, 5, or 6) heteroatoms as ring members. Preferred heteroatoms include N, O, S, N-oxides, sulfur oxides, and sulfur dioxides. The term “heterocyclyl” as used herein, unless otherwise indicated, means a monovalent group obtained by removal of a hydrogen atom on the ring carbon atom or the ring heteroatom from the heterocyclic ring defined in the present invention. The heterocyclic ring is interchangeable with the heterocyclyl ring in the present invention. In some embodiments, the heterocyclic ring is a three to twenty membered (such as 3-, 4-, 5-, 6-, 7-, 8-, 9-, 10-, 11-, 12-, 13-, 14-, 15-, 16-, 17-, 18-, 19- or 20-membered) heterocyclic ring and is either fully saturated or has one or more degrees of unsaturation. Multiple degrees of substitution, for example, one, two, three, four, five or six, are included within the present definition. The heterocyclic ring includes a heterocycloalkyl ring in which all ring carbon atoms are saturated, a heterocycloalkenyl ring which contains at least one double bond (preferred contain one double bond), and a heterocycloalkynyl ring which contains at least one triple bond (preferred contain one triple bond). The heterocyclyl ring includes a monocyclic heterocyclyl ring, and a bicyclic or polycyclic heterocyclyl ring in which one, two or three or more atoms are shared between the rings. The term “spirocyclic heterocyclic ring” refers to a heterocyclic ring in which each of the rings only shares one ring atom with the other ring. In some embodiments, the spirocyclic ring is bicyclic spirocyclic ring. The spirocyclic heterocyclic ring includes a spirocyclic heterocycloalkyl ring and a spirocyclic heterocycloalkenyl ring and a spirocyclic heterocycloalkynyl ring. The term “fused heterocyclic ring” refers to a heterocyclic ring in which each of the rings shares two adjacent ring atoms with the other ring. In some embodiments, the fused ring is a bicyclic fused ring. The fused heterocyclic ring includes a fused heterocycloalkyl ring and a fused heterocycloalkenyl ring and a fused heterocycloalkynyl ring. A monocyclic heterocyclic ring fused with an aromatic ring (such as phenyl) is included in the definition of the fused heterocyclic ring. The term “bridged heterocyclic ring” refers to a heterocyclic ring that includes at least two bridgehead ring atoms and at least one bridging atom. In some embodiments, the bridged ring is bicyclic bridged ring. The bridged heterocyclic ring includes a bicyclic bridged heterocyclic ring which includes two bridgehead atoms and a polycyclic bridged heterocyclic ring which includes more than two bridgehead atoms. The bridged heterocyclic ring includes a bridged heterocycloalkyl ring, a bridged heterocycloalkenyl ring and a bridged heterocycloalkynyl ring. Examples of such heterocyclyl include but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “aryl”, as used herein, unless otherwise indicated, refers to a mono or polycyclic aromatic ring system only containing carbon ring atoms. The preferred aryls are monocyclic or bicyclic 6-10 membered aromatic rings. Phenyl and naphthyl are preferred aryls.

The term “heteroaryl”, as used herein, unless otherwise indicated, refers to an aromatic ring containing carbons and one or more (such as 1, 2, 3 or 4) heteroatoms selected from N, O or S. The heteroaryl may be monocyclic or polycyclic. A monocyclic heteroaryl group may have 1 to 4 heteroatoms in the ring, while a polycyclic heteroaryl may contain 1 to 10 heteroatoms. A polycyclic heteroaryl ring may contain fused ring junction, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms), preferred monocyclic heteroaryl is 5 membered heteroaryl including 1, 2, 3 or 4 heteroatoms selected from N, O or S, or 6 membered heteroaryl including 1 or 2 heteroatoms selected from N. Examples of heteroaryl groups include, but not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladeninyl, quinolinyl or isoquinolinyl.

The term “one or more”, as used herein, unless otherwise indicated, refers to one or more than one. In some embodiments, “one or more” refers to 1, 2, 3, 4, 5 or 6. In some embodiments, “one or more” refers to 1, 2, 3 or 4. In some embodiments, “one or more” refers to 1, 2, or 3. In some embodiments, “one or more” refers to 1 or 2. In some embodiments, “one or more” refers to 1. In some embodiments, “one or more” refers to 2. In some embodiments, “one or more” refers to 3. In some embodiments, “one or more” refers to 4. In some embodiments, “one or more” refers to 5. In some embodiments, “one or more” refers to 6.

The term “substituted”, as used herein, unless otherwise indicated, refers to a hydrogen atom on the carbon atom or a hydrogen atom on the nitrogen atom is replaced by a substituent. When one or more substituents are substituted on a ring in the present invention, it means that each of substituents may be respectively independently substituted on every ring atom of the ring including but not limited to a ring carbon atom or a ring nitrogen atom. In addition, when the ring is a polycyclic ring, such as a fused ring, a bridged ring or a spiro ring, each of substituents may be respectively independently substituted on every ring atom of the polycyclic ring.

The term “oxo” refers to oxygen atom together with the attached carbon atom forms the group

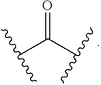 [SMILES: *C(*)=O]

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The term “pharmaceutically acceptable salt” refers to a salt prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrug of the compounds of this invention. In general, such prodrug will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers. The term “stereoisomer” as used in the present invention refers to an isomer in which atoms or groups of atoms in the molecule are connected to each other in the same order but differ in spatial arrangement, including conformational isomers and configuration isomers. The configuration isomers include geometric isomers and optical isomers, and optical isomers mainly include enantiomers and diastereomers. The invention includes all possible stereoisomers of the compound.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent.

The term “deuterated derivative”, used herein, unless otherwise indicated, refers to a compound having the same chemical structure as a reference compound, but with one or more hydrogen atoms replaced by a deuterium atom (“D”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivative described herein. Thus, unless otherwise stated, when a reference is made to a “deuterated derivative” of a compound of the disclosure, at least one hydrogen is replaced with deuterium at well above its natural isotopic abundance (which is typically about 0.015%) In some embodiments, the deuterated derivative of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at lease 6333.3 (95% deuterium incorporation, at least 6466.7 (97% deuterium incorporation, or at least 6600 (99% deuterium incorporation).

When a tautomer of the compound in the present invention exists, the present invention includes any possible tautomer and pharmaceutically acceptable salts thereof, and mixtures thereof, except where specifically stated otherwise.

The “conjugated form” refers to herein that the compound described herein is conjugated to another agent through a linker or not through a linker, wherein, the compound functions as a binder or a inhibitor of K-Ras protein (including K-Ras G12C, K-Ras G12D, K-Ras G12V, K-Ras G13D, K-Ras G12R, K-Ras G12S, K-Ras G12A, K-Ras Q61H mutant protein and K-Ras wild type protein) For example, the conjugated form is a PROTAC molecule, e.g. the compound is incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. Preferably, the portion of the PROTAC that engages the E3 ligase is connected to the portion of the PROTAC that engages the target protein via a linker which consists of a variable chain of atoms. Recruitment of K-Ras protein to the E3 ligase will thus result in the destruction of the K-Ras protein. The variable chain of atoms can include, for example, rings, heteroatoms, and/or repeating polymeric units. It can be rigid or flexible. It can be attached to the two portions described above using standard techniques in the art of organic synthesis.

The pharmaceutical compositions of the present invention comprise a compound in present invention (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, a compound of formula (I), a stereoisomer thereof, a pharmaceutically acceptable salt thereof, a pharmaceutically acceptable salt of the stereoisomer thereof, a prodrug thereof, a deuterated molecule thereof or a conjugated form thereof as defined herein can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as oil-in-water emulsion or as a water-in-oil liquid emulsion. In addition to the common dosage forms set out above, the compound in the present invention or a pharmaceutically acceptable salt thereof may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt. The compounds of the present invention or pharmaceutically acceptable salts thereof can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound in the present invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described herein or pharmaceutically acceptable salts thereof, may also be prepared in powder or liquid concentrate form.

Unless otherwise apparent from the context, when a value is expressed as “about” X or “approximately” X, the stated value of X will be understood to be accurate to ±10%, preferably, ±5%, ±2%.

The term “subject” refers to an animal. In some embodiments, the animal is a mammal. A subject also refers to for example, primates (e.g., humans), cows, sheep, goats, horses, dogs, cats, rabbits, rats, mice, fish, birds and the like. In certain embodiments, the subject is a human. A “patient” as used herein refers to a human subject. As used herein, a subject is “in need of” a treatment if such subject would benefit biologically, medically or in quality of life from such treatment. In some embodiments, the subject has experienced and/or exhibited at least one symptom of cancer to be treated and/or prevented. In some embodiments, the subject has been identified or diagnosed as having a cancer having wild type K-Ras or a K-Ras G12A, K-Ras G12C, K-Ras G12D, K-Ras G12R, K-Ras G12S, K-Ras G12V, K-Ras G13D and/or K-Ras Q61H mutation

The term “inhibition”, “inhibiting” or “inhibit” refers to the reduction or suppression of a given condition, symptom, or disorder, or disease, or a significant decrease in the baseline activity of a biological activity or process.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, “K-Ras G12A” refers to a mutant form of a mammalian K-Ras protein that contains an amino acid substitution of an alanine for a glycine at amino acid position 12. A “K-Ras G12A inhibitor” refers to a compound is capable of negatively modulating or inhibiting all or a portion of the function of K-Ras G12A. A “K-Ras G12A associated cancer” as used herein refers to a cancer associated with or mediated by or having a K-Ras G12A mutation.

As used herein, “K-Ras G12C” refers to a mutant form of a mammalian K-Ras protein that contains an amino acid substitution of a cysteine for a glycine at amino acid position 12. A “K-Ras G12C inhibitor” refers to a compound is capable of negatively modulating or inhibiting all or a portion of the function of K-Ras G12C. A “K-Ras G12C associated cancer” as used herein refers to a cancer associated with or mediated by or having a K-Ras G12C mutation.

As used herein, “K-Ras G12D” refers to a mutant form of a mammalian K-Ras protein that contains an amino acid substitution of an aspartic acid for a glycine at amino acid position 12. A “K-Ras G12D inhibitor” refers to a compound is capable of negatively modulating or inhibiting all or a portion of the function of K-Ras G12D. A “K-Ras G12D associated cancer” as used herein refers to a cancer associated with or mediated by or having a K-Ras G12D mutation.

As used herein, “K-Ras G12R” refers to a mutant form of a mammalian K-Ras protein that contains an amino acid substitution of an arginine for a glycine at amino acid position 12. A “K-Ras G12R inhibitor” refers to a compound is capable of negatively modulating or inhibiting all or a portion of the function of K-Ras G12R. A “K-Ras G12R associated cancer” as used herein refers to a cancer associated with or mediated by or having a K-Ras G12R mutation.

As used herein, “K-Ras G12S” refers to a mutant form of a mammalian K-Ras protein that contains an amino acid substitution of a serine for a glycine at amino acid position 12. A “K-Ras G12S inhibitor” refers to a compound is capable of negatively modulating or inhibiting all or a portion of the function of K-Ras G12S. A “K-Ras G12S associated cancer” as used herein refers to a cancer associated with or mediated by or having a K-Ras G12S mutation.

As used herein, “K-Ras G12V” refers to a mutant form of a mammalian K-Ras protein that contains an amino acid substitution of a valine for a glycine at amino acid position 12. A “K-Ras G12V inhibitor” refers to a compound is capable of negatively modulating or inhibiting all or a portion of the function of K-Ras G12V. A “K-Ras G12V associated cancer” as used herein refers to a cancer associated with or mediated by or having a K-Ras G12V mutation.

As used herein, “K-Ras G13D” refers to a mutant form of a mammalian K-Ras protein that contains an amino acid substitution of an aspartic acid for a glycine at amino acid position 13. A “K-Ras G13D inhibitor” refers to a compound is capable of negatively modulating or inhibiting all or a portion of the function of K-Ras G13D. A “K-Ras G13D associated cancer” as used herein refers to a cancer associated with or mediated by or having a K-Ras G13D mutation.

As used herein, “K-Ras Q61H” refers to a mutant form of a mammalian K-Ras protein that contains an amino acid substitution of a histidine for a glutamine at amino acid position 61. A “K-Ras Q61H inhibitor” refers to a compound is capable of negatively modulating or inhibiting all or a portion of the function of K-Ras Q61H.

A “K-Ras Q61H associated cancer” as used herein refers to a cancer associated with or mediated by or having a K-Ras Q61H mutation.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

These and other aspects will become apparent from the following written description of the invention.

### Methods of Preparation

Compounds of the present invention can be synthesized from commercially available reagents using the synthetic methods and reaction schemes described herein. The examples which outline specific synthetic route, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### EXAMPLES

The following examples are provided to better illustrate the present invention. All parts and percentages are by weight and all temperatures are degrees Celsius, unless explicitly stated otherwise. The following abbreviations in the Table 10 have been used in the examples:

TABLE 10

| | |
|---|---|
| DMF | N,N-dimethylformamide |
| EA/EtOAc | Ethyl acetate |
| Hex | Hexane |
| MeOH | Methanol |
| DCM | Dichloromethane |
| DCE | 1,2-dichloroethane |
| EtOH | Ethanol |
| THF | Tetrahydrofuran |
| DIEA/DIPEA | N,N-Diisopropylethylamine |
| Pd(PPh₃)₄ | Tetrakis(triphenylphosphine)palladium |
| Pd(dppf)Cl₂ | [1,1′-Bis(diphenylphosphino)ferrocene]dichloropalladium(II) |
| TFA | 2,2,2-trifluoroacetic acid |
| ACN/MeCN/CH₃CN | Acetonitrile |
| Et₃N/TEA | Triethylamine |
| NIS | N-Iodosuccinimide |
| DMSO | Dimethyl sulfoxide |
| NCS | N-chlorosuccinimide |
| TBSCI | Tert-butyldimethylsilyl chloride |
| TMSCl | Trimethylsilyl chloride |
| MOMCl | Methoxymethyl chloride |
| MsCl | Methanesulfonyl chloride |
| LAH | Lithium aluminum hydride |
| LDA | Lithium diisopropylamide |
| LIHMDS | Lithium hexamethyldisilazide |
| B₂(Pin)₂ | Bis(pinacolato)diboron |
| NFSI | N-Fluorobenzenesulfonimide |
| MTBE | Methyl tert-butyl ether |
| DMAP | N,N-dimethylpyridin-4-amine |
| DABCO | Triethylenediamine |
| TBAF | Tetrabutylammonium fluoride |
| m-CPBA | 3-Chloroperbenzoic acid |
| NMP | N-methylpyrrolidone |
| rt/RT/R.T | Room temperature |
| min(s) | minute(s) |
| h/hr(s) | hour(s) |
| aq | aqueous |
| Sat. | saturated |
| TLC | Thin layer chromatography |
| Prep - TLC | Preparative thin layer chromatography |
| MOMO | Methoxymethoxyl |
| TIPS | Triisopropylsilyl |
| IPA | Isopropyl alcohol |
| cataCXium A Pd G3 | Methanesulfonato(diadamantyl-n-butylphosphino)-2′-amino-1,1′- |
| | biphenyl-2-yl)palladium(II) |
| 4A MS | 4A Molecular sieve |

### Preparation of the Intermediates

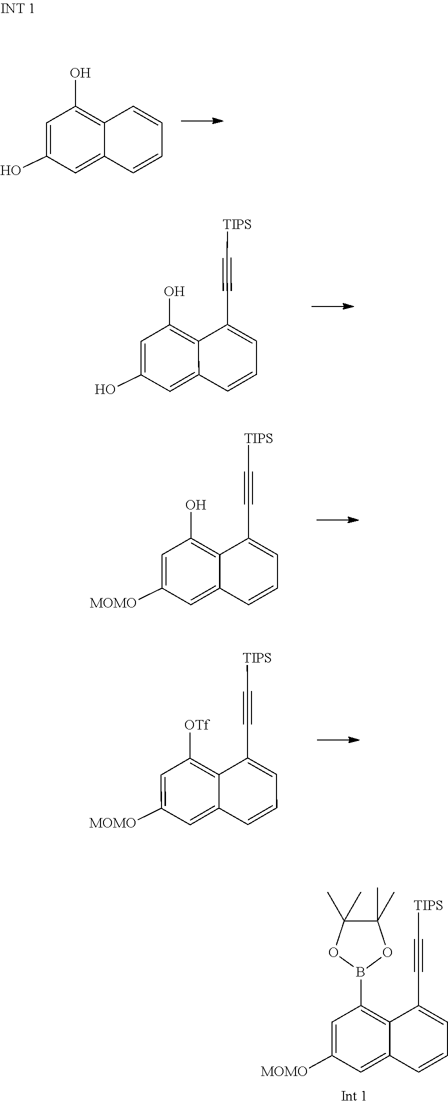

Following the procedure described in WO2021041671, INT 1 was synthesized with naphthalene-1,3-diol as starting material.

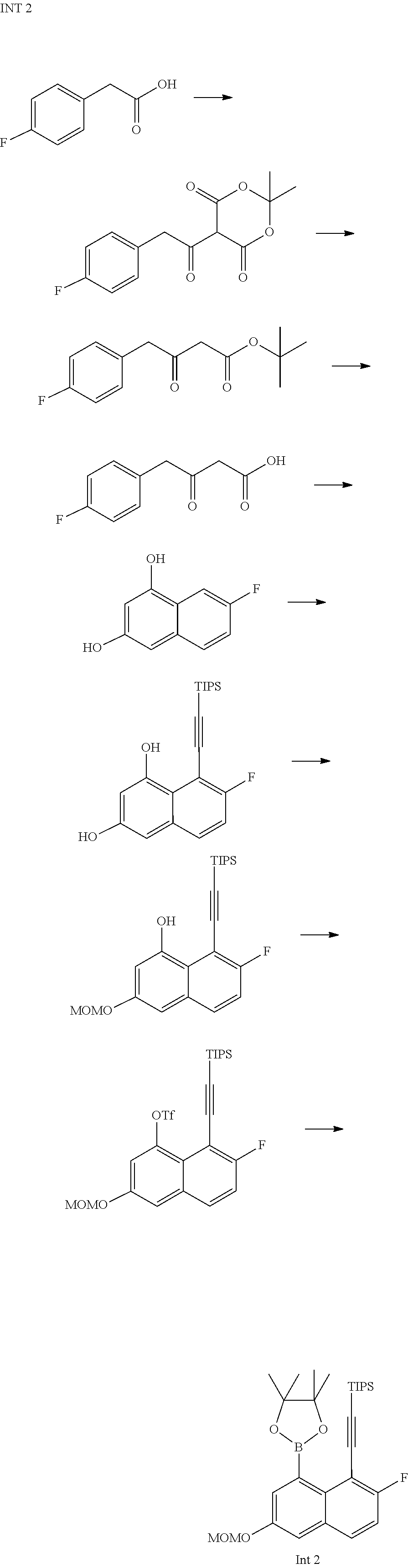

Following the procedure of WO2021041671, INT 2 was synthesized with 2-(4-fluorophenyl) acetic acid as starting material.

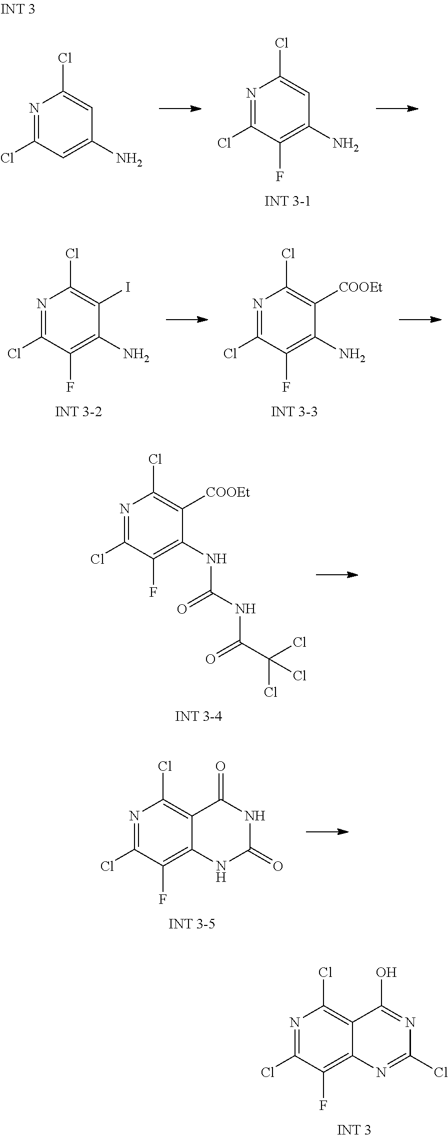

A mixture of 2,6-dichloropyridin-4-amine (35.7 g, 219.0 mmol), 1-(chloromethyl)-4-fluoro-1,4-diazoniabicyclo[2.2.2]octane ditetrafluoroborate (93.1 g, 262.8 mmol) in DMF (357 mL) and CH₃CN (357 mL) was stirred at 80° C. for 6 hours. The reaction mixture was quenched by water (400 mL), extracted with DCM (400 mL×3), the organic layer was combined, dried over anhydrous Na₂SO₄, filtered and concentrated under reduced pressure. The residue was purified by silica gel column (eluting with petroleum ether:EtOAc=30:1, v/v) to give INT 3-1 (12.6 g, purity: about 50%). MS (ESI, m/z): 181 [M+H]⁺.

A mixture of INT 3-1 (2.0 g, 11.05 mmol), NIS (2.98 g, 13.26 mmol) and p-toluenesulfonic acid monohydrate (105 mg, 0.55 mmol) in CH₃CN (8.4 mL) was stirred at 70° C. for 4 hours under nitrogen atmosphere. The reaction mixture was quenched by water (20 mL), extracted with EtOAc (20 mL×3), the organic layer was combined, dried over anhydrous Na₂SO₄, filtered and concentrated under reduced pressure. The residue was purified by silica gel column (eluting with petroleum ether:EtOAc=50:1˜ 20:1, v/v) to give INT 3-2 (3.6 g). MS (ESI, m/z): 307 [M+H]⁺.

A mixture of INT 3-2 (1.0 g, 3.26 mmol), Pd(PPh₃)₂Cl₂ (229 mg, 0.33 mmol) and Et₃N (1.19 g, 11.77 mmol) in EtOH (17.0 mL) was stirred at 80° C. for 20 hours under carbon monoxide atmosphere (1.5 MPa) in a sealed tube. The reaction mixture was filtered and the filtrate was concentrated under reduced pressure. The residue was purified by silica gel column to give INT 3-3 (1.2 g). MS (ESI, m/z): 253 [M+H]⁺.

A mixture of INT 3-3 (800 mg, 3.16 mmol), trichloroacetyl isocyanate (714 mg, 3.79 mmol) in THF (8 mL) was stirred at room temperature for 1 hour. The reaction mixture was concentrated under reduced pressure. The residue was triturated with MTBE to give INT 3-4 (880 mg).

A mixture of INT 3-4 (780 mg, 1.77 mmol), NH₃/MeOH (1.26 mL, 7M, 8.85 mmol) and MeOH (7.8 mL) was stirred at room temperature for 1 hour. The reaction mixture was concentrated under reduced pressure. The residue was triturated with MTBE to give INT 3-5 (550 mg). MS (ESI, m/z): 250 [M+H]⁺.

A mixture of INT 3-5 (375 mg, 1.50 mmol), DIPEA (595 mg, 4.60 mmol) and POCl₃ (15 mL) was stirred at 105° C. for 17 hours. The reaction mixture was concentrated under reduced pressure. The residue was diluted with 1,4-dioxane (5 mL) and the resulting solution was added dropwise to aq. K₂CO₃ (20%, 30 mL). The mixture was stirred for 2 hours at RT, adjust pH to 2˜3, filtered and the filter cake was collected and dried to give INT 3 (344 mg). MS (ESI, m/z): 268 [M+H]⁺.

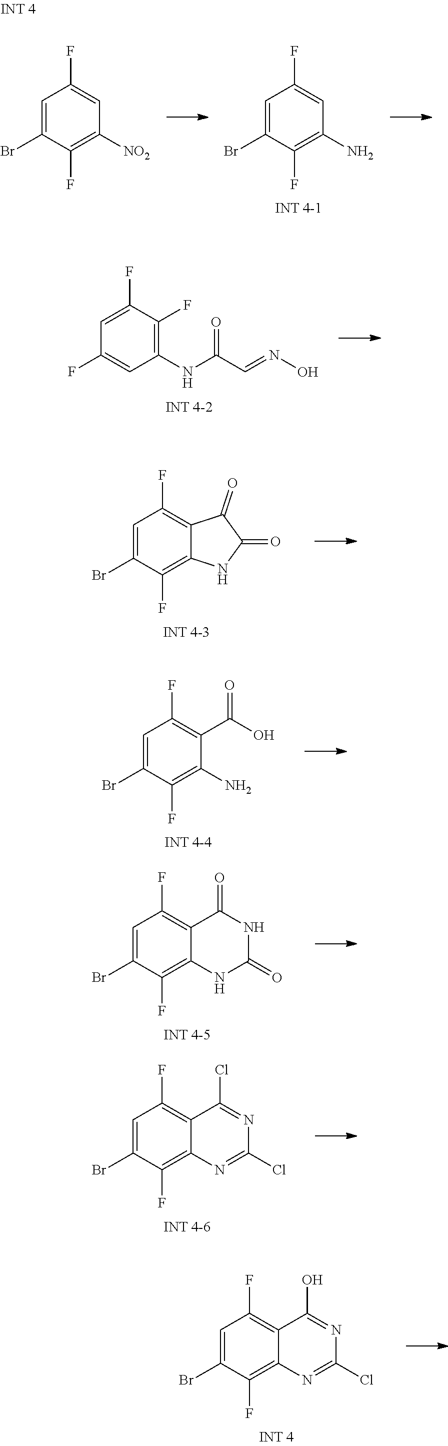

A solution of 1-bromo-2,5-difluoro-3-nitrobenzene (3.11 g, 13.06 mmol), iron (2.12 g, 37.96 mmol) and NH₄Cl (3.49 g, 65.24 mmol) in ethanol (60 mL) and water (12 mL) was stirred at 80° C. for 2 hours. The resulting mixture was filtered and the filtrate was concentrated under reduced pressure. The residue was dissolved in DCM (100 mL), washed with brine (2×30 mL), dried over anhydrous Na₂SO₄ and concentrated under reduced pressure to give INT 4-1 (2.49 g, 11.97 mmol, 91.6% yield). MS (ESI, m/z): 208 [M+H]⁺.

To a solution of INT 4-1 (2.49 g, 11.97 mmol), hydroxylammoniumchloride (2.49 g, 35.83 mmol), Na₂SO₄ (11.64 g, 95.76 mmol), chloral hydrate (2.56 g, 17.95 mmol) in water (50 mL) and ethanol (7 mL) was added hydrochloric acid (1.75 mL). The reaction mixture was stirred at 60° C. for 16 hours. The resulting mixture was cooled to room temperature and filtered, the filter cake was dried to give INT 4-2 (3.295 g, 11.80 mmol, 98.6% yield). MS (ESI, m/z): 279 [M+H]⁺.

INT 4-2 (3.295 g, 11.80 mmol) was added portion-wise to sulfuric acid (29.5 mL) at 60° C. The reaction was stirred at 90° C. for 1 hour. The resulting mixture was cooled to room temperature and added slowly to ice water. The resulting precipitate was collected by filtration, washed with water and dried under reduced pressure to give INT 4-3 (2.173 g, 8.29 mmol, 70.2% yield). MS (ESI, m/z): 262[M+H]⁺.

To a solution of INT 4-3 (2.173 g, 8.29 mmol) in aq. NaOH (2 M, 46 mL, 93.50 mmol) was added dropwise hydrogen dioxide (5.2 mL) at 0° C. The reaction mixture was stirred at room temperature for 16 hours. Excess hydrogen dioxide was quenched with excess sodium sulfite and the mixture was neutralized to pH=7. The mixture was filtered and the filtrate was acidified to pH=2 with concentrated hydrochloric acid, the resulting precipitate was collected by filtration, washed with water and dried under reduced pressure to give INT 4-4 (1.782 g, 7.07 mmol, 69.8% yield). MS (ESI, m/z): 252 [M+H]⁺.

To a solution of INT 4-4 (1.782 g, 7.07 mmol) in dichloromethane (20 mL) was added dropwise chlorosulfonyl isocyanate (1.33 g, 9.39 mmol) at 0° C. The reaction mixture was stirred at room temperature for 6 hours and concentrated under reduced pressure. Then concentrated hydrochloric acid (20 mL) was added and stirred at 100° C. for 16 hours. The resulting mixture was cooled to room temperature and filtered, the filter cake was washed by water and dried under reduced pressure to give INT 4-5 (0.83 g, 2.99 mmol, 75.5% yield). MS (ESI, m/z): 275 [M−1]⁻.

To a solution of INT 4-5 (0.83 g, 2.99 mmol) in POCl₃ (15 mL) was added N, N-diisopropylethylamine (2 mL). The reaction mixture was stirred at 105° C. for 2 hours. The resulting mixture was concentrated under reduced pressure and the residue was diluted with DCM (50 mL), washed with water (2×30 mL), dried over anhydrous sodium sulphate, concentrated under reduced pressure to give INT 4-6 (1.87 g, 5.95 mmol, 113.8% yield).

A suspension of INT 4-6 (4.96 g, 15.80 mmol) in 5% sodium hydroxide solution (150 mL) was stirred at room temperature for 4 hours. Upon completion, the mixture was adjusted to pH 9-10 with 5% NaHCO₃ and extracted with EA twice. The combined organic phases were washed with brine, dried over anhydrous Na₂SO₄, filtered and concentrated under reduced pressure. The residue was triturated with EA:Hex=1:8 (35 mL). The suspension was filtered and the filter cake was dried under reduced pressure to give INT 4 (3.76 g, 12.73 mmol). MS: m/z 295 [M+1]⁺.

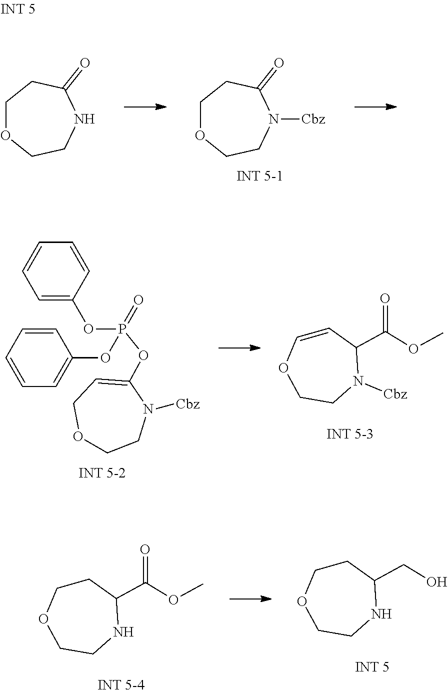 [SMILES: O=C1CCOCCN1 ; O=C1CCOCCN1C(=O)OCc1ccccc1 ; O=C(OCc1ccccc1)N1CCOCC=C1OP(=O)(Oc1ccccc1)Oc1ccccc1 ; COC(=O)C1C=COCCN1C(=O)OCc1ccccc1 ; COC(=O)C1CCOCCN1 ; OCC1CCOCCN1]

A solution of 1,4-oxazepan-5-one,1,4-oxazepan-5-one-1 (4.03 g, 35.00 mmol) in THF (80 mL) was cooled to −78° C., n-BuLi (15.5 mL) was added dropwise, stirred for 30 min, then phenylmethyl chloroformate (6.58 g, 38.57 mmol) was added under −70° C. and stirred for 1 h. The reaction mixture was quenched with sat. NH₄Cl (aq., 50 mL), extracted with EtOAc (2×100 mL). The organic layer was dried over anhydrous Na₂SO₄ and concentrated under reduced pressure. The residue was purified by silica gel column chromatography (eluted with 0-25% hex in EtOAc, v/v) to give INT 5-1 (4.09 g, 16.40 mmol, 46.9% yield). MS m/z: 250 [M+H]⁺.

A solution of INT 5-1 (4.09 g, 16.40 mmol) in THF (120 mL) was cooled to −70° C., KHMDS (1 M, 20 mL) was added dropwise, stirred for 90 min, then diphenyl chlorophosphate (5.18 g, 19.28 mmol) in THF (20 mL) was added and stirred for 60 min at −70° C. The reaction mixture was quenched with 5% NaOH (aq., 60 mL), extracted with EtOAc (2×200 mL). The combined organic layer was dried over anhydrous Na₂SO₄ and concentrated under reduced pressure. The residue was purified by silica gel column chromatography (eluted with 0-25% hex in EtOAc, v/v) to give INT 5-2 (5.82 g, 12.09 mmol, 73.7% yield). MS m/z: 482 [M+H]⁺.

A mixture of INT 5-2 (9 g, 18.7 mmol), Pd(OAc)₂ (420 mg, 1.87 mmol) and PPh₃ (981 mg, 3.74 mmol) in DMF (192 mL) was evacuated/backfilled with carbon monoxide for three times. The resulting mixture was stirred for 30 min at 25° C. TEA (3.80 g, 37.4 mmol) and MeOH (24 g, 748 mmol) was added and stirred for 1.5 hours at 45° C. The reaction mixture was diluted with water, extracted with EtOAc, the combined organic layer was washed with brine, dried over anhydrous Na₂SO₄, filtered and concentrated under reduced pressure. The residue was purified by silica gel column chromatography (eluted with petroleum ether:EtOAc=5:1, v/v) to give INT 5-3 (4.5 g). MS m/z: 292 [M+H]⁺.

To a solution of INT 5-3 (1.9 g, 6.52 mmol) in methanol (20 mL) was added 10% Pd/C (1.07 g, 1.00 mmol). The reaction mixture was stirred at room temperature for 3 hours under hydrogen atmosphere. The resulting mixture was filtered and the filtrate was concentrated under reduced pressure to give INT 5-4 (994 mg, 6.24 mmol, 95.7% yield). MS m/z: 160 [M+H]⁺.

A solution of INT 5-4 (994 mg, 6.24 mmol) in THF (10 mL) was cooled to 0° C., LiAlH₄ (1 M, 10 mL) was added dropwise and stirred for 90 min at room temperature. The reaction mixture was quenched with water (0.5 mL), followed with 15% NaOH (aq., 0.5 mL) and water (1.5 mL). The resulting mixture was filtered and the filtrate was concentrated under reduced pressure. The residue was purified by silica gel column chromatography (eluted with 0-10% methanol in DCM, v/v) to give INT 5 (419 mg, 3.19 mmol, 51.2% yield).

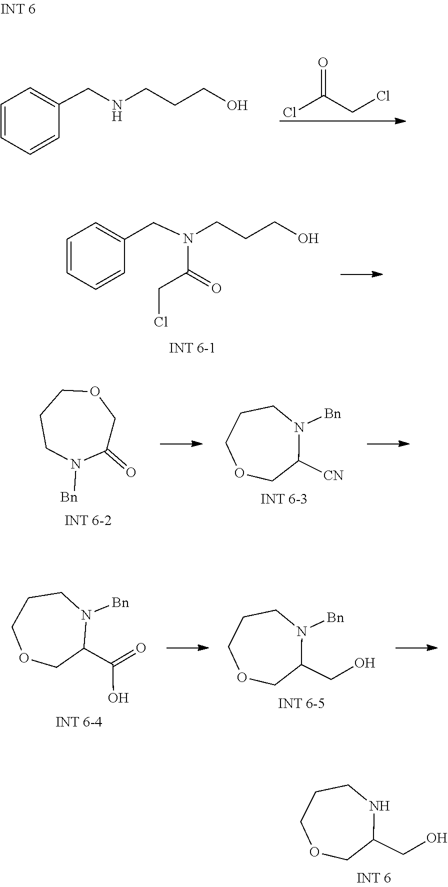

3-(benzylamino)propan-1-ol (18.0 g, 108 mmol) and TEA (12.1 g, 119 mmol) were added into DCM (126 mL). A solution of 2-chloroacetyl chloride (12.3 g, 108 mmol) in DCM (30.0 mL) was added dropwise at 0° C. The resulting mixture was stirred at 20° C. for 2 hours. The reaction mixture was concentrated under reduced pressure. The residue was purified by column chromatography (SiO₂, petroleum ether/ethyl acetate=30/1 to 1/1, v/v) to give INT 6-1 (21.0 g, 86.8 mmol, 79.7% yield). LCMS: 242[M+1].

¹H NMR (400 MHz, CDCl₃): δ 7.19-7.41 (m, 5H), 4.61-4.63 (m, 2H), 4.27 (s, 1H), 4.08 (s, 1H), 3.57-3.65 (m, 3H), 3.45-3.56 (m, 1H), 3.23-3.27 (m, 1H), 1.66-1.82 (m, 2H).

NaH (3.82 g, 95.5 mmol, 60% content) was added into a solution of INT 6-1 (21.0 g, 86.8 mmol) in THF (147 mL) at 0° C. The resulting mixture was stirred at 20° C. for 5 hours. NH₄Cl (aq., 50.0 mL) was added at 0° C., then extracted with EtOAc (3×100 mL), washed with brine (3×50.0 mL). The organic layer was combined, dried over with anhydrous Na₂SO₄ and concentrated under reduced pressure. The residue was purified by column chromatography (SiO₂, petroleum ether/ethyl acetate=40/1 to 1/1, v/v) to give INT 6-2 (12.2 g, 59.4 mmol, 68.4% yield). LCMS: 206[M+1].

¹H NMR (400 MHz, CDCl₃): δ 7.11-7.19 (m, 5H), 4.46 (s, 2H), 4.16 (s, 2H), 3.64-3.66 (m, 2H), 3.25-3.27 (m, 2H), 1.64-1.70 (m, 2H).

INT 6-2 (0.500 g, 2.44 mmol), TMDS (649 mg, 4.87 mmol), IrCl(CO[P(C₆H₅)₃])₂ (19.0 mg, 24.3 μmol), TMSCN (483 mg, 4.87 mmol) was added into toluene (5.00 mL) at 20° C. The resulting mixture was stirred at 20° C. for 16 hours. The reaction mixture was concentrated under reduced pressure. The residue was purified by prep-HPLC (TFA condition; Phenomenex Luna C18 75×30 mm×3 μm; mobile phase: [water (TFA)-CH₃CN]; Gradient: 20%-50%, 7 min) to give INT 6-3 (0.800 g, 3.70 mmol, 37.9% yield). LCMS: 217[M+1].

¹H NMR (400 MHz, CDCl₃): δ 7.33-7.39 (m, 5H), 3.76-3.94 (m, 7H), 2.92-2.96 (m, 2H), 1.96-2.41 (m, 2H).

A solution of INT 6-3 (251 mg, 1.16 mmol) in hydrochloric acid (3 mL) was stirred at 100° C. overnight. The mixture was concentrated to give INT 6-4 (357 mg, 1.52 mmol, crude) MS: m/z 236 (M+H)⁺.

To a 0° C. solution of INT 6-4 (357 mg, 1.52 mmol) in THF (10 mL) was added LiAlH₄ (221 mg, 5.8235 mmol). The mixture was stirred at 0° C. for 2 hours. The mixture was quenched with ice water (5 mL), filtered and the filtrate was concentrated. The residue was purified by pre-TLC to give INT 6-5 (145 mg, 655.23 μmol, 43.18% yield) MS: m/z 222 (M+H)⁺.

A solution of INT 6-5 (145 mg, 655.23 μmol) and Pd/C (10%, 185 mg, 1.74 mmol) in MeOH (5 mL) was stirred at 50° C. overnight under hydrogen atmosphere. The mixture was filtered and the filtrate was concentrated under reduced pressure to give INT 6 (92 mg, 701.37 μmol, 107.04% yield). MS: m/z 132 (M+H)⁺.

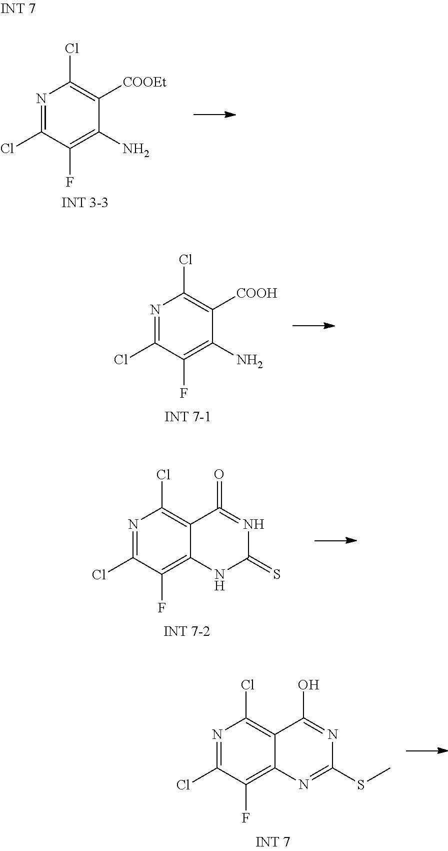

A mixture of INT 3-3 (4.53 g, 17.90 mmol), NaOH (2.99 g, 74.76 mmol) in EtOH (50 mL) and water (10 mL) was stirred for 4 hours at 50° C. The reaction mixture was concentrated under reduced pressure. The residue was dissolved in water (40 mL), extracted with EtOAc (2×30 mL). The water phase was adjusted pH to 2, extracted with EtOAc (2×30 mL), the organic layer was combined, dried over anhydrous Na₂SO₄, filtered and concentrated under reduced pressure to give INT 7-1 (2.68 g, 11.91 mmol, 66.54% yield). MS: m/z 225 (M+H)⁺.

A mixture of INT 7-1 (2.44 g, 10.84 mmol) and SOCl₂ (15 mL) was stirred for 2 hours at 70° C. The mixture was concentrated under reduced pressure. A solution of the obtained residue in CH₃CN (10 mL) was added dropwise to a mixture of ammonium thiocyanate (2.22 g, 29.16 mmol) in CH₃CN (40 mL), then stirred for 1.5 hours. The reaction mixture was filtered, the filter cake was collected, dried to give INT 7-2 (2.21 g, 8.31 mmol, 76.59% yield). MS: m/z 264 (M−H)⁻.

To a mixture of INT 7-2 (2.01 g, 7.55 mmol), NaOH (aq. 0.1 M, 150 mL) and MeOH (150 mL) was added CH₃I (2.22 g, 15.64 mmol). The mixture was stirred for 0.5 hour at RT. The water phase was adjusted pH to 3 by hydrochloric acid, extracted with EtOAc (1×200 mL, 1×100 mL), the organic layer was combined and concentrated under reduced pressure. The residue was triturated with water (20 mL) to give INT 7 (1.88 g, 6.71 mmol, 88.85% yield). MS: m/z 278 (M−H)⁻.

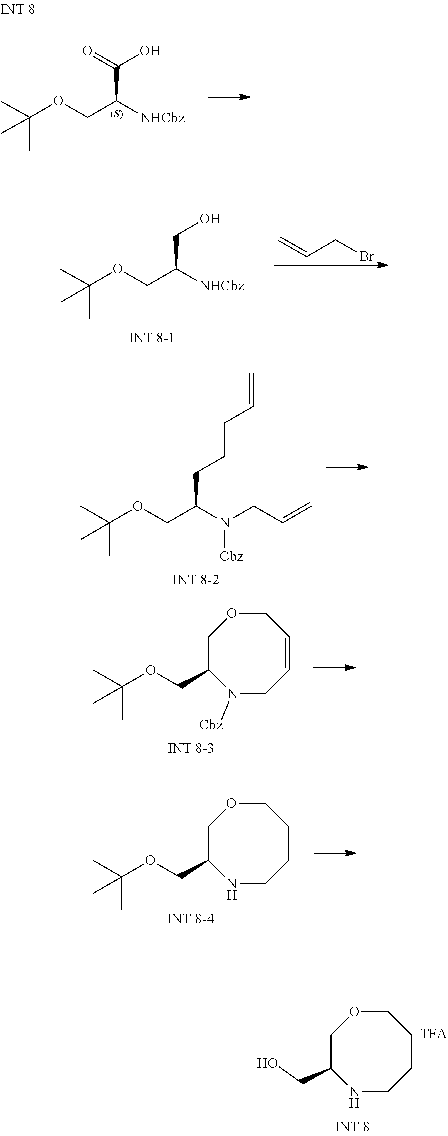

Following the procedure of WO2013064231, INT 8-1 was prepared with N-((benzyloxy)carbonyl)-O-(tert-butyl)-L-serine as starting material.

To a mixture of INT 8-1 (3.85 g, 13.68 mmol) in DMF (32 mL) was added 3-bromoprop-1-ene (6.96 g, 57.5 mmol) and TBAI (506 mg, 1.37 mmol). The resulting mixture was stirred at 0° C., NaH (2.74 g, 68.4 mmol, 60%) was added in portions. The reaction mixture was stirred for 1.5 hours at RT, then diluted with water (20 mL), extracted with EtOAc (3×20 mL), the combined organic layer was washed with brine, dried over anhydrous Na₂SO₄, filtered and concentrated under reduced pressure. The residue was purified by silica gel chromatography (eluted with petroleum ether:EtOAc=40:1, v/v) to give INT 8-2 (4.1 g). MS m/z: 362 [M+H]⁺.

A mixture of INT 8-2 (1.2 g, 3.32 mmol) and Grubs-I (120 mg) in DCM (86.3 mL) was stirred for 21 hour at reflux temperature. The reaction mixture was concentrated under reduced pressure. The residue was purified by silica gel chromatography (eluted with petroleum ether:EtOAc=40:1, v/v) to give INT 8-3 (550 mg). MS m/z: 334 [M+H]⁺.

To a solution of INT 8-3 (516 mg, 1.55 mmol) in MeOH (15 mL) was added Pd/C (467 mg). The reaction mixture was stirred at room temperature for 4 hours. The mixture was filtered and the filtrate was concentrated under reduced pressure to give INT 8-4 (459 mg, 2.28 mmol). MS m/z: 202 [M+H]⁺.

To a solution of INT 8-4 (438 mg, 2.18 mmol) in DCM (3 mL) was added TFA (3 mL). The reaction mixture was stirred at RT for 8 hours, then concentrated under reduced pressure. The residue was lyophilized to give INT 8 (434 mg, 1.67 mmol, TFA salt). MS m/z: 146 [M+H]⁺.

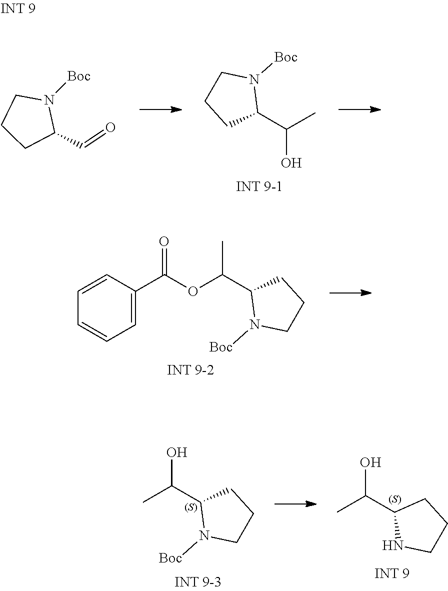

Into a 100 mL 3-necked round-bottom flask were added tert-butyl (2S)-2-formylpyrrolidine-1-carboxylate (3000 mg, 15.06 mmol) and THF (30 mL) at room temperature. To the above mixture was added MeMgBr (2693.09 mg, 22.58 mmol) dropwise at −78° C. under argon atmosphere. The resulting mixture was stirred for additional 3 hours at room temperature. The reaction was quenched with sat. NH₄Cl (aq.) at 0° C. The resulting mixture was extracted with DCM (3×50 mL). The combined organic layer was dried over anhydrous MgSO₄. After filtration, the filtrate was concentrated under reduced pressure. The residue was purified by reversed-phase flash chromatography (column, C18 silica gel; mobile phase, MeCN in water (0.1% FA), 10% to 50% gradient in 30 min; detector, UV 254 nm) to give INT 9-1 (2.4 g, 74.04%). LC-MS: (ES, m/z): [M+H-CH₃]⁺: 201.09.

¹H NMR (400 MHz, Chloroform-d) δ 3.98-3.90 (m, 1H), 3.79-3.63 (m, 1H), 3.60-3.43 (m, 1H), 3.26-3.23 (m, 1H), 1.97 (m, 1H), 1.85-1.76 (m, 3H), 1.47 (s, 9H), 1.15 (d, J=5.9 Hz, 2H), 1.09 (d, J=6.4 Hz, 1H).

Into a 250 mL round-bottom flask were added INT 9-1 (2200 mg, 10.22 mmol), DCM (30 mL), DIEA (3962.18 mg, 30.657 mmol) and DMAP (249.68 mg, 2.044 mmol) at room temperature. To the above mixture was added CbzCl (3486.39 mg, 20.438 mmol) dropwise at 0° C. The resulting mixture was stirred overnight at room temperature. The reaction mixture was quenched with water/ice at room temperature. The resulting mixture was extracted with DCM (3×40 mL). The combined organic layer was dried over anhydrous MgSO₄. After filtration, the filtrate was concentrated under reduced pressure. The residue was purified by reversed-phase flash chromatography (column, C18 silica gel; mobile phase, MeCN in water (0.1% FA), 10% to 100% gradient in 30 min; detector, UV 254 nm) to give INT 9-2 (2.51 g, 69.67%). LC-MS (ES, m/z): [M+H-Boc]⁺: 220.10.

¹H NMR (400 MHz, Chloroform-d) δ 8.06 (m, 2H), 7.56 (m, 1H), 7.45 (m, 2H), 5.56-5.22 (m, 1H), 4.18-4.01 (m, 1H), 3.90-3.21 (m, 2H), 2.13-1.77 (m, 4H), 1.34 (m, 12H).

A solution of sodium hydroxide (209 mg, 5.23 mmol) in water (4 mL) was added to INT 9-2 (300 mg, 939.28 mol) in methanol (10 mL). The mixture was stirred at 50° C. for 16 hours. Anhydrous sodium sulfate was added and the mixture was filtered, washed with DCM. The filtrate was concentrated under reduced pressure to give crude INT 9-3 (202 mg). MS: m/z: 216 [M+H]⁺.

A solution of INT 9-3 (202 mg) and HCl (4 M in 1,4-dioxane, 2 mL) in DCM (5 mL) was stirred at room temperature for 1.5 hours. The solution was concentrated under reduced pressure to give crude INT 9 (108 mg). MS: m/z: 116 [M+H]⁺.

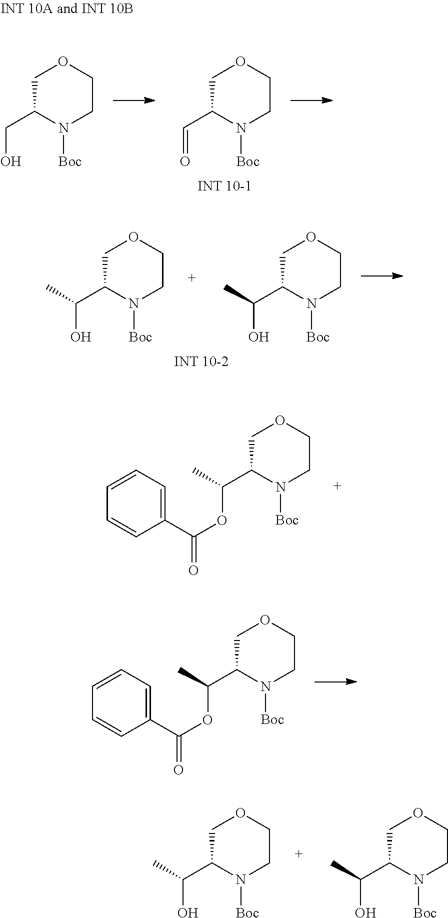

Into a 500 mL 3-necked round-bottom flask were added oxalic dichloride (7.01 g, 55.232 mmol) and DCM (75 mL) at room temperature under nitrogen atmosphere. To the above mixture was added a solution of DMSO (4.32 g, 55.232 mmol) in DCM (75 mL) at −78° C. The resulting mixture was stirred for additional 5 min at −78° C. To the above mixture was added tert-butyl (3R)-3-(hydroxymethyl)morpholine-4-carboxylate (6 g, 27.615 mmol) in DCM (50 mL). The resulting mixture was stirred for additional 15 min at −78° C. To the above mixture was added TEA (16.77 g, 165.696 mmol). The resulting mixture was stirred for additional 15 min at −78° C. and slowly warmed to room temperature and stirred for 1 hour. The reaction mixture was quenched with water at room temperature. The resulting mixture was extracted with DCM (3×100 mL). The combined organic layer was washed with brine (2×100 mL), dried over anhydrous MgSO₄. After filtration, the filtrate was concentrated under reduced pressure. This resulted in INT 10-1 (6.2 g crude) which was used in the next step directly without further purification. LC-MS: (ES, m/z): [M+H]⁺: 216, [M-CH₃+H]⁺: 201, [M-C₄H₉+H]⁺: 160, [M-C₅H₉O₂+H]⁺: 116.

Into a 500 mL 3-necked round-bottom flask were added INT 10-1 (6.2 g crude) and THF (50 mL) at room temperature under nitrogen atmosphere. The mixture was allowed to cool down to −78° C. To the above mixture was added MeMgBr in THF (10 mL, 30.0 mmol, 3 mol/L) dropwise at −78° C. The resulting mixture was stirred for additional 2 hours at room temperature. The reaction mixture was quenched with sat. NH₄Cl (aq.) at room temperature. The resulting mixture was concentrated under reduced pressure. The resulting mixture was extracted with EtOAc (3×60 mL). The combined organic layer was washed with brine (2×60 mL), dried over anhydrous Na₂SO₄. After filtration, the filtrate was concentrated under reduced pressure. The residue was purified by reversed-phase flash chromatography (column, C18 silica gel; mobile phase, MeCN in water, 0% to 100% gradient in 80 min; detector, UV 220 nm) to give INT 10-2 (4.000 g). LC-MS: (ES, m/z): [M+H]⁺: 232, [M-CH₃+H]⁺: 217, [M-C₄H₉+H]⁺: 176, [M-C₅H₉O₂+H]⁺: 132.

Into a 100 mL 3-necked round-bottom flask were added INT 10-2 (4 g, 17.294 mmol) and DCM (40 mL, 43.235 mmol), TEA (5.25 g, 51.882 mmol), DMAP (2.11 g, 17.294 mmol) at room temperature under nitrogen atmosphere. To the above mixture was added benzoyl chloride (6.08 g, 43.235 mmol) at 0° C. The resulting mixture was stirred for overnight at room temperature. The reaction was quenched with water/ice at room temperature. The resulting mixture was extracted with DCM (3×40 mL). The combined organic layer was washed with brine (2×40 mL), dried over anhydrous MgSO₄. After filtration, the filtrate was concentrated under reduced pressure. The residue was purified by reversed-phase flash chromatography (column, C18 silica gel; mobile phase, MeCN in water, 0% to 100% gradient in 80 min; detector, UV 254 nm) to give INT 10A-3 (the first peak, 1.627 g, 23.42%) and INT 10B-3 (the second peak, 0.890 g, 14.35%).

INT 10A-3 LC-MS: (ES, m/z): [M+H]⁺: 336, [M-CH₃+H]⁺: 321, [M-C₄H₉+H]⁺: 280, [M-C₅H₉O₂+H]⁺: 236, [M+Na]⁺: 358. ¹H NMR (300 MHz, Chloroform-d) δ 8.05 (d, J=7.6 Hz, 2H), 7.53 (m, 1H), 7.41 (m, 2H), 5.74 (m, 1H), 4.25-4.05 (m, 1H), 3.98 (d, J=12.1 Hz, 1H), 3.89-3.77 (m, 1H), 3.63 (d, J=12.9 Hz, 2H), 3.54-3.40 (m, 1H), 3.29 (m, 1H), 1.49-1.31 (m, 12H).

INT 10B-3 LC-MS: (ES, m/z): [M+H]⁺: 336, [M-CH₃+H]⁺: 321, [M-C₄H₉+H]⁺: 280, [M-C₅H₉O₂+H]⁺: 236, [M+Na]⁺: 358 ¹H NMR (300 MHz, Chloroform-d) δ 8.10-8.00 (m, 2H), 7.62-7.51 (m, 1H), 7.45 (dd, J=8.4, 7.0 Hz, 2H), 5.61 (dq, J=9.6, 6.4 Hz, 1H), 4.11 (s, 1H), 3.97 (d, J=11.9 Hz, 1H), 3.86 (d, J=11.7 Hz, 2H), 3.57-3.48 (m, 1H), 3.45 (dd, J=11.7, 3.1 Hz, 1H), 3.12 (d, J=12.9 Hz, 1H), 1.49 (s, 9H) 1.36 (d, J=6.3 Hz, 3H).

A solution of INT 10A-3 (510 mg, 1.52 mmol), NaOH (244 mg, 6.10 mmol) in MeOH (10 mL) was stirred at room temperature for 18 hours. The pH of the solution was adjusted to 7˜8 by 1 N hydrochloric acid at 0-10° C. and then concentrated under reduced pressure. The residue was extracted with EtOAc (20 mL×3), washed with NaCl (aq. 30 mL), dried over anhydrous Na₂SO₄, filtered and the filtrate was concentrated under reduced pressure to give INT 10A (393.8 mg, crude), which was used directly in the next step without purification. MS: m/z: 232 [M+H]⁺.

To a solution of INT 10B-3 (362 mg, 1.08 mmol) in MeOH (9 mL) was added a solution of NaOH (89 mg, 2.23 mmol) in water (3 mL) at room temperature and stirred for 5 hours. Then the mixture was quenched with hydrochloric acid (1 N) and adjusted pH=7. The resulting mixture was extracted with EtOAc (30 mL×2), the combined organic layer was dried over anhydrous Na₂SO₄, filtrated and concentrated under reduced pressure to give INT 10B (0.23 g, crude). MS: m/z: 232 [M+H]⁺.

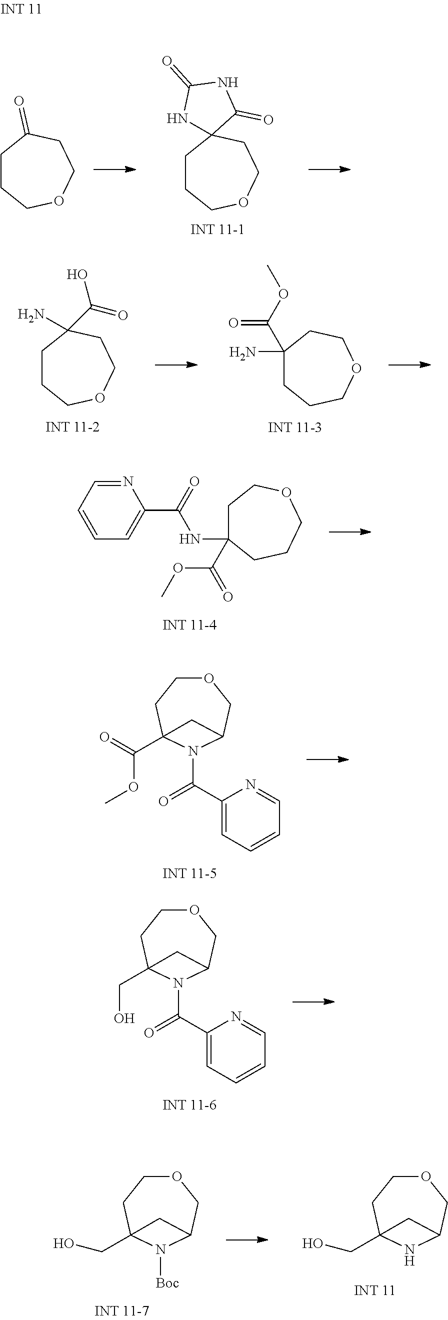

To a solution of oxepan-4-one (15.0 g, 131.4 mmol) in EtOH (150 mL) and H₂O (150 mL) was added (NH₄)₂CO₃ (37.8 g, 394.26 mmol) at room temperature. Then NaCN (9.7 g, 197.1 mmol) was added to the mixture under argon atmosphere. Then the mixture was heated to 65° C. and stirred for 12 hours. After cooled to room temperature, the reaction mixture was quenched with NaClO (450 mL) and stirred for 10 min. Then the mixture was cooled to 0° C. and adjusted to pH<2 with 6 N HCl. The resulting mixture was extracted with chloroform/isopropyl alcohol=5:1 (500 mL×3), the combined organic layer was dried over anhydrous Na₂SO₄ and concentrated under reduced pressure to give INT 11-1 (16.0 g, crude) and used directly for the next step without further purification. LCMS: 185.2 [M+H]⁺.

A solution of INT 11-1 (16.0 g, crude, 140.2 mmol) in 6 N KOH (160 mL) was stirred for 12 hours at 120° C. After completion, the solution was adjusted to pH 9 with 4 N HCl and the mixture was concentrated under reduced pressure to give INT 11-2 (65.0 g, crude, contain a lot of salt) and used directly for the next step without further purification. LCMS: 160.2 [M+H]⁺.

To a solution of INT 11-2 (65.0 g, crude, 140.2 mmol, contains a lot of salt, 1.0 eq) in MeOH (400 mL) was added concentrated H₂SO₄ (20 mL). Then the mixture was heated to 70° C. and stirred for 12 hours. After completion of the reaction, the mixture was cooled to 0° C., adjusted to pH=9 with 2 N NaOH and concentrated under reduced pressure to give crude product. The residue was purified by silica gel chromatography (eluted with MeOH/DCM=1:10, v/v) to give INT 11-3 (20.0 g, crude). LCMS: 174.2 [M+H]⁺.

To a solution of INT 11-3 (16.0 g, crude, 92.4 mmol) in THF (300 mL) was added TEA (47.0 g, 461.9 mmol) at room temperature. The reaction mixture was cooled to 0° C. under argon atmosphere, picolinoyl chloride (19.6 g, 138.5 mmol) in THF (300 mL) was added. The mixture was stirred for 1 hour at 25° C. under argon atmosphere. After completion, the reaction mixture was poured into water and the mixture was extracted with EtOAc (600 mL×3), the combined organic layer was washed with brine (500 mL×2), dried over anhydrous Na₂SO₄ and concentrated under reduced pressure. The residue was purified by silica gel chromatography (eluted with petroleum ether/EtOAc=5:1) to give the INT 11-4 (5.0 g, yield 13.7% for four steps). LCMS: 279.1 [M+H]⁺.

To a solution of INT 11-4 (100 mg, 0.36 mmol) in 1,1,2,2-tetrachloroethane (1.5 mL) was added Pd(OAc)₂ (8 mg, 0.04 mmol), AgOAc (180 mg, 1.08 mmol), 2,3,4,5,6-pentafluoro-1-iodobenzene (1.06 g, 3.6 mmol), 1,4-benzoquinone (20 mg, 0.18 mmol) and Na₃PO₄ (177 mg, 1.08 mmol-) at room temperature. The reaction mixture was degassed with argon for 5 min. The mixture was heated to 130° C. and stirred for 12 hours. After completion, the mixture was filtered through a Celite pad, and the filtrate was concentrated under reduced pressure. The residue was purified by silica gel chromatography on (eluted with petroleum ether/EtOAc=5:1) to give INT 11-5 (35 mg, yield 35.3%). LCMS: 277.2 [M+H]⁺.

A solution of INT 11-5 (1.5 g, 5.4 mmol) in EtOH (15 mL) and ether (15 mL) was cooled to 0° C. under argon atmosphere, then LiBH₄ (40.5 mL, 2 mol/L in THF, 81 mmol) was added in three portions over 30 min. The mixture was stirred for 2 hours at 0° C. After completion, the reaction mixture was quenched with 1 N HCl and stirred for 10 min. Then the mixture was cooled to 0° C., adjusted to pH=8 with 1 N NaOH. The mixture was extracted with EtOAc (80 mL×3), the combined organic layer was dried over anhydrous Na₂SO₄ and concentrated under reduced pressure to give INT 11-6 (1.0 g, crude, with salt) and used directly for the next step without further purification. LCMS: 249.2 [M+H]⁺.

To a solution of INT 11-6 (1.0 g, crude, with salt) in EtOH (10.2 mL) and H₂O (1.7 mL) was added NaOH (1.4 g, 35.0 mmol) at room temperature. The mixture was heated to 130° C. and stirred for 12 hours in a 30 mL of autoclave. After cooled to room temperature, excess (Boc)₂O was added and the mixture was stirred for 6 days at 40° C. Then the reaction mixture was poured into water and the mixture was extracted with EtOAc (10 mL×3), the combined organic layer was washed with brine (10 mL), dried over anhydrous Na₂SO₄ and concentrated under reduced pressure. The residue was purified by silica gel chromatography on (eluted with petroleum ether/EtOAc=15:1, v/v) to give INT 11-7 (360 mg, yield 27.3% for two steps).

¹H NMR (300 MHz, CDCl₃): δ 4.15-4.08 (m, 2H), 3.90 (d, J=12.5 Hz, 2H), 3.80 (d, J=11.7 Hz, 1H), 3.68 (d, J=12.3 Hz, 2H), 2.56 (d, J=14.1 Hz, 1H), 2.19 (t, J=8.7 Hz, 1H), 2.06-1.92 (m, 1H), 1.78-1.68 (m, 2H), 1.46 (s, 9H). LCMS: 188.2 [M−56+H]⁺.

To a solution of INT 11-7 (0.14 g, 0.58 mmol) in DCM (5 mL) was added TFA (1.5 mL). The reaction mixture was stirred at room temperature for 2 hours and then concentrated under reduced pressure to give INT 11 (crude). MS m/z: 144[M+H]⁺.

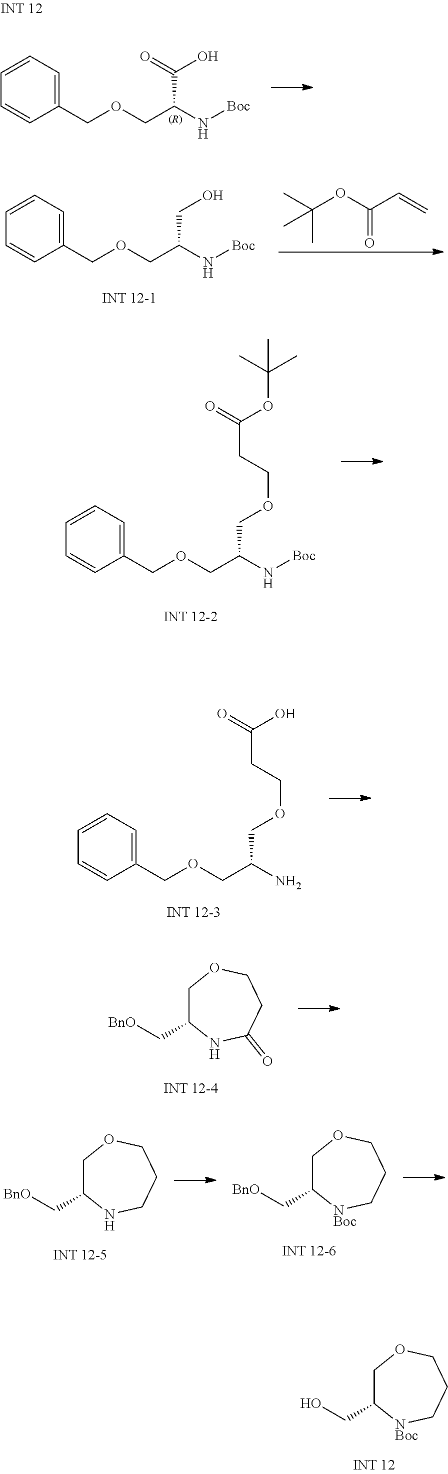

To a solution of N—BOC—O-Benzyl-D-serine (70.59 g, 239.02 mmol) and triethylamine (29.70 g, 293.50 mmol) in THF (1000 mL) was cooled to −15° C. and added isobutyl carbonochloridate (36.31 g, 265.85 mmol). The solution was stirred for 2 hours at the same temperature, then filtered. The filtrate was cooled to 0° C. and added to a solution of NaBH₄ (16.71 g, 441.68 mmol) in water (200 mL) dropwise. The solution was stirred for 1 hour at 0° C. The resulting mixture was quenched by water (200 mL), extracted with EtOAc (1000 mL), the organic layer was dried over anhydrous sodium sulphate and concentrated under reduced pressure. The residue was purified by silica gel column chromatography (eluted with 0-50% EtOAc in Hex) to give INT 12-1 (74.35 g, 264.26 mmol, 110.5% yield). MS (ESI, m/z): 282 [M+H]⁺.

A mixture of INT 12-1 (60.00 g, 213.26 mmol) and Cs₂CO₃ (70.76 g, 217.17 mmol) in tert-butanol (500 mL) and tert-butyl acrylate (500 mL) was stirred at room temperature for 16 hours. The resulting mixture was quenched by water (200 mL), extracted with EtOAc (500 mL), the organic layer was dried over anhydrous sodium sulphate and concentrated under reduced pressure. The residue was purified by silica gel column chromatography (eluted with 0-20% EtOAc in Hex) to give INT 12-2 (87.16 g, 212.83 mmol, 99.8% yield). MS (ESI, m/z): 410 [M+H]⁺.

To a solution of INT 12-2 (87.16 g, 212.83 mmol) in DCM (300 mL) was added TFA (300 mL). The solution was stirred at room temperature for 2 hours. The resulting mixture was concentrated under reduced pressure to give INT 12-3, the residue was used to next step directly without further purification.

To a solution of INT 12-3 (27 g, 106.59 mmol) in DCM (500 mL) were added HATU (49.19 g, 129.36 mmol) and TEA (48.04 g, 474.75 mmol). The solution was stirred at room temperature for 3 hours. Then the mixture was quenched with aq. NaHCO₃ (sat., 300 mL), extracted with DCM (200 mL), the organic layer was dried over anhydrous Na₂SO₄ and concentrated under reduced pressure. The residue was purified by silica gel chromatography (eluted with 10-100% EtOAc in hex) to give INT 12-4 (49.54 g, 210.55 mmol, 197.5% yield). MS (ESI, m/z): 236 [M+H]⁺.

A solution of LiAlH₄ (15.76 g, 415.28 mmol) in THF (200 mL) was cooled to 0° C., then a solution of INT 12-4 (49.54 g, 210.55 mmol) in THF (200 mL) was added dropwise. The reaction mixture was stirred at room temperature for 5 hours. The resulting mixture was quenched by water (15 mL), 15% oNaOH (15 mL), water (45 mL). The solution was filtered, the filtrate was concentrated under reduced pressure. The residue was purified by silica gel chromatography (eluted with 0-5% methanol in DCM) to give INT 12-5 (44.93 g, 203.03 mmol, 96.4% yield). MS (ESI, m/z): 222 [M+H]⁺.

To a solution of INT 12-5 (19.12 g, 86.40 mmol) in THF (200 mL) were added di-tert-butyl dicarbonate (21.05 g, 96.45 mmol) and N,N-diisopropylethylamine (16.17 g, 125.11 mmol). The reaction mixture was stirred at room temperature for 3 hours. The solution was diluted with water (100 mL), extracted with EtOAc (2×500 mL), the combined organic layer was dried over anhydrous Na₂SO₄ and concentrated under reduced pressure. The residue was purified by silica gel chromatography (eluted with 0-10% EtOAc in hex) to give INT 12-6 (18.02 g, 56.06 mmol, 64.8% yield). MS (ESI, m/z): 322 [M+H]⁺.

To a solution of INT 12-6 (7.00 g, 21.77 mmol) in methanol (140 mL) was added Pd/C (3.60 g, 3.38 mmol). The reaction mixture was stirred at room temperature for 16 hours under hydrogen atmosphere. The resulting mixture was filtered and the filtrate was concentrated under reduced pressure to give INT 12 (5.29 g, 22.87 mmol, 105.0% yield). MS (ESI, m/z): 232 [M+H]⁺.

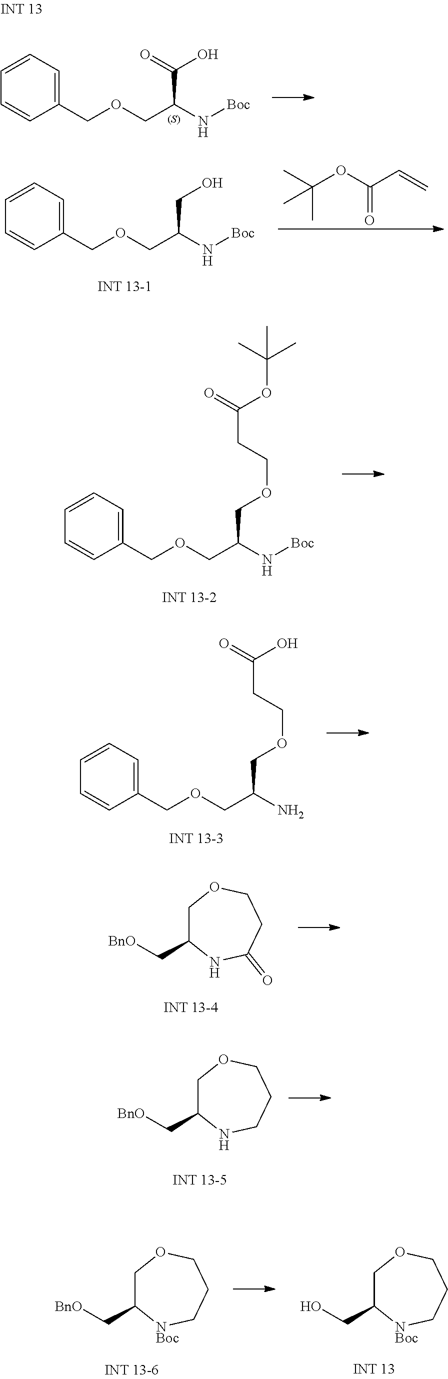

INT 13 was prepared following the procedure for the synthesis of INT 12 with O-benzyl-N-(tert-butoxycarbonyl)-L-serine.

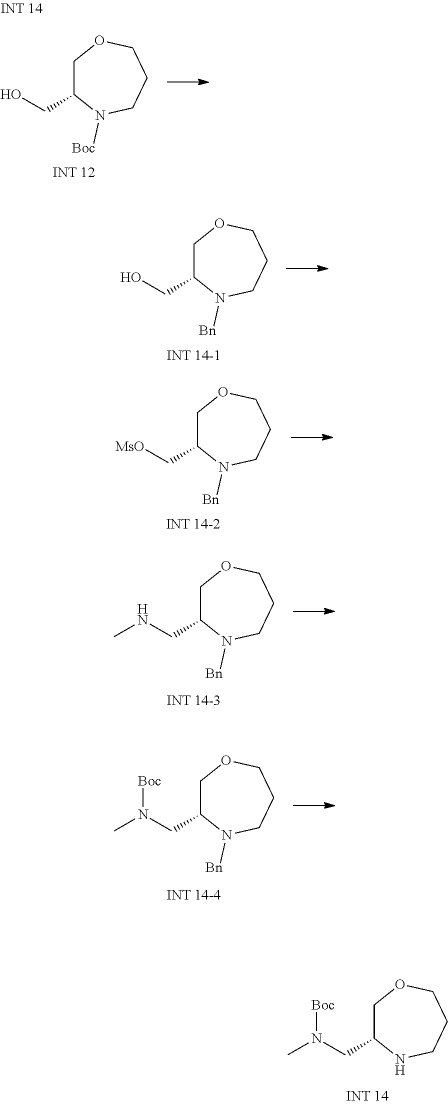 [SMILES: CC(C)(C)OC(=O)N1CCCOC[C@H]1CO ; OC[C@@H]1COCCCN1Cc1ccccc1 ; CS(=O)(=O)OC[C@@H]1COCCCN1Cc1ccccc1 ; CNC[C@@H]1COCCCN1Cc1ccccc1 ; CN(C[C@@H]1COCCCN1Cc1ccccc1)C(=O)OC(C)(C)C ; CN(C[C@@H]1COCCCN1)C(=O)OC(C)(C)C]

To a solution of INT 12 (1332 mg, 5.76 mmol) in DCM (15 ml) was added TFA (5 mL). The reaction mixture was stirred at room temperature for 2 hours. The solution was concentrated under reduced pressure. To a solution of the residue and DIEA (5 mL) in DCM (15 mL) was added (bromomethyl)benzene (1122 mg, 6.56 mmol). The reaction mixture was stirred at room temperature for 2.5 hours, then diluted with water (30 mL) and extracted with EtOAc (30 mL×2). The collected organic layer was washed with brine (30 mL), dried over anhydrous Na₂SO₄ and concentrated under reduced pressure. The residue was purified by silica gel column to give INT 14-1 (995 mg, 1.27 mmol). MS m/z: 222 [M+H]⁺.

To a solution of INT 14-1 (447 mg, 2.02 mmol), TEA (642 mg, 6.34 mmol) in THF (5 mL) was added MsCl (114 mg, 1.00 mmol) at −10° C. The reaction mixture was stirred at room temperature for 20 min, then diluted with water (30 mL) and extracted with EtOAc (30 mL×2). The collected organic layer was washed with brine (30 mL), dried over anhydrous Na₂SO₄ and concentrated under reduced pressure to give INT 14-2 (611 mg, 2.04 mmol).

To a solution of INT 14-2 (611 mg, 2.04 mmol), DIEA (1322 mg, 10.23 mmol) in acetonitrile (10 mL) was added methylamine hydrochloride (279 mg, 4.13 mmol). The reaction mixture was stirred at room temperature for 1.5 hours, then diluted with water (30 mL) and extracted with EtOAc (30 mL×2). The collected organic layer was washed with brine (30 mL), dried over anhydrous Na₂SO₄ and concentrated under reduced pressure to give INT 14-3 (crude). MS m/z: 235 [M+H]⁺.

To a solution of INT 14-3 (crude) and DIEA (0.5 mL) in THF (10 mL) was added di-tert-butyl dicarbonate (902 mg, 4.13 mmol). The reaction mixture was stirred at room temperature for 1 hour, diluted with water (30 mL) and extracted with EtOAc (30 mL×2). The collected organic layer was washed with brine (30 mL), dried over anhydrous Na₂SO₄ and concentrated under reduced pressure. The residue was purified by silica gel column to give INT 14-4 (127 mg, 0.38 mmol). MS m/z: 335 [M+H]⁺.

To a solution of INT 14-4 (107 mg, 0.32 mmol) in MeOH (5 mL) was added Pd(OH)₂/C (122 mg, 10% content). The reaction mixture was stirred at room temperature for 3 hours under hydrogen atmosphere. The mixture was filtered and the filtrate was concentrated under reduced pressure to give INT 14 (77 mg, 0.32 mmol). MS m/z: 245 [M+H]⁺.

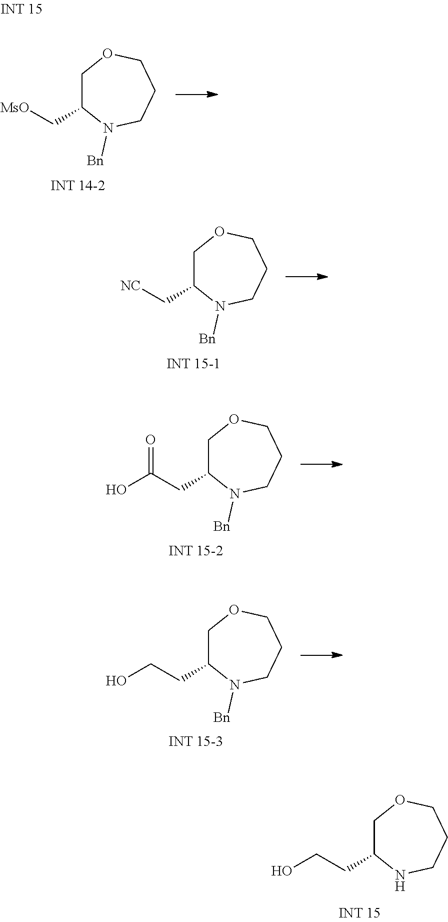 [SMILES: CS(=O)(=O)OC[C@@H]1COCCCN1Cc1ccccc1 ; N#CC[C@@H]1COCCCN1Cc1ccccc1 ; O=C(O)C[C@@H]1COCCCN1Cc1ccccc1 ; OCC[C@@H]1COCCCN1Cc1ccccc1 ; OCC[C@@H]1COCCCN1]

To a solution of INT 14-2 (0.5 g, 1.67 mmol), caesium fluoride (1.23 g, 8.10 mmol) in DMF (10 mL) was added cyanotrimethylsilane (0.74 g, 7.46 mmol). The reaction mixture was stirred at 80° C. for 4.5 hours, then diluted with water (30 mL) and extracted with EtOAc (30 mL×2). The collected organic layer was washed with brine (30 mL), dried over anhydrous Na₂SO₄ and concentrated under reduced pressure. The residue was purified by silica gel column to give INT 15-1 (257 mg, 1.12 mmol). MS m/z: 231 [M+H]⁺.

A solution of INT 15-1 (217 mg, 0.94 mmol) in concentrated hydrochloric acid (8 mL) was stirred at 80° C. for 3.5 hours. The reaction solution was concentrated under reduced pressure. To the mixture of the residue in THF (10 mL) was added TEA (2 mL). The mixture was concentrated under reduced pressure to give INT 15-2 (crude). MS m/z: 250 [M+H]⁺.

To a mixture of INT 15-2 (crude) in THF (10 mL) was added LiAlH₄ (216 mg, 5.69 mmol). The reaction mixture was stirred at room temperature for 1 hour, then quenched with water (0.3 mL), aq. NaOH (0.5 mL, 15% wt), water (1 mL). The mixture was filtered and the filtrate was concentrated under reduced pressure. The residue was purified by silica gel column to give INT 15-3 (141 mg, 0.60 mmol). MS m/z: 236 [M+H]⁺.

To a solution of INT 15-3 (141 mg, 0.60 mmol) in MeOH (15 ml) was added Pd(OH)₂/C (144 mg, 10% content). The reaction mixture was stirred at room temperature for 5.5 hours under hydrogen atmosphere. The mixture was filtered and the filtrate was concentrated under reduced pressure to give INT 15 (89 mg, 0.61 mmol). MS m/z: 146 [M+H]⁺.

### Example 1

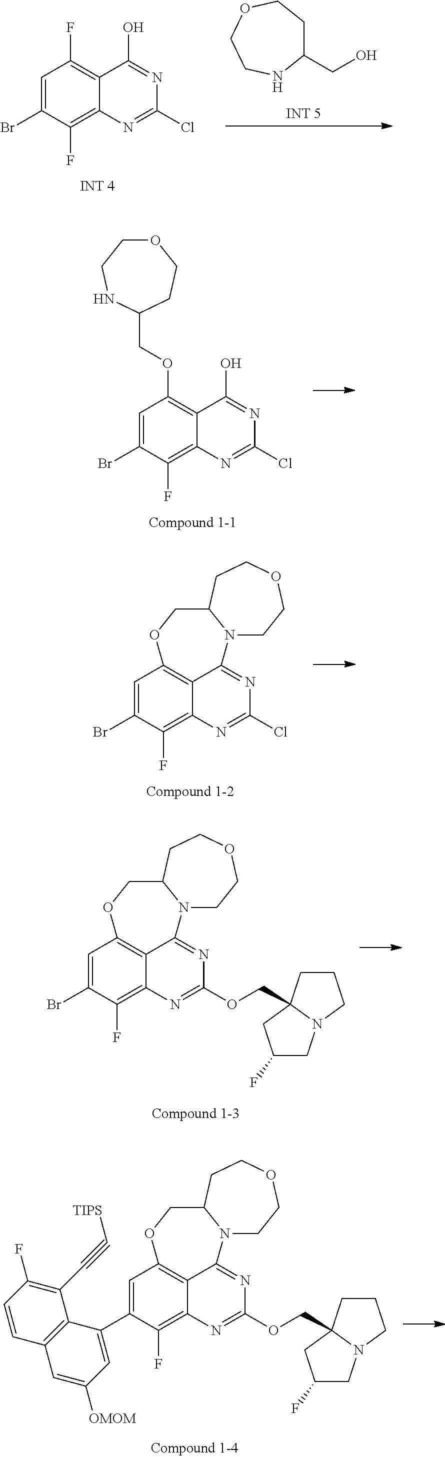

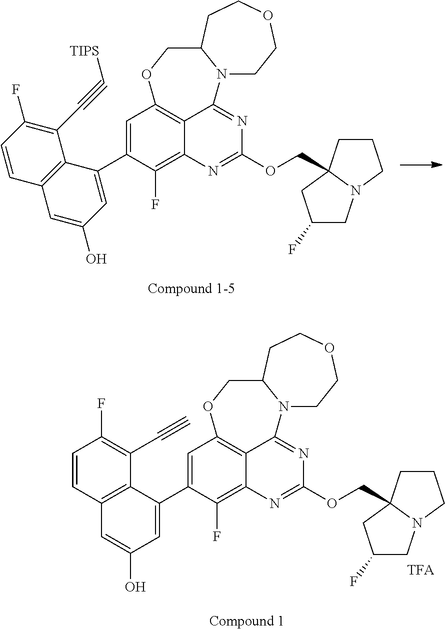

To a solution of INT 5 (280 mg, 2.13 mmol) in dry THF (25 mL) was added sodium hydride (60% in oil, 293 mg, 7.33 mmol) under nitrogen atmosphere at 0° C., and then stirred at room temperature for 30 minutes. The solution of INT 4 (629 mg, 2.13 mmol) in dry THF (5 mL) was added to the reaction mixture and stirred at room temperature for 24 hours. After completion, the reaction mixture was diluted with EtOAc (50 mL) and water (40 mL). The organic layer was separated and concentrated under reduced pressure. The residue was purified by Pre-TLC (eluted with DCM:MeOH=10:1, v/v) to give Compound 1-1 (377 mg, 0.93 mmol). MS: m/z 406 [M+H]⁺.

To a solution of Compound 1-1 (352 mg, 0.87 mmol) and DIEA (0.3 mL) in DCM (10 mL) was added phosphorus oxychloride (0.5 mL) under nitrogen atmosphere and then stirred for 1 hour. Upon completion, the residue was diluted with DCM (30 mL), quenched by saturated NaHCO₃ (50 mL) and separated. The collected organic layer was concentrated under reduced pressure. The residue was purified by silica gel chromatography (eluting with Hex:EtOAc=3:1, v/v) to give Compound 1-2 (157 mg, 0.40 mmol). MS: m/z 388 [M+H]⁺.

To a solution of Compound 1-2 (144 mg, 0.37 mmol) and ((2R,7aS)-2-fluorotetrahydro-1H-pyrrolizin-7a(5H)-yl) methanol (86 mg, 0.54 mmol) in THF (5 mL) and DMF (5 mL) were added DABCO (19 mg, 0.17 mmol) and Cs₂CO₃ (341 mg, 1.05 mmol), the mixture was purged with nitrogen followed by stirring at room temperature for 16 hours. The mixture was diluted with EtOAc (30 mL) and water (30 mL), the organic layer was separated, washed with brine (20 mL), dried over anhydrous Na₂SO₄, filtered and concentrated under reduced pressure. The residue was purified by Pre-TLC (eluted with DCM:MeOH=15:1, v/v) to give Compound 1-3 (142 mg, 0.28 mmol). MS: m/z 511 [M+H]⁺.

To a solution of Compound 1-3 (142 mg, 0.28 mmol), INT 2 (208 mg, 0.41 mmol) and Cs₂CO₃ (267 mg, 0.82 mmol) in toluene (8 mL) and water (2 mL) was added cataCXium A Pd G3 (22 mg, 0.030 mmol), the mixture was purged with nitrogen followed by stirring at 100° C. for 16 hours. Upon completion, the reaction mixture was concentrated under reduced pressure, the residue was diluted with EtOAc (40 mL) and water (30 mL), and the organic layer was separated. The organic layer was concentrated under reduced pressure. The residue was purified by Pre-TLC (eluted with DCM:MeOH=15:1, v/v) to give Compound 1-4 (197 mg, 0.24 mmol). MS: m/z 817 [M+1]⁺.

To a solution of Compound 1-4 (197 mg, 0.24 mmol) in CH₃CN (5 mL) was added HCl/1,4-dioxane (4 M, 2 mL). The reaction mixture was stirred at room temperature for 1 hour. After completion, the reaction mixture was concentrated under reduced pressure, the residue was diluted with EtOAc (30 mL) and water (20 mL), the mixture was adjusted to pH=8-9 with saturated NaHCO₃. The organic layer was separated and concentrated under reduced pressure to give Compound 1-5 (181 mg, crude). MS: m/z 773 [M+H]⁺.

To a mixture of Compound 1-5 (181 mg, crude) in DMF (5 mL) was added CsF (399 mg, 2.63 mmol). The mixture was stirred at room temperature for 4 hours. After completion, the mixture was diluted with EtOAc (30 mL) and water (20 mL), the mixture was adjusted to pH=8-9 with saturated NaHCO₃. The organic layer was separated and concentrated under reduced pressure. The residue was purified by Pre-HPLC (C18 column, A: 0.1% TFA in water, B: CH₃CN, Gradient: 15% B to 45% B in 40 min at a flow rate of 60 mL/min, 230 nm) and the product fractions were lyophilized to give Compound 1 (144.5 mg, 0.20 mmol, TFA salt). MS: m/z 617 [M+H]⁺.

¹H NMR (600 MHz, MeOD) δ 7.87-7.81 (m, 1H), 7.35-7.28 (m, 2H), 7.14-7.01 (m, 2H), 5.59-5.46 (m, 1H), 4.73-4.62 (m, 2H), 4.48-4.42 (m, 1H), 4.41-4.31 (m, 1H), 4.01-3.89 (m, 4H), 3.89-3.74 (m, 2H), 3.74-3.59 (m, 2H), 3.50-3.39 (m, 2H), 2.72-2.56 (m, 2H), 2.56-2.38 (m, 2H), 2.37-2.26 (m, 2H), 2.26-2.16 (m, 1H), 2.16-1.98 (m, 3H).

### Example 2

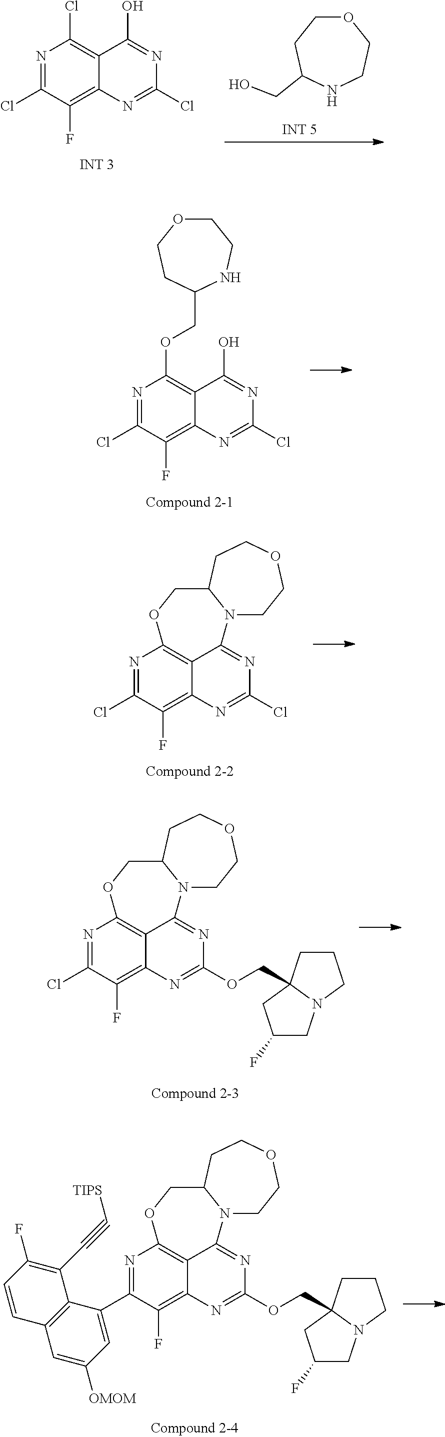

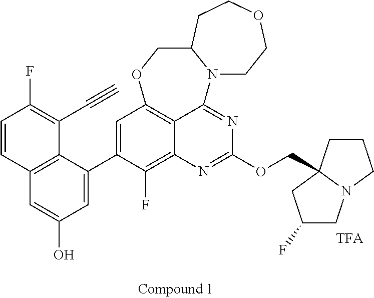 [SMILES: C#Cc1c(F)ccc2cc(O)cc(-c3cc4c5c(nc(OC[C@@]67CCCN6C[C@H](F)C7)nc5c3F)N3CCOCCC3CO4)c12]

A solution of INT 5 (127 mg, 968.19 μmol), NaH (170 mg, 4.25 mmol, 60% wt) in THF (5 mL) was stirred at 0° C. for 0.5 h. Then INT 3 (215 mg, 800.87 mol) was added. The mixture was stirred at 0° C. for 2.5 hours and quenched with water (1 mL). The solution was purified by reversed phase flash chromatography (20% MeCN/water) to give Compound 2-1 (242 mg, 666.35 mol). MS m/z: 363/365 [M+H]⁺.

To a solution of Compound 2-1 (229 mg, 630.56 μmol), DIEA (297 mg, 2.30 mmol) in DCM (8 mL) was added POCl₃ (493 mg, 3.22 mmol) and stirred at −10° C. for 1 hour. The mixture was quenched with sat. NaHCO₃ (20 mL) and separated with water and EtOAc. The organic layer was dried over anhydrous Na₂SO₄ and concentrated under reduced pressure. The residue was purified by Pre-TLC (eluted with MeOH:DCM=1:15, v/v) to give Compound 2-2 (98 mg, 283.93 mol). MS m/z: 345/347 [M+H]⁺.

A solution of Compound 2-2 (98 mg, 283.93 mmol), ((2R,7aS)-2-fluorotetrahydro-1H-pyrrolizin-7a(5H)-yl) methanol (73 mg, 458.54 μmol), KF (56 mg, 963.91 μmol) in DMSO (4 mL) was stirred at 90° C. overnight under nitrogen atmosphere. The mixture was separated with water and EtOAc. The organic layer was dried over anhydrous Na₂SO₄ and concentrated under reduced pressure. The residue was purified by Pre-TLC (eluted with MeOH:DCM=1:10, v/v) to give Compound 2-3 (28 mg, 59.84 μmol). MS m/z: 468/470 [M+H]⁺.

To a solution of Compound 2-3 (28 mg, 59.84 μmol), INT 2 (54 mg, 105.36 mmol) in toluene (5 mL) and water (1 mL) were added Cs₂CO₃ (63 mg, 193.36 μmol) and cataCXium A Pd G3 (12 mg, 16.48 μmol). The reaction mixture was stirred at 100° C. overnight under nitrogen atmosphere. The reaction was extracted with EtOAc (20 mL) and the filtrate was concentrated under reduced pressure. The residue was purified by Pre-TLC (eluted with DCM:MeOH=10:1, v/v) to give Compound 2-4 (57 mg, 69.68 mmol). MS m/z: 818 [M+H]⁺.

A solution of Compound 2-4 (57 mg, 69.68 mmol), HCl (1 mL, 4 M in dioxane) in MeCN (3 mL) was stirred at RT for 1 hour. The mixture was concentrated under reduced pressure. The residue was diluted with sat. NaHCO₃ (20 mL) and extracted with EtOAc (2×20 mL). The organic layer was combined, dried over anhydrous Na₂SO₄ and concentrated under reduced pressure. The residue was dissolved in DMF (3 mL) and CsF (181 mg, 1.19 mmol) was added. The reaction mixture was stirred at 45° C. for 2 hours under nitrogen atmosphere. The reaction mixture was purified by Prep-HPLC (C18 column, A: 0.1% TFA in water, B: CH₃CN, Gradient: 15% B to 35% B in 32 min at a flow rate of 40 mL/min, 240 nm) and the product fractions were lyophilized to give Compound 2 (10.2 mg, 0.789 mmol, TFA salt). MS m/z: 618 [M+H]⁺.

### Example 3

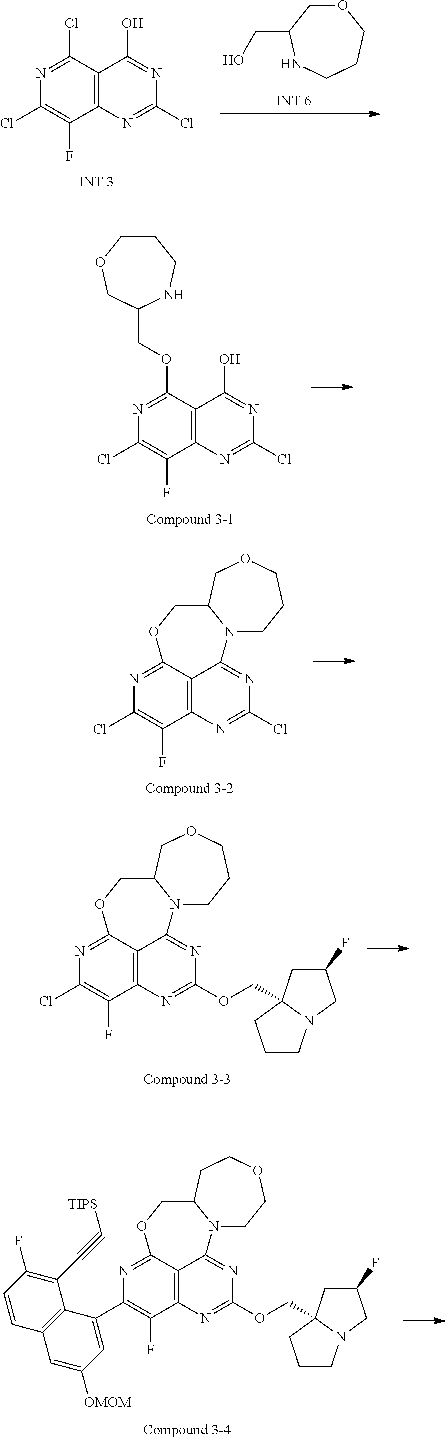

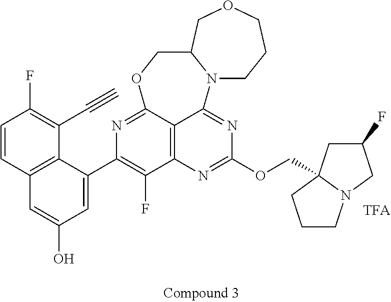 [SMILES: C#Cc1c(F)ccc2cc(O)cc(-c3nc4c5c(nc(OC[C@@]67CCCN6C[C@H](F)C7)nc5c3F)N3CCCOCC3CO4)c12]

To a 0° C. solution of INT 6 (92 mg, 701.37 mol) in THF (10 mL) was added NaH (106 mg, 4.42 mmol). The mixture was stirred at 0° C. for 1 hour. INT 3 (190 mg, 707.74 μmol) was added to the reaction mixture. The mixture was stirred at 0° C. for 1 hour. The mixture was quenched with ice water (5 mL), filtered and the filtrate was concentrated under reduced pressure. The residue was purified by reversed phase flash chromatography to give Compound 3-1 (175 mg, 481.87 μmol, 68.1% yield). MS: m/z 363 (M+H)⁺.

To a 0° C. solution of Compound 3-1 (142 mg, 391.00 mol) and DIEA (185 mg, 1.43 mmol) in DCM (5 mL) was added phosphorus oxychloride (400 mg, 2.61 mmol). The mixture was stirred at 0° C. for 1 hour. The mixture was quenched with sat. NaHCO₃ (aq., 20 mL), extracted with DCM (10 mL×2). The combined organic extracts were washed with brine (10 mL×3), dried over anhydrous Na₂SO₄. The mixture was concentrated under reduced pressure. The residue was purified by reversed phase flash chromatography to give Compound 3-2 (23 mg, 66.64 μmol, 17.04% yield). MS: m/z 345 (M+H)⁺.

A solution of Compound 3-2 (23 mg, 66.64 μmol), ((2R,7aS)-2-fluorotetrahydro-1H-pyrrolizin-7a(5H)-yl)methanol (61 mg, 383.16 μmol), DIEA (24 mg, 185.70 μmol) and some molecular sieves in 1,4-dioxane (5 mL) were stirred at 100° C. overnight. The mixture was concentrated and purified by reversed phase flash chromatography to give Compound 3-3 (82 mg, 175.25 mol, crude) MS: m/z 468 (M+H)⁺.

A solution of Compound 3-3 (82 mg, 175.25 μmol), INT 2 (241 mg, 470.21 μmol), cataCXium A Pd G3 (64 mg, 87.88 μmol) and Cs₂CO₃ (203 mg, 623.05 μmol) in toluene (10 mL) and H₂O (2 mL) were stirred at 100° C. overnight under nitrogen atmosphere. The mixture was diluted with water (10 mL), extracted with EtOAc (10 mL×3), and the organic phases were combined, washed with saturated sodium chloride (10 mL×3), dried over anhydrous sodium sulfate. The organic phase was filtered and concentrated, purified by flash chromatography to give Compound 3-4 (62 mg, 75.79 mol, 43.25% yield). MS: m/z 818 (M+H)⁺.

To a solution of Compound 3-4 (62 mg, 75.79 μmol) in MeCN (5 mL) was added HCl solution (1 mL, 4 M in dioxane). The mixture was stirred at RT for 2 hours. TEA was added to adjust to pH=8. The mixture was filtered and the filtrate was collected and concentrated under reduced pressure. The residue was dissolved in DMF (5 mL) and CsF (1656 mg, 10.90 mmol) was added. The reaction mixture was stirred at 45° C. for 2 hours. The mixture was filtered and the filtrate was concentrated under reduced pressure. The residue was purified by pre-HPLC (C18 column, A: 0.1% TFA in water, B: CH₃CN, Gradient: 15% B to 40% B in 36 min at a flow rate of 60 mL/min, 230 nm) and the product fractions were lyophilized to give Compound 3 (6.8 mg, 11.01 μmol, 14.52% yield, TFA salt). MS: m/z 618 (M+H)⁺.

### Example 4

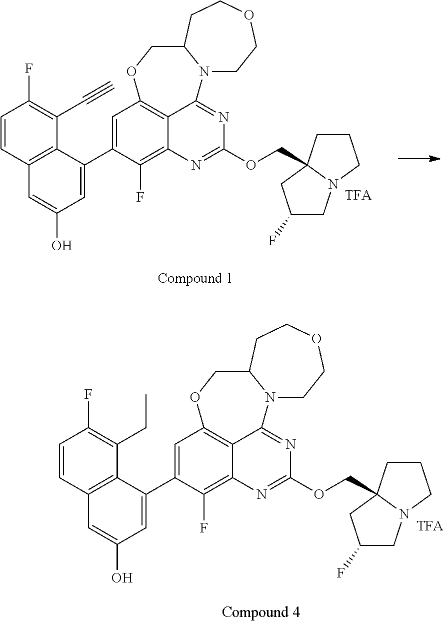 [SMILES: C#Cc1c(F)ccc2cc(O)cc(-c3cc4c5c(nc(OC[C@@]67CCCN6C[C@H](F)C7)nc5c3F)N3CCOCCC3CO4)c12 ; CCc1c(F)ccc2cc(O)cc(-c3cc4c5c(nc(OC[C@@]67CCCN6C[C@H](F)C7)nc5c3F)N3CCOCCC3CO4)c12]

To a solution of Compound 1 (37.1 mg, 0.052 mmol) in MeOH (10 mL) was added Pd(OH)₂/C (21 mg, 10% content). The suspension was degassed under reduced pressure and purged with hydrogen three times. The mixture was stirred at room temperature for 3 hours. After completion, the mixture was filtered and concentrated under reduced pressure. The residue was purified by Pre-HPLC (C18 column, A: 0.1% TFA in water, B: CH₃CN, Gradient: 15% B to 40% B in 30 min at a flow rate of 40 mL/min, 230 nm) and freeze-dried to give Compound 4 (18.1 mg, 24.64 μmol, TFA salt). MS: m/z 621 [M+H].

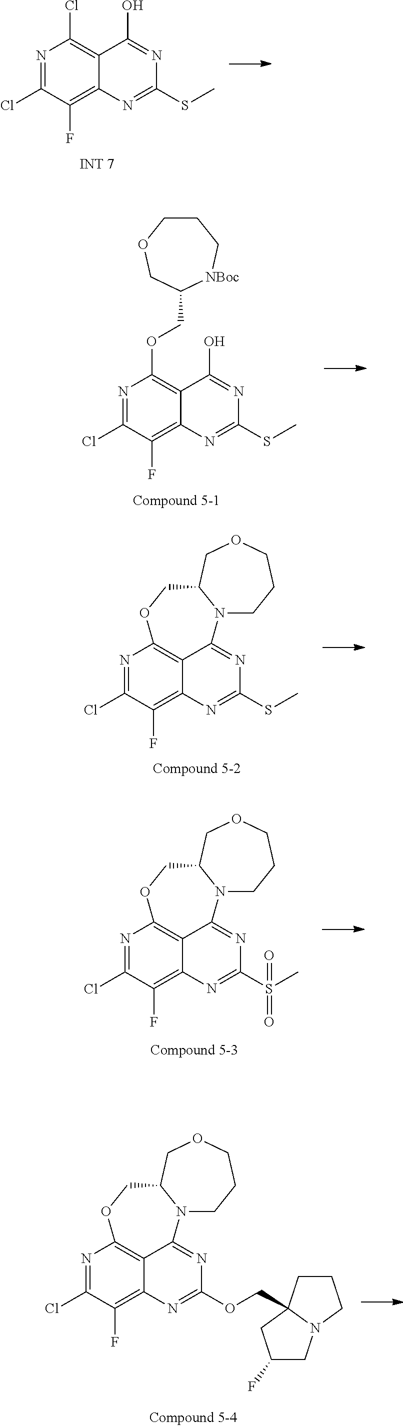 [SMILES: CSc1nc(O)c2c(Cl)nc(Cl)c(F)c2n1 ; CSc1nc(O)c2c(OC[C@@H]3COCCCN3C(=O)OC(C)(C)C)nc(Cl)c(F)c2n1 ; CSc1nc2c3c(nc(Cl)c(F)c3n1)OC[C@@H]1COCCCN21 ; CS(=O)(=O)c1nc2c3c(nc(Cl)c(F)c3n1)OC[C@@H]1COCCCN21 ; Fc1c(Cl)nc2c3c(nc(OC[C@@]45CCCN4C[C@H](F)C5)nc13)N1CCCOC[C@H]1CO2]

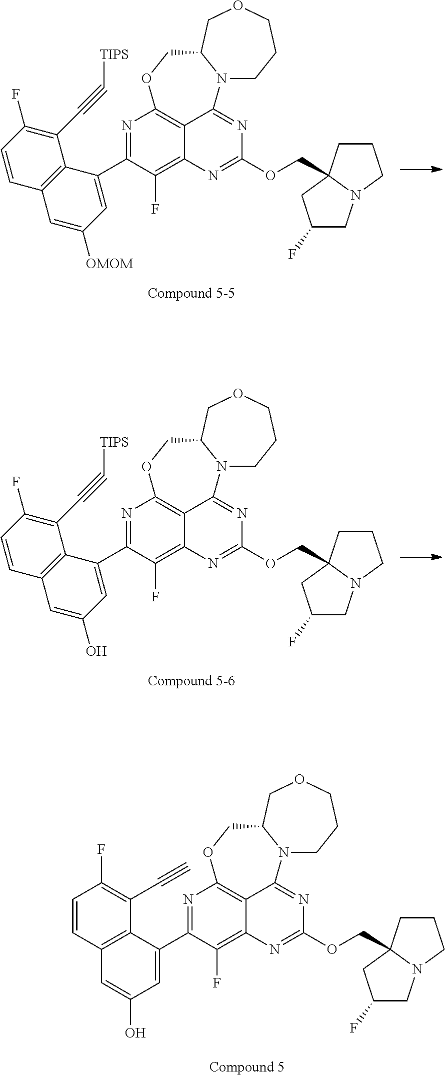

A solution of INT 7 (807 mg, 2.88 mmol) and INT 12 (799 mg, 3.45 mmol) in THF (20 mL) was cooled to −5° C., then NaH (451 mg, 11.27 mmol, 60%) was added. The reaction mixture was stirred for 2 hours at −5° C. Then the mixture was quenched with HCl (1 N), adjusted to pH=3 and extracted with EtOAc (100 mL). The organic layer was dried over anhydrous Na₂SO₄ and concentrated under reduced pressure. The residue was purified with reverse phase flash (eluted with 5-35% CH₃CN in water with 0.1% NH₃—H₂O) to give Compound 5-1 (993 mg, 2.09 mmol, 72.5% yield). MS (ESI, m/z): 475 [M+H]⁺.

To a solution of Compound 5-1 (870 mg, 1.83 mmol) in acetonitrile (20 mL) was added HCl (4 M in 1,4-dioxane, 5 mL). The reaction mixture was stirred at room temperature for 1 hour, then concentrated under reduced pressure. The residue was dissolved in toluene (20 mL), N,N-diisopropylethylamine (2.3675 g, 18.31 mmol) followed with POCl₃ (1.4044 g, 9.15 mmol) were added. The reaction mixture was stirred at room temperature for 1 hour. Then the mixture was quenched with aq. NaHCO₃ (sat., 30 mL), extracted with EtOAc (50 mL), the organic layer was dried over anhydrous Na₂SO₄ and concentrated under reduced pressure. The residue was purified by silica gel chromatography (eluted with 0-50% EtOAc in hex) to give Compound 5-2 (326 mg, 913.67 μmol, 49.8% yield). MS (ESI, m/z): 357 [M+H]⁺.

To a solution of Compound 5-2 (226 mg, 633.40 μmol) in DCM (15 mL) was added m-CPBA (272 mg, 1.57 mmol) at room temperature and stirred for 1 hour. Another batch of m-CPBA (51 mg, 295.53 mol) was added and stirred for 1 hour. Then the mixture was quenched with aq. NaHCO₃ (30 mL), extracted with DCM (2×50 mL), the organic layer was dried over anhydrous Na₂SO₄ and concentrated under reduced pressure. The residue was purified by Pre-TLC (EtOAc) to give Compound 5-3 (194 mg, 498.96 μmol, 78.7% yield). MS (ESI, m/z): 389 [M+H]⁺.

A solution of Compound 5-3 (174 mg, 447.52 μmol), ((2R,7aS)-2-fluorotetrahydro-1H-pyrrolizin-7a(5H)-yl)methanol (139 mg, 873.11 μmol) in THF (15 mL) was cooled to −30° C., then t-BuONa (48 mg, 499.46 μmol) was added. The reaction mixture was stirred for 0.5 hour at −30° C. Then the mixture was quenched with aq. NH₄Cl (sat., 30 mL), extracted with EtOAc (50 mL×2), the combined organic layer was dried over anhydrous Na₂SO₄ and concentrated under reduced pressure. The residue was purified by Pre-TLC (eluted with DCM:MeOH=15:1, v/v) to give Compound 5-4 (185 mg, 395.38 μmol, 88.3% yield). MS (ESI, m/z): 468 [M+H]⁺.

A solution of Compound 5-4 (185 mg, 395.38 μmol), INT 2 (404 mg, 788.23 μmol), cataCXium A Pd G3 (64 mg, 87.87 μmol), Cs₂CO₃ (378 mg, 1.16 mmol) in toluene (8 mL) and water (2 mL) was stirred at 100° C. for 16 hours under nitrogen atmosphere. The reaction mixture was diluted with EtOAc (50 mL) and washed with water (30 mL). The organic layer was dried over anhydrous Na₂SO₄ and concentrated under reduced pressure. The residue was purified by Pre-TLC (eluted with DCM:MeOH=15:1, v/v) to give Compound 5-5 (296 mg, 361.85 μmol, 91.5% yield). MS (ESI, m/z): 818 [M+H]⁺.

To a solution of Compound 5-5 (296 mg, 361.85 μmol) in CH₃CN (15 mL) was added HCl (4 M in 1,4-dioxane, 5 mL). The reaction mixture was stirred at room temperature for 1 hour. The resulting mixture was concentrated under reduced pressure. The residue was dissolved in EtOAc (50 mL) and washed with aq. NaHCO₃ (sat., 2×30 mL). The organic layer was dried over anhydrous Na₂SO₄ and concentrated under reduced pressure to give Compound 5-6 (316 mg, 408.29 mol, 112.8% yield). MS (ESI, m/z): 774 [M+H]⁺.

To a solution of Compound 5-6 (316 mg, 408.29 μmol) in DMF (4 mL) was added CsF (0.62 g, 4.08 mmol). The reaction mixture was stirred at 40° C. for 3 hours, then filtered to collect the filtrate. The filtrate was concentrated under reduced pressure. The residue was purified by Prep-HPLC (YMC-Triart C18-S12 nm, phase A: 0.05% NH₄OH in water, phase B: CH₃CN, Gradient: 15% B to 70% B in 25 min at a flow rate of 70 mL/min, 240 nm) and freeze-dried to give Compound 5 (112.9 mg, 182.79 μmol, 44.7% yield). MS (ESI, m/z): 618 [M+H]⁺.

¹H NMR (600 MHz, MeOD) δ 7.86-7.78 (m, 1H), 7.33-7.27 (m, 2H), 7.24-7.14 (m, 1H), 5.37-5.17 (m, 2H), 4.75-4.68 (m, 1H), 4.56 (dd, J=13.3, 3.1 Hz, 1H), 4.42-4.34 (m, 1H), 4.32-4.27 (m, 1H), 4.25-4.12 (m, 2H), 4.02-3.93 (m, 1H), 3.86-3.77 (m, 1H), 3.68-3.62 (m, 1H), 3.58-3.51 (m, 1H), 3.50-3.41 (m, 1H), 3.41-3.34 (m, 1H), 3.26-3.14 (m, 2H), 3.06-2.96 (m, 1H), 2.39-2.08 (m, 4H), 2.04-1.81 (m, 4H).

### Example 6

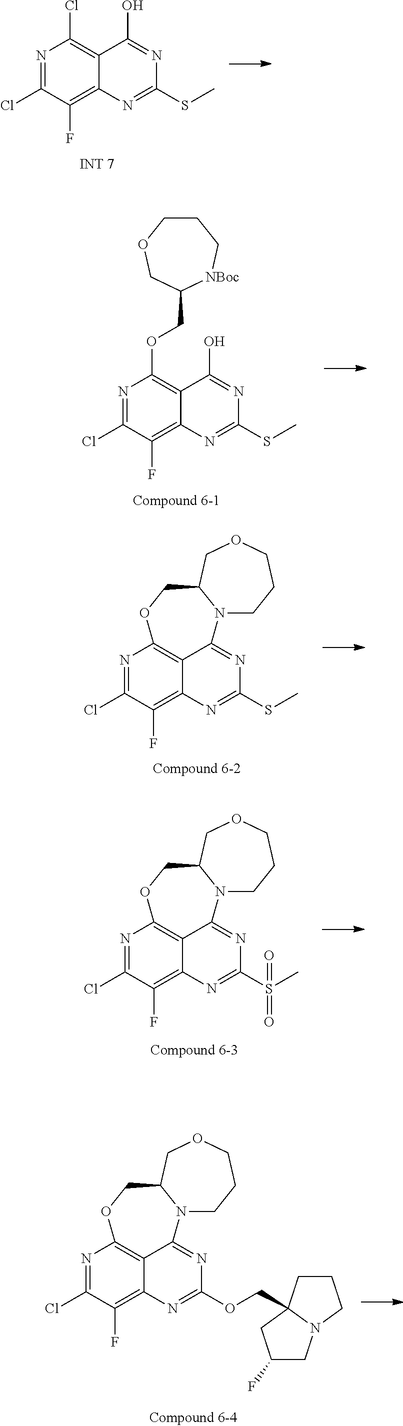 [SMILES: CSc1nc(O)c2c(Cl)nc(Cl)c(F)c2n1 ; CSc1nc(O)c2c(OC[C@H]3COCCCN3C(=O)OC(C)(C)C)nc(Cl)c(F)c2n1 ; CSc1nc2c3c(nc(Cl)c(F)c3n1)OC[C@H]1COCCCN21 ; CS(=O)(=O)c1nc2c3c(nc(Cl)c(F)c3n1)OC[C@H]1COCCCN21 ; Fc1c(Cl)nc2c3c(nc(OC[C@@]45CCCN4C[C@H](F)C5)nc13)N1CCCOC[C@@H]1CO2]

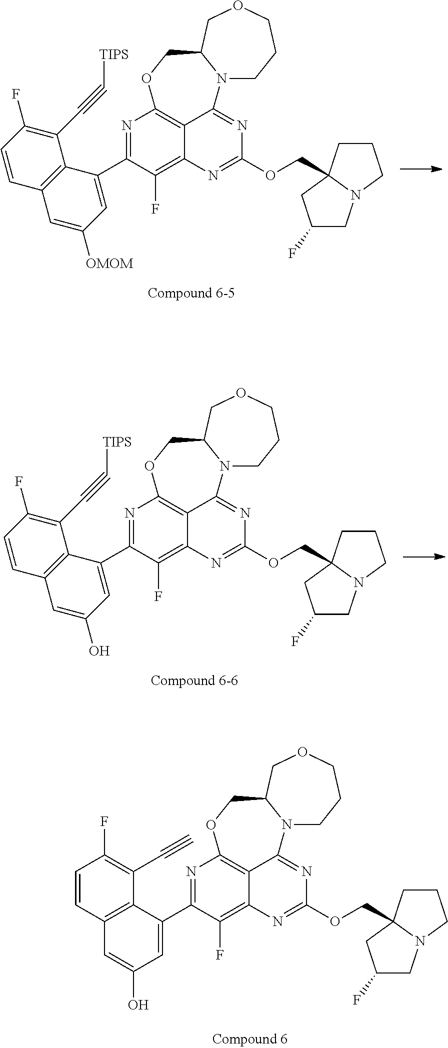

Compound 6 was prepared following the procedure for the synthesis of Compound 5 with INT 7 and INT 13.

### Example 7

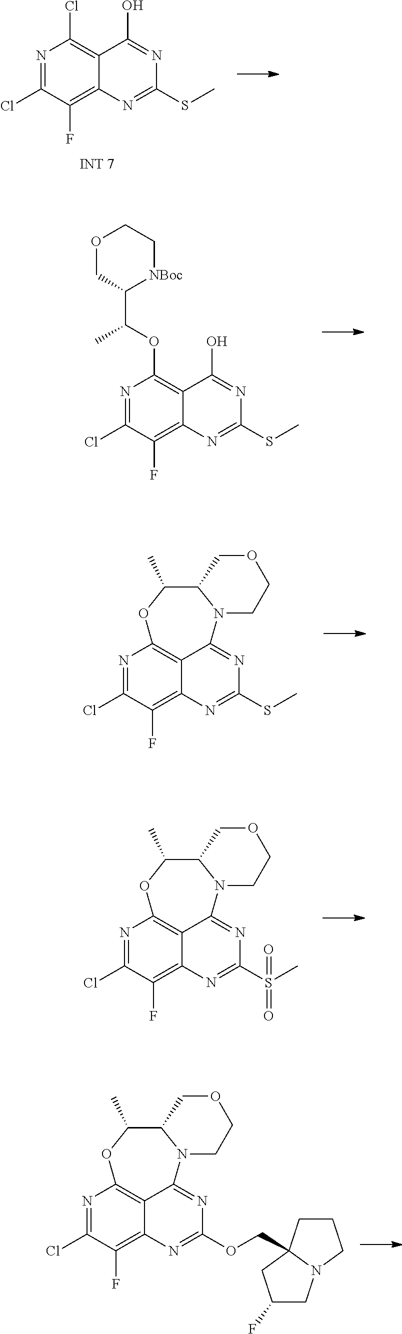 [SMILES: CSc1nc(O)c2c(Cl)nc(Cl)c(F)c2n1 ; CSc1nc(O)c2c(O[C@H](C)[C@@H]3COCCN3C(=O)OC(C)(C)C)nc(Cl)c(F)c2n1 ; CSc1nc2c3c(nc(Cl)c(F)c3n1)O[C@H](C)[C@@H]1COCCN21 ; C[C@H]1Oc2nc(Cl)c(F)c3nc(S(C)(=O)=O)nc(c23)N2CCOC[C@@H]12 ; C[C@H]1Oc2nc(Cl)c(F)c3nc(OC[C@@]45CCCN4C[C@H](F)C5)nc(c23)N2CCOC[C@@H]12]

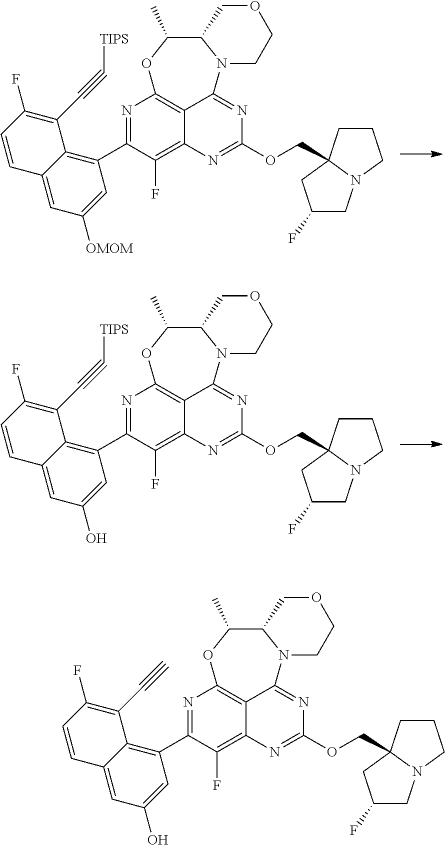

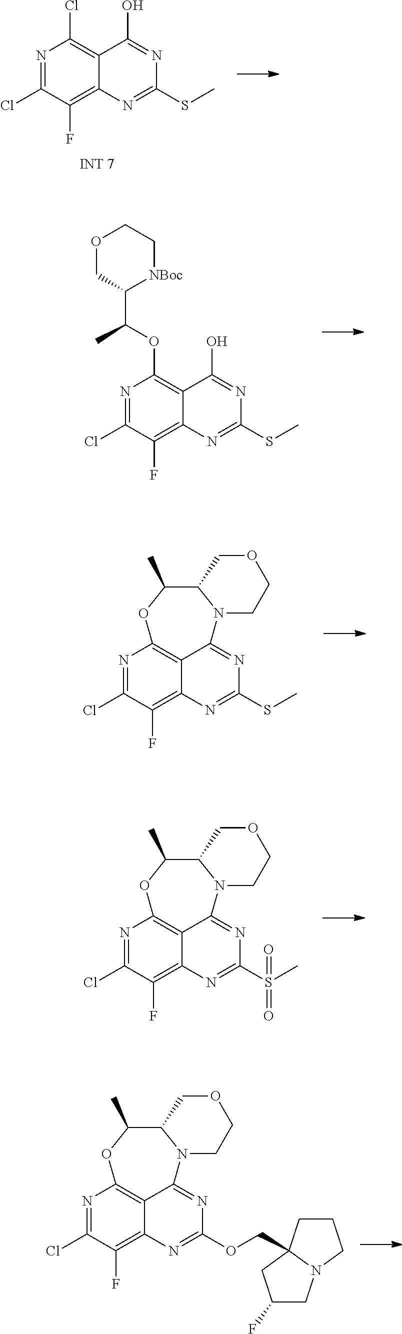 [SMILES: CSc1nc(O)c2c(Cl)nc(Cl)c(F)c2n1 ; CSc1nc(O)c2c(O[C@@H](C)[C@@H]3COCCN3C(=O)OC(C)(C)C)nc(Cl)c(F)c2n1 ; CSc1nc2c3c(nc(Cl)c(F)c3n1)O[C@@H](C)[C@@H]1COCCN21 ; C[C@@H]1Oc2nc(Cl)c(F)c3nc(S(C)(=O)=O)nc(c23)N2CCOC[C@@H]12 ; C[C@@H]1Oc2nc(Cl)c(F)c3nc(OC[C@@]45CCCN4C[C@H](F)C5)nc(c23)N2CCOC[C@@H]12]

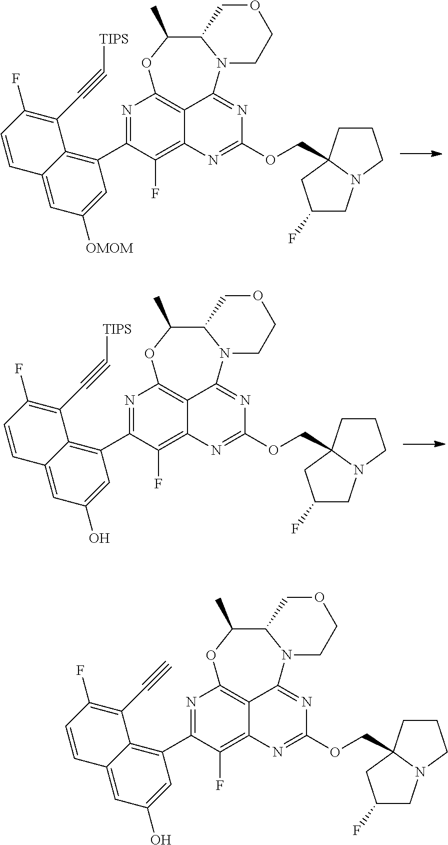

A solution of INT 7 (604 mg, 2.15 mmol) and INT 10A (412 mg, 1.78 mmol) in THF (100 mL) was cooled to −5° C., then NaH (387 mg, 9.68 mmol, 60% content) was added at 0-5° C. The reaction mixture was stirred for 19 hours at room temperature, quenched with hydrochloric acid (1 N), adjusted to pH=7 and extracted with EtOAc (30 mL×3). The combined organic layer was dried over anhydrous Na₂SO₄, filtered and concentrated under reduced pressure. The residue was purified by Prep-HPLC (C18 column, A: 0.1% NH₃·H₂O in water, B: CH₃CN, Gradient: 5% B to 85% B in 35 min at a flow rate of 60 mL/min, 254 nm) to give Compound 7A-1 (242.6 mg, 23.76% yield). MS: m/z: 475 [M+H]⁺.

A solution of Compound 7A-1 (242.6 mg, 510.81 μmol) in HCl (4 M in 1,4-dioxane, 5 mL) was stirred at room temperature for 1 hour and then concentrated under reduced pressure. To the residue, toluene (5 mL), N,N-diisopropylethylamine (1 mL) and POCl₃ (0.5 mL) were added. The reaction mixture was stirred at room temperature for 1 hour, then quenched with aq. NaHCO₃ (sat., 20 mL), extracted with EtOAc (20 mL×2), the combined organic layer was dried over anhydrous Na₂SO₄, filtered and the filtrate was concentrated under reduced pressure. The residue was purified by silica gel column chromatography (eluted with 0-50% EtOAc in hex) to give Compound 7A-2 (38.6 mg, 21.18% yield). MS: m/z: 357 [M+H]⁺.

To a solution of Compound 7A-2 (38.6 mg, 108.18 μmol) in DCM (15 mL) was added m-CPBA (36 mg, 208.62 μmol) at room temperature and stirred for 1 hour. Another batch of m-CPBA (9 mg, 52.15 μmol) was added and stirred for 1 hour. Then the mixture was quenched with aq. NaHCO₃ (30 mL), extracted with DCM (20 mL×2), the combined organic layer was dried over anhydrous Na₂SO₄, filtered and the filtrate was concentrated under reduced pressure to give Compound 7A-3 (31 mg, 73.70% yield), which was used directly in the next step without purification. MS: m/z: 389 [M+H]⁺.

A solution of Compound 7A-3 (31 mg, 79.73 μmol), ((2R,7aS)-2-fluorotetrahydro-1H-pyrrolizin-7a(5H)-yl)methanol (31 mg, 194.72 μmol) in THF (15 mL) was cooled to 0-5° C., then t-BuONa (11 mg, 114.46 μmol) was added. The reaction mixture was stirred for 1 hour at 0-5° C. The mixture was quenched with aq. NH₄Cl (sat., 10 mL), extracted with EtOAc (15 mL×2), the combined organic layer was dried over anhydrous Na₂SO₄, filtered and the filtrate was concentrated under reduced pressure to give Compound 7A-4 (34.7 mg, 93.01% yield) which was used directly in the next step without purification. MS: m/z: 468 [M+H]⁺.

To a solution of Compound 7A-4 (34.7 mg, 74.16 mol) in toluene (15 mL) and water (3 mL) were added INT 2 (99 mg, 193.16 mol), cataCXium A Pd G3 (50 mg, 68.66 μmol) and cesium carbonate (132 mg, 193.16 mol). The reaction mixture was stirred at 100° C. for 16 hours under nitrogen atmosphere. The mixture was cooled to room temperature, diluted with sat. NaHCO₃ (50 mL) and extracted with DCM (30 mL×2). The combined organic layer was washed with brine (50 mL), then dried over anhydrous Na₂SO₄, filtered and the filtrate was concentrated under reduced pressure. The residue was purified by Pre-TLC to give Compound 7A-5 (34.3 mg, 56.54% yield). MS: m/z: 818 [M+H]⁺.

A solution of Compound 7A-5 (34.3 mg, 41.93 μmol), HCl (4 M in 1,4-dioxane, 2 mL) in DCM (5 mL) was stirred at room temperature for 1 hour. The solution was diluted with 10% NaHCO₃ solution (50 mL) and extracted with DCM (30 mL×2). The combined organic layer was washed with sat. NaCl (aq. 50 mL), dried over anhydrous Na₂SO₄ and concentrated under reduced pressure to give Compound 7A-6 (18.2 mg, 56.08% yield). MS: m/z: 774 [M+H]⁺.

To a solution of Compound 7A-6 (18.2 mg, 23.52 μmol) in DMF (10 mL) was added CsF (59 mg, 388.40 μmol). The reaction mixture was stirred for 16 hours at 35° C. under nitrogen atmosphere. The solution was diluted with sat. NaHCO₃ (50 mL), extracted with EtOAc (30 mL×2), washed with sat. NaCl (aq. 50 mL×2), dried over anhydrous Na₂SO₄ and concentrated under reduced pressure. The residue was purified by Prep-HPLC (C18 column, phase A: 0.05% NH₃·H₂O in water, phase B: CH₃CN, Gradient: 20% B to 47% B in 30 min at a flow rate of 40 mL/min, 230 nm) to give Compound 7A (0.9 mg, 6.41% yield). MS: m/z: 618 [M+H]⁺.

Compound 7B was prepared following the procedure of the synthesis of Compound 7A with INT 7 and INT 10B as starting material.

### Example 8

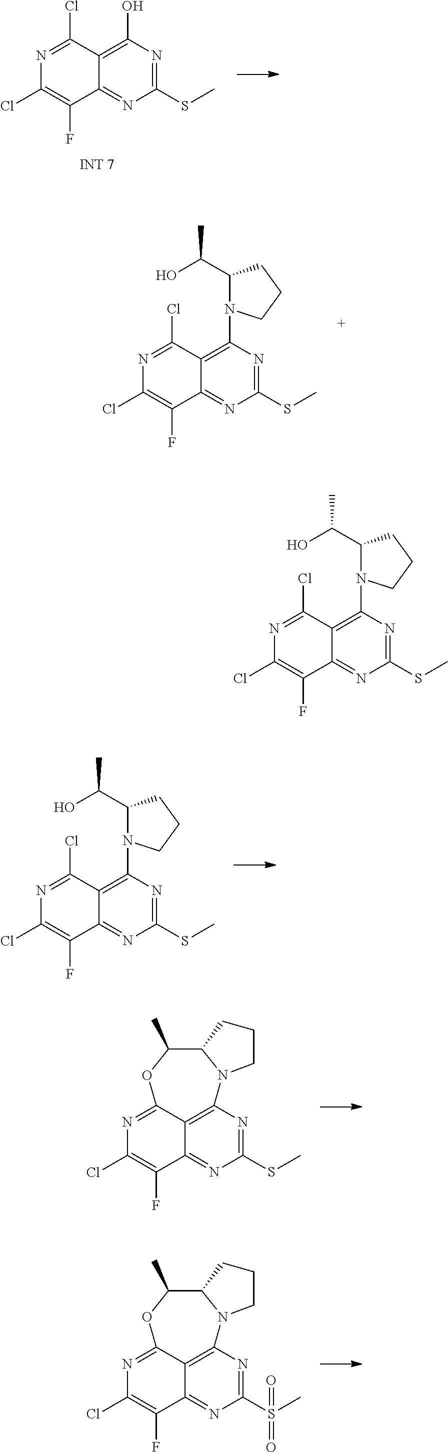 [SMILES: CSc1nc(O)c2c(Cl)nc(Cl)c(F)c2n1 ; CSc1nc(N2CCC[C@H]2[C@H](C)O)c2c(Cl)nc(Cl)c(F)c2n1 ; CSc1nc(N2CCC[C@H]2[C@@H](C)O)c2c(Cl)nc(Cl)c(F)c2n1 ; CSc1nc(N2CCC[C@H]2[C@H](C)O)c2c(Cl)nc(Cl)c(F)c2n1 ; CSc1nc2c3c(nc(Cl)c(F)c3n1)O[C@@H](C)[C@@H]1CCCN21 ; C[C@@H]1Oc2nc(Cl)c(F)c3nc(S(C)(=O)=O)nc(c23)N2CCC[C@@H]12]

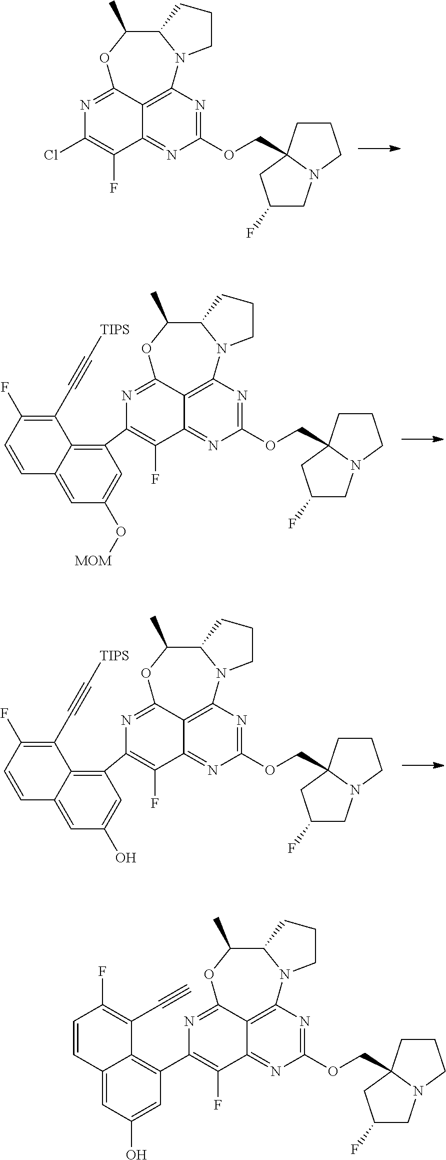

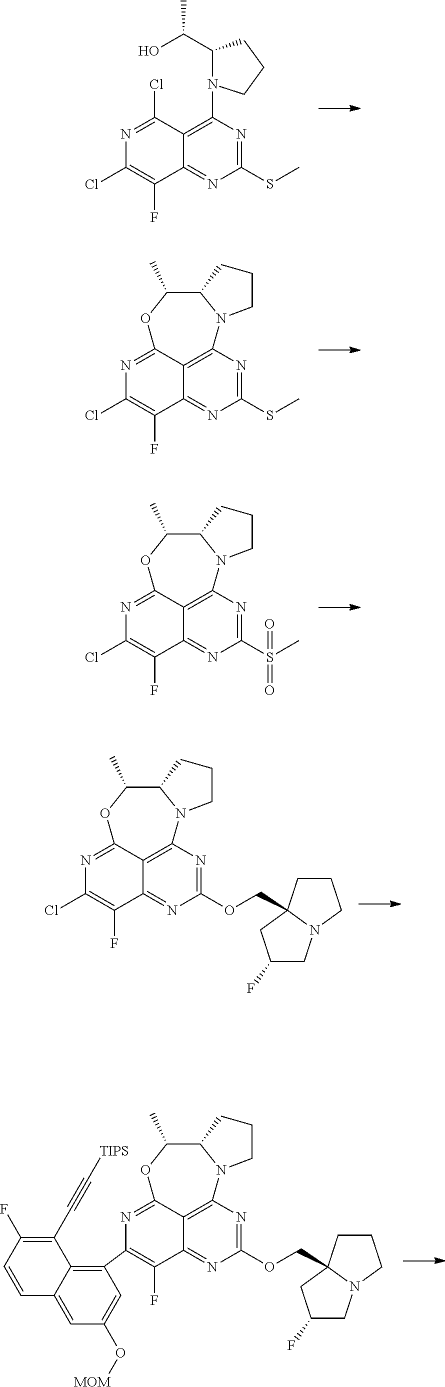

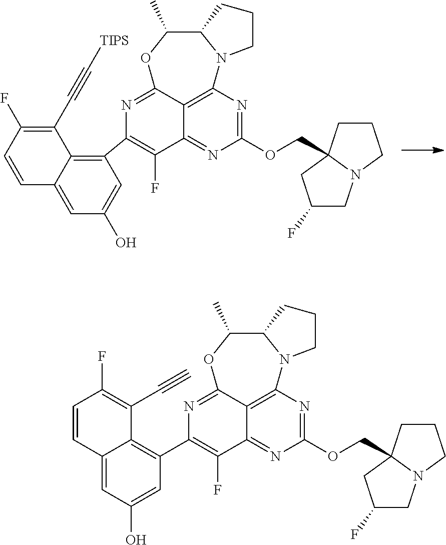

N,N-diisopropylethylamine (1.61 g, 12.45 mmol) was added to a solution of INT 7 (398 mg, 1.42 mmol), POCl₃ (1.88 g, 12.26 mmol) in toluene (5 mL). The reaction mixture was stirred at 80° C. for 1 hour and concentrated under reduced pressure. A solution of the residue in DCM (5 mL) and N,N-diisopropylethylamine (1.22 g, 9.44 mmol) was added to a solution of TNT 9 (131 mg, 1.14 mmol) in DCM (5 mL). The reaction mixture was stirred at 0° C. for 1 hour, then quenched with water and extracted with EtOAc (50 mL). The organic layer was dried over anhydrous Na₂SO₄, filtered and the filtrate was concentrated under reduced pressure. The residue was purified by Pre-TLC (Hex:EtOAc=1:2, v/v) to give Compound 8A-1 (153 mg, 405.55 μmol, 35.66% yield) and Compound 8B-1 (154 mg, 408.20 μmol, 35.89% yield). MS: m/z: 377 [M+H].

A solution of Compound 8A-1 (153 mg, 405.55 μmol) in THF (5 mL) was cooled to −5° C., then NaH (129 mg, 3.23 mmol, 600% content) was added. The reaction mixture was stirred for 1 hour at room temperature, then quenched with saturated ammonium chloride solution, extracted with EtOAc (100 mL). The organic layer was dried over anhydrous Na₂SO₄, filtered and the filtrate was concentrated under reduced pressure to give Compound 8A-2 (170 mg). MS: m/z: 341 [M+H]⁺.

To a solution of Compound 8A-2 (170 mg, 498.82 mol) in DCM (5 mL) was added m-CPBA (219.3 mg, 1.27 mmol) at room temperature and stirred for 1 hour. The mixture was quenched with aq. NaHCO₃ (30 mL) and extracted with EtOAc (50 mL). The organic layer was dried over anhydrous Na₂SO₄, filtered and the filtrate was concentrated under reduced pressure. The residue was purified by Pre-TLC (EtOAc) to give Compound 8A-3 (77 mg, 206.54 mol). MS: m/z: 373[M+H]⁺.

A solution of Compound 8A-3 (77 mg, 206.54 μmol), ((2R,7aS)-2-fluorotetrahydro-1H-pyrrolizin-7a(5H)-yl)methanol (59 mg, 370.60 μmol) in THF (5 mL) was cooled to −10° C., then t-BuONa (26 mg, 270.54 mol) was added. The reaction mixture was stirred for 0.5 h at −10° C., then quenched with sat. aq. NH₄Cl (30 mL) and extracted with EtOAc (50 mL×2). The combined organic layer was dried over anhydrous Na₂SO₄, filtered and the filtrate was concentrated under reduced pressure to give Compound 8A-4 (76 mg, 8143% yield). MS: m/z: 452 [M+H]⁺.

A solution of Compound 8A-4 (76 mg, 168.18 μmol), INT 2 (147 mg 286.81 mol), cataCXium A Pd G3 (67 mg 92.00 μmol), cesium carbonate (229 mg 702.84 μmol) in toluene (10 mL) and water (2 mL) was stirred at 100° C. for 16 hours under nitrogen atmosphere. The mixture was cooled to room temperature, diluted with sat. aq. NaHCO₃ (50 mL) and extracted with EtOAc (30 mL×2). The combined organic layer was washed with sat. aq. NaCl (50 mL), dried over anhydrous Na₂SO₄, filtered and the filtrate was concentrated under reduced pressure. The residue was purified by Pre-TLC (eluted with MeOH:DCM=1:17, v/v) to give Compound 8A-5 (134 mg, 167.08 μmol, 99.35% yield). MS: m/z: 802[M+H]⁺.

A solution of Compound 8A-5 (134 mg, 167.08 μmol) and HCl (4 M in 1,4-dioxane, 1 mL) in DCM (5 mL) was stirred at room temperature for 1 hour. The mixture was diluted with 10% NaHCO₃ solution (50 mL), and extracted with DCM (30 mL×2). The combined organic layer was washed with sat. aq. NaCl (50 mL), dried over anhydrous Na₂SO₄ and concentrated under reduced pressure to give crude Compound 8A-6 (144 mg, 189.98 μmol). MS m/z: 758[M+H]⁺.

A solution of Compound 8A-6 (144 mg, 189.98 μmol), CsF (204 mg 1.3430 mmol) in DMF (6 mL) was stirred for 16 hours at room temperature under nitrogen atmosphere. The solution was diluted with sat. aq. NaHCO₃ (50 mL) and extracted with EtOAc (30 mL×2). The combined organic layer was washed with sat. aq. NaCl (50 mL×2), dried over anhydrous Na₂SO₄, filtered and the filtrate was concentrated under reduced pressure. The residue was purified by Prep-HPLC (C18 column, A: 0.1% TFA in water, B: CH₃CN, Gradient: 15% B to 45% B in 40 min at a flow rate of 60 mL/min, 240 nm), the pH of the product fraction was adjusted to 10 and freeze-dried to give Compound 8A (31.6 mg, 27.65% yield). MS m/z: 602 [M+H]⁺.

Compound 8B was prepared following the procedure of the synthesis of Compound 8A (25.7 mg).

### Example 9

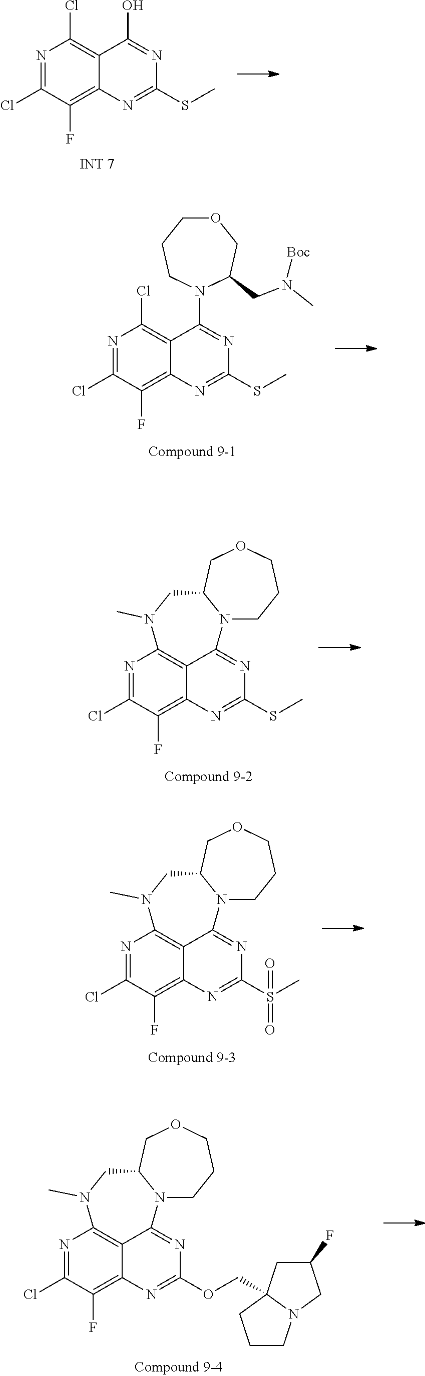 [SMILES: CSc1nc(O)c2c(Cl)nc(Cl)c(F)c2n1 ; CSc1nc(N2CCCOC[C@H]2CN(C)C(=O)OC(C)(C)C)c2c(Cl)nc(Cl)c(F)c2n1 ; CSc1nc2c3c(nc(Cl)c(F)c3n1)N(C)C[C@@H]1COCCCN21 ; CN1C[C@@H]2COCCCN2c2nc(S(C)(=O)=O)nc3c(F)c(Cl)nc1c23 ; CN1C[C@@H]2COCCCN2c2nc(OC[C@@]34CCCN3C[C@H](F)C4)nc3c(F)c(Cl)nc1c23]

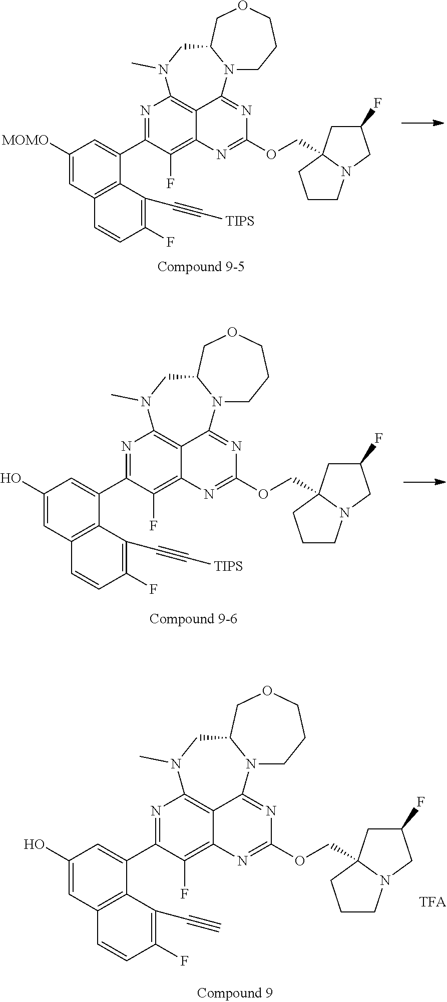

To a solution of INT 7 (90 mg, 0.32 mmol), DIEA (0.5 ml) in toluene (10 mL) was added POCl₃ (0.25 mL). The reaction mixture was stirred at 80° C. for 1.5 hours, then concentrated under reduced pressure. The residue in DCM (5 mL) was added to a solution of INT 14 (77 mg, 0.32 mmol) and DIEA (1 mL) in DCM (10 mL). The reaction mixture was stirred at room temperature for 0.5 h, then diluted with water (30 mL), extracted with EtOAc (30 mL×2). The collected organic layer was washed with brine (30 mL), dried over anhydrous Na₂SO₄ and concentrated under reduced pressure. The residue was purified by Pre-TLC to give Compound 9-1 (77 mg, 0.15 mmol). MS m/z: 506 [M+H]⁺.

To a solution of Compound 9-1 (77 mg, 0.15 mmol) in acetonitrile (3 mL) was added HCl (1 mL, 4 mol/L in dioxane). The reaction mixture was stirred at room temperature for 1 hour. The solution was concentrated under reduced pressure. To a solution of the residue in acetonitrile (5 mL) was added DIEA (1 mL). The reaction mixture was stirred at 80° C. for 1.5 hour, then diluted with water (30 mL), extracted with EtOAc (30 mL×2). The collected organic layer was washed with brine (30 mL), dried over anhydrous Na₂SO₄ and concentrated under reduced pressure. The residue was purified by Pre-TLC to give Compound 9-2 (51 mg, 0.14 mmol). MS m/z: 370 [M+H]⁺.

The crude Compound 9 was prepared following the procedure for the synthesis of Compound 5 in Example 5 with Compound 9-2 as starting material, purified by Prep-HPLC (C18 column, A: 0.1% TFA in water, B: CH₃CN, Gradient: 10% B to 33% B in 38 min at a flow rate of 40 mL/min, 285 nm) and freeze-dried to give Compound 9 (7.1 mg, 9.53 μmol, TFA salt).

### Example 10

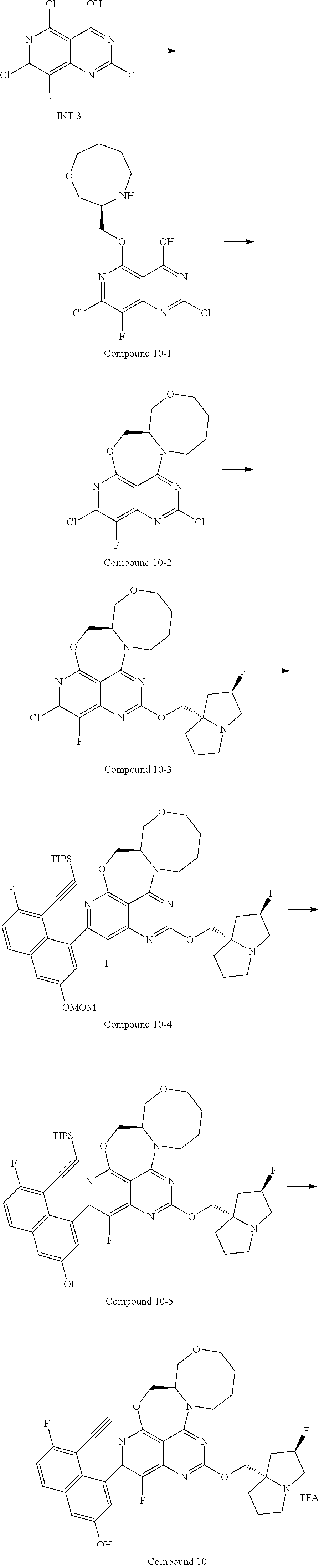

To a solution of INT 8 (375 mg, 1.45 mmol) in THF (10 mL) was added NaH (345 mg, 8.63 mmol, 60% content). After stirred for 10 min, INT 3 (382 mg, 1.42 mmol) was added. The reaction mixture was stirred at room temperature for 4 hours, then quenched with water (0.5 mL). The mixture was purified by reversed phase flash chromatography to give Compound 10-1 (367 mg, 0.97 mmol). LCMS: 377 [M+H]⁺.

To a solution of Compound 10-1 (315 mg, 0.84 mmol) and DIEA (3 ml) in DCM (9 mL) was added POCl₃ (15 drops) at −30° C. The reaction mixture was stirred at −30° C. for 3 hours, then quenched with sat.aq. NaHCO₃ (20 mL) and extracted with DCM (20 mL). The collected organic layer was washed with brine (20 mL), dried over anhydrous Na₂SO₄ and concentrated under reduced pressure. The residue was purified by Pre-TLC to give Compound 10-2 (47 mg, 0.13 mmol). LCMS: 359 [M+H]⁺.

A solution of Compound 10-2 (47 mg, 0.13 mmol), ((2R,7aS)-2-fluorotetrahydro-1H-pyrrolizin-7a(5H)-yl)methanol (44 mg, 0.28 mmol) and KF (33 mg, 0.57 mmol) in DMSO (5 mL) was stirred at 95° C. for 22 hours under nitrogen atmosphere. The mixture was allowed to cool to room temperature, diluted with water (30 mL) and extracted with EtOAc (2×30 mL). The organic layer was washed with aq. NaCl (30 mL), dried over anhydrous Na₂SO₄ and concentrated under reduced pressure. The residue was purified by Pre-TLC to give Compound 10-3 (17 mg, 35.28 mol). MS: m/z: 482 [M+H]⁺.

To a solution of Compound 10-3 (17 mg, 35.28 μmol), INT 2 (33 mg, 64.39 μmol) in toluene (6 mL) and water (1.5 mL) were added Cs₂CO₃ (34 mg, 104.35 mol) and cataCXium A Pd G3 (19 mg, 26.09 μmol). The mixture was stirred at 100° C. overnight under nitrogen atmosphere. The mixture was allowed to cool to room temperature, diluted with water (30 mL) and extracted with EtOAc (2×30 mL). The organic layer was washed with aq. NaCl (30 mL), dried over anhydrous Na₂SO₄ and concentrated under reduced pressure. The residue was purified by Pre-TLC (eluted with DCM:MeOH=15:1, v/v) to give Compound 10-4 (20 mg, 24.04 μmol). MS m/z: 832 [M+H]⁺.

A solution of Compound 10-4 (20 mg, 24.04 μmol), HCl (4 M in 1,4-dioxane, 1 mL) in CH₃CN (3 mL) was stirred at RT for 1 hour. The solution was concentrated under reduced pressure, diluted with sat. aq. NaHCO₃ (20 mL) and extracted with EtOAc (30 mL×2). The organic layer was washed with brine (30 mL), dried over anhydrous Na₂SO₄ and concentrated under reduced pressure to give Compound 10-5 (crude, 29 mg, 36.80 mol). MS m/z: 788 [M+H]⁺.

A solution of Compound 10-5 (29 mg, 36.80 μmol), CsF (0.26 g, 1.71 mmol) in DMF (5 mL) was stirred at 40° C. for 2 hour. The mixture was diluted with water (30 mL) and extracted with EtOAc (30 mL). The collected organic layer was dried over anhydrous Na₂SO₄ and concentrated under reduced pressure. The residue was purified by Prep-HPLC (C18 column, A: 0.1% TFA in water, B: CH₃CN, Gradient: 15% B to 40% B in 37 min at a flow rate of 40 mL/min, 235 nm), the product fractions were lyophilized to give Compound 10 (6.1 mg, 8.18 μmol, TFA salt). MS m/z: 632 [M+H]⁺.

¹H NMR (600 MHz, MeOD) δ 7.93-7.83 (m, 1H), 7.43-7.32 (m, 2H), 7.23 (m, 1H), 5.58 (d, 1H), 4.84-4.73 (m, 2H), 4.68-4.49 (m, 2H), 4.38-3.77 (m, 7H), 3.70-3.43 (m, 5H), 2.75-2.54 (m, 2H), 2.53-2.23 (m, 4H), 2.16 (s, 1H), 1.91-1.51 (m, 3H).

### Example 11

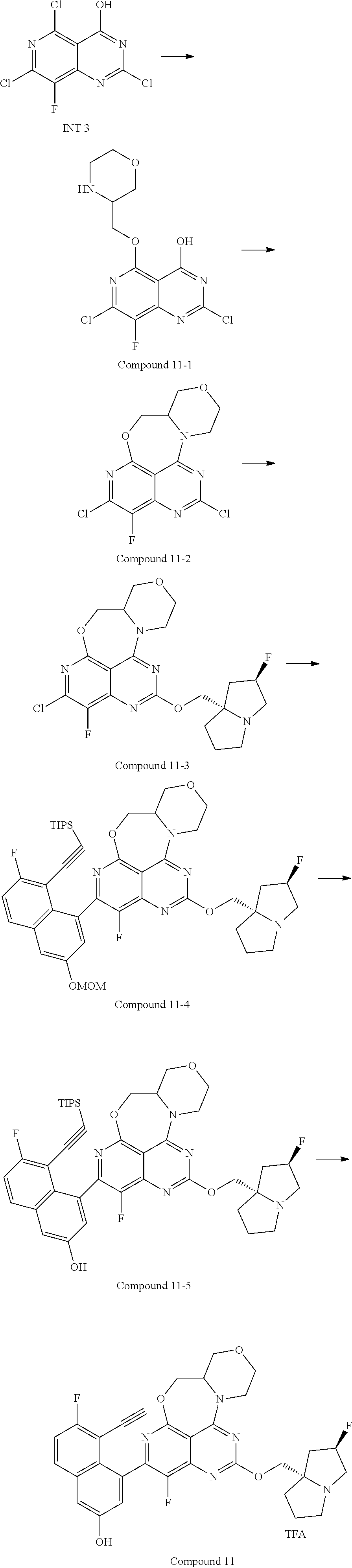

To a solution of morpholin-3-ylmethanol (81 mg, 0.69 mmol) in THF (5 mL) was added NaH (111 mg, 4.63 mmol, 60% content) at −10° C. After stirred for 10 min, INT 3 (152 mg, 0.57 mmol) was added to the mixture. The reaction mixture was stirred at room temperature for 2 hours, then quenched with water (0.5 mL). The mixture was purified by reversed phase flash chromatography to give Compound 11-1 (181 mg, 0.52 mmol). MS m/z: 349 [M+H]⁺.

To a solution of Compound 11-1 (168 mg, 0.48 mmol) and DIEA (379 mg, 2.93 mmol) in DCM (5 mL) was added POCl₃ (0.8 ml) at −30° C. The reaction mixture was stirred at −30° C. for 1.5 hours, then quenched with sat. aq. NaHCO₃ (30 mL) and extracted with DCM (30 mL). The collected organic layer was washed with brine (20 mL), dried over anhydrous Na₂SO₄ and concentrated under reduced pressure. The residue was purified by reversed phase flash chromatography to give Compound 11-2 (62 mg, 0.19 mmol). MS m/z: 331 [M+H]⁺.

Compound 11-5 was prepared following the procedure for the synthesis of Compound 12-5 in Example 12 with Compound 11-2.

A solution of Compound 11-5 (49 mg, 64.48 μmol), CsF (0.50 g, 3.29 mmol) in DMF (4 mL) was stirred at 40° C. for 20 hours. The mixture was diluted with water (30 mL) and extracted with EtOAc (30 mL). The collected organic layer was dried over anhydrous Na₂SO₄ and concentrated under reduced pressure. The residue was purified by Prep-HPLC (C18 column, A: 0.1% TFA in water, B: CH₃CN, Gradient: 15% B to 40% B in 31 min at a flow rate of 40 mL/min, 230 nm) and the product fractions were lyophilized to give Compound 11 (26.6 mg, 37.07 μmol, TFA salt). MS m/z: 604 [M+H]⁺.

¹H NMR (600 MHz, MeOD) δ 7.95-7.82 (m, 1H), 7.42-7.30 (m, 2H), 7.24 (s, 1H), 5.58 (d, 1H), 5.23 (d, 1H), 4.80-4.50 (m, 4H), 4.33-4.20 (m, 1H), 4.16-3.81 (m, 4H), 3.78-3.63 (m, 2H), 3.54 (s, 1H), 3.52-3.36 (m, 3H), 2.77-2.55 (m, 2H), 2.50-2.39 (m, 1H), 2.41-2.29 (m, 2H), 2.17 (s, 1H).

### Example 12

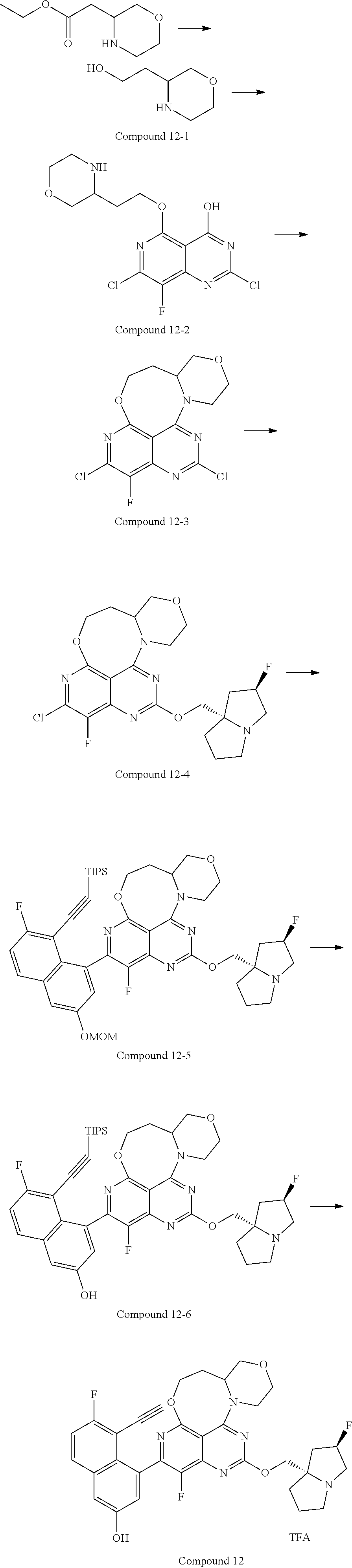

To a solution of ethyl 2-(morpholin-3-yl)acetate (314 mg, 1.81 mmol) in THF (10 mL) was added LiAlH₄ (132 mg, 3.48 mmol) in portions. The mixture was stirred at room temperature for 1.5 hours, then quenched with water (1 mL), filtered and concentrated under reduced pressure to give Compound 12-1 (164 mg, 1.25 mmol). MS m/z: 132 [M+H]⁺.

To a solution of Compound 12-1 (95 mg, 0.72 mmol) in THF (20 mL) was added NaH (142 mg, 3.55 mmol, 60% content) at 0° C. After stirred for 20 min, INT 3 (233 mg, 0.87 mmol) was added to the mixture. The reaction mixture was stirred at room temperature for 3 hours, then quenched with water (2 mL) and purified by reversed phase flash chromatography to give Compound 12-2 (185.9 mg, 0.51 mmol). MS m/z: 363 [M+H]⁺.

To a solution of Compound 12-2 (402 mg, 1.11 mmol), DIEA (454 mg, 3.51 mmol) in acetonitrile (20 mL) was added POCl₃ (552 mg, 3.60 mmol). The reaction mixture was stirred at 0° C. for 0.5 hour, then quenched with sat. aq. NaHCO₃ and extracted with EtOAc (30 mL×2). The collected organic layer was dried over anhydrous Na₂SO₄ and concentrated under reduced pressure. The residue was purified by Prep-TLC to give Compound 12-3 (29 mg, 0.084 mmol). MS m/z: 345 [M+H]⁺.

To a solution of Compound 12-3 (29 mg, 0.084 mmol), ((2R,7aS)-2-fluorotetrahydro-1H-pyrrolizin-7a(5H)-yl)methanol (19 mg, 0.012 mmol) in THF (5 mL) was added t-BuONa (15 mg, 0.016 mmol) at 0° C. The reaction mixture was stirred for 2 days at RT. The mixture was concentrated under reduced pressure and purified by Pre-TLC to give Compound 12-4 (20 mg, 0.043 mmol). MS: m/z: 468 [M+H]⁺.

Compound 12-6 was prepared following the procedure for the synthesis of Compound 12-5 in Example 12 with Compound 12-4.

A solution of Compound 12-6 (14 mg, 0.017 mmol), CsF (352 mg, 2.32 mmol) in DMF (5 mL) was stirred at RT for 16 hours. The mixture was diluted with water (20 mL) and extracted with EtOAc (2×20 mL). The combined organic layer was dried over anhydrous Na₂SO₄ and concentrated under reduced pressure. The residue was purified by Prep-HPLC (C18 column, A: 0.1% TFA in water, B: CH₃CN, Gradient: 10% B to 36% B in 36 min at a flow rate of 40 mL/min, 234 nm) and the product fractions were lyophilized to give Compound 12 (2.4 mg, 3.28 μmol, TFA salt). MS m/z: 618 [M+H]⁺.

### Example 13

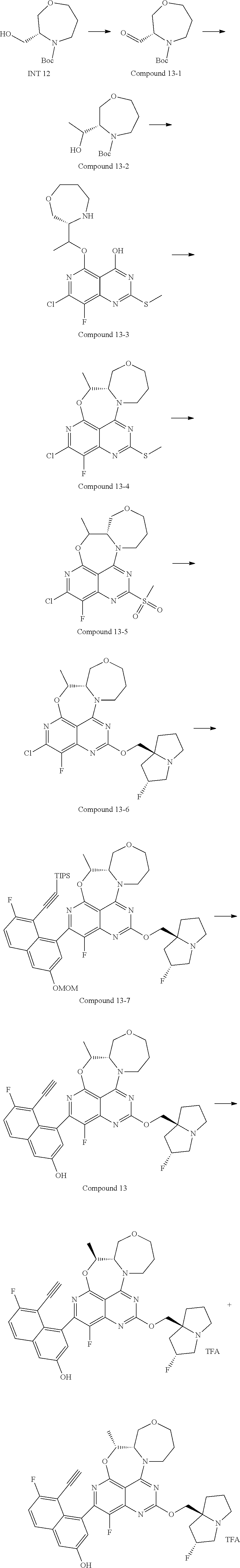

To a 0° C. solution of INT 12 (1059 mg, 4.58 mmol) in DCM (20 mL) was added Dess-Martin periodinane (2224 mg, 5.23 mmol). The mixture was stirred at room temperature for 2 hours. The mixture was quenched with sat. aq. NaHCO₃ (20 mL), extracted with DCM (10 mL×2). The combined organic extracts were washed with brine (10 mL×3), dried over anhydrous Na₂SO₄ and concentrated under reduced pressure. The mixture was purified by reverse phase chromatography to give Compound 13-1 (2210 mg, 9.64 mmol). MS: m/z 230 (M+H)⁺.

To a −78° C. solution of Compound 13-1 (946 mg, 4.13 mmol) in THF (20 mL) under nitrogen atmosphere was added methylmagnesium bromide (1.0 M in THF, 20 mL, 20 mmol). The mixture was stirred at −10° C. for 2 hours. The mixture was quenched with sat. aq. NH₄Cl (10 mL) and extracted with EtOAc (10 mL×2). The combined organic extracts were washed with brine (10 mL×3), dried over anhydrous Na₂SO₄ and concentrated under reduced pressure. The mixture was purified by reverse phase chromatography to give Compound 13-2 (711 mg, 70.24% yield). MS: m/z 246 (M+H)⁺.

To a solution of Compound 13-2 (617 mg, 2.52 mmol) in acetonitrile (20 mL) was added HCl (4 M in 1,4-dioxane, 5 mL). The reaction mixture was stirred at room temperature for 1 hour and concentrated under reduced pressure. The mixture was diluted with DCM:MeOH=10:1 (30 mL), adjusted to pH=8 with NaHCO₃, then filtered and the filtrate was concentrated under reduced pressure. To a 0° C. solution of the residue and INT 7 (734 mg, 2.62 mmol) in THF (20 mL) was added NaH (577 mg, 24.04 mmol). The mixture was stirred at room temperature for 2 hours. The mixture was quenched with ice/water (5 mL), filtered and the filtrate was concentrated under reduced pressure to give Compound 13-3 (1234 mg, crude). MS: m/z 389 (M+H)⁺.

To a solution of Compound 13-3 (1234 mg, 3.17 mmol) in toluene (10 mL) were added DIEA (1448 mg, 11.20 mmol) and POCl₃ (1807 mg, 11.7849 mmol). The mixture was stirred at room temperature for 2 hours. The mixture was quenched with sat. aq. NaHCO₃ (20 mL), extracted with DCM (10 mL×2). The combined organic extracts were washed with brine (10 mL×3), dried over anhydrous Na₂SO₄ and concentrated under reduced pressure. The residue was purified by reverse phase chromatography to give Compound 13-4 (141 mg, 11.98% yield). MS: m/z 371 (M+H)⁺.

The crude Compound 13 was prepared following the procedure for the synthesis of Compound 5 in Example 5 with Compound 13-4 as starting material, purified by pre-HPLC (C18 column, phase A: 0.1% TFA in water, phase B: CH₃CN, Gradient: 15% B to 30% B in 40 min at a flow rate of 60 mL/min, 230 nm) and freeze-dried to give Compound 13A (the first peak, 12.6 mg, TFA salt) and Compound 13B (the second peak, 19.0 mg, TFA salt). MS: m/z 632 (M+H)⁺.

### Example 14

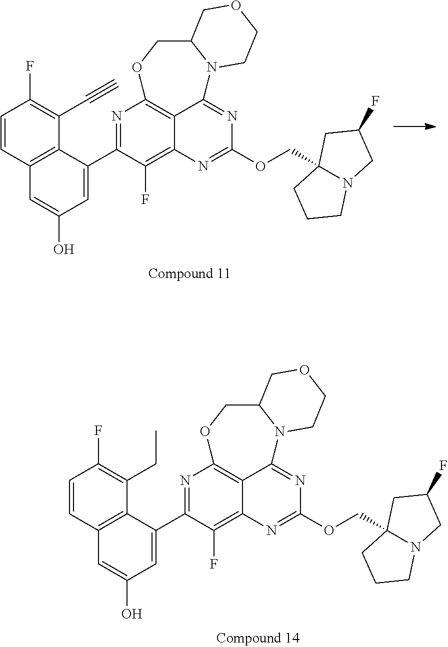 [SMILES: C#Cc1c(F)ccc2cc(O)cc(-c3nc4c5c(nc(OC[C@@]67CCCN6C[C@H](F)C7)nc5c3F)N3CCOCC3CO4)c12 ; CCc1c(F)ccc2cc(O)cc(-c3nc4c5c(nc(OC[C@@]67CCCN6C[C@H](F)C7)nc5c3F)N3CCOCC3CO4)c12]

To a solution of Compound 11 (11 mg, 0.016 mmol) in MeOH (5 ml) was added Pd(OH)₂/C (14 mg, 10% content). The reaction mixture was stirred at room temperature for 3 hours under hydrogen atmosphere. The mixture was filtered and the filtrate was concentrated under reduced pressure. The residue was purified by Prep-HPLC (C18 column, A: 0.1% TFA in water, B: CH₃CN, Gradient: 15% B to 45% B in 38 min at a flow rate of 40 mL/min, 230 nm) and freeze-dried to give Compound 14 (7.1 mg, 9.53 mol,). MS m/z: 608 [M+H]⁺.

### Example 15

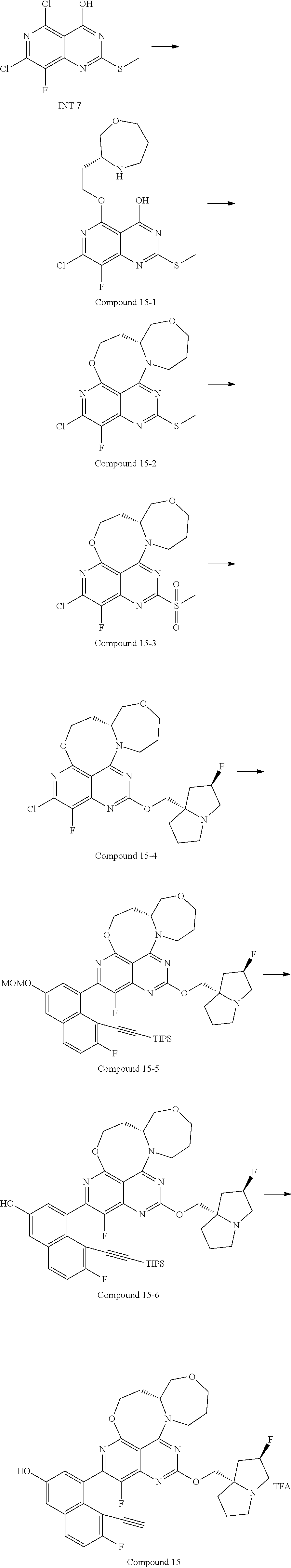

To a solution of INT 15 (89 mg, 0.61 mmol) in THF (5 mL) was added NaH (145 mg, 3.63 mmol, 60% content) at 0° C. After stirred for 10 min, INT 7 (165 mg, 0.59 mmol) was added. The reaction was stirred at room temperature for 3.5 hours, then quenched with water (0.5 mL). The resulting mixture was purified by reverse phase chromatography to give Compound 15-1 (158 mg, 0.41 mmol). MS m/z: 389 [M+H]⁺.

To a solution of Compound 15-1 (158 mg, 0.41 mmol) and DIEA (725 mg, 5.61 mmol) in DCM (30 mL) was added BOP—Cl (456 mg, 1.79 mmol). The reaction mixture was stirred at room temperature for 24 hours, then diluted with water (30 mL), extracted with EtOAc (30 mL×2). The collected organic layer was washed with brine (30 mL), dried over anhydrous Na₂SO₄ and concentrated under reduced pressure. The residue was purified by reverse phase chromatography to give Compound 15-2 (42 mg, 0.13 mmol). MS m/z: 371 [M+H]⁺.

The crude Compound 15 was prepared following the procedure for the synthesis of Compound 5 in Example 5 with Compound 15-2 as starting material and was purified by Prep-HPLC (C18 column, A: 0.1% TFA in water, B: CH₃CN, Gradient: 10% B to 34% B in 34 min at a flow rate of 40 mL/min, 240 nm) to freeze-dried to give Compound 15 (3.2 mg, 4.29 mol, TFA salt). MS m/z: 632 [M+H]⁺.

### Example 16

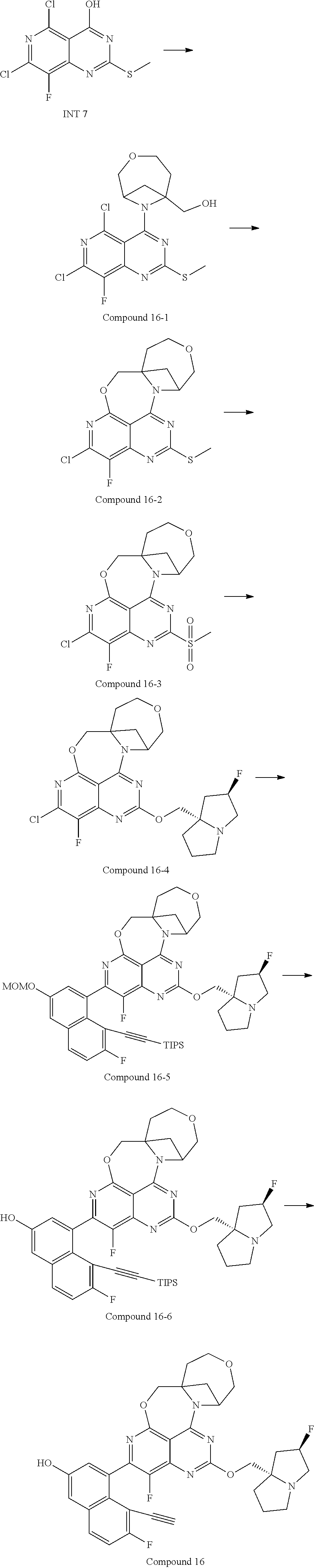

To a solution of INT 7 (165 mg, 0.59 mmol) and DIEA (1 ml) in toluene (10 mL) was added POCl₃ (0.5 ml). The reaction mixture was stirred at 85° C. for 3.5 hours and then concentrated under reduced pressure. A solution of the residue in DCM (5 ml) was added to a solution of INT 11 (crude) and DIEA (1 mL) in DCM (5 mL) at −30° C. The reaction mixture was stirred at −30° C. for 1 hour, then diluted with water (30 mL) and extracted with DCM (30 mL×2). The collected organic layer was washed with brine (30 mL), dried over anhydrous Na₂SO₄ and concentrated under reduced pressure. The residue was purified by Pre-TLC to give Compound 16-1 (73 mg, 0.18 mmol). MS m/z: 405 [M+H]⁺.

To a solution of Compound 16-1 (61 mg, 0.15 mmol) in DMF (4 mL) was added NaH (26 mg, 0.65 mmol, 60% content). The reaction was stirred at room temperature for 0.5 hour. The reaction solution was purified by reverse phase chromatography to give Compound 16-2 (58 mg, 0.16 mmol). MS m/z: 369 [M+H]⁺.

The crude Compound 16 was prepared following the procedure for the synthesis of Compound 5 in Example 5 with Compound 16-2 as starting material, purified by Prep-HPLC (C18 column, A: 0.05% NH₃·H₂O in water, B: CH₃CN, Gradient: 25% B to 53% B in 28 min at a flow rate of 40 mL/min, 232 nm) and freeze-dried to give Compound 16 (7.1 mg, 9.53 μmol). MS m/z: 630 [M+H]⁺.

### Example 17

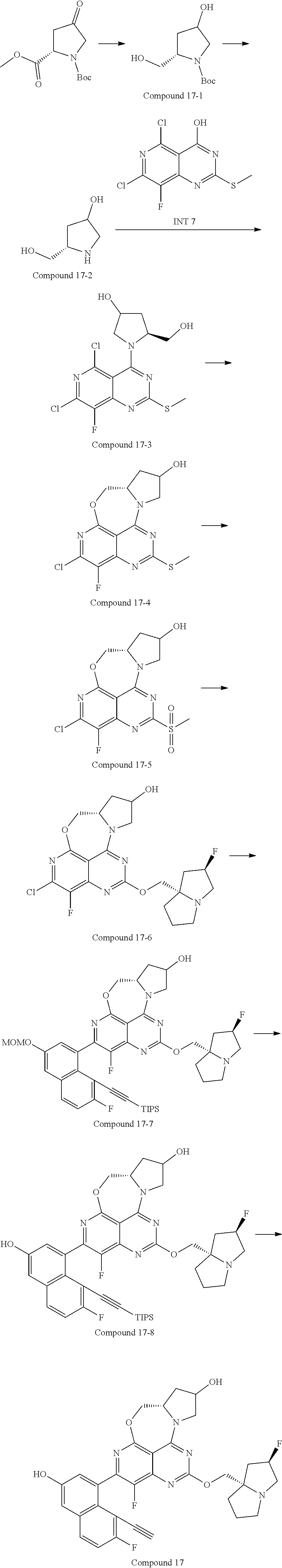

To a solution of 1-(tert-butyl) 2-methyl (S)-4-oxopyrrolidine-1,2-dicarboxylate (1.35 g, 5.55 mmol) in THF (20 mL) was added LiBH₄ (8 mL, 2 mol/L in THF). The reaction mixture was stirred at room temperature for 2.5 hours. The reaction mixture was quenched with water (30 mL), extracted with EtOAc (30 mL×2). The collected organic layer was washed with brine (30 mL), dried over anhydrous Na₂SO₄ and concentrated under reduced pressure. The residue was purified by silica gel column to give Compound 17-1 (1.095 g, 5.04 mmol). MS m/z: 218 [M+H]⁺.

To a solution of Compound 17-1 (1.095 g, 5.04 mmol) in DCM (5 mL) was added TFA (5 mL). The reaction mixture was stirred at room temperature for 1 hour and then concentrated under reduced pressure. The residue was dissolved in water (10 mL) and freeze-dried to give Compound 17-2 (crude). MS m/z: 118 [M+H]⁺.

To a solution of INT 7 (1038 mg, 3.71 mmol), DIEA (2 ml) in toluene (20 mL) was added POCl₃ (1 mL). The reaction mixture was stirred at 85° C. for 2 hours and then concentrated under reduced pressure. A solution of the residue in DCM (10 ml) was added to a solution of Compound 17-2 (crude) and DIEA (2 mL) in DCM (10 mL) at −30° C. The reaction mixture was stirred at −30° C. for 15 min, then diluted with water (30 mL) and extracted with DCM (30 mL×2). The collected organic layer was washed with brine (30 mL), dried over anhydrous Na₂SO₄ and concentrated under reduced pressure. The residue was purified by silica gel column to give Compound 17-3 (753 mg, 1.99 mmol). MS m/z: 379 [M+H]⁺.

To a solution of Compound 17-3 (753 mg, 1.99 mmol) in DMF (10 mL) was added NaH (248 mg, 6.20 mmol, 60% content) at 0° C. The reaction mixture was stirred at RT for 1 hour. The solution was diluted with water (30 mL) and extracted with DCM (50 mL×4). The collected organic layer was dried over anhydrous Na₂SO₄ and concentrated under reduced pressure. The residue was purified by reverse phase chromatography to give Compound 17-4 (130 mg, 0.38 mmol). MS m/z: 343 [M+H]⁺.

The crude Compound 17 was prepared following the procedure for the synthesis of Compound 5 in example 5 with Compound 17-4 as starting material and was purified by Prep-HPLC (C18 column, A: 0.05% NH₃·H₂O in water, B: CH₃CN, Gradient: 20% B to 50% B in 28 min at a flow rate of 40 mL/min, 225 nm) and freeze-dried to give Compound 17 (4.4 mg, 7.29 μmol). MS m/z: 604 [M+H]⁺.

### Example 18

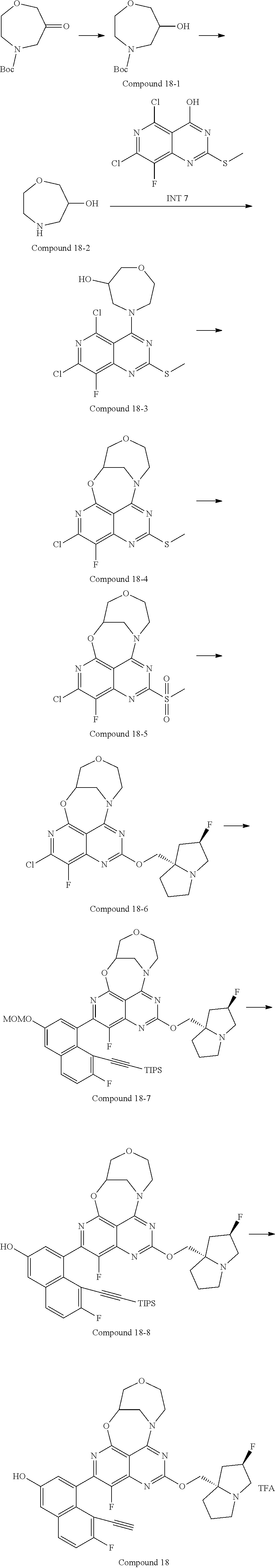

To a solution of tert-butyl 6-oxo-1,4-oxazepane-4-carboxylate (2.04 g, 9.48 mmol) in MeOH (20 mL) was added NaBH₄ (0.73 g, 19.30 mmol). The reaction mixture was stirred at room temperature for 2 hours. The reaction mixture was quenched with water (30 mL), the pH value was adjusted to 13 and extracted with EtOAc (30 mL×2). The collected organic layer was dried over anhydrous Na₂SO₄ and concentrated under reduced pressure to give Compound 18-1 (2.44 g, 11.23 mmol, HCl salt). MS m/z: 218[M+H]⁺.

To a solution of Compound 18-1 (1.31 g, 6.03 mmol) in acetonitrile (9 mL) was added HCl (3 mL, 4 mol/L in dioxane). The reaction mixture was stirred at room temperature for 4 hours, then concentrated under reduced pressure to give Compound 18-2 (0.87 g, 5.66 mmol). MS m/z: 118[M+H]⁺.

The crude Compound 18 was prepared following the procedure for the synthesis of Compound 19 in Example 19 with Compound 18-2 as starting material, purified by Prep-HPLC (C18 column, A: 0.1% TFA in water, B: CH₃CN, Gradient: 15% B to 35% B in 30 min at a flow rate of 40 mL/min, 235 nm) and freeze-dried to give Compound 18 (1.7 mg, 2.37 mol, TFA salt). MS m/z: 604 [M+H]⁺.

### Example 19

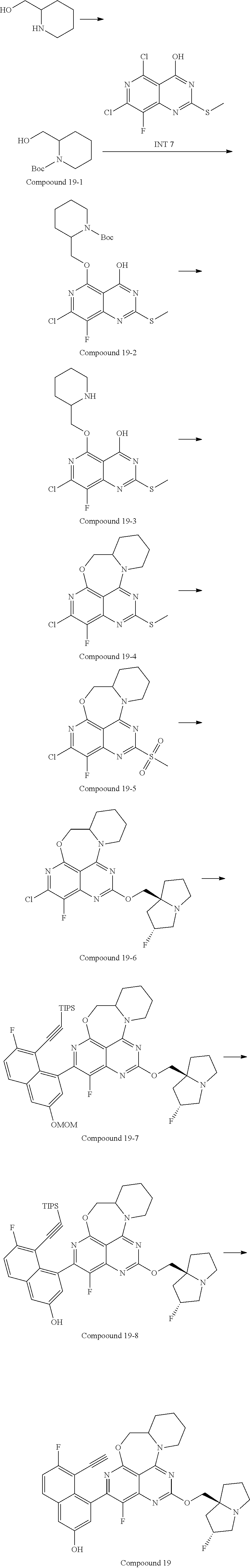

To a solution of piperidin-2-ylmethanol (1.07 g, 9.29 mmol), triethylamine, (2.70 g, 26.68 mmol) in DCM (20 mL) was added di-tert-butyl dicarbonate (2.41 g, 11.04 mmol) at 0° C. and stirred at room temperature overnight. The mixture was concentrated under reduced pressure and purified by reverse phase chromatography (C18 column, A: water, B: CH₃CN, Gradient: 10% B to 100% B in 30 min at a flow rate of 60 mL/min, 220 nm) to give Compound 19-1 (1.47 g, 6.83 mmol, 73.500% yield). MS: m/z: 216[M+H]⁺.

The crude Compound 19 was prepared following the procedure for the synthesis of Compound 5 in Example 5 with Compound 19-1 as starting material, purified by Prep-HPLC (C18 column, A: 0.05% ammonium hydroxide in water, B: CH₃CN, Gradient: 35% B to 72% B in 34 min at a flow rate of 70 mL/min, 230 nm) and freeze-dried to give Compound 19 (42.4 mg, 70.48 mol, 35.61% yield). MS: m/z: 602 [M+H]⁺.

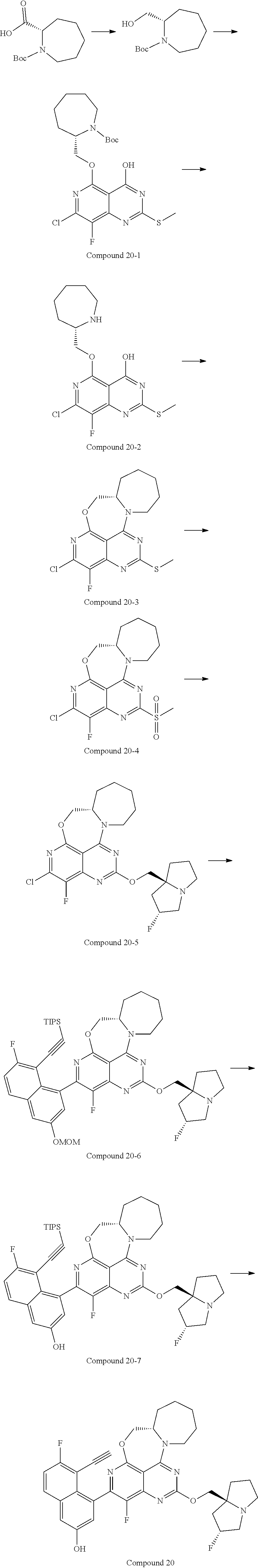

A solution of (S)-1-(tert-butoxycarbonyl)azepane-2-carboxylic acid (0.57 g, 2.34 mmol) in THF (10 mL), borane-tetrahydrofuran complex (5 mL) was added at −10° C. and stirred at −10° C. overnight. Methanol (15 mL) was added to the reaction solution, then heated to 60° C. and stirred for 0.5 h. The reaction solution was concentrated under reduced pressure to give Compound 20-1 (673 mg). MS: m/z: 230 [M+H]⁺.

The crude Compound 20 was prepared following the procedure for the synthesis of Compound 5 in Example N with Compound 20-1 as starting material, purified by Prep-HPLC (C18 column, A: 0.1% TFA in water, B: CH₃CN, Gradient: 15% B to 40% B in 50 min at a flow rate of 40 mL/min, 250 nm) and freeze-dried to give Compound 20 (1.6 mg, 2.5989 μmol, 7.4308% yield, TFA salt). MS: m/z: 616 [M+H]⁺.

### Example 21

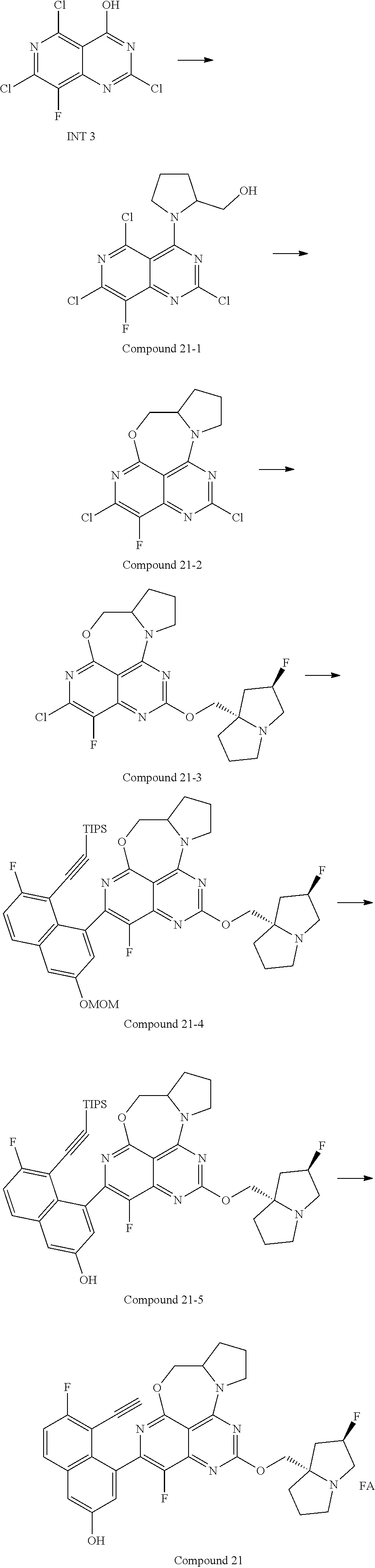

To a solution of INT 3 (500 mg, 1.86 mmol) in POCl₃ (6.75 mL) was added DIEA (0.68 mL). The reaction mixture was stirred at 110° C. for 1.5 h. Upon completion, the reaction mixture was concentrated under reduced pressure. To a solution of the residue (crude, 1.86 mmol) in DCM (10 mL) at −40° C. was added DIEA (1.6 g, 12.5 mmol), pyrrolidin-2-ylmethanol (188.4 mg, 1.86 mmol). The reaction mixture was stirred at −40° C. for 30 min. Upon completion, H₂O (15 mL) was added to the reaction mixture, extracted with DCM (15 mL×3). The combined organic phases were dried over anhydrous Na₂SO₄ and concentrated to give a residue, purified by silica gel column chromatography (petroleum ether/ethyl acetate=8:1 to 1:1) to give Compound 21-1 (370 mg, 1.05 mmol, 56.5% yield). LCMS: 351.0 [M+H]⁺.

To a solution of Compound 21-1 (370 mg, 1.05 mmol) in THF (9.5 mL) was added NaH (60%, 210 mg, 5.25 mmol) at 0° C. The reaction mixture was stirred at 0° C. for 1 h under nitrogen atmosphere. Upon completion, H₂O (15 mL) was added to the reaction mixture, extracted with EtOAc (15 mL×3). The combined organic phases were dried over anhydrous Na₂SO₄ and concentrated to give Compound 21-2 (200 mg, 0.63 mmol, crude). LCMS: 315.0 [M+H]⁺.

To a solution of Compound 21-2 (crude, 200 mg, 0.63 mmol) and ((2R,7aS)-2-fluorotetrahydro-1H-pyrrolizin-7a(5H)-yl) methanol (202.2 mg, 1.27 mmol) in dioxane (10 mL) were added DIEA (246.2 mg, 1.91 mmol), 4A molecular sieves (200 mg). The mixture was stirred at 90° C. overnight under N₂ atmosphere. Upon completion, the reaction mixture was filtered and concentrated to give a residue, purified by pre-TLC (Dichloromethane/methanol=15:1) to give Compound 21-3 (20 mg, 0.045 mmol, 7.1% yield). LCMS: 438.1 [M+H]⁺.

The crude Compound 21 was prepared following the procedure for the synthesis of Compound 5 in Example 5 with Compound 21-3 as starting material and was purified by Prep-HPLC (acetonitrile with 0.1% FA in water) to give Compound 21 (3.9 mg, 0.007 mmol, 20.7% yield, FA salt).

¹H NMR (300 MHz, DMSO-d₆): δ 10.13 (s, 1H), 7.97-7.92 (m, 1H), 7.48-7.41 (m, 1H), 7.36-7.35 (m, 1H), 7.19-7.09 (m, 1H), 5.36-5.19 (m, 1H), 4.71-4.62 (m, 1H), 4.25-3.97 (m, 5H), 3.85-3.82 (m, 2H), 3.11-3.09 (m, 3H), 3.00 (s, 1H), 2.86-2.78 (m, 1H), 2.03-1.75 (m, 9H). LCMS: 588.2 [M+H]⁺.

### Example 22

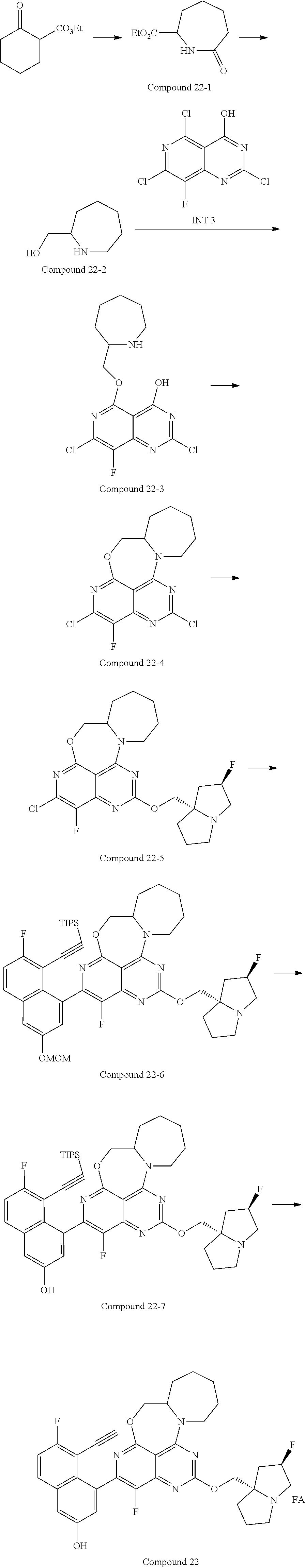

To a solution of ethyl 2-oxocyclohexane-1-carboxylate (5.0 g, 29.4 mmol) in CHCl₃ (60 mL) was added MeSO₃H (28 g, 294 mmol) at 0° C. Then the mixture was added NaN₃ (3.8 g, 58.8 mmol), heated and refluxed for 4 h. The mixture was added ice slowly and ammonia (20 mL). The mixture was extracted with DCM (50 mL). The organic layer was quenched with water (5 mL), brine (5 mL), dried with anhydrous Na₂SO₄ and filtered, the filtrate was concentrated to dryness. The residue was purified by column chromatography on silica gel (eluted with PE/EtOAC=1:1) to give Compound 22-1 (3.5 g, 64.3% yield). LCMS: 186.2 [M+H]⁺.

To a solution of Compound 22-1 (1.0 g, 5.4 mmol) in THF was added LiAlH₄ (863 mg, 21.6 mmol) at 0° C. under N₂ atmosphere. Then the mixture was heated and refluxed for 5 h. After that, the mixture was added H₂O (0.8 mL) at 0° C., stirred for 15 min. The mixture was added saturated aqueous 15% NaOH (0.8 mL). Then the mixture was added H₂O (2.4 mL) and Na₂SO₄ at room temperature. Finally, the mixture was filtered, washed with EtOAc and the filtrate was concentrated to give Compound 22-2 (600 mg, 86.0% yield). LCMS: 130.2 [M+H]⁺.

To a solution of INT 3 (300 mg, 1.1 mmol) in THF (3 mL) were added NaH (89.5 mg, 2.2 mmol) and Compound 22-2 (433 mg, 3.3 mmol) at 0° C. under N₂ atmosphere, stirred for 3 h. The mixture was poured into water (3 mL) and extracted with EtOAc (5 mL×2). The organic layer was washed with water (5 mL), brine (5 mL), dried over anhydrous Na₂SO₄ and filtered, the filtrate was concentrated to dryness. The residue was purified by Pre-TLC (eluted with DCM/MeOH=10:1) to give Compound 22-3 (100 mg, 24.8% yield). LCMS: 361.1 [M+H]⁺.

To a solution of Compound 22-3 (80 mg, 0.22 mmol) in DCM were added DIEA (100 mg, 0.77 mmol), POCl₃ (170 mg, 1.1 mmol) at 0° C. under N₂ atmosphere. The mixture was stirred for 3 h. After that, the mixture was concentrated directly to give residue. The residue was purified by Pre-TLC (eluted with PE/EtOAc=10:1) to give Compound 22-4 (30 mg, 39.4% yield). LCMS: 343.1 [M+H]⁺.

Compound 22-4 (30 mg, 0.08 mmol), ((2R,7aS)-2-fluorotetrahydro-1H-pyrrolizin-7a(5H)-yl)methanol (28 mg, 0.17 mmol), DIEA (34 mg, 0.26 mmol), 4A Ms (10 mg) were added into dioxane (6 mL) under N₂ atmosphere. The mixture was heated to 85° C. and stirred for 8 h. After that, the mixture was cooled to room temperature, poured into water (10 mL) and extracted with DCM (8 mL×2). The organic layer was washed with water (5 mL), brine (5 mL), dried with anhydrous Na₂SO₄ and filtered, the filtrate was concentrated to dryness. The residue was purified by Pre-TLC (eluted with DCM/MeOH=10:1) to give Compound 22-5 (30 mg, yield 73.7%). LCMS: 466.2 [M+H]⁺.

The crude Compound 22 was prepared following the procedure for the synthesis of Compound 5 in Example 5 with Compound 22-5 as starting material and was purified by Prep-HPLC (FA) to give Compound 22 (1.1 mg, FA salt). LCMS: 616.2 [M+H]⁺.

### Example 23

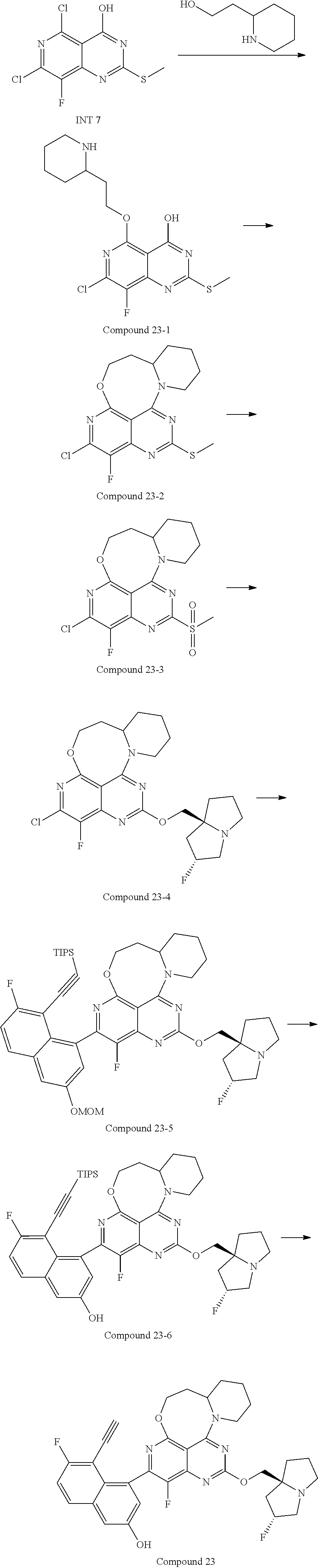

The Compound 23 (1.3 mg) was prepared following the procedure for the synthesis of Compound 15 in Example 15 with 2-(piperidin-2-yl)ethan-1-ol as starting material. LCMS: 616 [M+H]⁺.

### Example 24

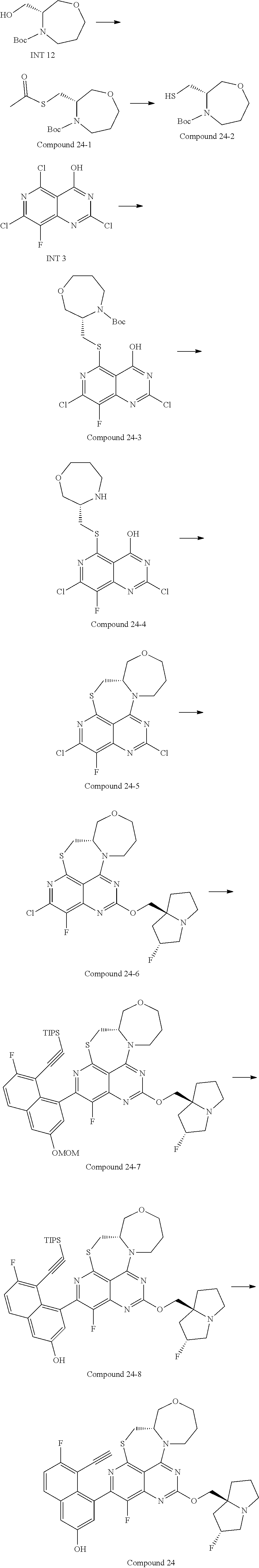

Diisopropyl azodicarboxylate (1.34 g, 6.63 mmol) was added to a solution of INT 12 (509 mg, 2.20 mmol) and triphenylphosphine (1.70 g, 6.48 mmol) in THF (20 mL) at −10° C. with nitrogen atmosphere. The mixture was stirred at −10° C. for 0.5 hour, then ethanethioic acid (342 mg, 4.49 mmol) was added and stirred at −10° C. for 2 hours. The reaction solution was concentrated under reduced pressure and the residue was purified by column chromatography (SiO₂, n-hexane/ethyl acetate=30/1 to 10/1, v/v) to give Compound 24-1 (564 mg, 88.56% yield). MS: m/z: 290 [M+H]⁺.

A solution of Compound 24-1 (411 mg, 1.42 mmol), sodium hydroxide (164 mg, 4.10 mmol) in water (2 mL) and THF (15 mL) was stirred at room temperature for 1 hour. The mixture was concentrated under reduced pressure and the residue was diluted with water, adjusted to pH=3 with HCl (1 N) and extracted with EtOAc (100 mL×2). The combined organic layers were dried over Na₂SO₄ and concentrated under reduced pressure. The residue was purified by reverse phase chromatography (C18) to give Compound 24-2 (229 mg, 65.17% yield). MS: m/z: 248 [M+H]⁺.

A solution of Compound 24-2 (205 mg, 828.77 μmol), INT 3 (270 mg, 1.01 mmol), LiOH (40 mg, 1.67 mmol) in THF (10 mL) was stirred at 50° C. for 4 hours under nitrogen atmosphere. The mixture was concentrated under reduced pressure and the residue was purified by reverse phase chromatography (C18 column) to give Compound 24-3 (161 mg, 40.53% yield). MS: m/z: 479 [M+H]⁺.

A solution of Compound 24-3 (146 mg, 304.58 μmol), HCl (1 mL, 4 M in dioxane) in acetonitrile (10 mL) was stirred at room temperature for 1 hour. The reaction solution was concentrated under reduced pressure to give Compound 24-4 (267 mg, crude). MS: m/z: 379 [M+H]⁺.

A solution of Compound 24-4 (267 mg, 704.05 μmol), N,N-diisopropylethylamine (3 mL), phosphorus oxychloride (0.5 mL) in toluene (10 mL) was stirred at room temperature for 1 hour. The solution was diluted with sat. NaHCO₃ (50 mL) and extracted with EtOAc (30 mL). The organic layer was dried over Na₂SO₄ and concentrated under reduced pressure. The residue was purified by reverse phase chromatography (C18 column) to give Compound 24-5 (61 mg, 23.99% yield). MS: m/z: 361 [M+H]⁺.

The Compound 24 was prepared following the procedure for the synthesis of Compound 10 in example 10. LCMS: 634 [M+H]⁺.

### Pharmacological Experiments

### 1. SOS1 Catalyzed Nucleotide Exchange Assay

The inhibition activity of each of compounds on GDP form K-Ras was evaluated by SOS1 catalyzed nucleotide exchange assays. K-Ras G12D, K-Ras G12V, K-Ras G12C, K-Ras G13D, K-Ras G12A, K-Ras G12R, K-Ras Q61H and K-Ras WT proteins were used in this assay.

Briefly, K-Ras (His tag, aa 1-169) pre-loaded with GDP was pre-incubated with each of compounds in the presence of 10 nM GDP in a 384-well plate (Greiner) for 15-60 mins, then purified SOS1 ExD (Flag tag, aa 564-1049), BODIPY™ FL GTP (Invitrogen) and monoclonal antibody anti 6HIS—Tb cryptate Gold (Cisbio) were added to the assay wells and incubated for 4 hours at 25° C. (Specially, we did not add SOS1 in the K-Ras G13D assay). Wells containing the same percent of DMSO served as vehicle control, and wells without K-Ras served as low control. TR-FRET signals were read on Tecan Spark multimode microplate reader. The parameters were F486: Excitation 340 nm, Emission 486 nm, Lag time 100 μs, Integration time 200 s; F515: Excitation 340 nm, Emission 515 nm, Lag time 100 μs, Integration time 200 μs. TR-FRET ratios for each individual wells were calculated by equation: TR-FRET ratio=(Signal F515/Signal F486)*10000. The percent of activation of compounds treated wells were normalized between vehicle control and low control (% Activation=(TR-FRET ratio_{Compound treated}−TR-FRET ratio_{Low control})/(TR-FRET ratio_{Vehicle control}−TR-FRET ratio_{Low control})*1000). Then the data were analyzed either by fitting a 4-parameter logistic model or by Excel to calculate IC₅₀ values. The results are shown in the following Table 11.

2. GTP-K-Ras and cRAF Interaction Assay

The inhibition activity of each of compounds on GTP form K-Ras was evaluated by GppNp-K-Ras and cRAF interaction assays. GppNp is an analog of GTP. K-Ras G12D, K-Ras G12V, K-Ras G12C, K-Ras G13D, K-Ras G12A, K-Ras G12R, K-Ras Q61H and K-Ras WT proteins were used in this assay.

Briefly, K-Ras (His tag, aa 1-169) pre-loaded with GppNp was pre-incubated with each of compounds in the presence of 200 M GTP in a 384-well plate (Greiner) for 15-60 mins, then cRAF RBD (GST tag, aa 50-132, CreativeBioMart), monoclonal antibody anti GST-d2 (Cisbio) and monoclonal antibody anti 6HIS—Tb cryptate Gold (Cisbio) were added to the assay wells and incubated for 2 hours at 25° C. Wells containing same percent of DMSO served as vehicle control, and wells without K-Ras served as low control. HTRF signals were read on Tecan Spark multimode microplate reader and HTRF ratios were calculated under manufacturer's instructions. The percent of activation of compounds treated wells were normalized between vehicle control and low control (% Activation=(HTRF ratio_{Compound treated}−HTRF ratio_{Low control})/(HTRF ratio_{Vehicle control}−HTRF ratio_{Low control})*100%). Then the data were analyzed either by fitting a 4-parameter logistic model or by Excel to calculate IC₅₀ values. The results are shown in the following Table 11.

TABLE 11

| | Biochemical assays, IC₅₀ (nM) | | | |
|---|---|---|---|---|
| | KRAS G12D | | KRAS G12V | |
| Compound | GDP | GppNp | GDP | GppNp |
| Compound 1 | 6.43 | 298 | 3.87 | 992 |
| Compound 2 | 1.58 | 41.9 | 1.94 | 93.2 |
| Compound 3 | 1.43 | 6.35 | 1.44 | 9.29 |
| Compound 4 | 32.7 | >1000 | 14.7 | >1000 |
| Compound 5 | 0.658 | 5.33 | 0.575 | 11.3 |
| Compound 6 | 0.871 | 12.9 | 1.36 | 11.0 |
| Compound 7A | 10.8 | 319 | 6.07 | ~1000 |
| Compound 7B | 2.31 | 775 | 1.55 | 531 |
| Compound 8B | 27.0 | ~1000 | 20.9 | >1000 |
| Compound 9 | 2.60 | 15.4 | 1.67 | 47.2 |
| Compound 10 | 192 | >1000 | 88.8 | >1000 |
| Compound 11 | 1.62 | 274 | 1.07 | 307 |
| Compound 12 | 9.24 | 170 | 6.09 | 648 |
| Compound 13A | 1.83 | 19.5 | 1.23 | 25.0 |
| Compound 13B | 1.44 | 21.4 | 0.699 | 49.3 |
| Compound 14 | 4.32 | >1000 | 4.84 | >1000 |
| Compound 16 | 57.4 | >1000 | 43.5 | >1000 |
| Compound 17 | 80.3 | >1000 | 17.0 | >1000 |
| Compound 18 | 49.3 | >1000 | 31.1 | >1000 |
| Compound 19 | 1.53 | 178 | 1.15 | 287 |
| Compound 20 | 1.07 | 34.5 | 0.444 | 73.2 |
| Compound 21 | 2.34 | 705 | 4.16 | >1000 |
| Compound 22 | 1.22 | 24.2 | 0.922 | 7.00 |
| Compound 23 | 2.46 | 674 | 3.76 | >1000 |
| Compound 24 | 1.85 | 18 | 3.79 | 14.3 |

### 3. Phospho-ERK1/2(THR202/TYR204) HTRF Assay

p-ERK (MAPK pathway) inhibition activity of each of compounds in a variety of K-Ras mutant and K-Ras WT cell lines indicated in Table 12 was evaluated. MKN-1 with K-Ras WT amplification is also a K-Ras dependent cell line.

TABLE 12

| | | Cell seeding | | | |
|---|---|---|---|---|---|
| | K-Ras | density, | Culture | Assay | Cell |
| Cell lines | mutation | cells/well | medium | medium | incubator |
| AGS | G12D | 60000 | F-12K, 10% FBS | F-12K, | 37° C., |
| | | | | 0.1% FBS | 5% CO₂ |
| SW620 | G12V | 60000 | L-15, 10% FBS | L-15, | 37° C., |
| | | | | 0.1% FBS | 100% Air |

Each of cells in culture medium was seeded in 96-well plates at density indicated in Table 16 and then put in a cell incubator to incubate overnight. The next day, the culture medium was removed and the compound diluted in assay medium was added in each well. After 2 hours incubation in a cell incubator, the assay medium in 96-well plates was removed, then 50 μL of 1× blocking reagent-supplemented lysis buffer (Cisbio) was added and the plates were incubated at 25° C. for 45 min with shaking. 10 μL of cell lysates from the 96-well plates were transferred to a 384-well plate (Greiner) containing 2.5 t/well HTRF® pre-mixed antibodies (Cisbio 64AERPEH). The plate was incubated 4 hours at 25° C. and then read HTRF signals on Tecan Spark multimode microplate reader. The data were analyzed using a 4-parameter logistic model to calculate IC₅₀ values. The results are shown in the following Table 13:

TABLE 13

p-ERK, IC₅₀(nM)

| Compound | AGS | SW620 |
|---|---|---|
| Compound 1 | 16.2 | 38.2 |
| Compound 2 | 2.33 | 5.20 |
| Compound 3 | 2.02 | 4.61 |
| Compound 5 | 0.525 | 1.03 |
| Compound 6 | 1.50 | 10.0 |
| Compound 7A | | 36.4 |
| Compound 7B | 21.1 | 26.4 |
| Compound 9 | 6.58 | 18.6 |
| Compound 11 | 26.3 | 16.8 |
| Compound 12 | 48.0 | 128 |
| Compound 13A | 6.89 | 6.74 |
| Compound 13B | 1.68 | 1.19 |
| Compound 14 | 39.4 | 42.3 |
| Compound 19 | 2.32 | 5.13 |
| Compound 20 | 0.691 | 2.01 |
| Compound 21 | 19.7 | 60.6 |
| Compound 22 | 1.38 | 3.05 |
| Compound 23 | 23.0 | 50.1 |

### 4. Cell Growth Inhibition Assay

The cell growth inhibition activity of each of compounds was tested by performing cell growth inhibition assays on a variety of K-Ras mutant and K-Ras WT cell lines indicated in Table 14.

TABLE 14

| | | Cell seeding | | | |
|---|---|---|---|---|---|
| | K-Ras | density, | Culture | Assay | Cell |
| Cell lines | mutation | cells/well | medium | format | incubator |
| AGS | G12D | 500 | F-12K, 10% FBS | 2D | 37° C., 5% CO₂ |
| AsPC-1 | G12D | 1000 | RPMI 1640, | 2D | 37° C., 5% CO₂ |
| | | | 10% FBS | | |
| SW620 | G12V | 1500 | L-15, 10% FBS | 2D | 37° C., 100% Air |

### 2D Cell Growth Inhibition Assays

Each of cells in culture medium was plated in TC-treated 96-well plates at a density indicated in Table 14 and incubated in a cell incubator overnight. The next day, each of compounds was diluted in culture medium and added to the plates. After 6 days incubation in cell incubator, the cell viability was detected by CellTiter-Glo® Cell Viability Assay kit (Promega). Luminescent signals were read on Tecan Spark multimode microplate reader and analyzed using a 4-parameter logistic model to calculate absolute IC₅₀ values. The results are shown in the following Table 15.

### 3D Cell Growth Inhibition Assays

Each of cells in culture medium was plated in ultra-low attachment-coated 96-well plates at a density indicated in Table 14 and incubated in a cell incubator overnight. The next day, each of compounds was diluted in culture medium and added to the plates. After 6 days incubation in cell incubator, the cell viability was detected by CellTiter-Glo® 3D Cell Viability Assay kit (Promega). Luminescent signals were read on Tecan Spark multimode microplate reader and analyzed using a 4-parameter logistic model to calculate absolute IC₅₀ values. The results are shown in the following Table 15.

TABLE 15

| | Cell Growth Inhibition, IC₅₀(nM) | | |
|---|---|---|---|
| Compound | AGS | AsPC-1 | SW620 |
| Compound 1 | 326 | 987 | 100 |
| Compound 2 | 5.07 | 134 | 13.1 |
| Compound 3 | 1.73 | 19.6 | 3.32 |
| Compound 5 | 0.466 | 9.28 | 0.819 |
| Compound 6 | 7.57 | 80.1 | 5.61 |
| Compound 7B | 75.1 | 812 | 21.0 |
| Compound 9 | 6.80 | 64.4 | 7.67 |
| Compound 11 | 31.8 | 346 | 25.0 |
| Compound 12 | 83.4 | 810 | 91.2 |
| Compound 13A | 15.7 | 224 | 13.2 |
| Compound 13B | 4.01 | 40.0 | 1.91 |
| Compound 14 | 105 | 702 | 23.9 |
| Compound 20 | 1.23 | 10.2 | 1.49 |
| Compound 21 | 99.0 | 547 | 35.1 |
| Compound 22 | 2.59 | 15.6 | 1.32 |

What is claimed is:

1-47. (canceled)

48. A compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof: 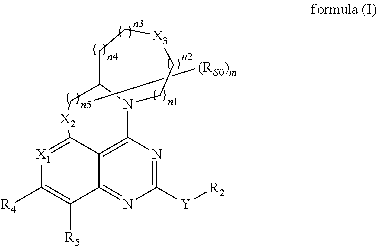
Wherein,
X₁ is selected from CR₃ or N;
R₃ is selected from hydrogen, deuterium, halogen, —C₁₋₆alkyl, haloC₁₋₆alkyl, haloC₁₋₆alkoxy, —C₂₋₆alkenyl, —C₁₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered heterocyclyl, 6-10 membered aryl or 5-10 membered heteroaryl, is independently optionally substituted with 1, 2 or 3 R₃ₐ;
each of R₃ₐ at each occurrence is independently selected from deuterium, halogen, —C₁₋₃alkyl, haloC₁₋₃ alkyl, haloC₁₋₃alkoxy, —C₂₋₃alkenyl, —C₂₋₃alkynyl, —CN, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —S(═O)₂NH₂, —S(═O)₂NH(C₁₋₃alkyl), —S(═O)₂N(C₁₋₃ alkyl)₂, —NHS(═O)₂(C₁₋₃alkyl), —N(C₁₋₃alkyl)S(═O)₂(C₁₋₃alkyl), 3-6 membered cycloalkyl, 3-6 membered heterocyclyl, phenyl or 5-6 membered heteroaryl;
X₂ is selected from NR₁, O or S;
R₁ is selected from hydrogen, deuterium, —C₁₋₆alkyl or 3-6 membered cycloalkyl; said —C₁₋₆alkyl and 3-6 membered cycloalkyl are each independently optionally substituted with one or more (such as 1, 2, 3, 4, 5 or 6) substituents selected from —OH, deuterium, halogen, —CN, oxo, —C₁₋₆alkoxy, —NH₂, —NHC₁₋₆alkyl, or —N(C₁₋₆ alkyl)₂;
X₃ is selected from CR₇₁R₇₂, C═O, NR₇₁, O, S, S=O, or O═S═O;
Each of (R₇₁, R₇₂) is independently selected from hydrogen, deuterium, —C₁₋₆alkyl or 3-6 membered cycloalkyl; said —C₁₋₆alkyl and 3-6 membered cycloalkyl are each independently optionally substituted with 1, 2, 3, 4, 5 or 6 substituents selected from —OH, deuterium, halogen, —CN, oxo, —C₁₋₆alkoxy, —NH₂, —NHC₁₋₆alkyl, or —N(C₁₋₆ alkyl)₂;
Each of (n₁, n2, n₃, n₄ and n₅) at each occurrence is independently selected from 0, 1, 2 or 3;
Each of R_{S0} at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)H, —C(═O)(C₁₋₆alkyl), —C(═O)OH, —C(═O)(OC₁₋₆alkyl), —OC(═O)(C₁₋₆alkyl), —C(═O)NH₂, —NO₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆ alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 R₁ₐ;
Optionally, two R_{S0} together with the carbon atom to which they are both attached form 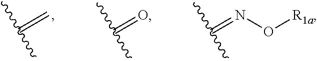
a 3-10 membered carbocyclic ring or a 3-10 heterocyclic ring; wherein, said 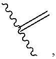
3-10 membered carbocyclic ring or 3-10 heterocyclic ring is optionally substituted with one or more R₁ₐ;
Optionally, two adjacent R_{S0} together with the carbon atoms to which they are respectively attached form a 3-10 membered carbocyclic ring or 3-10 membered heterocyclic ring, wherein, each of rings is independently optionally substituted with one or more R₁ₐ;
Each of R₁ₐ is independently selected from deuterium, halogen, —C₁₋₆alkyl, haloC₁₋₆alkyl, —CN, oxo, —OH, —NH₂, —NH(C₁₋₆alkyl), —N(C₁₋₆alkyl)₂, —OC₁₋₆alkyl, or —C₁₋₆alkyl substituted with 1, 2 or 3 substituents selected form deuterium, halogen, haloC₁₋₆alkyl, —CN, —OH, —NH₂, —NH(C₁₋₆alkyl), —N(C₁₋₆alkyl)₂, —OC₁₋₆alkyl or cyclopropyl;
m is selected from 0, 1, 2, 3, 4, 5 or 6;
Y is a bond, O, S, S(═O), S(═O)₂ or NR₈₁;
R₂ is selected from -L-(3-12 membered heterocyclyl), -L-(3-12 membered cycloalkyl), -L-(6-12 member aryl), -L-(5-12 membered heteroaryl), -L-NR₉₁R₉₂, 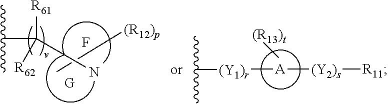
Each L is independently selected from a bond or C₁₋₁₀ alkylene optionally substituted with one or more R₁₆;
Each (R₉₁, R₉₂) is independently selected from hydrogen, deuterium, or —C₁₋₁₀alkyl optionally substituted with one or more R₁₆;
Said 3-12 membered heterocyclyl in -L-(3-12 membered heterocyclyl) is optionally substituted with one or more R₁₆;
Said 3-12 membered cycloalkyl in -L-(3-12 membered cycloalkyl) is optionally substituted with one or more R₁₆;
Said 6-12 member aryl in -L-(6-12 member aryl) is optionally substituted with one or more R₁₆;
Said 5-12 membered heteroaryl in -L-(5-12 membered heteroaryl) is optionally substituted with one or more R₁₆;
Y₁ or Y₂ at each occurrence is independently selected from —C(R₆₁R₆₂)—;
Ring F or ring G is a 3-10 membered heterocyclic ring which is optionally further contains 1, 2, or 3 heteroatoms selected from N, O or S;
Ring A is a 3-10 membered carbocyclic ring or a 3-10 membered heterocyclic ring; wherein the moiety of —(Y₁)ᵣ— and —(Y₂)ₛ—R₁₁ are attached to the same atom or different atoms of the ring A;
R₁₁ is selected from —NR₈₁R₈₂, —OR₈₁, —SR₈₁, 3-10 membered heterocyclyl, or 5-10 membered heteroaryl, wherein said 3-10 membered heterocyclyl or 5-10 membered heteroaryl is optionally independently substituted with one or more R₁₆;
Each of R₁₂ and R₁₃ at each occurrence is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₁R₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, two R₁₂ together with the carbon atom to which they are both attached form 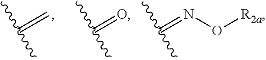
a 3-10 membered carbocyclic ring or a 3-10 heterocyclic ring; wherein, said 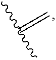
3-10 membered carbocyclic ring or 3-10 heterocyclic ring is optionally substituted with one or more R₂ₐ;
Optionally, two adjacent R₁₂ together with the carbon atoms to which they are respectively attached form 3-10 membered carbocyclic ring, 3-10 membered heterocyclic ring, 6-10 membered aryl ring or 5-10 membered heteroaryl ring, wherein, each of rings is independently optionally substituted with one or more R₂ₐ;
Each of R₂ₐ is independently selected from deuterium; halogen; —C₁₋₆alkyl; haloC₁₋₆alkyl; —CN; oxo; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form deuterium, halogen, haloC₁₋₆alkyl, —CN, —OH, —NH₂, —NH(C₁₋₆alkyl), —N(C₁₋₆alkyl)₂, —OC₁₋₆alkyl or cyclopropyl;
Each of (r, s, t, p and v) at each occurrence is independently selected from 0, 1, 2, 3, 4, 5 or 6;
R₄ is selected from 6-10 membered aryl, 5-10 membered heteroaryl, 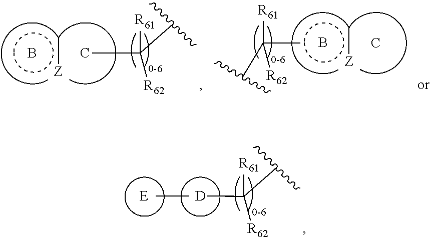
wherein said 6-10 membered aryl, 5-10 membered heteroaryl, 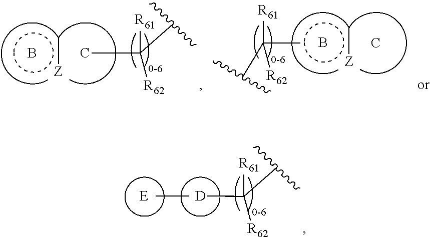
is optionally independently substituted with one or more R₄₁;
Z at each occurrence is independently selected from C or N;
Ring B at each occurrence is independently selected from a 6 membered aryl ring or a 5-6 membered heteroaryl ring and ring C at each occurrence is a 3-10 membered carbocyclic ring or a 3-10 membered heterocyclic ring when Z is selected from C;
Ring B at each occurrence is selected from a 5-6 membered heteroaryl ring and ring C at each occurrence is a 3-10 membered heterocyclic ring when Z is selected from N;
Ring D at each occurrence is selected from a 3-10 membered carbocyclic ring, a 3-10 membered heterocyclic ring, a 6 membered aryl ring or a 5-6 membered heteroaryl ring;
Ring E at each occurrence is selected from a 3-10 membered carbocyclic ring, a 3-10 membered heterocyclic ring, a 6-membered aryl ring or a 5-6 membered heteroaryl ring;
R₄₁ at each occurrence is independently selected from deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-10 membered cycloalkyl, 3-10 membered cycloalkenyl, 3-10 membered cycloalkynyl, 3-10 membered heterocyclyl, 6-10 membered aryl or 5-10 membered heteroaryl or R₄ₐ, wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
R₄ₐ is a structure capable of forming a prodrug;
R₅ is selected from hydrogen, deuterium, halogen, —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, haloC₂₋₁₀alkenyl, —C₂₋₁₀alkynyl, haloC₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —NR₈₁R₈₂, —OR₈₁, —SR₈₁, —S(═O)R₈₁, —S(═O)₂R₈₁, —C(═O)R₈₁, —C(═O)OR₈₁, —OC(═O)R₈₁, —C(═O)NR₈₁R₈₂, —NR₈₁C(═O)R₈₂, —OC(═O)OR₈₁, —NR₈₁C(═O)OR₈₂, —OC(═O)NR₈₁R₈₂, —NR₈₁C(═O)NR₈₁R₈₂, —S(═O)OR₈₁, —OS(═O)R₈₁, —S(═O)NR₈₁R₈₂, —NR₈₁S(═O)R₈₂, —S(═O)₂OR₈₁, —OS(═O)₂R₈₂, —S(═O)₂NR₈₁R₈₂, —NR₈₁S(═O)₂R₈₂, —OS(═O)₂OR₈₁, —NR₈₁S(═O)₂OR₈₂, —OS(═O)₂NR₈₁R₈₂, —NR₈₁S(═O)₂NR₈₁R₈₂, —PR₈₁R₈₂, —P(═O)R₈₁R₈₂, 3-6 membered cycloalkyl, 3-6 membered cycloalkenyl, 3-6 membered cycloalkynyl, 3-6 membered heterocyclyl, 6-10 membered aryl or 5-10 membered heteroaryl;
R₆₁ or R₆₂ at each occurrence is independently selected from hydrogen, deuterium, halogen, —C₁₋₁₀ alkyl, haloC₁₋₁₀alkyl, haloC₁₋₁₀alkoxy, —C₂₋₁₀alkenyl, —C₂₋₁₀alkynyl, —CN, —NO₂, —N₃, oxo, —NRₐR_{b}, —ORₐ, —SRₐ, —S(═O)Rₐ, —S(═O)₂Rₐ, —C(═O)Rₐ, —C(═O)ORₐ, —OC(═O)Rₐ, —C(═O)NRₐR_{b}, —NRₐC(═O)R_{b}, —OC(═O)ORₐ, —NRₐC(═O)OR_{b}, —OC(═O)NRₐR_{b}, —NRₐC(═O)NRₐR_{b}, —S(═O)ORₐ, —OS(═O)Rₐ, —S(═O)NRₐR_{b}, —NRₐS(═O)R_{b}, —S(═O)₂ORₐ, —OS(═O)₂Rₐ, —S(═O)₂NRₐR_{b}, —NRₐS(═O)₂R_{b}, —OS(═O)₂ORₐ, —NRₐS(═O)₂OR_{b}, —OS(═O)₂NRₐ, —NRₐS(═O)₂NRₐR_{b}, —PRₐR_{b}, —P(═O)RₐR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NR_{c}R_{d}, —OR_{c}, —SR_{c}, —S(═O)R_{c}, —S(═O)₂R_{c}, —C(═O)R_{c}, —C(═O)OR_{c}, —OC(═O)R_{c}, —C(═O)NR_{c}R_{d}, —NR_{c}C(═O)R_{d}, —OC(═O)OR_{c}, —NR_{c}C(═O)OR_{d}, —OC(═O)NR_{c}R_{d}, —NR_{c}C(═O)NR_{c}R_{d}, —S(═O)OR_{c}, —OS(═O)R_{c}, —S(═O)NR_{c}R_{d}, —NR_{c}S(═O)R_{d}, —S(═O)₂OR_{c}, —OS(═O)₂R_{c}, —S(═O)₂NR_{c}R_{d}, —NR_{c}S(═O)₂R_{d}, —OS(═O)₂OR_{c}, —NR_{c}S(═O)₂OR_{d}, —OS(═O)₂NR_{c}, —NR_{c}S(═O)₂NR_{c}R_{d}, —PR_{c}R_{d}, —P(═O)R_{c}R_{d}, 3-6 membered cycloalkyl, 3-6 membered cycloalkenyl, 3-6 membered cycloalkynyl, 3-6 membered heterocyclyl, 6-10 membered aryl or 5-10 membered heteroaryl;
Optionally, R₆₁ and R₆₂ together with the carbon atom to which they are both attached form a 3-10 membered carbocyclic ring or a 3-10 membered heterocyclic ring, wherein, said 3-10 membered carbocyclic ring or 3-10 membered heterocyclic ring is optionally independently substituted with one or more R₁₆;
R₈₁ or R₈₂ at each occurrence is independently selected from hydrogen, deuterium, —C₁₋₁₀alkyl, haloC₁₋₁₀alkyl, haloC₁₋₁₀alkoxy, —C₂₋₁₀alkenyl, —C₂₋₁₀alkynyl, —S(═O)Rₐ, —S(═O)₂Rₐ, —C(═O)Rₐ, —C(═O)ORₐ, —C(═O)NRₐR_{b}, —S(═O)ORₐ, —S(═O)NRₐR_{b}, —S(═O)₂ORₐ, —S(═O)₂NRₐR_{b}, —P(═O)RₐR_{b}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₁₀alkyl, haloC₁₋₁₀alkyl, haloC₁₋₁₀alkoxy, —C₂₋₁₀alkenyl, —C₂₋₁₀alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is optionally independently substituted with one or more substituents selected from deuterium, halogen, —C₁₋₆ alkyl, haloC₁₋₆alkyl, haloC₁₋₆alkoxy, —C₂₋₆alkenyl, —C₂₋₆alkynyl, —CN, —NO₂, —N₃, oxo, —NR_{c}R_{d}, —OR_{c}, —SR_{c}, —S(═O)R_{c}, —S(═O)₂R_{c}, —C(═O)R_{c}, —C(═O)OR_{c}, —OC(═O)R_{c}, —C(═O)NR_{c}R_{d}, —NR_{c}C(═O)R_{d}, —OC(═O)OR_{c}, —NR_{c}C(═O)OR_{d}, —OC(═O)NR_{c}R_{d}, —NR_{c}C(═O)NR_{c}R_{d}, —S(═O)OR_{c}, —OS(═O)R_{c}, —S(═O)NR_{c}R_{d}, —NR_{c}S(═O)R_{d}, —S(═O)₂OR_{c}, —OS(═O)₂R_{c}, —S(═O)₂NR_{c}R_{d}, —NR_{c}S(═O)₂R_{d}, —OS(═O)₂OR_{c}, —NR_{c}S(═O)₂OR_{d}, —OS(═O)₂NR_{c}, —NR_{c}S(═O)₂NR_{c}R_{d}, —PR_{c}R_{d}, —P(═O)R_{c}R_{d}, 3-6 membered cycloalkyl, 3-6 membered cycloalkenyl, 3-6 membered cycloalkynyl, 3-10 membered heterocyclyl, 6-10 membered aryl or 5-10 membered heteroaryl;
Optionally, R₈₁ and R₈₂ together with the nitrogen atom to which they are both attached form a 3-10 membered heterocyclic ring or a 5-10 membered heteroaryl ring, wherein, said 3-10 membered heterocyclic ring or 5-10 membered heteroaryl ring is optionally independently substituted with one or more R₁₆;
Rₐ, R_{b}, R_{c} or R_{d} at each occurrence is independently selected from hydrogen, deuterium, —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally independently substituted with one or more R₁₆;
Optionally, (Rₐ and R_{b}) or (R_{c} and R_{d}) together the atom to which they are both attached form a 3-6 membered heterocyclic ring, wherein said 3-6 membered heterocyclic ring is independently optionally substituted with one or more R₁₆;
R₁₆ at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆ alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-6 membered cycloalkyl, 3-6 membered cycloalkenyl, 3-6 membered cycloalkynyl, 3-6 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-6 membered cycloalkyl, 3-6 membered cycloalkenyl, 3-6 membered cycloalkynyl, 3-6 membered heterocyclyl, 6-10 membered aryl or 5-10 membered heteroaryl is optionally substituted with one or more substituents selected from deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃ alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-6 membered cycloalkyl, 3-6 membered cycloalkenyl, 3-6 membered cycloalkynyl, 3-6 membered heterocyclyl, 6 membered aryl or 5-6 membered heteroaryl;
Each of (heterocyclyl and heteroaryl) at each occurrence is independently contain 1, 2, 3 or 4 heteroatoms selected from N, O, S, S(═O) or S(═O)₂.

49. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, X₁ is N.

50. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, X₂ is O, S, NH or NCH₃.

51. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, X₃ is selected from CR₇₁R₇₂ or O.

52. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, each of (R₇₁ and R₇₂) is independently selected from hydrogen, deuterium, —CH₃, —CH₂CH₃, —CH₂CH₂CH₃, —CH(CH₃)₂, —F, —Cl, —CN, —CH₂OH, —OH, —OCH₃, —OCH₂CH₃, —CF₃, —CHF₂, —S—CH₃, —S—CHF₂, —S—CF₃,

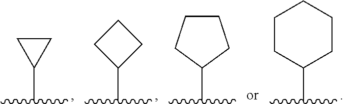

53. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, n₁, n₄, and n₅ are 1 respectively, or n₁ and n₄ are 1 respectively and n₅ is 0.

54. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, n₂ and n₃ are 1 respectively, or n₃ is 0 and n₂ is 2, or n₃ is 0 and n₂ is 1, or n₃ is 1 and n₂ is 0, or n₃ is 0 and n₂ is 0.

55. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, each of R_{S0} at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, oxo, —NH₂, —NH(C₁₋₆ alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)H, —C(═O)(C₁₋₆alkyl), —C(═O)OH, —C(═O)(OC₁₋₆alkyl), —OC(═O)(C₁₋₆alkyl), —C(═O)NH₂, —NO₂, —C(═O)NH(C₁₋₆alkyl), —C(═O)N(C₁₋₆alkyl)₂, —NHC(═O)(C₁₋₆alkyl), —N(C₁₋₆ alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₁₋₆alkynyl, 3-10 membered cycloalkyl, 3-10 membered heterocyclyl, 6-10 membered aryl or 5-10 membered heteroaryl is independently optionally substituted with 1, 2 or 3 R₁ₐ;
Optionally, two R_{S0} together with the carbon atom to which they are both attached form 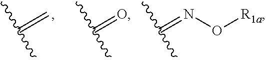
a 3-10 membered carbocyclic ring or a 3-10 heterocyclic ring; wherein, said 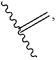
3-10 membered carbocyclic ring or 3-10 heterocyclic ring is optionally substituted with one or more R₁ₐ;
Optionally, two adjacent R_{S0} together with the carbon atoms to which they are respectively attached form a 3-10 membered carbocyclic ring or 3-10 membered heterocyclic ring, wherein, each of rings is independently optionally substituted with one or more R₁ₐ;
Optionally, two nonadjacent R_{S0} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S=O or S(═O)₂; the hydrogen on the each of carbon atoms or N atoms is independently unsubstituted or substituted with R₁ₐ;
Each of R₁ₐ is independently selected from deuterium, halogen, —C₁₋₆alkyl, haloC₁₋₆alkyl, —CN, oxo, —OH, —NH₂, —NH(C₁₋₆alkyl), —N(C₁₋₆alkyl)₂, —OC₁₋₆alkyl, or —C₁₋₆alkyl substituted with 1, 2 or 3 substituents selected form deuterium, halogen, haloC₁₋₆alkyl, —CN, —OH, —NH₂, —NH(C₁₋₆alkyl), —N(C₁₋₆alkyl)₂, —OC₁₋₆ alkyl or cyclopropyl;
m is independently selected from 0, 1, 2 or 3.

56. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, the compound is selected from the formulas in Table 1 as shown in the description.

57. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, the moiety of —Y—R₂ or 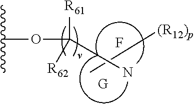
is selected from

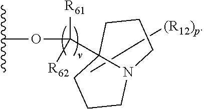

58. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, the moiety of 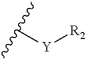
is selected from any one moiety in the Table 2 as shown in the description.

59. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, the moiety of 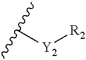
is selected from

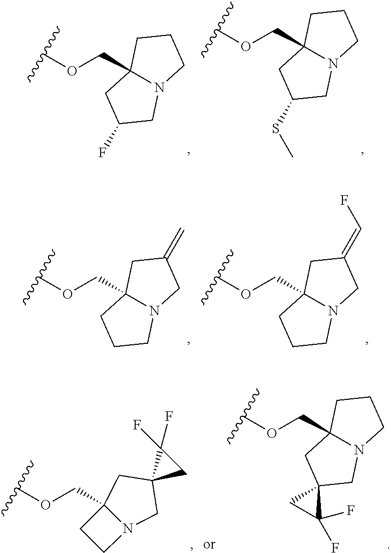

60. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, the compound is selected from the formulas in Table 3 as shown in the description.

61. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, R₄ is selected from any one moiety in the Table 4 as shown in the description,
Wherein said R₄ is independently optionally substituted with 1, 2, 3, 4, 5 or 6 R₄₁;
Each of R₄₁ is independently selected from deuterium, —F, —Cl, —C₁₋₃alkyl, haloC₁₋₃alkyl, haloC₁₋₃alkoxy, —C₂₋₃alkenyl, —C₂₋₃alkynyl, —CN, —NH₂, —NH(C₁₋₃alkyl), —N(C₁₋₃alkyl)₂, —OH, —O(C₁₋₃alkyl), —SH, —S(C₁₋₃alkyl), 3-6 membered cycloalkyl or 3-6 membered heterocyclyl or R₄ₐ, wherein said —C₁₋₃alkyl, haloC₁₋₃alkyl, haloC₁₋₃alkoxy, —C₂₋₃alkenyl, —C₂₋₆alkynyl, 3-6 membered cycloalkyl or 3-6 membered heterocyclyl is independently optionally substituted with 1, 2 or 3 substituents selected from —F; —C₁₋₃ alkyl; haloC₁₋₃alkyl; —CN; —OH; —NH₂; —NH(C₁₋₃alkyl); —NH(C₁₋₃alkyl)₂; —OC₁₋₃alkyl; or —C₁₋₃alkyl substituted with 1, 2 or 3 substituents selected from —F, haloC₁₋₃alkyl, —CN, —OH, —NH₂, —NH(C₁₋₃alkyl), —NH(C₁₋₃alkyl)₂ or —OC₁₋₃alkyl.

62. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 61, wherein, R₄ is selected from any one moiety in the Table 5 as shown in the description.

63. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, R₄ is

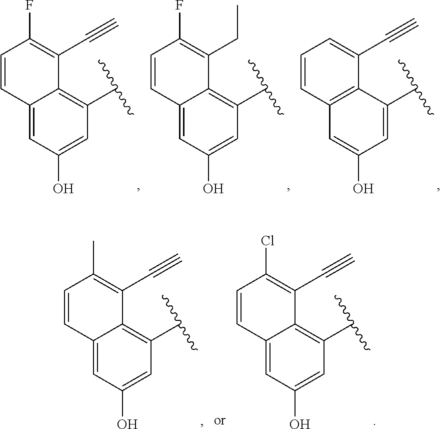

64. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, the compound is selected from the formulas in Table 6 as shown in the description.

65. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, RR₅ is —F.

66. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of claim 48, wherein, the compound is selected from any one in Table 8 as shown in the description.

67. A pharmaceutical composition, comprising a therapeutically effective amount of the compound of formula (I), the stereoisomer thereof, the pharmaceutically acceptable salt thereof, the pharmaceutically acceptable salt of the stereoisomer thereof, the prodrug thereof, the deuterated molecule thereof or the conjugated form thereof of claim 48, and a pharmaceutically acceptable excipient.
